(19) United States
(12) Patent Application Publication
(10) Pub. No.: US 2023/0346538 A1
(43) Pub. Date: Nov. 2, 2023

(54) PROVIDING MEDICAL DEVICES WITH SENSING FUNCTIONALITY

(71) Applicant: CANARY MEDICAL SWITZERLAND AG, Baar (CH)

(72) Inventors: Mark A. Adler, Carlsbad, CA (US); Jeffrey M. Gross, Carlsbad, CA (US); William L. Hunter, Vancouver (CA); Kimberly Salant, Asbury, NJ (US); Peter J. Schiller, Shakopee, MN (US); David Zarbatany, Laguna Niguel, CA (US)

(21) Appl. No.: 18/030,047

(22) PCT Filed: Oct. 7, 2021
(86) PCT No.: PCT/US2021/053972

§ 371 Date: Apr. 3, 2023

Related U.S. Application Data
(60) Provisional application No. 63/088,824, filed on Oct. 7, 2020.

(51) Int. Cl. A61F 2/07; A61B 5/00; A61B 5/05; A61F 2/966; H01Q 1/36; H01Q 1/27

(52) U.S. Cl. CPC A61F 2/07; A61B 5/0031; A61B 5/05; A61B 5/6862; A61F 2/966; H01Q 1/36; H01Q 1/273; A61F 2250/0002

(57) ABSTRACT

Auxiliary components for medical devices, and more specifically, sensing constructs that may be added to a medical device such as an implantable medical device to provide the medical device with sensing functionality. The auxiliary component is not a part of the medical device, but rather is associated with an existing medical device in a secure manner, and provides information about the medical device and/or the environment around the medical device when the device is implanted in a patient, and then transmits that information to a location outside of the patient for evaluation. Delivery systems for delivering the auxiliary components.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### FIELD

The present disclosure relates generally to auxiliary components for medical devices and systems for delivering auxiliary components, and more specifically, the disclosure relates to sensing constructs that may be added to a medical device such as an implantable medical device to provide the medical device with sensing functionality.

### BACKGROUND

Treatment modalities for people having an injury or degenerative condition may frequently involve implantation of a medical device. For example, some people develop an aneurysm, which can be life-threatening, and are treated by implantation of an endovascular graft or endovascular stent graft in the region of the aneurysm sac. Commonly, aneurysms are a bulging and weakness in the wall of the aorta, but they can occur anywhere in the human arterial vascular system. This bulging creates a widening in the diameter of the aorta, which creates what is known as an aneurysm sac. Most aortic aneurysms occur in the abdominal aorta (abdominal aortic aneurysms or AAA), but they can also occur in the thoracic aorta (thoracic aortic aneurysms or TAA) or in both the thoracic and abdominal segments of the aorta. Other examples of aneurysms include a femoral aneurysm, which is a bulging and weakness in the wall of the femoral artery (located in the thigh), an iliac aneurysm which occurs upon weakness in the wall of the iliac artery (a group of arteries located in the pelvis), a popliteal aneurysm which occurs when there is weakness in the wall of the popliteal artery which supplies blood to the knee joint, thigh and calf, a subclavian aneurysm which is weakness or bulging in the wall of the subclavian artery (located below the collarbone), a supra-renal aneurysm of the aorta located above the kidneys, and a visceral aneurysm which occurs within abdominal cavity arteries and includes the celiac artery, the superior mesenteric artery, the inferior mesenteric artery, the hepatic artery, the splenic artery and the renal arteries.

The endovascular graft or endovascular stent graft is a tubular structure that is inserted above and below the aneurysm sac and thus extends through the aneurysm sac. The graft or stent graft captures the blood that would ordinarily flow into the aneurysm sac, and retains that blood within the graft or stent graft. The consequence is that the pressure on the wall of the blood vessel that surrounds the aneurysm sac is reduced. This reduced pressure, in turn, reduces the likelihood that the wall surrounding the aneurysm sac will burst.

Unfortunately, there is no easy way for a treating physician to completely monitor a conventional graft or conventional stent graft after it has been implanted into the patient, nor to completely monitor the region around the implanted device, e.g., monitor the integrity of the aneurysm sac. The present disclosure addresses this need.

### SUMMARY

Certain aspects of the present disclosure are directed toward systems and methods for delivering any of the implantable devices described herein (also referred to as sensing attachments, sensing constructs, auxiliary components, or scaffolds) to a blood vessel, for example an aneurysmal sac of an abdominal aorta. Challenges may arise from advancing the delivery system through the vasculature and deploying an implantable device having an elongated shape. For example, the implantable devices described herein may have a length of at least about 12.0 inches, at least about 14.0 inches, or at least about 18.0 inches when loaded into the delivery system. Further, because the implantable device may provide sensing, communication, powering, and/or charging functions, the implantable device may need to be properly oriented and positioned within the implantation site. Thus, among other advantages, the delivery systems described herein enable the release of the implantable device from the delivery system and orient the implantable device at the implantation site.

The delivery system may include a handle, an outer sheath, a pusher shaft slidably disposed within a lumen of the outer sheath, and/or a release shaft slidably disposed within a lumen of the pusher shaft. The outer sheath may be actively or passively deflected in at least one direction, for example in only one direction or all directions. The release shaft may be capable of releasing a distal tip from the outer sheath. The distal tip may be a distal portion of the release shaft or a distal portion of the implantable device.

The pusher shaft may be capable of advancing the implantable device and/or re-sheathing the implantable device. A distal portion of the pusher shaft may be shaped to interface with a proximal portion of the implantable device. For example, the distal portion of the pusher shaft may be shaped to form a press-fit with a lumen of the implantable device. The pusher shaft may be rotated to apply torque to the implantable device. For example, the pusher shaft may apply torque when at least a portion or substantially the entire implantable device is still disposed within the outer sheath. This may properly orient the implantable device prior to partial or full deployment. To release the implantable device from the pusher shaft, the pusher shaft may apply torque to the implantable device, for example after the proximal portion of the implantable device has been advanced distally of a distal end of the outer sheath. In other implementations, the implantable device may be released from the pusher shaft as soon as the proximal portion of the implantable device has been advanced distally of the distal end of the outer shaft.

The release shaft may include an enlarged distal end. The enlarged distal end may form an atraumatic tip of the delivery system. Alternatively, the enlarged distal end may be disposed within a lumen of the implantable device. The enlarged distal end may act on the implantable device to release a distal portion of the implantable device from the outer sheath. The release shaft may include a guidewire lumen. The guidewire lumen may extend through the distal tip of the delivery system whether the distal tip is the enlarged distal end of the release shaft or the distal portion of the implantable device. The guidewire lumen may be able to be moved freely or adjusted/telescoped, independently of the other shafts enabling the distal portion to be pulled proximally to support the delivery of the implant and antenna systems.

The delivery system may include one or more locking mechanisms to prevent relative movement between various components of the delivery system during transport, for example between the outer sheath, pusher shaft, and/or release shaft having a guidewire lumen. The locking mechanisms may include seals to prevent the backflow of fluid. For example, the delivery system may include a first locking mechanism to prevent movement of the pusher shaft relative to the outer sheath and/or a second locking mechanism to prevent movement of the release shaft relative to the pusher shaft.

Certain aspects of the present disclosure are directed toward a combination of any of implantable devices and any of the delivery systems described herein, for example the delivery system described above. At least a portion or the entirety of the implantable device may be carried within the outer sheath. A distal portion of the implantable device may project from a distal end of the outer sheath of the delivery system. The distal portion of the implantable device may form the distal tip of the system. Alternatively, the distal portion of the implantable device may be positioned between the distal tip of the delivery system and the distal end of the outer sheath. For example, the distal portion of the release shaft may form the distal tip of the system. The release shaft may be slidably disposed within a lumen of the implantable device.

When the implantable device is loaded in the outer sheath, the distal portion of the implantable device may be coupled to the distal end of the outer sheath, for example by a press-fit. In other implementations, the distal portion of the implantable device may abut the distal end of the outer sheath, but not coupled to the distal end of the outer sheath. The one or more locking mechanisms may be used to maintain a position of the pusher shaft relative to the outer sheath to stabilize a position of the implantable device relative to the delivery system during transport and navigation through the vasculature.

To release the distal portion of the implantable device from the outer sheath, the release shaft may push on an internal feature of the implantable device. In other implementations, the pusher shaft may act on a proximal portion of the implantable device to release the distal portion of the implantable device. The pusher shaft may be releasably coupled to the proximal portion of the implantable device, for example using a press-fit.

The distal portion of the implantable device may transition between a first configuration during transport and a second configuration when deployed. In the first configuration, the distal portion may be compressed or rolled into a cylindrical, conical or other three-dimensional shape. In the second configuration, the distal portion may be expanded or unrolled into a substantially flattened shape compared to the first configuration. In some implementations, the distal portion of the implantable device may include an antenna, power or recharging capabilities, or other circuitry to enable the sensing and communication functions of the implantable device.

Certain aspects of the disclosure are directed toward a handle for controlling one or more features of the delivery systems described herein. The handle may include a body and one or more user-actuatable controls. For example, the handle may include a first user-actuatable control capable of deflecting an outer sheath in at least one direction. The handle may include a second user-actuatable control capable of providing rotation or torque control for a pusher shaft. The handle may include a third user-actuatable control capable of advancing and/or retracting the pusher shaft. The handle may include a fourth user-actuatable control capable of advancing a release shaft.

The first user-actuatable control may actuate a pulley or one or more worm gears to deflect a distal portion of the outer sheath. A position of one or more internal components of the deflection control may be visible in a window of the handle body corresponding to an amount of deflection of the outer sheath.

Certain aspects of the present disclosure are directed toward method of delivering any of the implantable devices described herein. The method may include advancing a delivery system over a guidewire. The delivery system may include an outer sheath carrying the implantable device. The implantable device may be disposed radially between a release shaft and an outer sheath of the delivery system. Substantially the entire implantable device may be disposed distally of the pusher shaft. Each of the delivery system and the implantable device may include a lumen for the guidewire. The method may include deflecting a distal portion of the outer sheath to or within the implantation site to properly orient the implantable device.

The method may include releasing a distal tip of the implantable device from the outer sheath using a release shaft or the pusher shaft. In some implementations, the distal tip may be a distal portion of the implantable device or a distal portion of the release shaft. The release shaft may be advanced through the lumen of the implantable device. The release shaft may act on an internal feature of the implantable device to release or advance the distal portion of the implantable device. In other implementations, the distal tip of the implantable device may form a loose fit with the distal end of the outer sheath. Advancing the pusher shaft against the proximal end of the implantable device may release the distal end of the implantable device from the outer sheath.

The method may include advancing and/or re-sheathing the implantable device using a pusher shaft. The method may include releasing a proximal portion of the implantable device from the pusher shaft. The method may include applying a torque to the implantable device using the pusher shaft. For example, a torque may be applied when the implantable device is at least partially loaded or substantially entirely loaded within the outer sheath and/or to release the implantable device from the pusher shaft.

The method may include deploying a second implantable device adjacent the implantable device. The second implantable device may or may not be coupled or in contact with the implantable device. The second implantable device may be deployed within an interior space of the implantable device or surround the implantable device. The second implantable device may include a treatment device such as a graft. The second implantable device may be deployed prior to deploying the implantable device, simultaneously with the implantable device, after partially deploying the implantable device, or after fully deploying the implantable device. The two implantable devices may be deployed during the same procedure or different procedures.

Certain aspects of the present disclosure are directed toward delivering an implantable device to an aneurysmal sac in an abdominal aorta. The method may include advancing a first delivery system carrying the implantable device through a contralateral iliac artery. The method may include deflecting a distal portion of the first delivery system to or within the aneurysmal sac. The method may include partially deploying the implantable device from the first delivery system in the aneurysmal sac. The implantable device may form a coil as the implantable device is released from the first delivery system. Partial deployment may include deploying less than or equal to two turns or less than or equal to one turn of the coiled implantable device. Prior to fully deploying the implantable device from the first delivery system, the method may include re-sheathing the implantable device if the implantable device is not properly positioned or oriented within the aneurysmal sac.

The method may include advancing a second delivery system carrying a stent graft through an ipsilateral iliac artery. After at least partially or fully deploying the implantable device, the method may include deploying the stent graft within an interior space defined by the coil or around an exterior of the coil. The method may include releasing the implantable device from the first delivery system, which may occur prior to or following the deployment of the stent graft. When released, a distal portion of the implantable device may be positioned in a posterior region of the aneurysmal sac.

Certain aspects of the present disclosure are directed toward an implantable device that may be delivered percutaneously using any of the delivery systems described herein. The implantable device may be an implantable sensing construct including a sensor and a body. The implantable device may include an antenna providing any of the communications functions described herein. The method may include any of the powering or charging functions described herein. The body of the implantable device may include a first configuration having a substantially linear shape for transport in a delivery system and a second configuration having a coiled shape when released from the delivery system.

In the coiled configuration, the body may be able to withstand a compression load sufficient to maintain an internal diameter of the coiled implantable device in the aneurysmal sac. For example, the body may withstand a compressive force of at least about 5.0 N and/or less than or equal to about 25.0 N, for example up to 5.0 N, up to 20.0 N, or up to 25.0 N. The body may be able to withstand a compression force from about 1.0 N to about 25.0 N, for example, from about 1.0 N to about 5.0 N, from about 5.0 N to about 20.0 N, from about 20.0 N to about 25.0 N, or ranges in between.

The body may be able to withstand a tension force needed to pull the body straight within the delivery system. For example, the body may be able to withstand a tension force of at least about 5.0 N and/or less than or equal to about 105.0 N, for example up to 8.0 N, up to 15.0 N, up to 25.0 N, or up to 105.0 N. The body may be able to withstand a tension force from about 5.0 N to about 15.0 N, from about 15.0 N to about 25.0 N, from about 25.0 N to about 105.0 N, or ranges in between.

An internal diameter of the body in the coil configuration may be less than or equal to about 50.0 mm, or less than or equal to about 25.0 mm. An outer diameter of the body in the coil configuration may be less than or equal to about 50.0 mm.

Certain aspects of the present disclosure are directed toward an implantable system for use with a stent graft. The system can include a helix antenna supported by a non-conductive substrate. The system can include a communications and processing circuitry electrically connected to the helix antenna via an antenna feed. The communications and processing circuitry can be supported by a substrate including a ground plane to which the helix antenna is electrically connected. The communications and processing circuitry can include at least one sensor.

The system can include a body configured to be attached to or positioned adjacent to the stent graft. The body can be made at least partially of conductive material. The helix antenna can be further electrically connected to the body such that the body provides an additional ground for the helix antenna.

The helix antenna can be configured to transmit and receive in a Bluetooth frequency band. The range of the helix antenna in the Bluetooth frequency band is about 1 foot or more. The range of the helix antenna in the Bluetooth frequency band can be between about 1 foot and 2 feet.

The communications and processing circuitry can include matching circuitry electrically connected to the helix antenna. The matching circuitry can include a series capacitor and a shunt capacitor. The matching circuitry can further include a low-pass filter.

The non-conductive substrate can provide structural support for the helix antenna. The helix antenna can be wound on the non-conductive substrate. The non-conductive substrate can include a polymer. The polymer can include polytetrafluoroethylene (PTFE). The stent graft can be an abdominal aortic aneurysm (AAA) stent graft.

Certain aspects of the present disclosure are directed toward a method of radio frequency (RF) testing an antenna of an implantable system. The method can include determining one or more RF properties of the antenna of the implantable system positioned in a first container at least partially filled with a first composition configured to simulate electromagnetic properties of blood. The first container can be positioned in a second container at least partially filed with a second composition configured to simulate electromagnetic properties of one or more tissues.

One or more tissues can include at least two of bone, muscle, fat, and skin. Electromagnetic properties of at least two of bone, muscle, fat, and skin can be averaged to create the second composition. Electromagnetic properties of the one or more tissues can include relative permittivity and conductivity.

First composition can include sodium chloride (NaCl), diacetin, and distilled water. Second composition can include diacetin and distilled water.

The antenna can include a helix antenna. The helix antenna can be supported by a non-conductive substrate. The implantable system can be configured to be used with a stent graft. The stent graft can be an abdominal aortic aneurysm (AAA) stent graft.

Certain aspects of the present disclosure are directed toward an implantable system for use with a stent graft. The system can include a first antenna including a straight conductor and a helical conductor. The straight conductor can be electrically connected to the helical conductor. The system can include a communications and processing circuitry electrically connected to the first antenna via an antenna feed. The communications and processing circuitry can be supported by a substrate comprising a ground plane to which the first antenna is electrically connected. The communications and processing circuitry can include at least one sensor.

The system can include a body configured to be attached to or positioned adjacent to the stent graft. The body can be made at least partially of conductive material. The first antenna can be supported by the body. The first antenna can be further electrically connected to the body such that the body provides an additional ground for the first antenna. The straight conductor can be a monopole antenna.

The first antenna can be a dual-band antenna that transmits and receives in first and second frequency bands. The first antenna can resonate at center frequencies of the first and second frequency bands. The first frequency band can be medical device radiocommunications service (MICS) band and the second frequency band can be industrial, scientific, and medical (ISM) band. A range in the first frequency band can be at least about 20 feet, and a range in the second frequency band can be at least about 15 feet. A range in the first frequency band can be about 1 foot or more, and a range in the second frequency band can be about 1 foot or more.

The communications and processing circuitry can be configured to transition from a first power state to a second power state in which more power is consumed responsive to the first antenna receiving a command in the second frequency band. The first power state can be a sleep state, and the second power state can be an operational state in which the communications and processing circuitry can be configured to at least one of transmit or receive data. Data can include data sensed by the at least one sensor, and the communications and processing circuitry can be configured to cause the first antenna to transmit the data in the first frequency band. The communications and processing circuitry can be configured to transmit data sensed by the at least one sensor in the second power state and not in the first power state.

The communications and processing circuitry can include a matching circuitry electrically connected to the first antenna. The first antenna can be configured to at least one of receive or transmit in first and second frequency bands, the second frequency band associated with one or more higher frequencies than the first frequency band. The matching circuitry can include a first matching circuitry for signals in the first frequency band and a second matching circuitry for signals in the second frequency band. The first matching circuitry can include a band-stop filter configured to remove one or more signal components in second frequency band. The first matching circuitry can include a step-up impedance low pass filter, and the second matching circuitry can include a setup-up impedance high pass filter. The second matching circuitry may not include a band-stop filter configured to remove one or more signal components in the first frequency band.

The system can include a rechargeable power source and a second antenna configured to receive power for recharging the rechargeable power source. The second antenna can include a coil configured to be inductively coupled with a coil of an external power transfer device.

The system can include a body configured to be attached to or positioned adjacent to the stent graft. The first antenna can be supported by the body at a first end of the body, and the second antenna can be supported by the body at a second end of the body opposite the first end.

The system can include a second antenna. The system can include a body configured to be attached to or positioned adjacent to the stent graft. The first antenna can be supported by the body at a first end of the body, and the second antenna can be supported by the body at a second end of the body opposite the first end.

The length of the first antenna can be at most about 40 mm. The width of the first antenna can be at most about 5 mm. Spacing between turns of the helical conductor can be about 1.7 mm.

The helical conductor can be wound around the straight conductor. The helical conductor can be electrically insulated from the straight conductor in a region where the helical conductor is wound around the straight conductor. The straight conductor and the helical conductor can be electrically connected to the antenna feed. The stent graft can be an abdominal aortic aneurysm (AAA) stent graft.

Certain aspects of the present disclosure are directed toward an implantable system for use with a stent graft. The system can include a first antenna including a loop. The system can include communications and processing circuitry electrically connected to the first antenna via an antenna feed. The communications and processing circuitry can be supported by a substrate comprising a ground plane to which the first antenna is electrically connected. The communications and processing circuitry can include at least one sensor. The system can include matching circuitry, which can be part of the communications and processing circuitry. The matching circuitry can be electrically connected to the first antenna. The matching circuitry can include a plurality of capacitors configured to match impedance of the first antenna in a first frequency band.

The matching circuitry further can include a plurality of inductors configured to match impedance of the first antenna in a second frequency band. The second frequency band can be associated with one or more higher frequencies than the first frequency band.

The system can include a body configured to be attached to or positioned adjacent to the stent graft. The body can be made at least partially of conductive material. The first antenna can be supported by the body. The first antenna can be further electrically connected to the body such that the body provides an additional ground for the first antenna.

The first antenna can be a dual-band antenna that transmits and receives in the first frequency band and in a second frequency band. The first antenna can resonate in the second frequency band. The second frequency band can be associated with one or more higher frequencies than the first frequency band. The first frequency band can include medical device radiocommunications service (MICS) band, and the second frequency band can include industrial, scientific, and medical (ISU) band. A range in the first frequency band can be at least about 20 feet, and a range in the second frequency band can be at least about 7 feet. A range in the first frequency band can be about 1 foot or more, and a range in the second frequency band can be about 1 foot or more.

The communications and processing circuitry can be configured to transition from a first power state to a second power state in which more power is consumed responsive to the first antenna receiving a command in the second frequency band. The first power state can be a sleep state, and the second power state can be an operational state in which the communications and processing circuitry can be configured to at least one of transmit or receive data. Data can include data sensed by the at least one sensor, and the communications and processing circuitry can be configured to cause the first antenna to transmit the data in the first frequency band. The communications and processing circuitry can be configured to transmit data sensed by the at least one sensor in the second power state and not in the first power state.

The matching circuitry can include a band-stop filter configured to remove one or more signal components in the second frequency band. The matching circuitry may not include a band-stop filter configured to remove one or more signal components in the first frequency band.

The system can include a rechargeable power source and a second antenna configured to receive power for recharging the rechargeable power source. The second antenna can include a coil configured to be inductively coupled with a coil of an external power transfer device.

The system can include a body configured to be attached to or positioned adjacent to the stent graft. The first antenna can be supported by the body at a first end of the body, and the second antenna can be supported by the body at a second end of the body opposite the first end.

The system can include a second antenna. The system can include a body configured to be attached to or positioned adjacent to the stent graft. The first antenna can be supported by the body at a first end of the body, and the second antenna can be supported by the body at a second end of the body opposite the first end.

The diameter of the loop can be at most about 40 mm. The width of the first antenna can be at most about 5 mm. The stent graft can be an abdominal aortic aneurysm (AAA) stent graft.

Certain aspects of the disclosure are directed toward a delivery system for delivering an implantable device. The delivery system can include a handle enclosure and a handle driver having a collar rotatably coupled to the handle enclosure. For example, the handle enclosure may include a groove configured to capture the collar of the handle driver and limit an axial position of the handle driver, but in other configurations, the handle driver may be axially movable relative to the handle enclosure. The delivery system may include first lead screw and a second lead screw. An inner surface of the collar can include a threaded pattern configured to interface with the first lead screw and/or the second lead screw. The threaded pattern may include a non-continuous threaded pattern, for example a pattern of diamond-shaped recesses. The first lead screw may be disposed at least partially within the handle driver. The first lead screw may be threaded in a first direction. The second lead screw may be disposed at least partially within the handle enclosure. The second lead screw may be axially offset from the first lead screw. The second lead screw may be threaded in a second direction opposite from the first direction. Rotation of the handle driver in a first direction advances the implantable device. Rotation of the handle driver in the opposite direction retracts the implantable device.

A distal portion of the first lead screw may abut a proximal portion of the second lead screw when the implantable device is loaded in the delivery system, for example, the distal portion of the first lead screw may overlap the proximal portion of the second lead screw or the first lead screw may be in end-to-end contact with the second lead screw. Rotation of handle driver may drive the first lead screw in a first direction and drive the second lead screw in a second direction opposite the first direction. Rotation of the handle driver may drive the first lead screw and the second lead screw the same distance, but in other configurations, may drive the first and second lead screws different distances. The first and lead screws may be partial body screws, for example half body screws. Each screw may extend less than 360 degrees around a longitudinal axis of the screw. The first lead screw may be circumferentially offset from the second lead screw.

The delivery system an indicator to provide an indication of a location of the implantable device relative to the outer sheath. The indicator may be movable along a slot in the handle enclosure. The indicator may be fixed to one of the lead screws.

Certain aspects of the disclosure are related to a method of delivering an implantable device to a patient using any of the delivery systems described herein. The delivery system may include a handle and an outer sheath carrying the implantable device. An intermediate tube may extend through the outer sheath. The intermediate tube may be coupled to the implantable device. The outer sheath may include a tension wire to deflect the distal portion of the outer sheath. The handle may include a first actuator. The first actuator, for example a collar as described herein, may be translatable and/or rotatable to control a distal portion of the outer sheath. The handle may include a second actuator to deploy or retract the implantable device relative to the outer sheath. The second actuator, for example a handle driver as described herein, may be translatable and/or rotatable.

The method may include advancing a delivery system to a target location. The method may include actuating a first actuator on the handle to deflect a distal portion of the outer sheath to a deflected configuration. The method may include actuating the first actuator to lock the distal portion of the outer sheath in the deflected configuration. Actuating the first actuator may rotate a cam to lock the distal portion of the outer sheath in the deflected configuration. The method may include actuating a second actuator in a first direction to advance an intermediate tube relative to the outer sheath. Actuating the second actuator in a second direction, opposite from the first direction, may retract the intermediate tube. The method may include withdrawing an inner tube to release the implantable device from the intermediate tube. A release pin may be removed from the handle to enable withdrawal of the inner tube. This step may occur after one or more turns of the implantable device are deployed from the outer sheath. The method may include rotating the inner tube to release the inner tube from the implantable device. Movement of the implantable device may cause an indicator to travel along a slot in the handle of the delivery system.

Certain aspects of the disclosure are related to delivery system for delivering an implantable device. The delivery system may include a handle having a handle enclosure, a first actuator movable relative to the handle enclosure, and a second actuator movable relative to the handle enclosure. The delivery system may include an outer sheath extending from the handle, an intermediate tube extending through the outer sheath, and/or an inner tube extending through the intermediate tube. The intermediate tube may be configured to engage the implantable device. The inner tube may be configured to maintain the intermediate tube in engagement with the implantable device when the inner tube extends through the implantable device. The delivery system may include an indicator visible through a slot in the handle enclosure. The indicator indicative of a location of the implantable device relative to the outer sheath.

The first user actuator may configured to deflect a distal portion of the outer sheath from an undeflected configuration to a deflected configuration and/or lock the distal portion of the outer sheath in the undeflected configuration or the deflected configuration. For example, actuation of the first user actuator may tension a wire to deflect the distal portion of the outer sheath from the undeflected configuration to the deflected configuration. Actuation of the first actuator in a different manner may rotate a cam to lock the distal portion of the outer sheath in the undeflected configuration or the deflected configuration.

The second actuator may be configured to advance the intermediate tube relative to the outer sheath. For example, actuation of the second actuator in a first direction may advance the intermediate tube, while actuation of the second actuator in the opposite direction may retract the intermediate tube.

The delivery system a release pin at a proximal end of the inner tube to seal the handle. Rotation of the release pin may release a distal portion of the implantable device. The release pin may be removable from the inner tube.

The delivery system may include a disconnect assembly at a distal end of the intermediate tube to releasably engage the implantable device. The disconnect assembly may include one or more deflectable tabs configured to engage the implantable device when the inner tube extends through the inner component.

Certain aspects of the disclosure are related to implantable sensing construct configured to be percutaneously implanted in an aneurysmal sac. The implantable sensing construct may include a sensor and a tubular body. The tubular body may include a first configuration and a second configuration. The tubular body may include a plurality of cutouts in a circumferential direction, each of the plurality of cutouts comprising a first end, a second end, and in intermediate portion therebetween. Each of the plurality of cutouts may include a generally dogbone shape with a width of each of the first ends and the second ends of the plurality of cutouts being greater than a width of the intermediate portions. The plurality of cutouts may be equally spaced apart along a length of the tubular body. In the first configuration, the body may include a substantially linear shape for transport in a delivery system. In the second configuration, the body may include a coiled shape when released from the delivery system.

The tubular body may include a plurality of tubular segments. The plurality of tubular segments may be spaced apart from each other and interconnected by a spine. Each of the plurality of tubular segments having one or more of the plurality of cutouts. When the tubular body is laid flat as a flattened body with the spine forming opposite lateral edges, the flattened body may form a non-rectangular shape. The lateral edges may form an oblique angle relative to an end of the flattened body. The plurality of tubular segments may include a first tubular segment at a first end of the tubular body, a second tubular segment at a second end of the tubular body, and at least one tubular segment between the first tubular segment and the second tubular segment. The at least one tubular segment may be shorter than the first tubular segment and the second tubular segment.

Disclosed are methods of using or operating the system of any of the preceding paragraphs and/or any of the systems disclosed herein.

Any feature, structure, or step disclosed herein can be replaced with or combined with any other feature, structure, or step disclosed herein, or omitted. Further, for purposes of summarizing the disclosure, certain aspects, advantages, and features of the embodiments have been described herein. It is to be understood that not necessarily any or all such advantages are achieved in accordance with any particular embodiment disclosed herein. No individual aspects of this disclosure are essential or indispensable.

### BRIEF DESCRIPTION OF THE DRAWINGS

Example features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 is a front perspective view showing an example body of a sensing attachment, the body in the form of a filament and the shape of a ring with undulations.

FIG. 2A is a front view and FIG. 2B is a top right perspective view, each showing an example body of a sensing attachment, the body in the form of a plurality of adjacent rings. FIG. 2A shows a portion of the body. FIG. 2B shows a portion of the body in the shape of a clamp, also known as a cuff bracelet shape.

FIGS. 3A, 3B and 3C are each front views showing example bodies of sensing attachments, each in the form of a clip. FIG. 3A shows a filament in the shape of a classic paper clip, FIG. 3B shows a filament in the shape of a paper clip, and FIG. 3C shows a sheet that has been cut into the shape of a paper clip.

FIGS. 4A and 4B are each front right perspective views showing example bodies of sensing attachments, each in the form of a clamp. FIG. 4A shows a sheet in the shape of a clamp, while FIG. 4B shows a filament in the shape of a clamp, where this clamp shape may also be referred to as a cuff bracelet shape.

FIG. 5A is a perspective view showing an example body of a sensing attachment, the body in the form of a filament and the shape of a spring.

FIG. 5B shows a cross-sectional view of the filament of FIG. 5A, and in particular shows the circular cross-section of the filament of FIG. 5A.

FIG. 5C is a perspective view showing an example body of a sensing attachment, the body in the form of a filament and the shape of a spring.

FIG. 5D shows a cross-sectional view of the filament of FIG. 5C, and in particular shows the flat cross-section of the filament of FIG. 5C having rounded edges.

FIG. 6 is a bottom right perspective view showing an example body of a sensing attachment, the body in the form of a hollow filament with cuts made therein, and the shape of a spring.

FIG. 7A is a front view showing the body of the sensing attachment of FIG. 1 in a natural, non-compressed and non-expanded size.

FIG. 7B is a front view showing the same body of FIG. 7A in a radially expanded size.

FIG. 8 is a block diagram showing components of an example implantable reporting processor (IRP) including a sensor.

FIG. 9A, FIG. 9B, FIG. 9C and FIG. 9D are each front left perspective views, each view showing an embodiment for fixedly attaching a sensor to a support.

FIG. 10 is a front perspective view showing a construct comprising a sensor fixedly attached to a support.

FIG. 11 is a front view which shows another view of a construct comprising a sensor fixedly attached to a support.

FIG. 12 is a detailed view showing an expanded view of a portion of FIG. 11, illustrating the relatively placement of a support element and the sensor.

FIGS. 13A and 13B are front views that show that a construct may be adjusted to be in either an expanded form as in FIG. 13B or compact form as in FIB. 13A.

FIG. 14 is a top view showing sensors and other components of a sensing attachment securely affixed to the spline 63 of the body of FIG. 6.

FIG. 15 is a partial cross-sectional view of a blood vessel, within which is a front view of an assembly comprising a sensor, a support for the sensor, and a medical device, wherein the sensor is in direct contact with and is fixedly attached to the support, and wherein the support is in direct contact with and is securely engaged with the medical device.

FIG. 16 is a partial cross-sectional view of a blood vessel, within which is a front view of a stent graft to which is associated two sensing attachments, one (420) in a clip shape and the other (422) in a clamp shape, each sensing attachment securely associated with the stent graft.

FIG. 17 is a partial cross-sectional view of a blood vessel, within which is a front view of a stent graft to which is associated a sensing attachments having a spring shape of the present disclosure shown in a perspective view, securely associated with the stent graft.

FIG. 18 is a partial cross-sectional view of a blood vessel, within which is a stent graft shown in a front view, to which is associated a sensing attaching shown in a bottom right perspective view, the sensing attachments having a hollow filament form with multiple cuts to provide a spring shape of the present disclosure, securely associated with a stent graft.

FIG. 19 is a partial cross-sectional view of a blood vessel, within which is a stent graft shown in a front view, and also showing an assembly comprising a construct, the construct comprising a sensor and a support, the construct in close association with a medical device, in this case an endovascular graft.

FIG. 20 is an isometric view of a delivery system configured to deliver a sensing attachment, or a combination of a sensing attachment associated with a medical device, to a patient.

FIG. 21 is a side view of a delivery catheter of the delivery system of FIG. 20, showing the location of the sensing attachment, or a combination of a sensing attachment associated with a medical device, as contained within the delivery catheter.

FIG. 22 is a context diagram of a sensing attachment environment in a patient's home.

FIG. 23 illustrates a sensing attachment with a plurality of antennas.

FIG. 24 illustrates placement in a blood vessel of a medical device and a sensing attachment with a plurality of antennas.

FIG. 25 illustrates a sensing attachment with a loop antenna.

FIG. 26 illustrates a plot of s-parameters of a loop antenna for a sensing attachment without use of matching circuitry.

FIG. 27 illustrates matching circuitry for a loop antenna for a sensing attachment.

FIG. 28 illustrates a plot of s-parameters of a loop antenna for a sensing attachment with use of matching circuitry.

FIG. 29 illustrates a monopole helix antenna for a sensing attachment.

FIG. 30 illustrates a sensing attachment with a monopole helix antenna.

FIG. 31 illustrates matching circuitry for a monopole helix antenna for a sensing attachment.

FIG. 32 illustrates a plot of s-parameters of a monopole helix antenna for a sensing attachment with use of a matching circuitry.

FIG. 33 illustrates a spiral helix antenna for a sensing attachment.

FIGS. 34 and 35 illustrate testing of communications performance of a sensing attachment with an antenna.

FIG. 36 illustrates antennas for wireless transmission of power to a sensing attachment.

FIG. 37 illustrates a delivery system for delivering an implantable device.

FIG. 38 illustrates a distal portion of the delivery system shown in FIG. 37 following release of a distal portion of an implantable device.

FIG. 39 illustrates a distal portion of the delivery system shown in FIG. 37.

FIG. 40 illustrates a distal portion of the pusher shaft shown in FIG. 37.

FIG. 41 illustrates a proximal portion of the delivery system shown in FIG. 37.

FIG. 42 illustrates a handle that can be used in connection with the delivery system shown in FIG. 37.

FIG. 43 illustrates an exploded view of the handle shown in FIG. 42.

FIG. 44A illustrates a distal portion of another delivery system for delivering an implantable device.

FIG. 44B illustrates a schematic representation of the distal portion shown in FIG. 44A.

FIGS. 45A, 45B, 45C, and 45D illustrate a method of delivering an implantable device.

FIG. 46A illustrates a human body model for testing communications performance of a sensing attachment with an antenna.

FIGS. 46B, 46C, 46D, 46E, and 46F illustrate development of blood phantom and conglomerate phantom for testing communications performance of a sensing attachment with an antenna.

FIG. 47 illustrates a sensing attachment with a helix antenna.

FIG. 48 illustrates a setup for testing a sensing attachment with a helix antenna.

FIG. 49 illustrates a plot of s-parameters of a helix antenna for a sensing attachment.

FIG. 50 illustrates matching circuitry for a helix antenna for a sensing attachment.

FIGS. 51A and 51B illustrate plots of testing the range of a helix antenna for a sensing attachment.

FIG. 52 illustrates a delivery system carrying an implantable device.

FIG. 53A illustrates a handle of the delivery system shown in FIG. 52.

FIG. 53B illustrates a partial exploded view of the handle shown in FIG. 53A.

FIGS. 54A and 54B illustrate actuation of a handle driver to advance an implantable device.

FIGS. 54C, 54D, and 54E illustrate various views of a proximal portion of the handle shown in FIG. 53A.

FIGS. 55A and 55B illustrate actuation of a collar to deflect a distal portion of an outer sheath.

FIGS. 56A, 56B, and 56C illustrates rotation of the collar to lock the distal portion of the outer sheath in a deflected configuration.

FIG. 57 illustrates removal of a release pin.

FIGS. 58A, 58B, and 58C illustrates a disconnect assembly of the delivery system shown in FIG. 52.

FIGS. 59A and 59B illustrate a body of a sensing attachment.

FIGS. 60A, 60B, and 60C illustrate another body of a sensing attachment.

### DETAILED DESCRIPTION

The present disclosure may be understood more readily by reference to the following detailed description of embodiments and the Examples included herein. In reading this detailed description, and unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In one aspect, the present disclosure provides an independent sensing attachment and related systems, which works in conjunction with approved medical devices, treatment methods and procedures. The sensing attachment is independent of a medical device in that the sensing attachment is not necessarily a component or integrated part of the medical device, but is instead attached to or otherwise secured to an independent and fully functioning medical device, where the attachment is secured in a reversible manner. The sensing attachment includes a sensor that can detect and/or measure features in the vicinity of the attachment. For example, the sensing attachment may measure any one or more of fluid dynamics attributes such as flow and/or pressure, the presence of biologic markers such as a marker for infection and/or a marker for inflammation, and/or detection of particles within the human arterial or venous vessel system. In one aspect, the data obtained from the sensor, or a modified form of the data, is communicated to an external receiver for data integration and analysis.

In one aspect, the present disclosure provides a sensing attachment, where the attachment may be used in conjunction with a medical device, optionally a medical device that has been implanted into a patient, i.e., an implanted medical device. The sensing attachment includes a sensor, i.e., includes one or more sensors, where the sensor may detect and/or measure a condition, i.e., one or more conditions, characteristic of a feature in the vicinity of the sensing attachment. In one embodiment the sensing attachment may be in direct contact with the medical device. In one embodiment the sensing attachment is very close to the medical device, such as with a few centimeters, i.e., 1 or 2 or 3 centimeters, of the medical device. In addition to a sensor, the sensing attachment includes a body which functions to maintain the sensing attachment in a desired location. The sensor may be directly affixed to the body, e.g., by gluing or welding the sensor to the body. In one embodiment, the sensor is contained in a specially designed housing that provides for secure fixing of the sensor to the sensing attachment, e.g., to the body of the sensing attachment.

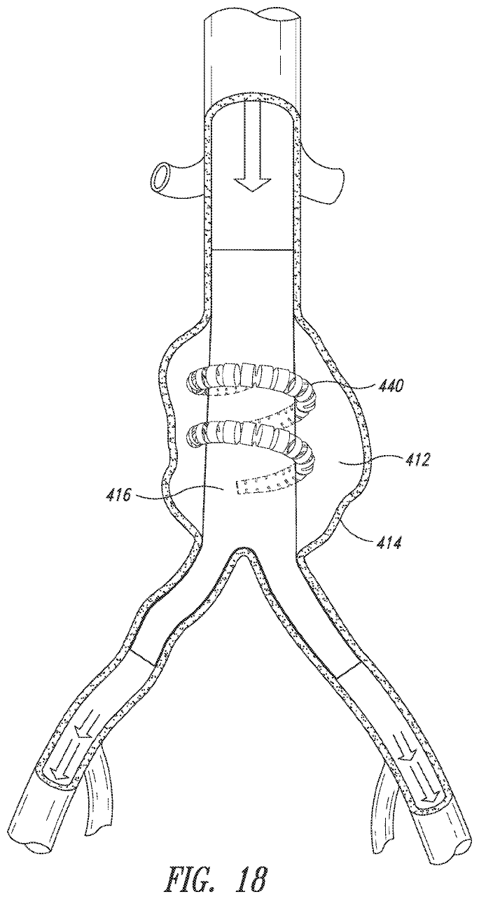

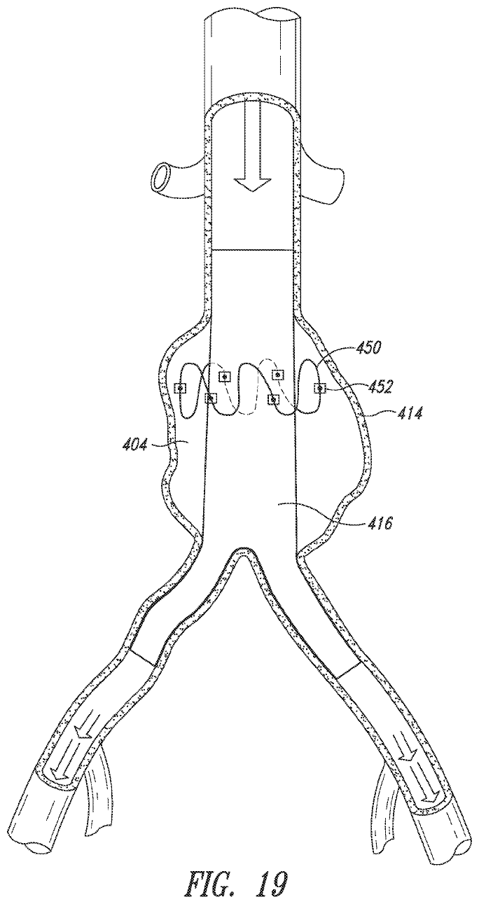

As mentioned above, in one embodiment the sensing attachment is very close to the medical device, however it is not necessarily in direct contact with the medical device. In one embodiment, the sensing attachment is sized so that is fits around a tubular shaped medical device but does not fit snugly against the outer wall of the tubular medical device. Instead, the sensing attachment fits around the outside of the tubular medical device but leaves a gap between the outer surface of the medical device and the inner surface of the sensing attachment. For example, the sensing attachment may be bound about the treatment device and within a wall of the vessel but not in contact at the acute point of the treatment. In this way, the sensing attachment does not rub against, and possibly cause degradation of, the outer surface of the medical device. For example, when the sensing attachment is intended to be associated with a tubular medical device, e.g., a graft or stent graft, that has an outer diameter (or outer cross-sectional distance) of 35 mm, then the sensing attachment may have an inner diameter (or inner cross sectional distance) of more than 35 mm, e.g., exactly or about any of 36 mm, or 37 mm, or 38 mm, or 39 mm, or 40 mm, or 41 mm, or 42 mm, or 43 mm, or 44 mm, or 45 mm, up to about 50 mm. The sensing attachment needs to fit within the body cavity where it is being located, and to that end the sensing attachment may have an outer diameter (or outer cross sectional distance) of less than the inner diameter (or inner cross sectional distance) of the body cavity, e.g., the aneurysm sac. The sensing attachment, when coiled, may have an outer diameter of at least about 25 mm and/or less than or equal to about 70 mm, for example, between about 25 mm and about 35 mm, between about 30 mm and about 40 mm, between about 35 mm and about 45 mm, between about 40 mm and about 50 mm, between about 45 mm and about 55 mm, between about 50 mm and about 60 mm, between about 55 mm and about 65 mm, or between about 60 mm and about 70 mm. If the body cavity, e.g., aneurysm sac, has an inner diameter (or inner cross section distance) of about 50 mm, then the sensing attachment may have an outer diameter (or outer cross sectional distance) of less than about 50 mm, e.g., exactly or about any of 49 mm, or 48 mm, or 47 mm, or 46 mm, or 45 mm, or 44 mm, or 43 mm, or 42 mm, etc. In one embodiment, the sensing attachment has an inner cross sectional distance, which may be an inner diameter of the sensing attachment, where that inner cross sectional distance is in the range of about 35 mm to 45 mm. When the medical device has an outer cross sectional distance in the range of 20 mm to 35 mm, then a sensing attachment may have an inner cross sectional distance which is 1-5 mm greater than the outer cross sectional distance of the medical device, e.g., the sensing attachment may have an inner cross sectional distance of 21 mm to 40 mm. In embodiments, the sensing attachment has an inner cross sectional distance of from 15 mm to 20 mm, or from 20 to 25 mm, or from 25 to 30 mm, or from 30 to 35 mm, or from 35 to 40 mm. In embodiments, the sensing attachment has an inner cross sectional distance, which may be a diameter if the inner cross section is a circle or essentially a circle, selected from the group consisting of 15 mm to 20 mm, 20 mm to 25 mm, 25 mm to 30 mm, 30 mm to 35 mm, 35 mm to 40 mm, and 40 mm to 45 mm. The inner cross sectional distance, in the event the inner cross section is not a circle or essentially a circle, is the shortest distance directly across from a point on the inner surface as seen in cross section of the sensing attachment. The outer cross sectional distance, in the event the outer cross section is not a circle or essentially a circle, is the furthest distance between a reference point on the outer surface as seen in cross section of the sensing attachment, and another point directly across from the reference point. In embodiments, the sensing attachment has an outer cross sectional distance of from 20 mm to 50 mm, or from 20 to 25 mm, or from 25 to 30 mm, or from 30 to 35 mm, or from 35 to 40 mm, or from 40 mm to 45 mm, or from 45 mm to 50 mm. In embodiments, the sensing attachment has an outer cross sectional distance, which may be a diameter if the outer cross section is a circle or essentially a circle, selected from the group consisting of 20 mm to 25 mm, 25 mm to 30 mm, 30 mm to 35 mm, 35 mm to 40 mm, 40 mm to 45 mm, and 45 mm to 50 mm. In one embodiment, the sensing attachment having the afore-mentioned size, has the shape of a spring as shown in FIG. 6, and in vivo surrounds a tubular medical device such as shown in FIG. 18, where the sensing attachment is selected to have a sufficiently large inner cross section that it does not compress against the outer surface of the tubular medical device. The inner and outer cross sectional distances of a spring being determined by looking down the axis of the spring, where the axis identifies the center of the circle or other cross sectional shape of the spring, and the cross sectional distance is the length of a straight line that intersects that center point.

In one aspect, the body of the sensing attachment is or comprises a filament. As used herein, a filament refers to a form that is very long as compared to its width and height. Optionally, the filament has the same width and height, in which case the filament has a circular cross-section such as present in a typical wire having a round cross-section. However, a filament of the present disclosure does not necessarily have equal width and height dimensions, i.e., is not necessarily round. In one embodiment, the width is relatively small and the height is relatively large, so that the filament has a cross-section that may be described as flat. In this case, the filament may be described as a flat filament having two sides. Such a form is well known in the wire industry as flat wire. In a flat filament, the edges may be rounded, or they may be sharp, i.e., the flat wire has square edges. The opposing sides of the flat filament may or may not have the same profile.

The filament may optionally be a solid filament, such as a wire. The filament may optionally be a hollow filament, such as a tube. The filament may be a monofilament, rather than, for example, a multifilament. Thus, in aspects, the present disclosure provides a body in the form of a solid monofilament, and a body in the form of a hollow monofilament. The present disclosure also provides a body in the form of a multifilament.

In one embodiment, the body is formed from a single filament, such as a single hollow monofilament. In one embodiment, the body is formed from multiple filaments, such as a mixture of solid monofilaments and hollow monofilaments. To clarify, in a multifilament, each filament of the multifilament follows the same spatial path since the individual filaments of the multifilament are joined together all along their lengths. In contrast, each of the individual filaments present in a body formed from multiple filament can follow its own spatial path since the individual filaments in this case are not joined together all along their lengths.

In one embodiment, the body is formed in whole or part from a single filament. In one embodiment the body is formed in whole or part from a single monofilament. In one embodiment, the body is formed in whole or part from a single solid monofilament. In one embodiment, the body is formed in whole or part from a single hollow monofilament. In one embodiment, the body is formed in whole or part from a multifilament. In one embodiment the body is formed in whole or part from a single multifilament. In one embodiment, the body is formed in whole or part from a single multifilament comprising multiple solid monofilaments. In one embodiment, the body is formed in whole or part from a single multifilament comprising multiple hollow monofilaments.

For example, a body made from multiple monofilaments may have the form of multiple rings, each ring being made from a monofilament, where the rings are locked together. For instance, a center ring may be joined to two adjacent rings, where each of the adjacent rings is further attached to another new ring, etc., to provide a form in the shape of a plurality of rings joined together. This form may be described as a chain, where each monofilament provides a link for the chain.

In one embodiment, the body is formed in whole or part from a sheet, which refers to a form that is very thin as compared with its length and width.

The body of the sensing attachment may be described in terms of its shape. The body, e.g., the filament or sheet, may take various shapes. In one embodiment, the shape provides the sensing attachment with a size-conforming body that can conform to a size and shape of the medical device with which the sensing attachment is associated. In one embodiment, the shape provides the sensing attachment with a size-adjustable body that can adjust to a size and shape of the medical device with which the sensing attachment is associated in the event that the medical device undergoes changes in size and/or shape during operation of the medical device within the patient. In one embodiment, the shape provides the feature that the sensing attachment may be reversibly attached to and detached from the medical device, i.e., the body holds the sensing attachment in a desired location without any physical mechanical joining of the sensing attachment to the medical device.

In one embodiment, the body has or includes the shape of an undulating filament in the overall shape of a ring, i.e., the filament does not have a beginning or an end. Such a body is illustrated in FIG. 1, which shows a body 10 made from a filament 12, the filament following an undulating path as it creates the shape of a ring. The undulating path may also be described as sinusoidal in the sense that the path turns right, then after a distance turns left, then after a further distance turns right again, etc.

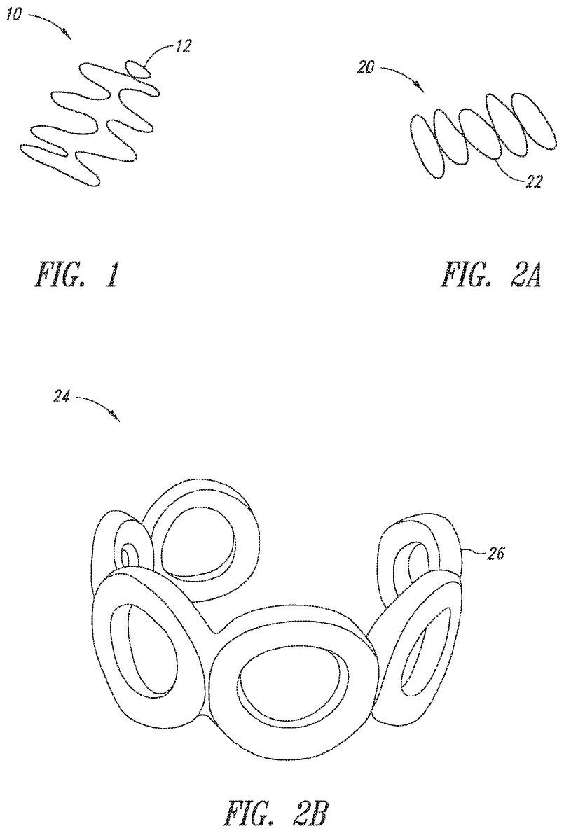

In one embodiment, the body has the shape of plurality of rings that are joined together to form a chain of rings. Optionally, each ring may pass through two adjacent rings, as links do to form a flexible chain. Optionally, each ring is fixedly attached to two adjacent, where such a body is illustrated in FIG. 2A, which shows a body 20 made from a filament 22, the filament 22 in the shape of a ring, the body 20 having a plurality of rings (five rings being shown for illustration in FIG. 2A) that are fixedly joined together.

In one embodiment (not shown), a series of adjacent rings form a circular chain, in that no specific ring can be said to be the first or last ring, where such a shape may also be referred to as a bangle bracelet shape. In another embodiment, as illustrated in FIG. 2B, a chain of adjacent rings 24 is not entirely circular, but instead there is a beginning ring and an ending ring, with a plurality of rings 26 in-between. In FIG. 2B, a series of rings is formed into the shape of a clamp, also known as a cuff bracelet shape. In another embodiment (not shown) the plurality of rings are in the form of a spring.

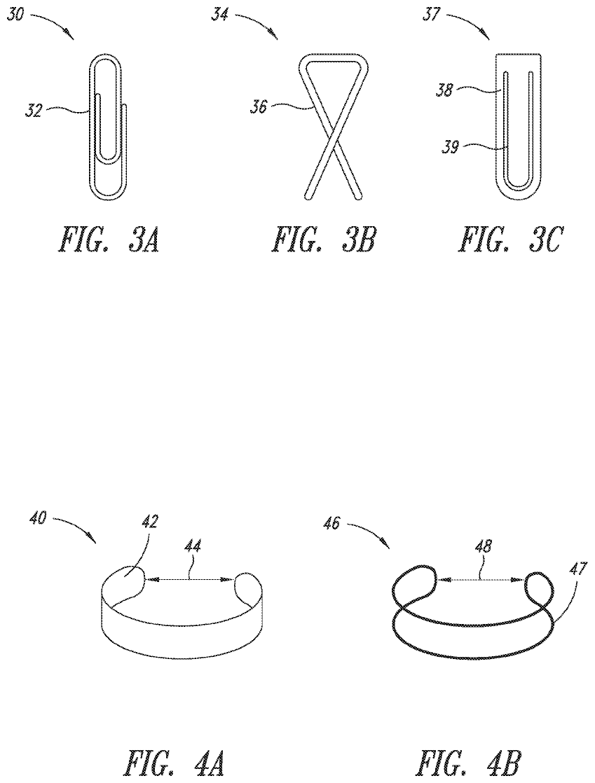

In one embodiment, the body has or includes the shape of a clip. The clip is designed to fix or attach onto an edge of a medical device in a secure manner. Example shapes of a clip are shown in FIGS. 3A, 3B and 3C. These clips effectively function in the same way as a paper clip which can be attached to a sheet of paper.

FIG. 3A shows a body 30 made from a filament 32, in the shape of a classic paper clip. FIG. 3B shows a body 32 made from a filament 34 in the shape of a commonly seen paper clip shape. FIG. 3C shows a body 37 made from a sheet 38 that includes a cut 39 to provide a body in the shape of a paper clip.

In one embodiment, the support structure has or includes the shape of a clamp. An example clamp shape is shown in FIG. 4A. The body 40 in the shape of a clamp FIG. 4A has the form of a strip of material, where that form has been shaped into a semi-circle, where the semi-circle extends more than 180 degrees but less than 360 degrees so that the semi-circular clamp 40 includes a gap 44. The clamp 46 illustrated in FIG. 4B is made from a filament 48 rather than a sheet of material, where the filament 478 effectively traces the edges of the clamp of FIG. 4A, and likewise includes a gap 48.

In one embodiment, the body has or includes the shape of a spring. A spring has a surface in the shape of a coiled tube, generated by sweeping a circle about the path of a helix. In one embodiment the helix runs in a clockwise direction. In one embodiment, the helix runs in a counter-clockwise direction. The direction may be selected depending, e.g., on the intended route a percutaneous delivery of the sensing attachment may take when it is being implanted.

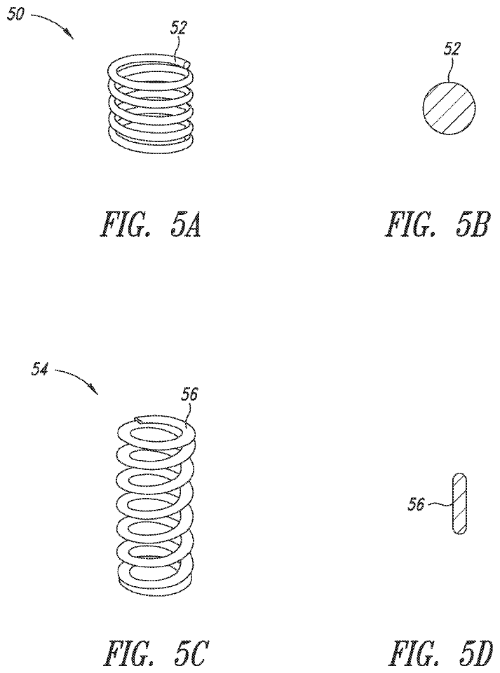

An example spring is shown in FIG. 5A. The body 50 in FIG. 5A is made from a round monofilament 52, where the monofilament 52 is shown in cross-section in FIG. 5B, where that cross-section is circular. Thus, the spring 50 is made from a solid monofilament 52. Another example spring is shown in FIG. 5C. The spring 54 of FIG. 5C is made from a flat monofilament 56, where the monofilament 56 is shown in cross-section in FIG. 5D, where that cross-section is essentially flat as opposed to circular. Thus, the spring 54 is made from a flat solid monofilament 56.

In FIG. 5A and FIG. 5C, the body in the form of a spring is shown as being formed from a solid filament, either a solid circular filament as shown in FIG. 5A, or an essentially flat filament as shown in FIG. 5C. However, the spring shape is not limited to being formed from a solid or flat filament. In another embodiment, the spring is formed from a hollow filament, e.g., a hollow filament with a circular cross section.

In FIG. 6, a body in the shape of a spring is shown as being formed from a hollow circular filament. In the body 60 illustrated in FIG. 6, a hollow filament 61 has been cut in multiple places along its length to provide a plurality of cuts, where cuts 62a, 62b and 62c are example. These cuts provide the filament with enhanced compliancy. It will be appreciated that within the context of the present disclosure that the term “cutting” includes any process used to impart a specific pattern of tines into a hollow filament, by cutting, etching, grinding or any other method. In one particular form, such cutting is achieved through laser cutting. In one embodiment, the support structure is in the shape of a spring, the spring being formed from a hollow filament, the hollow filament having cuts which pass part way through the hollow filament to provide a spline to the filament. Cuts may likewise be added to a solid circular filament or a flat filament, in order to enhance compliancy.

When cuts are made in a filament, in one option the cuts are identical cuts made along the length of the filament. That is, each cut begins at the same side of the filament, and each cuts extends into the filament for a fixed distance, the distance being less than the diameter of the filament. This option may be referred to as a straight cut hollow tube and is illustrated in FIG. 6. In this option, the hollow filament with cuts has a spine 63, also known as a spline or a slat, where these terms are each referring to a long, narrow, thin strip of the material from which the tube is formed and where no cuts are present. The greater the depth of the cut, the narrower the spline. In embodiments, the spline has a width of less than 25% of the circumference of the filament, or less than 20%, or less than 15%, or less than 10% of the circumference of the filament.

Referring again to FIG. 6, extending from the spline are a series of loops, where three such loops are shown as features 64a, 64b and 64c in FIG. 6. The loops may be defined, in part, by their length. In one embodiment, cuts are made in the hollow filament every 6 mm, so that the loops have a length of about 6 mm (slightly less than 6 mm, since the cut will remove a small amount of material). In general, all other factors being constant, greater compliancy is achieved when the loop length is shorter. In embodiments, the loop length is less than 20 mm, or less than 15 mm, or less than 10 mm, or less than 8 mm. However, if the loop length is too short relative to the diameter of the hollow monofilament, then the resulting spring does not have much strength to retain its shape. In embodiments, the loops have a length of at least 4 mm, or at least 5 mm, or at least 6 mm, or at least 7 mm, or at least 8 mm, or at least 9 mm, or at least 10 mm. In embodiments, the hollow filament has a plurality of loops, the plurality of loops having a length of 1-20 mm, or 2-10 mm, or 3-8 mm, or 5-7 mm. In embodiments, the hollow filament has a diameter of less than 10 mm, or less than 9 mm, or less than 8 mm, or less than 7 mm, or less than 6 mm, or less than 5 mm, or less than 4 mm, including ranges formed by any two listed values, e.g., a diameter in the range of 4-6 mm.

The cuts may be regularly and identically made along the length of the hollow filament in order to provide a body of the present disclosure, and this situation is illustrated in FIG. 6. However, the cuts may be in a pattern such that each cut is not identical to the previous (adjacent) cut, but instead varies by some fixed parameter along the length of the hollow filament. For example, the beginning of a cut may be offset by a fixed number of degrees compared to the previous cut. Such a structure may be envisioned as being formed by rotating the hollow filament around its longitudinal axis by a fixed amount after each cut is made, so that the resulting spline has a helical shape, also referred to as a corkscrew or sinusoidal shape. The resulting pattern of cuts is an example of a cross-articulating pattern, where cross articulation is known in the art of laser cutting of hollow monofilaments, and provides a large variation in cuts and cut patterns. In general, the hollow monofilament of the present disclosure may be cut into any cross articulation pattern to provide a body for a sensing attachment of the present disclosure.

In one aspect, the body of the sensing attachment of the present disclosure conforms to the shape and/or the size of a medical device against which the construct is placed. Thus, if the medical device is, for example, a graft having a tubular shape, and the body is wrapped around the exterior of the tubular graft in a helical fashion, the body of the present disclosure may contract in size so it lies directly against the fabric of the graft, and adopts the shape and size of the tubular graft. This property of a body of the present disclosure will be referred to as compliancy, and in one aspect the body of the present disclosure is compliant.

In one aspect, the body of the present disclosure adapts to a change in the shape and/or the size of a medical device against which the construct is placed. Thus, if the medical device is, for example, a graft having a tubular shape, which is implanted into, e.g., a vessel of a patient, and the body is wrapped around the exterior of the tubular graft in a helical fashion, the body of the present disclosure may increase and/or decrease in size in direct response to changes in the size of the graft. While implanted in the patient, the graft may change in size due to changes in pressure within the vessel that cause the diameter of the graft to increase (expand) or decrease (contract) in diameter. Thus, in one embodiment the body has the ability to resume its normal shape after being stretched or compressed. This property of a body of the present disclosure will be referred to as elasticity, or elastic compliance, and in one aspect the body of the present disclosure is elastic, or elastically compliant. The construct may alternatively be referred to as resiliently deformable.

In one aspect, the body of the present disclosure undergoes a change in size and/or shape upon heating, such as from 25° C. to 37° C. This property of a construct of the present disclosure will be referred to as shape memory, and in one aspect the construct of the present disclosure has shape memory.

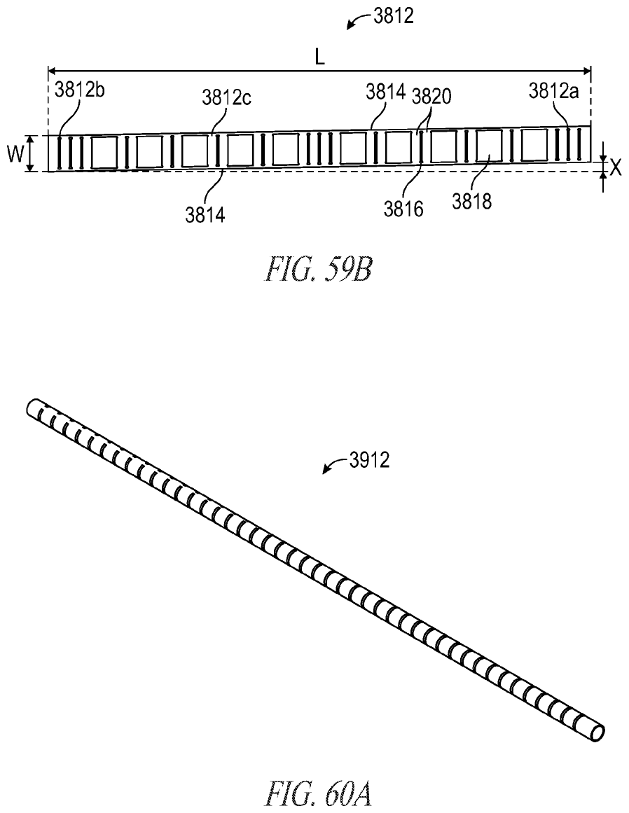

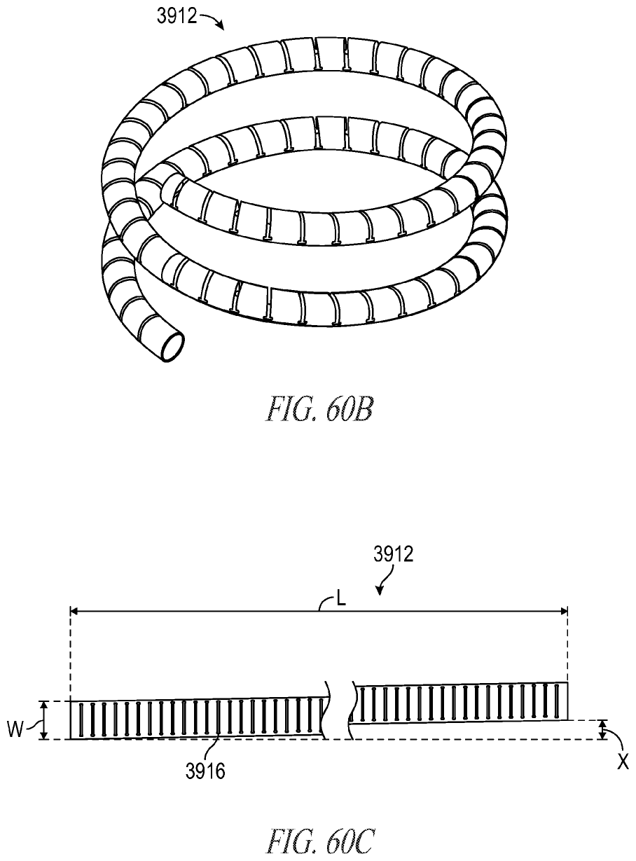

FIGS. 59A and 59B and 60A, 60B, and 60C illustrate variations of the body 60 that may be incorporated into any of the implantable devices or constructs described herein. The body structures described herein may be coated or covered with an outer polymeric layer as described further below.

FIGS. 59A and 59B illustrates a body 3812 that can transition between the linear configuration shown in FIG. 59A to a coil configuration as shown in FIG. 6. The body 3812 can include a hollow filament or tubular body with a plurality of cutouts 3816 to enable the body 3812 to form the coil. Fully circumferential loop portions 3820 are formed between adjacent cutouts 3816. The cutouts 3816 can be formed using any of the cutting methods described above.

The body 3812 may include a plurality of tubular segments 3812a, 3812b, 3812c. The plurality of segments 3812a, 3812b, 3812c may be spaced apart from each other by open spaces 3818. The plurality of segments 3812a, 3812b, 3812c may be interconnected only by the spine 3814. The open spaces 3818 decrease the total amount of metal present that may interfere with imaging equipment. The open spaces 3818 may also provide access to sensors housed within the body 3812.

The body 3812 may include a first segment 3812a at a first end of the body 3812 and a second segment 3812b at the opposite end of the body 3812. The body 3812 may include one or more intermediate segments 3812c between the first segment 3812a and the second segment 3812. Each segment 3812a, 3812b, 3812c may have the same length or different lengths. For example, the first segment 3812a and the second segment 3812b may be the same length, but longer than one or more intermediate segments 3812c. Electronic circuitry as described herein may be disposed within or supported by the larger end segments 3812a, 3812b. Each intermediate segment 3812c may have the same length or differ in length. For example, as illustrated, a central segment has the same length as the first and second segments 3812a, 3812b, but is longer than the remaining intermediate segments 3812c. Each of the plurality of tubular segments 3812a, 3812b, 3812c may include one or more of the cutouts 3816, for example one, two, or three cutouts, with longer segments having a greater number of cutouts 3816 compared to shorter segments. The tubular segments 3812a, 3812b, 3812c may have a generally constant diameter.

FIG. 59B illustrates a flattened version of the body 3812 cut along the spine 3814 such that the spine 3814 forms lateral edges of the flattened body 3812. A length L of the body 3812, measured from one end to the opposite end, is at least about 10 cm and/or less than or equal to about 40 cm, for example, between 10 cm and 20 cm, between 15 cm and 25 cm, between 20 cm and 30 cm, or between 25 cm and 35 cm, inclusive of the ends of the ranges. A width of the flattened body 3812 (or circumference of the tubular body) may be at least about 5 mm and/or less than or equal to about 20 mm, for example between 5 mm and 10 mm, between 7.5 mm and 12.5 mm, or between 10 mm and 15 mm, inclusive of the ends of the ranges. The flattened body 3812 may have two pairs of parallel sides, but have a non-rectangular shape. Lateral edges of the flattened body 3812 formed by the spine 3814 may be formed at an oblique angle x relative to horizontal, perpendicular to the edges at either end of the flattened body 3812, for example an angle x between 0.5 degrees and 3.0 degrees.

Each of the plurality of segments 3812a, 3812b, 3812c may have a length of at least about 5 mm and/or less than or equal to about 30 mm, for example between 5 mm and 10 mm, between 7.5 mm and 12.5 mm, between 10 mm and 15 mm, between 12.5 mm and 17.5 mm, or between 15 mm and 20 mm, inclusive of the ends of the ranges. The tubular segments 3812a, 3812b, 3812c may be separated by open spaces 3818 having a length of at least 1.0 mm and/or less than or equal to about 15 mm, for example between 2.5 mm and 7.5 mm, or between 5 mm and 10 mm, or between 7.5 mm and 12.5 mm. The length of the open spaces may be at least 3×, at least 5×, or at least 10× greater, than the length of the cutouts 3816. Adjacent cutouts 3816 on the same tubular segment 3812a, 3812b, 3812c may separate loop structures 3820 having a length of at least about 1 mm and/or no more than about 10 mm, for example between 2.5 and 5 mm, between 5 mm and 7.5 mm, or between 7.5 mm and 10 mm. Each cutout 3816 may have a generally dog bone shape in the circumferential direction with first and second ends of the cutout 3816 having a greater length than an intermediate portion between the first and second ends. A length of the intermediate portion of each cutout 3816 may be no more than about 5.0 mm, no more than about 3.0 mm, no more than about 2.0 mm, no more than about 1.0 mm, or no more than about 0.5 mm. A width of each of the cutouts 3816, measured in the circumferential direction, may be at least about 5.0 mm and/or less than or equal to about 15 mm, for example between 7.5 mm and 10 mm, or between 10 mm and 12.5 mm. The cutouts may extend across at least 75% of a width of the body 3812, at least about 80% of a width of the body 3812, or at least 85% of a width of the body.

FIGS. 60A, 60B, and 60C illustrate another body 3912 that can include any of the features described above with respect to the body 3812 except as described below. Unlike body 3812, the body 3912 is a continuous body without open spaces dividing tubular segments. The cutouts 3916 may be evenly spaced apart along the length of the body 3912. For example the cutouts 3916 may be separated by a distance of at least about 1 mm and/or less than or equal to about 5 mm. Each of the cutouts 3916 may be similarly shaped and sized.

In the coiled configuration shown in FIG. 60B, the body 3912 can have a pitch of at least about 5 mm and/or less than or equal to about 15 mm, for example between 5 mm and 10 mm, between 7.5 mm and 12.5 mm, or between 10 mm and 15 mm. A diameter of the coiled body can be at least about 10 mm and/or less than or equal to about 60 mm, for example between about 20 mm and 30 mm, between about 25 mm and 35 mm, between 30 mm and 40 mm, between 35 mm and 45 mm, or between about 40 mm and 50 mm, or between 45 mm and 55 mm. In the coiled configuration, the body 3912 may include one or more circumferential turns, for example one turn, two turns, or three turns.

Although certain cut patterns are described above for the body, in other configurations, the body may be braided.

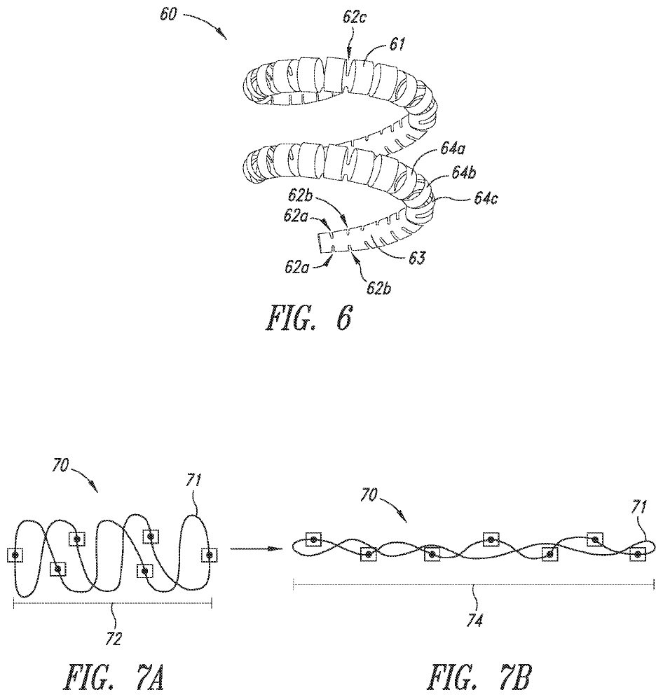

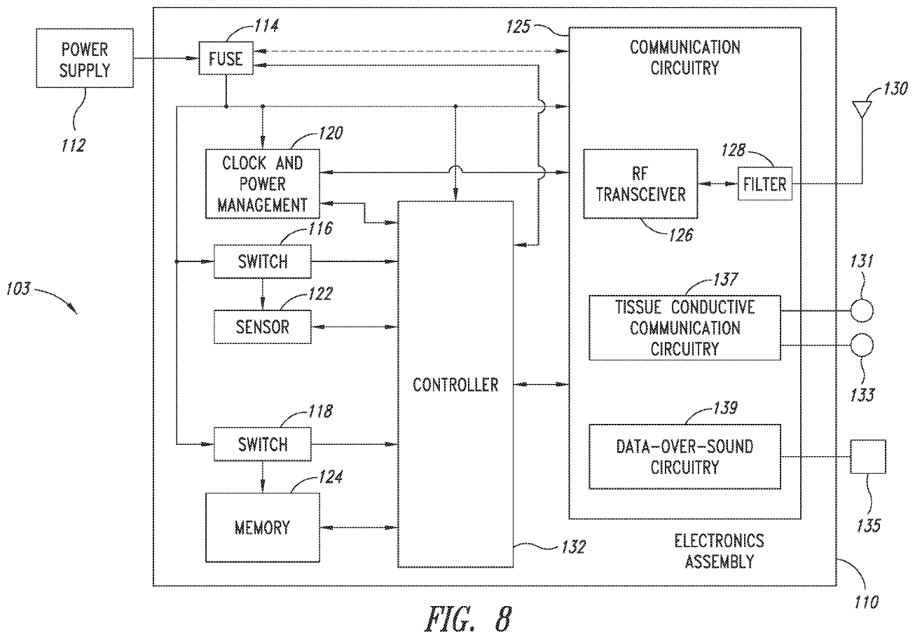

Whether a construct of the present disclosure is one or more of compliant, elastic, or has shape memory, may depend on the material or materials from which the construct is made as discussed below, and/or the shape selected for the body as discussed above. FIG. 7A show a body 70 in the shape of a ring made from an undulating filament 71 such as illustrated in FIG. 1, in a contracted form with a diameter 72. As shown in FIG. 7B, upon radial expansion 73, the body 70 made from the undulating filament 71 adopts an expanded form having a diameter 74. This change in diameter is facilitated by the selection of the shape of the body, where in FIGS. 7A and 7B it is seen that undulations of the filament 71 become less sharp, or less pronounced, as the body expands to a diameter 74. In one embodiment, the body of the sensing attachment of the present disclosure has a shape that can expand and contract, such as the rings, clips, clamps and springs illustrated herein.

In one aspect, the body of the sensing attachment is made in whole or part from metal, including metal alloy. Example metals are platinum, alloys of platinum and iridium, and alloys of nickel and titanium. In one aspect, the metal is nitinol. Nitinol refers to a super elastic metal alloy of nickel and titanium. In one embodiment, the two elements are present in roughly equal atomic percentage (e.g., Nitinol 55, Nitinol 60). Nitinol exhibit two closely related and unique properties: shape memory effect (SME) and superelasticity (SE; also called pseudoelasticity, PE). Shape memory is the ability of nitinol to undergo deformation at one temperature, then recover its original, undeformed shape upon heating above its “transformation temperature”. Superelasticity occurs at a narrow temperature range just above its transformation temperature; in this case, no heating is necessary to cause the undeformed shape to recover, and the material exhibits enormous elasticity, some 10-30 times that of ordinary metal. In one aspect, the metal is a non-magnetic alloy of cobalt, chromium, nickel and molybdenum. Such a metal alloy is known as Elgiloy™ metal alloy, and is available from Elgiloy Specialty Metals (Elgin, IL, USA). In one aspect, the metal is stainless steel, an alloy of chromium, nickel and iron.

In one aspect, the support of the construct is made in whole or part from organic polymer. Example polymers include, without limitation, polypropylene, polyethylene including high density polyethylene, and polyester such as formed from ethylene glycol and terephthalic acid (e.g., Dacron™ polyester, PET). In one aspect, the organic polymer is an elastomer, such as silicone, polyurethane, polyurethane siloxane copolymers, and styrene isoprene rubber (e.g., SIS).

In one aspect, the body is formed from a round or elliptical cross-section structure that can be solid or tubular base shape, where the material properties are super-elastic, shape, material, encompassing a metallic or a metallic and polymer combination, such that the mechanical properties are within ratios for proper processing, handling and treatment management to the human body from 32° C.-39° C. and allows fabrication of the body with an allowable strain of 8.5% or less for processing and treatment deliverability.

In one aspect, the body of the sensing attachment has a coat that covers at least a portion of the body. The term coat is intended to encompass both a coating, such as a polymeric coating sitting on and adhering to a surface of the sensing attachment, as well as a sleeve, such as sleeve that is pulled onto a sensing attachment and sits around and on top of the surface of the sensing attachment, as well as a modification made to the surface of the sensing attachment that causes the surface to have different properties than the properties of the underlying material from which the body of the sensing attachment is formed.

The coat or coating may confer desirable properties to the body and/or sensing attachment. In one aspect, the coating enhances the mechanical properties of the body. In one aspect, the coating enhances the electrical properties of the body. In one aspect, the coating enhances the biocompatibility properties of the body. In one embodiment, the sensing attachment may be covered partially or completely in a soft complying material, woven cloth, polymer, or combination of such, to ensure no mechanical damage occurs when interacting with the stent graft.

In one embodiment, the coat may function to reduce the wear that can occur when the sensing attachment changes size in response to changes in size of the associated implant with which the sensing attachment is in contact. For example, if the implant is a stent graft, which repeatedly increases and decreases in diameter due to pulsation within the vessel where the stent graft is located, and the sensing attachment is expanding and contracting in response to this movement of the stent graft, then there may be some rubbing between the graft and the sensing attachment. The graft in a stent graft is often made from a fiber than can abrade upon being rubbed. In one aspect, the present disclosure provides a sensing attachment with a body having a coat, where the coat is less abrasive to the associated medical device than the underlying material thereby minimizing the potential for stent graft abrasion. The coat may partially or completely cover the body in a soft complying material, including woven cloth, polymer, or combination of such, to ensure no mechanical damage occurs when interacting with the stent graft.

In one aspect, the coating is created by adding a metallic element to the surface of the body. Optionally, in this case, the surface has a composition that is a variation on the composition that underlies the surface coat, where the coat contains one or more elements not present in the composition that underlies the coat. Optionally, the added metallic element is present in sufficient quantity and thickness that the entire coat is made from the additional metallic element.

In one embodiment, the coat is an organic polymer, which includes a single polymers as well as a mixture of polymers. In one embodiment, the coat or coating, is biocompatible. In one embodiment, the coat or coating, is non-biodegradable. For example, the coating on the surface of the sensing attachment may be or comprise poly(tetraflororethene, e.g., Teflon™ polymer. Other suitable coatings may comprise one or more of epoxy, silicone, urethane, and acrylic resin. Poly(p-xylylene) coatings, such a prepared from parylene, may also be present on the surface of the sensing attachment.

The coat may be integrated with the body of the sensing attachment, such as when the coat is created by adding a metallic element to the surface of the body, or created by applying an organic polymer to the surface of the body, in which case the coat may be referred to as a coating. Alternatively, the coat may be a separate feature of the sensing attachment. For example, the coat may be in the form of a sleeve that is slipped over and around some or all of the body of the sensing attachment. When a sleeve is used to provide a coat on some or all of the body, that sleeve may optionally incorporate passive or active components that function in conjunction with the sensor or other component of the sensing attachment. Those components that are present in or on the sleeve may be prepared by nano- or micro-electromechanical systems fabrication technology.

In one embodiment, the coat or coating includes a bioactive agent. The bioactive agent may be released into the vicinity of the attachment so as to provide a therapeutic benefit to the patient that has received the medical implant. For example, the bioactive agent may be an anti-proliferative drug that causes a reduction in host endothelialization and/or tissue overgrowth that may accompany implantation of the medical device and/or the sensing attachment. As another example, the bioactive agent may be an anti-fouling agent that protects the surface of the sensing attachment from bacterial deposition.

In one embodiment, the coat or coating includes a chemical that enhances the lubricity of the coating, e.g., the coat or coating may include a lubricious component such as a polyalkylene oxide.

In one embodiment, the final shape of the support structure is achieved by a process known as shape setting. Shape setting is particularly useful when the support structure is formed from a shape memory alloy. After cutting and cleaning the monofilament, the resulting structure is shaped into the desired shape, in case of shape memory alloys followed by cold work, mostly combined with a heat treatment with a mechanical means holding all tines and the base tube constrained in or on a mandrel or fixture in the proper geometry. This is called “shape setting”.

The shape of the stylet can be set with varying degrees of shape setting/training heat treatments (temperature, time, the amount of prior cold work, Bend and Free Recovery (“BFR”) testing, which determine the shape memory alloy's final mechanical properties, austenite finish, transformation temperature, and alloy composition.

The sensing attachment will have a size and shape at body temperature, i.e., at or about 37° C. This size and shape, when no external forces are acting on the sensing attachment, may be referred to as its natural size and natural shape. An elastic or super-elastic sensing attachment may be acted upon by an external force or external forces to cause compression or expansion of the sensing attachment. The compressed or constrained state of the sensing attachment occupies less volume than the non-constrained state, where volume refers to the space contained within the exterior surfaces of the sensing attachment. For example, a sensing attachment may be compressed to fit into a delivery catheter, and constrained to maintain that fit in the delivery catheter. When present within a delivery catheter, the sensing attachment may be described as being in a constrained or compressed form or state. At body temperature, when a constraining feature of the delivery catheter is removed, or the sensing attachment is expelled from the delivery catheter, then the constrained sensing attachment is free to spontaneously adopt a natural or unconstrained or uncompressed form or state.

This technology, of having a constrained state of an article during delivery to a patient, and an unconstrained state after delivery of the article to a desired location in the patient, is well known in the fields of stent delivery and stent graft delivery, particularly when delivery is done percutaneously, i.e., via needle puncture of the skin. In analogy to procedures used to prepare stents and stent grafts for percutaneous stent and stent graft delivery, in one embodiment of the present disclosure, the sensing attachment is prepared from nitinol, and is fabricated into a compressed form during shape setting, and delivered to a patient in the compressed form, and adopts a non-compressed form after delivery to a desired location in a patient. Thus, in one embodiment, the present disclosure provides a method of preparing a sensing attachment in a compressed form from nitinol, using shape setting techniques.

In describing the sensing attachment of the present disclosure, including kits, system and methods of making and using that include the sensing attachment, reference may be made to the diameter of the sensing attachment. Strictly speaking, a diameter is a feature only of a perfect circle, and the sensing attachment of the present disclosure may not have a perfectly circular form. In some embodiments it may have a non-circular form which may be close to but not identical with a circular form. When the sensing attachment is not perfectly circular, the reference to a diameter may be understood to be reference to a distance across the sensing attachment as viewed from a top view of the sensing attachment, where a graft or stent graft may be located either outside or inside of the sensing attachment as viewed from a top view. When the sensing attachment is perfectly circular, then the top view of the sensing attachment will appear as a circle. For example, when the sensing attachment has the shape of a cuff bracelet as shown in FIG. 2B, the inner diameter of the sensing attachment refers to the distance between a first point on an inside surface of the cuff bracelet and a second point which is directly across the interior of the sensing attachment, as determined by reference to the first point. As another example, when the sensing attachment has the shape of a spring as shown in FIG. 6, the diameter of the sensing attachment is determined by reference to a top of view of the sensing attachment, which will have the appearance of circle, where the inner diameter of the sensing attachment refers to the distance between a first point on an inside surface of the circle and a second point which is directly across the interior of the sensing attachment, as determined by reference to the first point, i.e., a standard diameter if the top view of the spring shows the spring as a perfect circle. For sensing attachments that do not form a perfect circle when viewed from a top view, the inner diameter might alternatively be referred to as the internal cross distance, and the outer diameter might alternatively be referred to as the outer cross distance.

When the sensing attachment is intended to be located around the outer surface of the medical device, and be held in place with the aid of hoop stress forces, then the inner diameter or inner cross distance of the sensing attachment refers to the minimum distance between opposing surfaces within the sensing attachment. This minimum distance should be essentially the same, which includes just slightly less than, the outer diameter of the stent graft or graft in order that the sensing attachment exerts a slight force on the medical device. Likewise, when the sensing attachment is intended to be located within the inner surface of the medical device, and be held in place with the aid of hoop stress forces, then the outer diameter or outer cross distance of the sensing attachment refers to the maximum distance between opposing surfaces of the sensing attachment. This maximum distance should be essentially the same, which includes just slightly greater than, the inner diameter of the stent graft or graft in order that the sensing attachment exerts a slight force on the medical device. The inner cross distance is the inner diameter when the device form a perfect circle when viewed from a top view. The outer cross distance is the outer diameter when the device forms a perfect circle when viewed from a top view.

In reference to a graft and a stent graft, each of these has a lumen, and each has a tubular shape when fluid completely fills the lumen, as is typically the case when the medical device has been deployed in a patient and fluid is flowing through the device. The inner diameter and outer diameter of a graft and a stent graft refers to the state of the device when fluid is fully flowing through the lumen of the device. In this state, the graft and stent graft each has an inner diameter (maximum distance across the lumen) and outer diameter (maximum distance between two opposite points on the surface of the graft, as measured across the lumen), where these distances can be observed from a top view of the stent graft or graft, as viewed down the lumen.

In one embodiment, the present disclosure provides a method for associating a sensing attachment to a medical device in a secure manner in vitro, the method comprising: selecting a medical device from the group consisting of a graft and a stent graft, where the medical device has an inner diameter and an outer diameter; selecting a sensing attachment having an inner diameter (or inner cross distance) and an outer diameter (or outer cross distance), where at least one of (i) the inner diameter (or inner cross distance) of the sensing attachment is essentially the same as the outer diameter of the medical device; and (ii) the outer diameter (or outer cross distance) of the sensing attachment is essentially the same as the inner diameter of the medical device; and placing the sensing attachment either within or outside of the medical device in vitro, where hoop stress secures the sensing attachment to the medical device. The sensing attachment may be selected such that it has a size and shape that allows it to be held securely adjacent to an associated stent graft or graft by way of hoop stress. Optionally, when the sensing attachment is a clip, the sensing attachment may be clipped onto the stent graft or graft, in order to associate the sensing attachment to the stent or stent graft.

In one embodiment, the present disclosure provides a method for making a system comprising a medical device having a sensing attachment located within the medical device, the method comprising: providing a medical device selected from the group consisting of a graft and a stent graft, the medical device having an inside (luminal side) and an outside; determining an inner diameter of the medical device; selecting a sensing attachment having an inside and an outside, the outside having an outer diameter (or outer cross distance), where the outer diameter of the sensing attachment is essentially the same as the inner diameter of the medical device; compressing the sensing attachment from a non-compressed state to a compressed state to thereby decrease the inner diameter (or inner cross section) of the sensing attachment and provide a compressed state of the sensing attachment; placing the sensing attachment in the compressed state inside the medical device at a location having the inner diameter; returning the sensing attachment to a non-compressed state, so that the outside of the sensing attachment contacts the inside of the medical device, to provide a system comprising a medical device having a sensing attachment located within the medical device. The sensing attachment may be selected such that it has a size and shape that allows it to be held securely adjacent to an associated stent graft or graft by way of hoop stress. Optionally, when the sensing attachment is a clip, the sensing attachment may be clipped onto the stent graft or graft, in order to associate the sensing attachment to the stent or stent graft.

In one embodiment, the present disclosure provides a method for making a system comprising a medical device and a sensing attachment located external to the medical device, the method comprising: providing a medical device selected from the group consisting of a graft and a stent graft, the medical device having an inner surface (the luminal surface) and an outer surface; selecting a sensing attachment having an inside and an outside, the inside having an inner diameter (or inner cross distance), where the inner diameter (or inner cross distance) of the sensing attachment is larger than the outer diameter of the medical device; and placing the sensing attachment around the medical device. The sensing attachment may be selected such that it has a size and shape that allows it to be held securely adjacent to an associated stent graft or graft by way of hoop stress. Optionally, when the sensing attachment is a clip, the sensing attachment may be clipped onto the stent graft or graft, in order to associate the sensing attachment to the stent or stent graft.

In one embodiment, the present disclosure provides a method for associating a sensing attachment to a stent graft in a secure manner in vivo, the method comprising: implanting a stent graft into a blood vessel of a patient during a medical procedure, the stent graft having an outer diameter; providing a sensing attachment having an inner diameter (or inner cross distance), where the inner diameter (or inner cross distance) of the sensing attachment is essentially the same as the outer diameter of the stent graft; and placing the sensing attachment around the stent graft in vivo during the medical procedure, where hoop stress secures the sensing attachment to the stent graft. The sensing attachment may be selected such that it has a size and shape that allows it to be held securely adjacent to an associated stent graft or graft by way of hoop stress.

In one embodiment, the present disclosure provides a method for associating a sensing attachment to a stent graft in a secure manner in vivo, the method comprising: selecting a stent graft having an outer diameter; implanting the stent graft into a blood vessel of a patient during a medical procedure; selecting a sensing attachment having an inner diameter (or inner cross distance), where the inner diameter (or inner cross distance) of the sensing attachment is essentially the same as the outer diameter of the stent graft; and placing the sensing attachment around the stent graft in vivo during the medical procedure, where hoop stress secures the sensing attachment to the stent graft. The sensing attachment may be selected such that it has a size and shape that allows it to be held securely adjacent to an associated stent graft or graft by way of hoop stress.

The sensing attachment of the present disclosure incudes a sensor, i.e., has one or more sensors that are either directly or indirectly fixed in a secure manner to the body of the sensing attachment. The term “sensor” refers to a device that can be utilized to measure one or more different aspects of a body tissue (anatomy, physiology, metabolism, and/or function) and/or one or more aspects of the medical device. Representative examples of sensors suitable for use within the present disclosure include, for example, fluid pressure sensors, fluid volume sensors, contact sensors, position sensors, pulse pressure sensors, blood volume sensors, blood flow sensors, chemistry sensors (e.g., for blood and/or other fluids), metabolic sensors (e.g., for blood and/or other fluids), accelerometers, gyroscopes, displacement sensors, pressure sensors, fluid sensors, mechanical stress sensors and temperature sensors. Any one or more of these sensors may be included on a sensing attachment. Within further embodiments one or more (including all) of the sensors can have a Unique Sensor Identification number (“USI”) which specifically identifies the sensor.

A sensor may be utilized to detect, measure and/or monitor information relevant to the state of the associated medical device after implantation. The state of the medical device may include the integrity of the device, the movement of the device, the forces exerted on the device and other information relevant to the implanted medical device. Examples of these types of sensors 1022 include pressure sensors, fluid sensors, flow sensors, gyroscopes, accelerometers, displacement sensors and temperature sensors, as well as other sensors mentioned herein.

A sensor may be utilized to detect, measure and/or monitor information relevant to the state of a body or body segment after implantation of the associated medical device. The state of the body or a body segment may include kinematic information of the body or a body segment. Examples of these types of sensor 1022 include fluid flow sensors, pressure sensors, gyroscopes, accelerometers, displacement sensors, impedance sensors and temperature sensors, any one or more of which may be coupled to the processor.

A sensor may be utilized to detect, measure and/or monitor information relevant body tissue after implantation of the associated medical device. Body tissue monitoring may include blood pressure, pH level and flow rate. Examples of this type of sensor 1022 include fluid pressure sensors, fluid volume sensors, pulse pressure sensors, blood volume sensors, blood flow sensors, chemistry sensors (e.g., for blood and/or other fluids), metabolic sensors (e.g., for blood and/or other fluids).

A sensor may be used to monitor and/or measure displacement of a stent graft relative to the vessel within which the stent graft is positioned. For example, a stent graft may have a contact sensor and the sensing attachment placed external to the stent graft may likewise have a contact sensor, where the two contact sensor are sensing one another. If the stent graft moves in a longitudinal direction, the sensing attachment may resist such movement when the sensing attachment is held by hoop stress forces against the outer surface of the stent graft (and also contained with the semi-solid material typically present within an aneurysm sac), or may not undergo any similar movement in the event the sensing attachment is located around the stent graft but not physically contacting the surface of the stent graft. That difference in movement may be recorded as a change in the contact between the two contact sensors (the contact sensor on the stent graft and the contact sensor on the sensing attachment). This change in contact may be communicated externally to a physician, who will become aware that the stent graft has moved, and remedial action can be considered.

Within certain embodiments the sensor can be a wireless sensor, or, within other embodiments, a sensor connected wirelessly to a microprocessor. Within further embodiments one or more (including all) of the sensors can have a Unique Sensor Identification number (“USI”) which specifically identifies the sensor and/or a Unique Device Identification number (“UDI”) with which the sensors can provide unique information of the associated medical device for tracking purposes of the medical device manufacturer, the health care system, and regulatory requirements.

In one embodiment, a Microelectromechanical Systems or “MEMS”, or Nanoelectromechanical Systems or “NEMS”, and BioMEMS or BioNEMS, see generally https://en.wikipedia.org/wiki/MEMS) can be utilized within the present disclosure as the sensor. Representative patents and patent applications include U.S. Pat. Nos. 7,383,071, 7,450,332; 7,463,997, 7,924,267 and 8,634,928, and U.S. Publication Nos. 2010/0285082, and 2013/0215979. Representative publications include “Introduction to BioMEMS” by Albert Foch, CRC Press, 2013; “From MEMS to Bio-MEMS and Bio-NEMS: Manufacturing Techniques and Applications by Marc J. Madou, CRC Press 2011; “Bio-MEMS: Science and Engineering Perspectives, by Simona Badilescu, CRC Press 2011; “Fundamentals of BioMEMS and Medical Microdevices” by Steven S. Saliterman, SPIE—The International Society of Optical Engineering, 2006; “Bio-MEMS: Technologies and Applications”, edited by Wanjun Wang and Steven A. Soper, CRC Press, 2012; and “Inertial MEMS: Principles and Practice” by Volker Kempe, Cambridge University Press, 2011; Polla, D. L., et al., “Microdevices in Medicine,” Ann. Rev. Biomed. Eng. 2000, 02:551-576; Yun, K. S., et al., “A Surface-Tension Driven Micropump for Low-voltage and Low-Power Operations,” J. Microelectromechanical Sys., 11:5, October 2002, 454-461; Yeh, R., et al., “Single Mask, Large Force, and Large Displacement Electrostatic Linear Inchworm Motors,” J. Microelectromechanical Sys., 11:4, August 2002, 330-336; and Loh, N. C., et al., “Sub-10 cm3 Interferometric Accelerometer with Nano-g Resolution,” J. Microelectromechanical Sys., 11:3, June 2002, 182-187; all of the above of which are incorporated by reference in their entirety.

In one embodiment, the sensor is a flow sensor. The flow sensor may be used to measure the flow that passes by the sensor when the sensor is present in a vessel of a host, e.g., a blood vessel. The flow sensor may be used to detect and/or measure variation in flow that passes by the sensor. The flow sensor may be able to detect disruption in flow of a fluid, e.g., disruption of blood flow in a blood vessel. The flow sensor may have single or multiple membranes.

In one embodiment, the sensor is a pressure sensor. The present sensor is able to measure the pressure, and measure and/or detect changes in the pressure, in the vicinity of the sensor when located within a host. The pressure sensor may be used to measure the pressure present within a vessel of a host, e.g., a blood vessel. The pressure sensor may be used to detect and/or measure variation in pressure that is present within a vessel of a host. The pressure sensor may have single or multiple membranes.

In one embodiment, the sensor is an ultrasonic sensor which obtains information via an ultrasonic transducer. The ultrasonic transducer may be configured to receive and/or transmit ultrasonic signals. An ultrasonic sensor may be used for measuring fluid flow or detection of large particulate material, where large refers to an aggregation of more than one red blood cell (RBC), white blood cell (WBC), and/or platelet. In some embodiments, an ultrasonic transducer may be disposed in the implantable reporting processor along with ultrasonic sensors to obtain ultrasonic imaging of a desired region of the body, e.g., the region of the body near the implanted medical device.

In one embodiment, the sensor is an acoustic sensor. Optionally, the acoustic sensor has a substantially flat sensitivity between about 20 Hz and about 20 kHz.

In one embodiment, the sensor is an IMU, more completely named an inertial measurement unit. An IMU is an electronic device that measures and reports a body's specific force, angular rate, and sometimes the magnetic field surrounding the body, using a combination of accelerometers and gyroscopes.

The sensor may be associated with one or more other components of the sensing attachment, which may be referred to as auxiliary components, where together these provide an implantable reporting processor (IRP). An example sensor and auxiliary components may be bundled together and include a sensor, a battery, an inertial measurement unit (IMU); pedometer, radio and an antennae. The components may be welded together and hermetically sealed. Coating, such as anticoagulation coating, can be added to protect the one or more components (such as, the sensor). The coating can be applied to the surface (such as, external surface) of the one or more components (and/or to a housing). In one embodiment, the auxiliary components comprise one or more of a hermetically sealed battery, microprocessor, memory, and radio with a least one antenna. The memory may have the capacity to store data generated over a 1 to 90 day period. In one embodiment, the sensor is a wired sensor. In this case, the sensor is wired to a power supply, e.g., a battery. Optionally, the wired sensor is a capacitive pressure sensor. In one embodiment, the sensor in a wireless sensor. When the sensor is a wireless sensor, the power supply for the sensor is not physically connected to the sensor. The power supply can be placed near the sensor, e.g., it may be implanted into the abdomen of the patient receiving the graft. The power supply may be of the type used to power a pacemaker or an implantable defibrillator, which is a known type of power supply. The power supply will be physically connected to at least one antennae that is used to transmit power wirelessly to the sensor. The power supply may also be physically connected to an antennae that is used to receive information from the sensor. Thus, in one embodiment, the present disclosure provides a wireless sensor integrated with a medical device.

FIG. 8 is a diagram of an implantable reporting processor (IRP) 103 that may be associated with a sensing attachment (not shown in FIG. 8). As illustrated and described herein, the IRP 103 includes electronic circuitry. The components of the implantable reporting processor 103 include a power supply 112, an electronics assembly 110 having various electronic circuitry powered by the power supply, and one or more of components of a communication interface, e.g., an antenna 130, electrodes 131, 133, and an acoustic transducer 135. The acoustic transducer can include one or more microphones. The circuitry of the electronics assembly 110 may include a fuse 114, switches 116, 118, a clock generator and power management unit 120, one or more sensors 122, a memory 124, a controller 132, and communication circuitry 125. The communication circuitry 125 may include one or more of a radio frequency (RF) transceiver 126 and a filter 128, that couple with the antenna 130; tissue conductive communication circuitry 137 that coupled with a pair of electrodes 131, 133; or data-over-sound circuitry 139 that couples with an acoustic transducer 135. Examples of some or all of these components are described elsewhere in this application or in U.S. Ser. No. 16/084,544, which is incorporated by reference in all jurisdictions which allow incorporation by reference.

Referring to FIG. 8, in an IRP of a sensing attachment, a sensor 122 may be located on a printed circuit board of the electronics assembly 110, or in or on another structure of the sensing attachment separate from the implantable reporting processor 103, but electrically coupled to the electronics assembly. Within certain embodiments a sensor 122 may comprise a processor or may couple to a processor located on a printed circuit board of the electronics assembly 110. In other embodiments, the sensor can be a wireless sensor. Within further embodiments one or more (including all) of the sensors can have a Unique Sensor Identification number (“USI”) which specifically identifies the sensor.

Referring to FIG. 8, the power supply 112 is configured to generate a regulated supply signal in an approximate range of 1-24 Volts (V) to power the components of the implantable reporting processor 103. The power supply 112 may include one or more of a battery, a rechargeable power device (e.g., a rechargeable battery or a super capacitor), and an energy harvester.

In one embodiment, the power supply 112 may be any suitable battery, such as a Lithium Carbon Monofluoride (LiCFx) battery, or other storage cell configured to store energy for powering components of the electronics assembly 110 for an expected lifetime (e.g., 5-25+ years) of the sensing attachment.

In one embodiment, the power supply 112 may be a rechargeable power device, such as a lithium-ion battery or a supercapacitor. In this case, the power supply 112 includes additional components for charging the power supply by an external recharge unit (for instance, utilizing wireless power charging as described herein). These additional components include a power coil configured to generate a voltage and current in response to a near magnetic field generated by an external recharge unit.

In one embodiment, the power supply 112 may be an energy harvester. The energy harvester is configured to convert an environmental stimulus into an energy for charging a rechargeable power device. For example, the harvester may convert, into a battery-charging electrical current or voltage or a supercapacitor-charging, one or more of body heat from the subject in which the implantable reporting processor 103 is implanted, kinetic energy generated by the subject's movement, changes in pressure (e.g., barometric pressure or pressure within the subject, such as the subject's blood pressure), energy generated by an electrochemical reaction within the subject's body, energy generated by radio-frequency (RF) fields, and light.

Still referring to FIG. 8, the fuse 114 can be any suitable fuse (e.g., permanent) or circuit breaker (e.g., resettable) configured to prevent the power supply 112, or a current flowing from the power supply, from injuring the patient and damaging one or more components of the electronics assembly 110. For example, the fuse 114 can be configured to prevent the power supply 112 from generating enough heat to burn the patient, to damage the electronics assembly 110 or to damage structural components of the sensing attachment.

In FIG. 8, the switch 116 is configured to couple the power supply 112 to, or to uncouple the power supply from, the one or more sensors 122 in response to a control signal from the controller 132. For example, the controller 132 may be configured to generate the control signal having an open state that causes the switch 116 to open, and, therefore, to uncouple power from the one or more sensors 122, during a sleep mode or other low-power mode to save power, and, therefore, to extend the life of the power supply 112. Likewise, the controller 132 also may be configured to generate the control signal having a closed state that causes the switch 116 to close, and therefore, to couple power to the one or more sensors 122, upon “awakening” from a sleep mode or otherwise exiting another low-power mode. Such a low-power mode may be for only the one or more sensors 122 or for the sensors and one or more other components of the electronics assembly 110.

The switch 118 is configured to couple the power supply 112 to, or to uncouple the power supply from, the memory 124 in response to a control signal from the controller 132. For example, the controller 132 may be configured to generate the control signal having an open state that causes the switch 118 to open, and, therefore, to uncouple power from the memory 124, during a sleep mode or other low-power mode to save power, and, therefore, to extend the life of the power supply 112. Likewise, the controller 132 also may be configured to generate the control signal having a closed state that causes the switch 118 to close, and therefore, to couple power to the memory 124, upon “awakening” from a sleep mode or otherwise exiting another low-power mode. Such a low-power mode may be for only the memory 124 or for the memory and one or more other components of the electronics assembly 110.

As shown in FIG. 8, the clock and power management unit 120 can be configured to generate a clock signal for one or more of the other components of the electronics assembly 110, and can be configured to generate periodic commands or other signals (e.g., interrupt requests) in response to which the controller 132 causes one or more components of the implantable reporting processor 103 to enter or to exit a sleep, or other low-power, mode. The clock and power management unit 120 also can be configured to regulate the voltage from the power supply 112, and to provide a regulate power-supply voltage to some or all of the other components of the electronics assembly 110.

In FIG. 8, the memory 124 may include volatile memory and non-volatile memory. For example, the volatile memory may be configured to store the operating system and one or more applications executed by the controller 132. The non-volatile memory may be configured to store configuration information for the implantable reporting processor 103 and to store data written by the controller 132, and to provide data in response to a read command from the controller.

In one aspect, the implantable reporting processor 103 includes a communication interface which facilitates communication between the sensing attachment (not shown in FIG. 8) and another device. The other device may be, for example, an external device, e.g., a base station, that is located outside of or away from the patient who has received the sensing attachment, or it may be an internal device that is located in the patient who has received the sensing attachment. In either case, communication between an implanted sensing attachment and another device, whether internal or external, is referred to as intra-body communication. One or more of intra-body communication may be enabled by the communication interface of the implantable reporting processor 103. Exemplary modes of intra-body communication include: 1) RF telemetry communication, 2) tissue conductive communication, e.g., galvanic coupling communication, and 3) data-over-sound communication, e.g., ultrasound or acoustic communication.

The communication interface includes communication circuitry 125 that is generally, but not necessarily, associated with the electronics assembly 110 of the implantable reporting processor 103. The communication circuitry 125 may include any hardware, firmware, software or any combination thereof suitable for enabling one or more modes of intra-body communication. To this end, the communication circuitry 125 may include, for example, voltage regulators, current generators, oscillators, or circuitry for generating a signal, resistors, capacitors, inductors, and other filtering circuitry for processing received signals, as well as circuitry for modulating and/or demodulating a signal according to a communication protocol.

Depending on the mode of intra-body communication, the communication circuitry 125 may also include transistors or other switching circuitry for selectively coupling transmitted signals to or receiving signals from a desired transceiver, such as an antenna 130 (which may be used for electromagnetic communication, e.g., RF telemetry communication) or electrodes 131, 133 (which may be used for tissue conductive communication) or an acoustic transducer 135 (which may be used for data-over-sound communication). Under the control of the controller 132, communication circuitry 125 may receive downlink communication signals from, as well as send uplink communication signals to, an external device or another implanted device. In addition, communication circuitry 125 may communicate with a networked computing device via an external device and a computer network, such as the Medtronic CareLink® Network developed by Medtronic, plc, of Dublin, Ireland.

Additional details on each of the RF telemetry communication, tissue conductive communication, and data-over-sound communication modes of intra-body communication follow, with reference to FIG. 8.

In one embodiment, the communication interface includes an RF telemetry mode of intra-body communication which is enabled by an RF communication interface that includes an antenna 130 and RF telemetry circuitry, e.g., an RF transceiver 126 and optionally a filter 128. The RF transceiver 126 can be configured to allow the controller 132 (and optionally the fuse 114) to communicate with another implanted medical device (not shown in FIG. 8), with a base station (not shown in FIG. 8) configured for use with the sensing attachment, or with another remote electronic device. For example, the RF transceiver 126 can be any suitable type of transceiver (e.g., Bluetooth, Bluetooth Low Energy (BTLE), and WiFi®), can be configured for operation according to any suitable protocol (e.g., MICS, ISM, Bluetooth, Bluetooth Low Energy (BTLE), and WiFi®), and can be configured for operation in a frequency band that is within a range of 1 MHz-5.4 GHz, or that is within any other suitable range.

The filter 128 can be any suitable bandpass filter, such as a surface acoustic wave (SAW) filter or a bulk acoustic wave (BAW) filter. The antenna 130 can be any antenna suitable for the frequency band in which the RF transceiver 126 generates signals for transmission by the antenna, and for the frequency band in which a base station (not shown in FIG. 8) generates signals for reception by the antenna.

In one embodiment, the communication interface can include a tissue conductive communication (TCC) mode of intra-body communication which is enabled by a TCC interface that includes TCC circuitry 137 and a pair of electrodes 131, 133. The TCC interface allows the controller 132 to communicate with another device having a same TCC interface as the implantable reporting processor 103. The other device may be an implanted medical device (not shown in FIG. 8), or a base station (not shown in FIG. 8) configured for use with the sensing attachment (not shown in FIG. 8).

Tissue conductive communication relies on the ion content of body tissue of a patient within which the sensing attachment has been implanted, and is thus frequently referred to as galvanic communication. The ion content of the body tissue provides an electrical communication medium over which to send and receive information to and from the sensing attachment. To communicate in a transmit mode, the TCC circuitry 137 applies a voltage across the electrodes 131, 1033 to cause current to flow between the electrodes and a corresponding electrical signal to propagate through the body tissue. The propagating current may be detected by a receiving device (not shown in FIG. 8) by measuring the voltage generated between two electrodes. To communicate in a receive mode, the TCC circuitry 137 measures voltage across the electrodes 131, 133.

When tissue conductive communication is employed to facilitate communication, the sensing attachment and the other device that receives and/or sends information to the sensing attachment, have associated hardware, firmware, software or any combination thereof suitable for providing such communication. TCC transmission and associated hardware, firmware, software have been described and may be included in the intelligent implantable device of the present disclosure. See, e.g., U.S. Patent Publication Nos. US2016213939, US2018207429, US2019160290, US2019160291, US2019160292, US2019184181. For example, in one aspect, the TCC circuitry 137 may be coupled to one or more electrodes 131, 133, and configured with circuitry that enables the TTC interface to switch between a transmit mode during which TCC signals are transmitted, and a receive mode during which TCC signals are received from another similarly configured device.

In one embodiment, the communication interface includes a data-over-sound mode of intra-body communication which is enabled by a data-over-sound communication interface that includes data-over-sound circuitry 139 and at least one acoustic transducer 135. The data-over-sound communication interface allows the controller 132 to communicate with another device having a same data-over-sound communication interface as the implantable reporting processor 103. The other device may be an implanted medical device (not shown in FIG. 8), or a base station (not shown in FIG. 8) configured for use with the sensing attachment.

Data-over-sound communication relies on the body of a patient within which the sensing attachment has been implanted to provide a medium over which to send and receive information to and from the implanted sensing attachment. To communicate in a transmit mode, the data-over-sound circuitry 139 outputs a mechanical soundwave through the acoustic transducer 135 that propagates through the body. The soundwave may be in the ultrasound range, e.g., above 20 KHz. The propagating mechanical soundwave may be detected by a receiving device (not shown in FIG. 8) having an acoustic transducer. To communicate in a receive mode, the data-over-sound circuitry 139 receives and measures soundwaves.

When data-over-sound communication is employed to facilitate communication, the implanted sensing attachment 1002 and the other device that receives and/or sends information to the implanted sensing attachment, have associated hardware, firmware, software or any combination thereof suitable for providing such communication. Data-over-sound communication transmission and associated hardware, firmware, software have been described and may be included in the sensing attachment of the present disclosure. See, e.g., U.S. Pat. No. 7,489,967 and U.S. Patent Publication Nos. U520100249882 and US20130033966. For example, in one aspect, the data-over-sound circuitry 139 may be coupled to an acoustic transducer 135 and configured with circuitry that enables the data-over-sound communication interface to switch between a transmit mode during which ultrasound signals are transmitted, and a receive mode during which ultrasound signals are received from another similarly configured device.

With reference to FIG. 8, the controller 132, which can be any suitable microcontroller or microprocessor, is configured to control the configuration and operation of one or more of the other components of the electronics assembly 110. For example, the controller 132 is configured to control the one or more sensors 122 to sense relevant measurement data, to store the measurement data generated by the one or more sensors in a memory component. The controller 132 is also configured to generate message for communication over one or more types of communication interfaces. For example, in the case of RF telemetry communication, the controller 132 generates messages that include the stored data as a payload, packetizes the messages, and provides the message packets to the RF transceiver 126 for transmission to the base station (not shown in FIG. 8). The controller 132 also can be configured to execute commands received from a base station (not shown in FIG. 8) via a communication interface, e.g., the antenna 130, filter 128, and RF transceiver 126. For example, the controller 132 can be configured to receive configuration data from the base station, and to provide the configuration data to the component of the electronics assembly 110 to which the base station directed the configuration data. If the base station directed the configuration data to the controller 132, then the controller is configured to configure itself in response to the configuration data.

Still referring to FIG. 8, operation of an implantable reporting processor (IRP) 1003 is described in relation to an implanted sensing attachment in which the IRP is disposed, or with which the IRP is otherwise associated.

The fuse 114, which is normally electrical closed, is configured to open electrically in response to an event that can injure the patient in which the implantable reporting processor 103 resides, or damage the power supply 112 of the implantable circuit, if the event persists for more than a safe length of time. An event in response to which the fuse 114 can open electrically includes an overcurrent condition, an overvoltage condition, an overtemperature condition, an over-current-time condition, and over-voltage-time condition, and an over-temperature-time condition. An overcurrent condition occurs in response to a current through the fuse 114 exceeding an overcurrent threshold. Likewise, an overvoltage condition occurs in response to a voltage across the fuse 114 exceeding an overvoltage threshold, and an overtemperature condition occurs in response to a temperature of the fuse exceeding a temperature threshold. An over-current-time condition occurs in response to an integration of a current through the fuse 114 over a measurement time window (e.g., ten seconds) exceeding a current-time threshold, where the window can “slide” forward in time such that the window always extends from the present time back the length, in units of time, of the window. Alternatively, an over-current-time condition occurs if the current through the fuse 114 exceeds an overcurrent threshold for more than a threshold time. Similarly, an over-voltage-time condition occurs in response to an integration of a voltage across the fuse 114 over a measurement time window, and an over-temperature-time condition occurs in response to an integration of a temperature of the fuse over a measurement time window. Alternatively, an over-voltage-time condition occurs if the voltage across the fuse 114 exceeds an overvoltage threshold for more than a threshold time, and an over-temperature-time condition occurs if a temperature associated with the fuse 114, power supply 112, or electronics assembly 110 exceeds an overtemperature threshold for more than a threshold time. But even if the fuse 114 opens, thus uncoupling power from the electronics assembly 110, the mechanical and structural components of the intelligent implant (not shown in FIG. 8) are still fully operational.

The controller 132 can be configured to cause the one or more sensors 122 to make a detection or measurement, for example a pressure or fluid flow detection or measurement, to determine if the measurement is a qualified or valid measurement, to store the data representative of a valid measurement, and to cause the RF transceiver 126 to transmit the stored data to a base station or other source external to the prosthesis.

Still referring to FIG. 8, in response to being polled by a base station (not shown in FIG. 8) or by another device external to the implanted device, the controller 132 can generate messages having payloads and headers. The payloads can include the stored samples of the signals that the one or more sensors 122 generated, and the headers can include the sample partitions in the payload, a time stamp indicating the time at which the sensor 122 acquired the samples, an identifier (e.g., serial number) of the implantable prosthesis, and a patient identifier (e.g., a number or name).

The controller 132 can generate data packets that include the messages according to a data-packetizing protocol. Each packet can also include a packet header that includes, for example, a sequence number of the packet so that the receiving device can order the packets properly even if the packets are transmitted or received out of order.

The controller 132 can encrypt some or all parts of each of the data packets, for example, according to an encryption algorithm, and error encodes the encrypted data packets. For example, the controller 132 encrypts at least the sensing attachment and patient identifiers to render the data packets compliant with the Health Insurance Portability and Accountability Act (HIPAA).

The controller 132 can provide the encrypted and error-encoded data packets to the RF transceiver 126, which, via the filter 128 and antenna 130, transmits the data packets to a destination, such as the home base station 104, external to the sensing attachment. The RF transceiver 126 can transmit the data packets according to any suitable data-packet-transmission protocol.

Still referring to FIG. 8, alternate embodiments of the implantable reporting processor 103 are contemplated. For example, the RF transceiver can perform encryption or error encoding instead of, or complementary to, the controller 132. Furthermore, one or both of the switches 116 and 118 can be omitted from the electronics assembly 110. Moreover, the implantable reporting processor 103 can include components other than those described herein and can omit one or more of the components described herein.

Within certain embodiments, the sensing attachment is provided with a specific unique device identifying number (“UDI”), and within further embodiments, each of the sensors on the sensing attachment each have either a specific unique sensor identification number (“USI”), or a unique group identification number (“UGI”, e.g., an identification number that identifies the sensor as one of a group of sensors such as a fluid pressure sensor, contact sensor, position sensor, pulse pressure sensor, blood volume sensor, blood flow sensor, blood chemistry sensor, blood metabolic sensor, and/or mechanical stress sensor). Within yet further embodiments, the USI is specifically associated with a position on the sensing attachment.

In one embodiment, the sensor is attached either directly or indirectly to the body of the sensing attachment. For example, the sensor may be contained within a housing, where the housing is fixed in place on the body, thereby securing the sensor in place on the sensing attachment. In one embodiment, the housing is not a hermetically sealed housing. In one embodiment, the housing is a hermetically sealed housing which does not interfere with the operation of the sensor and the auxiliary components.

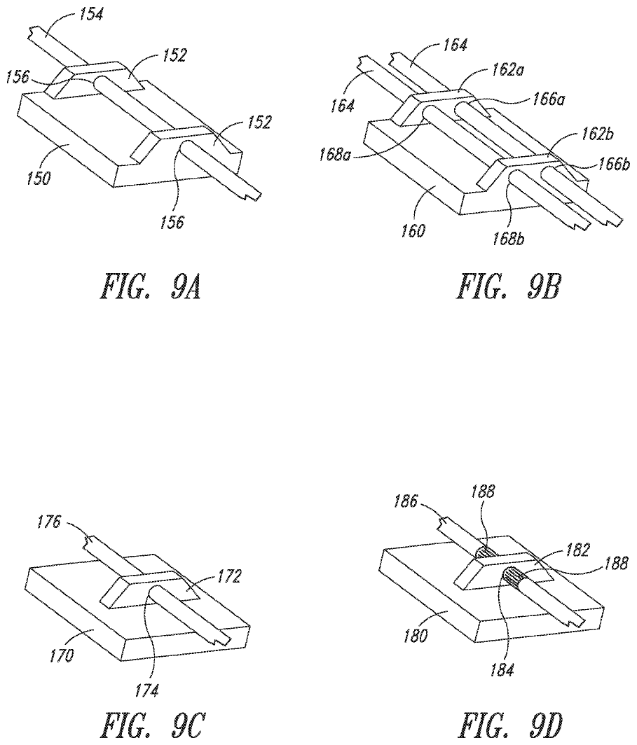

FIG. 9A shows an approach according to the present disclosure for attaching a sensor to a support in the form of a filament. In FIG. 9A, a sensor housing 150 is shown with two extensions 152, each extension 152 having one hole. A piece of the support filament 154, which may be a wire strut support such as shown in FIG. 1, 2A, 2B, 2C, 3A, 3B, 4B, 5A or 5B, is threaded through a hole in the extension. The hole is filled by the wire strut 154, but the location of the hole is shown as feature 156. In this way, the sensor housing, and according the sensor itself, is attached to a support to provide a construct of a body and a sensor of the present disclosure.

FIG. 9B shows another approach according to the present disclosure for attaching a sensor to a support. In FIG. 9B, a sensor housing 160 is shown with two extensions 162a and 162b, each extension 162a and 162b having two holes. A piece of the support filament 164, which may be a wire strut support such as shown in FIG. 1, 2A, 2B, 2C, 3A, 3B, 4B, 5A or 5B, is threaded through one hole in each extension, e.g., hole 166a in extension 162a and hole 166b in extension 166b, while another wire strut 164 is threaded through hole 168a in extension 162a and hole 168b in extension 166b. In this way, the sensor housing, and according the sensor itself, is attached to a support to provide a construct of a body and a sensor of the present disclosure.

FIG. 9C shows yet another approach according to the present disclosure for attaching a sensor to a support. In FIG. 9C, a sensor housing 170 is shown with one extension 172, where extension 172 has one hole 174. A piece of the support monofilament 176, which may be a wire strut support as shown in FIG. 1, 2A, 2B, 2C, 3A, 3B, 4B, 5A or 5B, is threaded through the hole 174 in the extension. In this way, the sensor housing, and according the sensor itself, is attached to a support to provide a construct of a body and a sensor of the present disclosure.

FIG. 9D shows a further approach according to the present disclosure for attaching a sensor to a support. In FIG. 9D, a sensor housing 180 is shown with one extension 182, where extension 182 has one hole 184. A piece of the monofilament support 186, which may be a wire strut support as shown in FIG. 1, 2A, 2B, 2C, 3A, 3B, 4B, 5A or 5B, is threaded through the hole 184 in the extension. In addition, crimping is applied at locations 188 on either side of the extension 182, where the crimping assists in attaching the sensor to the monofilament support is a fixed location. In this way, the sensor housing, and according the sensor itself, is attached to a support to provide a construct of a body and a sensor the present disclosure.

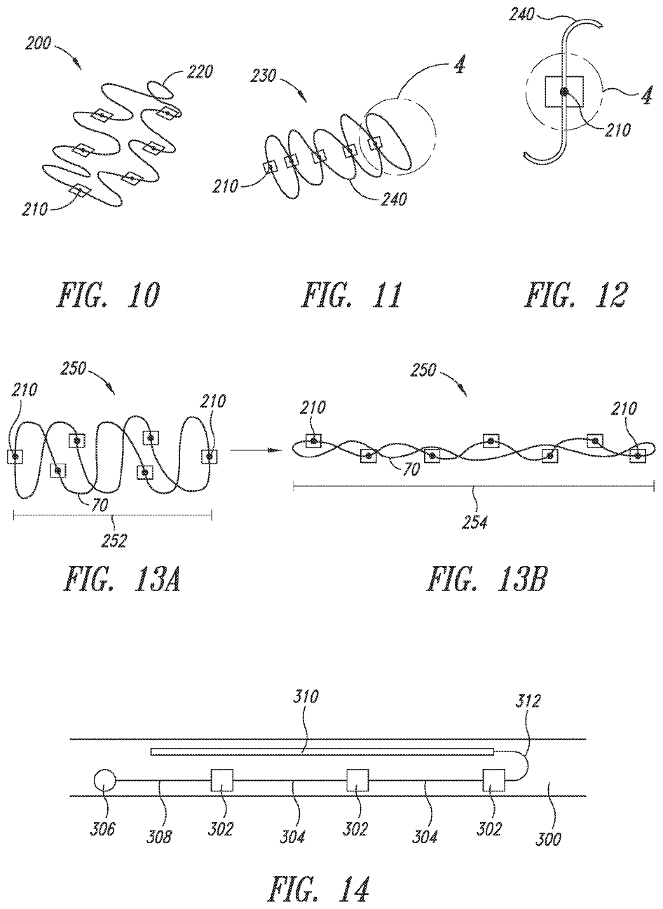

FIGS. 10, 11 and 12 illustrate constructs wherein a body and a sensor within a housing have been combined. Although the sensor may be contained within a housing such as shown in FIGS. 9A, 9B, 9D and 9D, the sensor may alternatively be combined with a body using other fixation techniques, such as chip stacking and bonding attachment consisting of low temperature or non-damaging temperature processes. Ambient humidity, super saturated humidity or non-humidity bonding processes may also be employed to secure a sensor to a body of a sensing attachment.

FIG. 10 shows a construct 200 comprising a support strut 220 in the form of a wire ring, on which are located a plurality of sensors 210. This construct 200 may be referred to herein as a CSR2. The CRS2 include wireless capacitive pressure sensors and may also include accelerometers if being used internally to a stent graft. The sensors are mounted onto at least one sinusoidal strut 220 that can be expanded to conform to the available intravascular geometry. The construct 200 may be secured around the stent graft via hoop stress against the mating surface. The construct 200 thus abuts and is held in place next to the medical device, but does not mechanically attach to the medical device. The sensor shape and dimensions are preferably minimized so as to present a minimal cross sectional area to blood flow thereby reducing the risk of hemolysis and thrombus formation. The construct may comprise a plurality of struts 220 in order to provide additional stability for orientation of the sensors and/or to provide additional compression against the lumen of an endograft or arterial vessel. The latter may be necessary to obviate migration of the CRS2 when subjected to forces within the vascular system. Each CRS2 is designed to cover a minimum and maximum range of expansion to cover a range of vessel diameters. For example, one CRS2 could cover a diametric range of 3 mm to 6 mm whereas the next larger size may cover from 5 mm to 10 mm. Such schemes can be used to cover vessel lumen diameters commonly found in the cardiovascular system or in aneurysmal geometries.

FIG. 11 is another view of a construct 230 comprising a wire strut support 240 on which are attached a plurality of sensors 210.

FIG. 12 is an expanded view of a portion 4 of wire of support 240 from FIG. 11, whereupon a sensor 210 is attached.

The sensor may be attached to each rail at either a single point or multiple points via interconnecting holes integrated into the sensor housing (FIG. 10) and/or be welded or glued in place. Alternatively, they may be fixed in place with crimping, glue, or other attached stops that hold the sensor in place (FIG. 11) along a stent rail.

The placement of the sensors on the body should not interfere with the ability of the body to have one or more of compliance, elasticity, or has shape memory, as described herein.

FIG. 13A and FIG. 13B show a body 70 as illustrated in FIG. 7A, having sensors 210 attached thereto to provide a construct 250. The construct 250 may comprise a wire rail in the compacted geometry 252 or in an extended geometry 254, where in each case the rail is attached to a plurality of sensor 210. The extended form is useful if using a laparoscopic or open surgical approach in which the CRS2 is placed external to a vessel/conduit, otherwise the CRS2 may be of an open or compacted configuration so as to fit around the vessel. In this case, the ring may be left open or compressed to form a closed loop with the aid of external fixation devices such as clips, glue, or other crimping technology known to those skilled in the art.

In the event that the body has a portion that will not change significantly in size or shape during use, the sensor and auxiliary components may be attached to this portion of the body. For example, as shown in FIG. 14, the body illustrated in FIG. 6 has a spline 300 (shown as feature 63 in FIG. 6) that maintains a constant dimension during use. Onto this spline 300 may be placed a sensor 302 (three sensors 302 being shown in FIG. 14) which may be in wired communication via wire 304. A power supply 306 may likewise be fixed to the spline 300 to provide power to the sensor 302 via a wire 308. Also shown in FIG. 14 is an antenna 310 to provide communication between the outside world and the implanted sensing attachment. The antenna 310 may be in wired communication with the sensor 302 and/or power supply 306 via wire conduit 312. The antenna 310 may be fixed to the spline 300 in the longitudinal and/or radial axes, or it may be attached only to a wire 312, in which case the antenna is free to move away from the sensor attachment. The attachments may be made by, e.g., welding or gluing.

Fabrication of the body may be effected by standard methods known in the art. For example, methods for making objects from nitinol are well known and may be utilized to make the body of the present disclosure. For example, a hollow filament make from nitinol may be cut multiple times to provide a body comprising a plurality of cuts. This body may be secured to a mandrel so that it adopts a desired shape and size, which is the shape and size that is ultimately desired when the sensing attachment is associated with a medical device. While attached to the mandrel, the body is taken to high temperature, e.g., 550° C. for a time and then cooled, and the mandrel removed, whereupon the body maintains the size and shape it had while secured to the mandrel, referred to herein as its natural state. The body may then be cooled, often referred to as super-cooled, and compressed to from a smaller volume state, i.e., a compressed state. When this compressed state of the body is brought to room temperature of about 25° C., it maintains its compressed state. However, when it is heated further, to body temperature of about 37° C., it will spontaneously decompress and return to its natural state. The compressed state may be further compressed when the body, as part of a sensing attachment, is placed within a delivery catheter, where this further compression is sometimes referred to as crimping. Upon being released from the delivery catheter at body temperature of about 37 C, the sensing attachment will decompress, going to its natural state. This or similar technology may be used for other metallic bodies, such as prepared from platinum or alloys of platinum and iridium.

In one embodiment, the sensing attachment is associated with, or in combination with, or intended to be associated with, a medical device. The medical device of the present disclosure is a graft or a stent graft. Representative stent grafts to which a sensing attachment of the present disclosure may be associated include vascular (e.g., endovascular) stent grafts, gastro-intestinal (e.g., esophageal) stent grafts, and urinary stent grafts. A stent graft is a tube made of a thin metal mesh (the stent), covered with a thin layer of fabric (the graft).

Unless the context indicates otherwise, reference to a graft does not refer to a stent graft, but rather refers to a graft without a stent. The graft is a tubular structure which has a lumen and a surrounding wall, where the wall may be referred to as a side wall. The wall has an inner surface, which faces the lumen, i.e., an adluminal surface, and also has an outer or exterior surface which faces away from the lumen, i.e., an abluminal surface. In one embodiment the graft is a vascular graft. In one embodiment, the graft may be made from a synthetic material, such as polyester fabric. Expanded polytetrafluoroethylene, Dacron® or other polyethylene terephthalate, and polyurethane are currently used to make synthetic vascular grafts, and may be used to make a graft of the present disclosure. In one embodiment, the graft has only two holes: a hole to allow fluid into the graft and a hole to allow fluid to exit the graft, where the graft provides a conduit for the fluid. When the graft is intended for vascular grafting, i.e., is a synthetic vascular grant, in one embodiment the graft has a diameter of greater than 8 mm, e.g., 8-10 mm, and may be used in, e.g., aortoiliac substitute, or may have a diameter of about 6-8 mm and may be used in, e.g., carotid or common femoral artery replacements. https://www.ncbi.nlm.nih.gov/pnc/articles/PMC4753638/⋅B19

In one embodiment, the medical device is suitable for endovascular treatment or repair. For example, the graft or stent graft may be suitable for treating or repairing an endovascular aneurysm. In general, aneurysms are a bulging and weakness in the wall of the aorta, but can occur anywhere in the human arterial vascular system. The aorta is the largest blood vessel in the body, and it delivers blood from the heart to the rest of the body. Most aortic aneurysms occur in the abdominal aorta (abdominal aortic aneurysms or AAA), but they can also occur in the thoracic aorta (thoracic aortic aneurysms or TAA) or in both the thoracic and abdominal segments of the aorta. Other examples of aneurysms that may be treated or repaired by a stent graft of the present disclosure include a femoral aneurysm, which is a bulging and weakness in the wall of the femoral artery (located in the thigh), an iliac aneurysm which occurs upon weakness in the wall of the iliac artery (a group of arteries located in the pelvis), a popliteal aneurysm which occurs when there is weakness in the wall of the popliteal artery which supplies blood to the knee joint, thigh and calf, a subclavian aneurysm which is weakness or bulging in the wall of the subclavian artery (located below the collarbone), a supra-renal aneurysm of the aorta located above the kidneys, and a visceral aneurysm which occurs within abdominal cavity arteries and includes the celiac artery, the superior mesenteric artery, the inferior mesenteric artery, the hepatic artery, the splenic artery and the renal arteries.

For example, the stent graft may be used for treating or repairing an abdominal aortic aneurysm (AAA), where such a device sometimes referred to as an AAA endovascular repair graft. An endovascular repair may be done to treat an aneurysm located below the arteries to the kidney. Using a needle puncture or small incision in one or both of the patient's groin arteries, a thin tube (catheter) is inserted and advanced to the aneurysm site, typically guided by X-ray images. Then a guide wire and an expandable stent graft (a fabric-covered wire frame) are advanced through the thin tube. After being located in the correct position, the stent graft is allowed to expand within the artery. The wire frame pushes against the healthy portion of the aorta to seal the device in place. Once in place, blood flows through the stent graft and does not have access to the aneurysm. The procedure is efficiently done, using taking 1.5-3.5 hours, and most patients leave the hospital in 1-5 days.

In some situations, an aneurysm affects one or more of the important arteries that branch off the aorta. In this situation, a different type of graft is placed, called a fenestrated graft or a fenestrated stent graft. A fenestrated graft gets its name from tiny cutouts that allow the graft to flex and align with the branching of arteries, and also be modified to accommodate your specific anatomy. Implantation of a fenestrated graft usually takes from 3-8 hours. As used herein, a stent graft refers to fenestrated grafts as well as grafts that do not contain the tiny cutouts. In one embodiment, the medical device is suitable for treating or repairing an abdominal aortic aneurysm (AAA).

As another example, the stent graft may be used for treating or repairing a thoracic aortic aneurysm (TAA). The procedure whereby a TAA is repaired with a stent graft is typically referred to as a thoracic endovascular aneurysm repair (TEVAR). Thoracic aortic aneurysms are subdivided into three categories, which are based on their location: aortic arch, ascending aortic, and descending thoracic aneurysms. The TAA may be a thoraco-abdominal aortic aneurysm, which is a bulging and weakness in the wall of the aorta that extends from the chest into the abdomen. Using a surgical method, a thoracic aneurysm is replaced with a synthetic graft. In the TEVAR procedure, a thoracic stent graft is inserted into the aneurysm through small incisions in the groin. In one embodiment, the medical device of the present disclosure is suitable for treating or repairing a thoracic aortic aneurysm (TAA). In one embodiment, the medical device is a stent graft for TEVAR. In another embodiment, the medical device is a graft for the surgical treatment of a TAA as mentioned above.

Example grafts and stent grafts suitable for use as a medical device according to the present disclosure are provided in CN105832332; CN107440816; CN202207217U; CN204049932U; CN207085001U; GB201517623; GB201519983; GB2515731; GB2517689; RE39,335; US20100324650; US20120239131; US20120271399; US20130073027; US20130261731; US20140018902; US20140052231; US20140121761; US20140135898; US20140277335; US20150088244; US20150127086; US20150202065; US20150250626; US20150250629; US20150335290; US20160038085; US20160100969; US20160113796; US20160120638; US20160184076; US20160184077; US20160184078; US20160250395; US20160302950; US20170000630; US20170007391; US20170135806; US20170209254; US20170231749; US20170231751; US20170239035; US20170281331; US20170281332; US20170290654; US20170319359; US20170340462; US20170360993; US20180071076; U.S. Pat. Nos. 7,290,494; 8,118,856; 8,728,145; 8,870,938; 8,888,837; 8,945,200; 8,945,203; 8,951,298; 8,998,972; 9,101,457; 9,168,162; 9,345,594; 9,468,517; 9,486,341; 9,603,697; 9,629,705; 9,687,366; 9,808,334; 9,811,613; 9,833,341; 9,839,540; 9,861,503; 9,907,642; 9,918,825; 9,925,032; WO11158045; WO13130390; WO15047094; WO16123676; WO17060738; WO17064484; WO2013167491; WO2013167492; WO2013167493; WO2016008944; WO2017114879; WO2017134198; and WO2017187174.

To perform endovascular stent graft implantations, a surgeon will insert the stent graft into the blood vessel at the location of the aneurism in order to reduce the pressure on the blood vessel walls at the site of the aneurism. Such stent grafts have been used widely for many years and are well known. Unfortunately, such endovascular stent grafts are sometimes subject to failure. One failure that may occur is leaking of blood into the aneurysm sac; a condition referred to as an endoleak, of which there are 5 different types. A Type I Endoleak occurs when blood flows between the stent graft and the blood vessel wall; typically at the proximal (often renal) or distal (often iliac) end of the graft. This complication may also occur as a result of movement of the graft away from the desired location, sometimes called migration. Type II Endoleaks occur when blood flows backwards (retrograde) into the aneurysm sac from arteries originating from the aneurysm sac itself (typically the lumbar, testicular or inferior mesenteric arteries). Type III endoleaks occur when blood leaks between the junction sites of “articulated” or “segmented” stent grafts; these multi-component stent grafts are inserted as separate segments which are then assembled inside the artery into their final configuration. Detecting and confirming accurate assembly and fluid-tight contact between the different segments is difficult and current verification methods of correct assembly are suboptimal. Type IV Endoleaks occur when cracks or defects develop in the stent graft fabric and blood is able to leak directly through the graft material. Lastly, Type V Endoleaks are leakage of blood into the aneurysm sac of an unknown origin. Regardless of their cause, endoleaks are frequently a medical emergency and early detection, characterization and monitoring of them is an important unmet medical need.

Other complications of stent graft placement include partial blockage of the blood flowing through the stent graft (stenosis), detachment, rupture, fabric wear (durability), kinking, malpositioning, and systemic cardiovascular disorders (myocardial infarction, congestive heart failure, arrhythmias, renal failure). Presently, detecting such complications prior to their occurrence or early in their development is difficult or, in many cases, impossible. The present disclosure addresses these problems by associating a sensing attachment with a convention implanted stent graft, or a convention implanted graft.

In one aspect, the medical device is an implantable medical device, where an example implantable medical device is a stent graft which is implanted into a patient during a surgical procedure to treat an aneurysm. Aneurysm refers to an undesired dilation of a blood vessel, e.g., a dilation of at least 1.5 times above the vessel's normal diameter. The dilated vessel may have a bulge known as an aneurysmal sac that can weaken vessel walls and eventually rupture. Aneurysms are most common in the arteries at the base of the brain (i.e., the Circle of Willis) and in the largest artery in the human body, i.e., the aorta. The abdominal aorta, spanning from the diaphragm to the aortoiliac bifurcation, is the most common site for aortic aneurysms. Such abdominal aortic aneurysms (AAAs) typically occur between the renal and iliac arteries.

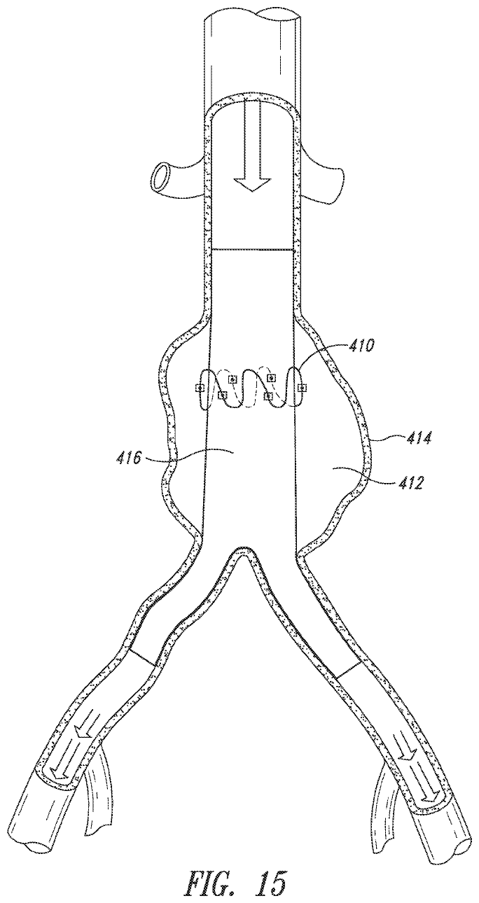

The sensing attachment may be associated at various locations of the stent graft, where examples as shown in FIGS. 15-18. In FIGS. 15-18, the sensing attachment is shown for illustrative purposes as being associated with a AAA stent graft. However, the sensing attachment could likewise be associated with a different stent graft, for example, a different (not AAA) vascular (e.g., endovascular) stent grafts, a gastro-intestinal (e.g., esophageal) stent graft, or a urinary stent graft. Also, instead of being associated with a stent graft, the sensing attachment may be associated with a graft. When associated with a graft, the sensing attachment may be associated intra-luminally, a.k.a. adluminally, i.e., inside the graft.

As shown in FIG. 15, the sensing attachment 410 in the form of a filament as previously illustrated in FIG. 7A and FIG. 7B may be deployed within the aneurysmal sac 412 of a blood vessel 414, and in contact with the external surface of the endograft 416.

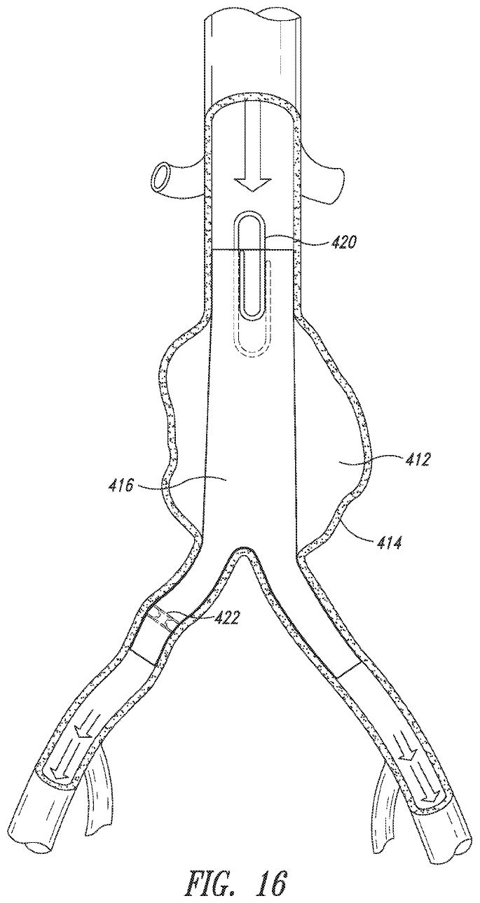

As shown in FIG. 16, the sensing attachment 420 in the form of a clip as previously illustrated in FIG. 3A may be deployed at the entrance to the aneurysmal sac 412 of a blood vessel 414, and in contact with both an internal and external surface of the endograft 116. As also shown in FIG. 16, the sensing attachment 422 in the form of a clamp as previously shown in FIG. 4A may be deployed at the exit of the aneurysmal sac 412 of a blood vessel 414, and in contact with an internal surface (as shown in FIG. 16) of the endograft 416. In one embodiment, the sensing attachment includes a pressure sensor, which refers to one or more pressure sensors. The pressure sensors may have a preferred orientation depending on how they are placed. A sensing attachment intended to contact a lumen (a vessel's or a synthetic graft) would have the pressure sensors directed radially inward away from the lumen. Sensing attachments having a ring form may also be placed as a ring external and in apposition to the endovascular graft. In this case, the hoop stress of the endovascular graft would contact the inner diameter of the sensing attachment and hold it in place. The sensors in this case would be directed radially outward.

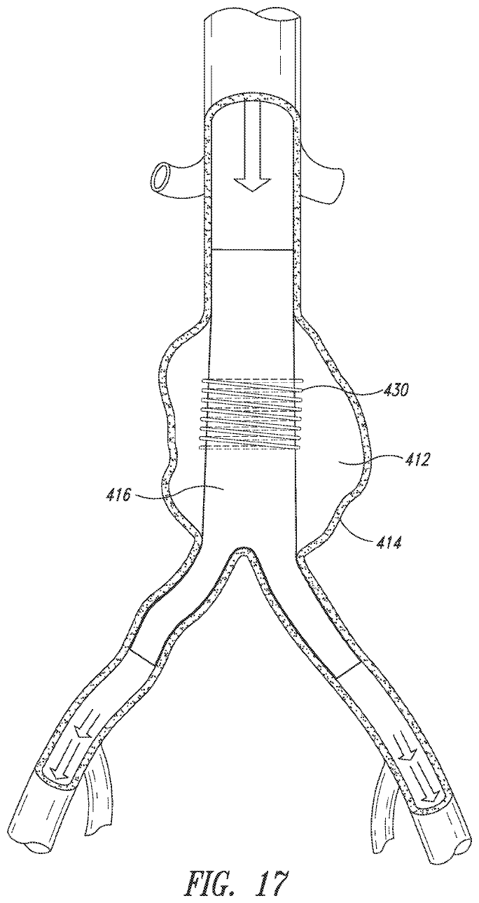

As shown in FIG. 17, the sensing attachment 430 in the form of a spring as previously illustrated in FIG. 5A and FIG. 5C, may be deployed within the aneurysmal sac 412 of a blood vessel 414, and in contact with the external surface of the endograft 416.

As shown in FIG. 18, the sensing attachment 440 in the form of a spring as previously illustrated in FIG. 6, may be deployed within the aneurysmal sac 412 of a blood vessel 414, and in contact with the external surface of the endograft 416.

In addition to the long term monitoring of hemodynamic and other parameters, the sensing attachment described herein offers the advantage of being generic to any endovascular graft and may be assembled onto the grafts percutaneously at the time of the procedure either abluminally or adluminally without affecting the design of the grafts.

Alternatively, a sensing attachment may be located within the aneurysmal sac such that it neither touches (nor minimally touches) the endovascular graft nor appreciably contacts the lumen of the aneurysmal sac. This option is illustrated in FIG. 19. Once the endovascular graft 416 is deployed within a blood vessel 414, the sensing attachment 450 comprising sensors 452 is captured within the aneurysmal sac 412 due to the endograft's proximal and distal seals of the artery relative to the aneurysmal sac. In one embodiment, the sensing attachment surrounds a length of the stent graft, but has a non-compressed size which is larger in inner diameter than is the outer diameter of the sent graft. In this way, the sensing attachment effectively floats in the aneurysm sac rather than pressing against the surface of the stent graft and being held in place by hoop stress.

In one embodiment the sensing attachment is very close to the medical device, such as within a few centimeters, i.e., 1 or 2 or 3 centimeters, of the medical device. In one embodiment, the sensing attachment is sized so that is fits around the medical device but does not fit snugly against the outer wall of the medical device. Instead, the sensing attachment fits around the medical device but leaves a gap between the outer surface of the medical device and the inner surface of the sensing attachment. In this way, the sensing attachment does not rub against, and possibly cause degradation of, the outer surface of the medical device. For example, when the sensing attachment is intended to be associated with a medical device, e.g., a graft or stent graft, that has an outer diameter (or outer cross-sectional distance) of 35 mm, then the sensing attachment may have an inner diameter (or inner cross sectional distance) of more than 35 mm, e.g., exactly or about any of 36 mm, or 37 mm, or 38 mm, or 39 mm, or 40 mm, or 41 mm, or 42 mm, or 43 mm, or 44 mm, or 45 mm. The sensing attachment needs to fit within the body cavity where it is being located, and to that end the sensing attachment may have an outer diameter (or outer cross sectional distance) of less than the inner diameter (or inner cross sectional distance) of the body cavity, e.g., the aneurysm sac. If the body cavity, e.g., aneurysm sac, has an inner diameter (or inner cross section distance) of about 50 mm, then the sensing attachment may have an outer diameter (or outer cross sectional distance) of less than about 50 mm, e.g., exactly or about any of 49 mm, or 48 mm, or 47 mm, or 46 mm, or 45 mm, or 44 mm, or 43 mm, or 42 mm, etc. In one embodiment, the sensing attachment has an inner cross sectional distance, which may be an inner diameter of the sensing attachment, where that inner cross sectional distance is in the range of about 35 mm to 45 mm. When the medical device has an outer cross sectional distance in the range of 20 mm to 35 mm, then a sensing attachment may have an inner cross sectional distance which is 1-5 mm greater than the outer cross sectional distance of the medical device, e.g., the sensing attachment may have an inner cross sectional distance of 21 mm to 40 mm. In embodiments, the sensing attachment has an inner cross sectional distance of from 15 mm to 20 mm, or from 20 to 25 mm, or from 25 to 30 mm, or from 30 to 35 mm, or from 35 to 40 mm. In embodiments, the sensing attachment has an inner cross sectional distance, which may be a diameter if the inner cross section is a circle or essentially a circle, selected from the group consisting of 15 mm to 20 mm, 20 mm to 25 mm, 25 mm to 30 mm, 30 mm to 35 mm, 35 mm to 40 mm, and 40 mm to 45 mm. The inner cross sectional distance, in the event the inner cross section is not a circle or essentially a circle, is the shortest distance directly across from a point on the inner surface as seen in cross section of the sensing attachment. The outer cross sectional distance, in the event the outer cross section is not a circle or essentially a circle, is the furthest distance between a reference point on the outer surface as seen in cross section of the sensing attachment, and another point directly across from the reference point. In embodiments, the sensing attachment has an outer cross sectional distance of from 20 mm to 50 mm, or from 20 to 25 mm, or from 25 to 30 mm, or from 30 to 35 mm, or from 35 to 40 mm, or from 40 mm to 45 mm, or from 45 mm to 50 mm. In embodiments, the sensing attachment has an outer cross sectional distance, which may be a diameter if the outer cross section is a circle or essentially a circle, selected from the group consisting of 20 mm to 25 mm, 25 mm to 30 mm, 30 mm to 35 mm, 35 mm to 40 mm, 40 mm to 45 mm, and 45 mm to 50 mm. In one embodiment, the sensing attachment having the afore-mentioned size, has the shape of a spring as shown in FIG. 6, and in vivo surrounds a tubular medical device such as shown in FIG. 18, where the sensing attachment is selected to have a sufficiently large inner cross section that it does not compress against the outer surface of the tubular medical device. The inner and outer cross sectional distances of a spring being determined by looking down the axis of the spring, where the axis identifies the center of the circle or other cross sectional shape of the spring, and the cross sectional distance is the length of a straight line that intersects that center point.

In one embodiment, the sensing attachment in the situation illustrated in FIG. 19 contains a plurality of sensors, where each of the sensors has a controlled orientation relative to the stent graft. Because the sensing attachment extends entirely around the stent graft, and the stent graft fixedly contacts the blood vessel both above and below the aneurysm sac, the sensing attachment within the aneurysm sac cannot flip or turn up-side-down: it must remain in a fixed orientation relative to the stent graft. Because the relative orientation of the stent graft and the sensing attachment is fixed, and because the sensors maintain a fixed orientation on the sensing attachment, the sensors have a constant, controlled and known orientation relative to the stent graft.

In one embodiment, the present disclosure provides a system including a stent graft and a sensing attachment, where the stent graft has an outer diameter as determined in a non-compressed state of the stent graft, and the sensing attachment has an inner diameter as determined in a non-compressed and non-expanded state of the sensing attachment, where the inner diameter of the sensing attachment is greater than the outer diameter of the stent graft so that the sensing attachment fits around but does not contact the outer surface of the stent graft. The sensing attachment has a plurality of sensors which are in a fixed orientation relative to the body of the sensing attachment, where the sensors may be, for example, pressure sensors or flow sensors. In one embodiment, the present disclosure provides a method, wherein this system is implanted into a patient, where the stent graft transverses an aneurysm sac, and the sensing attachment surrounds the outside of the stent graft and is located within the aneurysm sac, such as shown in FIG. 19. In embodiments, the sensing attachment has an inner cross sectional distance of from 15 mm to 20 mm, or from 20 to 25 mm, or from 25 to 30 mm, or from 30 to 35 mm, or from 35 to 40 mm. In embodiments, the sensing attachment has an inner cross sectional distance, which may be a diameter if the inner cross section is a circle or essentially a circle, selected from the group consisting of 15 mm to 20 mm, 20 mm to 25 mm, 25 mm to 30 mm, 30 mm to 35 mm, 35 mm to 40 mm, and 40 mm to 45 mm. The inner cross sectional distance, in the event the inner cross section is not a circle or essentially a circle, is the shortest distance directly across from a point on the inner surface as seen in cross section of the sensing attachment. The outer cross sectional distance, in the event the outer cross section is not a circle or essentially a circle, is the furthest distance between a reference point on the outer surface as seen in cross section of the sensing attachment, and another point directly across from the reference point. In embodiments, the sensing attachment has an outer cross sectional distance of from 20 mm to 50 mm, or from 20 to 25 mm, or from 25 to 30 mm, or from 30 to 35 mm, or from 35 to 40 mm, or from 40 mm to 45 mm, or from 45 mm to 50 mm. In embodiments, the sensing attachment has an outer cross sectional distance, which may be a diameter if the outer cross section is a circle or essentially a circle, selected from the group consisting of 20 mm to 25 mm, 25 mm to 30 mm, 30 mm to 35 mm, 35 mm to 40 mm, 40 mm to 45 mm, and 45 mm to 50 mm. In one embodiment, the sensing attachment having the afore-mentioned size, has the shape of a spring as shown in FIG. 6, and in vivo surrounds a tubular medical device such as shown in FIG. 18, where the sensing attachment is selected to have a sufficiently large inner cross section that it does not compress against the outer surface of the tubular medical device, but instead there is a gap between the sensing attachment and the tubular medical device, e.g., a gap as illustrated in FIG. 19 for the sensing attachment having the shape shown in FIG. 13A.

The present disclosure provides the following exemplary embodiments, which are numbered for convenience.
1) A sensing attachment for a tubular medical device, the sensing attachment comprising:
a. a sensor;
b. a communication interface configured to provide intra-body communication to another device; and at least one of:
i. an elastic or super-elastic body having a shape that fits around the tubular medical device selected from a graft and a stent graft, where the shape has an inner cross sectional distance that is optionally in the range of 15 mm to 45 mm and an outer cross sectional distance that is optionally in the range of 20 mm to 50 mm; and
ii. a body in the shape of a spring formed from nitinol, where the spring has an inner cross sectional distance that is optionally in the range of 15 mm to 45 mm and an outer cross sectional distance that is optionally in the range of 20 mm to 50 mm.
2) The sensing attachment of embodiment 1 wherein the body is in a form of a solid or hollow filament.
3) The sensing attachment of embodiment 1 wherein the body is in a form of a monofilament or multifilament.
4) The sensing attachment of embodiment 1 wherein the body is in a form of a hollow filament.
5) The sensing attachment of embodiment 1 wherein the body is in a form of a hollow filament comprising nitinol, where the hollow filament has a lumen.
6) The sensing attachment of embodiment 1 wherein the body is in a form of a hollow filament comprising nitinol, where the hollow filament has a lumen surrounded by a wall of the hollow filament, where the wall has an inner surface facing the lumen and an outer surface facing away from the lumen, and where the hollow filament has a plurality of cuts along its length, each cut extending from the outer surface of the hollow filament into the lumen of the hollow filament.
7) The sensing attachment of embodiment 1 wherein the body is in a form of a hollow filament comprising nitinol, where the hollow filament has a lumen surrounded by a wall of the hollow filament, where the wall has an inner surface facing the lumen and an outer surface facing away from the lumen, and where the hollow filament has a plurality of cuts along its length, each cut extending from the outer surface of the hollow filament into the lumen of the hollow filament, wherein the plurality of cuts are separated from one another by 1 to 20 mm.
8) The sensing attachment of embodiment 1 wherein the body is in a shape of a spring.
9) The sensing attachment of embodiment 1 wherein the body is in a shape of a spring, and the spring has an inner cross sectional distance of 15 mm to 20 mm.
10) The sensing attachment of embodiment 1 wherein the body is in a shape of a spring, and the spring has an inner cross sectional distance of 20 mm to 25 mm.
11) The sensing attachment of embodiment 1 wherein the body is in a shape of a spring, and the spring has an inner cross sectional distance of 25 mm to 30 mm.
12) The sensing attachment of embodiment 1 wherein the body is in a shape of a spring, and the spring has an inner cross sectional distance of 30 mm to 35 mm.
13) The sensing attachment of embodiment 1 wherein the body is in a shape of a spring, and the spring has an inner cross sectional distance of 35 mm to 40 mm.
14) The sensing attachment of embodiment 1 wherein the body is in a shape of a spring, and the spring has an inner cross sectional distance of 40 mm to 45 mm.
15) The sensing attachment of embodiment 1 wherein the body is in a shape of a spring, and the spring has an outer cross sectional distance of 20 mm to 25 mm.
16) The sensing attachment of embodiment 1 wherein the body is in a shape of a spring, and the spring has an outer cross sectional distance of 25 mm to 30 mm.
17) The sensing attachment of embodiment 1 wherein the body is in a shape of a spring, and the spring has an outer cross sectional distance of 30 mm to 35 mm.
18) The sensing attachment of embodiment 1 wherein the body is in a shape of a spring, and the spring has an outer cross sectional distance of 35 mm to 40 mm.
19) The sensing attachment of embodiment 1 wherein the body is in a shape of a spring, and the spring has an outer cross sectional distance of 40 mm to 45 mm.
20) The sensing attachment of embodiment 1 wherein the body is in a shape of a spring, and the spring has an outer cross sectional distance of 45 mm to 50 mm.
21) The sensing attachment of embodiment 1 wherein the body is in a shape of a spring running in a clockwise direction.
22) The sensing attachment of embodiment 1 wherein the body is in a shape of a spring running in a counter-clockwise direction.
23) The sensing attachment of embodiment 1 wherein the body is in a shape of a ring.
24) The sensing attachment of embodiment 1 wherein the body comprises a hollow monofilament in a shape of a spring.
25) The sensing attachment of embodiment 1 wherein the sensing attachment is biocompatible.
26) The sensing attachment of embodiment 1 wherein the body is elastic or super-elastic.
27) The sensing attachment of embodiment 1 wherein the body comprises a shape-memory material.
28) The sensing attachment of embodiment 1 wherein the body comprises nitinol.
29) The sensing attachment of embodiment 1 wherein the body comprises an elastomeric plastic.
30) The sensing attachment of embodiment 1 wherein the body comprises a polymeric coating on a surface of the body.
31) The sensing attachment of embodiment 1 wherein the body comprise a lubricious coating on a surface of the body.
32) The sensing attachment of embodiment 1 wherein a sleeve is positioned around at least a portion of the surface of the body.
33) The sensing attachment of embodiment 1 wherein the sensor is selected from a fluid pressure sensor, fluid volume sensor, contact sensor, position sensor, pulse pressure sensor, blood volume sensor, blood flow sensor, chemistry sensor (e.g., for blood and/or other fluids), metabolic sensor (e.g., for blood and/or other fluids), accelerometer, mechanical stress sensor and temperature sensor.
34) The sensing attachment of embodiment 1 wherein the sensor is a pressure sensor.
35) The sensing attachment of embodiment 1 wherein the sensor is a plurality of pressure sensors.
36) The sensing attachment of embodiment 1 wherein the sensor is a MEMS sensor.
37) The sensing attachment of embodiment 1 wherein the sensor is hermetically sealed.
38) The sensing attachment of embodiment 1 further comprising a power supply.
39) The sensing attachment of embodiment 1 further comprising a power supply and an electronics assembly having various circuitry powered by the power supply, the electronics assembly comprising one or more of components selected from a fuse, a switch, a clock generator and power management unit, a memory and a controller.
40) The sensing attachment of embodiment 1 wherein the communication interface comprises a radio frequency (RF) transceiver and a filter, that couple with an antenna.
41) The sensing attachment of embodiment 1 wherein the communication interface comprises tissue conductive communication circuitry that couples with a pair of electrodes.
42) The sensing attachment of embodiment 1 wherein the communication interface comprises data-over-sound circuitry that couples with an acoustic transducer.
43) A kit comprising the sensing attachment of any of embodiments 1-42 and a stent graft, where the stent graft has an outer cross sectional distance which is less than the inner cross sectional distance of the sensing attachment by at least about 1 mm.
44) A kit comprising the sensing attachment of any of embodiments 1-42 and a graft, where the graft has an outer cross sectional distance which is less than the inner cross sectional distance of the sensing attachment by at least about 1 mm.
45) A system comprising the sensing attachment of any of embodiments 1-42 associated with a stent graft.
46) A system comprising the sensing attachment of any of embodiments 1-42 associated with a graft.
47) A method for making a system comprising a medical device and a sensing attachment located external to the medical device, the method comprising:
a. providing a medical device selected from the group consisting of a graft and a stent graft, the medical device having an inner surface and an outer surface;
b. selecting a sensing attachment of embodiment 1 having an inside and an outside, the inside having an inner diameter, where the inner diameter of the sensing attachment is at least 1 mm larger than the outer diameter of the medical device; and
c. either placing the sensing attachment around the medical device, or placing the medical device through the sensing attachment.
48) A method for making a system comprising a medical device and a sensing attachment located external to the medical device, the method comprising:
a. providing a medical device selected from the group consisting of a graft and a stent graft, the medical device having an inner surface and an outer surface;
b. selecting a sensing attachment of embodiment 1 having an inside and an outside, the inside having an inner diameter, where the inner diameter of the sensing attachment is selected from 15 mm to 20 mm, 20 mm to 25 mm, 25 mm to 30 mm, 30 mm to 35 mm, 35 mm to 40 mm and 40 mm to 45 mm and is larger than the outer diameter of the medical device; and either placing the sensing attachment around the medical device, or placing the medical device through the sensing attachment.

In FIGS. 15 and 19, the sensing attachment is shown as having sensors 103. For ease of viewing, the sensors are not shown in the drawings of FIGS. 16, 17 and 18. However, when a sensing attachment is associated with an implanted stent graft as illustrated in FIGS. 16, 17 and 18, the sensing attachments would have at least one sensor as discussed herein. Also, an example sensing attachment placed internal to a AAA graft, i.e., adluminally, is illustrated in FIG. 16, where sensing attachment 122 is entirely within the stent graft at a distal location, and sensing attachment 120 is placed partially adluminally and partially abluminally, i.e., on the outer surface of the stent graft, at a proximal location, where blood flows from the proximal end to the distal end of the stent graft. Although FIG. 15, FIG. 17 and FIG. 18 illustrate the sensing attachment located entirely on the abluminal surface of the stent graft, the sensing attachment could alternatively be located on the adluminal surface of the of the stent graft. Also, although FIG. 15, FIG. 17 and FIG. 18 illustrate the sensing attachment located at about the center or body of the stent graft, within the aneurysm sac, the sensing attachment could alternatively be located at a proximal end and/or a distal end of the stent graft.

In an alternative embodiment, a sensing attachment with wireless accelerometer(s) and wireless capacitive pressure sensors may be used in conjunction with a sensing attachment located external to the endograft in the aneurysmal sac to obtain transluminal pressure measurements in the aneurysmal sac region and within the vessel. The pressure in the aneurysmal sac would be much lower than the vessel as it has been excluded from flow by the endograft. Aneurysmal sac pressures well sealed by an endograft are typically in the 10-30 mmHg range with a pulse pressure of 5-10 mmHg vs. an arterial pressure of 60-140 mmHg and pulse pressures of 40-60 mmHg. If there was an endoleak, aneurysmal sac pressure would increase causing a decrease in the mean transluminal pressure and pulse pressure. This in turn would cause a segmental change in the graft's wall motion resulting in a change in the accelerometer signal. Having the accelerometer signal in addition to the change in the transluminal pressure would guard against a false positive indicative of drift in the pressure sensors indicating an EL as both sensors (accelerometer and pressure) would be needed to diagnose the presence of an endoleak.

For coronary applications, a sensing attachment would be implanted proximal and distal to a lesion avoiding any contact with the actual coronary stent. Through measurement of pressure at each location, detailed information on the coronary vessel's flow rate, pressure, pulse pressure changes over time may be monitored alerting the patient and clinicians to changes with much more fidelity as compared to discrete monitoring every 6 months to a year which is standard of care.

In the case of an implantable medical device, the sensing attachment may be associated with the medical device either prior to the implantation, i.e., pre-operatively, or during the implantation, i.e., intra-operatively, or after the implantable medical device has been implanted in the patient, i.e., post-operatively.

In one aspect, the sensing attachment is associated with the medical device prior to the procedure whereby the medical device is implanted into the patient, i.e., pre-operatively. In one embodiment, the sensing attachment is associated with the medical device in the operating room but before the start of the operation. In one embodiment, the sensing attachment is associated with the medical device prior to the time the medical device is packaged for shipment to the surgical center, so that the medical device arrives in the operating room with the sensing attachment already associated with the medical device.

In one embodiment, the sensing attachment is associated with a graft. A graft is typically implanted into a patient during a surgery, where the graft is placed interpositionally, i.e., a portion of a tubular structure in a patient is cut out and the graft is located interpositionally, i.e., in the location where the tube was cut away. In one embodiment, the graft with an associated sensing attachment is used in interpositional vascular grafting. For an interpositional surgery, a sensing attachment may be associated with the graft prior to the beginning of the surgery. In one embodiment, the sensing attachment is associated with the inside of the graft, i.e., the sensing attachment is placed wholly or partially inside (adluminally) the graft. In this way, the sensor attached to the sensing attachment will, after implantation of the graft with associated sensing attachment in a patient, be able to make detections and/or measurements which characterize fluid that flows through the graft. In the case where the sensor should detect fluid pressure and/or fluid flow, the sensor should be located on the inside of the sensing attachment, i.e., on the side of the sensing attachment that faces towards the lumen of the graft. In one embodiment, a graft is associated with two sensing attachments, one located at the entrance and the other located at the exit of the graft, where the sensors on the sensing attachment are in contact with the fluid that flows through the lumen of the graft.

A sensing attachment may be associated with the inside of a graft by compressing the sensing attachment from a non-compressed state, i.e., a natural state, to a compressed state, maintaining the sensing attachment in the compressed state, placing the sensing attachment at a desired location within the graft while maintaining the sensing attachment in the compressed state, and then releasing the sensing attachment from the compressed state so that the sensing attachment returns to its natural, i.e., non-compressed, state. The non-compressed state should have a size such that the outer surfaces of the sensing attachment touches the inner surface of the fabric of the graft with an amount of pressure. The amount of pressure should be sufficient to maintain the sensing attachment in place within the graft. The pressure of the sensing attachment pushing against the inner wall of the graft will create a hoop stress, where this hoop stress should be sufficient to hold the sensing attachment in place within the graft. A delivery system as described herein may be used to transfer the compressed sensing attachment to a site with the graft, and then to release the sensing attachment from the compressed state at a desired time and allow it to adopt its natural state.

In one embodiment, the present disclosure provides a graft associated with a sensing attachment. Optionally, the association may place the sensing attachment wholly or partially within the lumen of the graft. In one embodiment, the sensor on the attachment may not face towards i.e., contact, the graft, in order that when the graft is implanted in patient, the sensor will contact fluid that passes through the graft. Optionally, the sensing attachment may be two sensing attachments, one placed at each end of the graft, in each case the sensing attachment is placed within the graft. In one embodiment the present disclosure provides a method of associating a sensing attachment with a graft, where the method includes placing the sensing attachment within the lumen of the graft. Optionally, the sensing attachment is in a compressed state when it is placed within the graft, and then is released from the compressed state after it is located at a desired position within the graft, and held in place within the graft by hoop stress. In one embodiment, the present disclosure provides a method of monitoring fluid within a vessel, the method including interpositional grafting of a graft that is associated with a sensing attachment according to the present disclosure, and then monitoring fluid that flows within the graft using the sensor of the sensing attachment, as described herein.

In one embodiment, the present disclosure provides a stent graft associated with a sensing attachment. The association of a sensing attachment with a stent graft will be described in detail using a AAA stent graft as an example. However, the same disclosure applies to other stent grafts, e.g., other endovascular stent grafts, as well as gastro-intestinal stent grafts and urinary stent grafts.

There are two primary treatments for AAAs, which are known as open surgical repair and endovascular aneurysm repair (EVAR). Surgical repair typically includes opening the dilated portion of the aorta, inserting a synthetic tube, and closing the aneurysmal sac around the tube. In the case of surgical repair, the sensing attachment of the present disclosure may be associated with the stent graft in the operating room. For example, a sensing attachment having the shape of a spring may be fitted around the outer circumference of the stent graft, and the combination of sensing attachment and medical device is inserted into the dilated portion of the aorta, following by closing the aneurysmal sac around the combination. The same procedure may be used when the sensing attaching has any other shape, e.g., the sensing attachment may be clipped onto the stent graft in the case where the sensing attachment has the shape of a clip, or it may be clamped onto the stent graft in the case where the sensing attachment has the shape of a clamp (e.g. a cuff bracelet shape), where in any event the combination of sensing attachment associated with a stent graft is inserted into the aneurysmal sac.

Minimally invasive endovascular aneurysm repair (EVAR) treatments that implant stent grafts across aneurysmal regions of the aorta have been developed as an alternative or improvement to open surgery. EVAR typically includes inserting a delivery catheter into the femoral artery, guiding the catheter to the site of the aneurysm via X-ray visualization, and delivering a synthetic stent graft to the AAA via the catheter. The stent graft is contained within the delivery catheter, in a compressed form. Upon reaching the site of the AAA, the compressed stent graft is expelled from the delivery catheter, whereupon the stent graft expands to its desired shape and size due to the elastic nature of the stent graft. According to the present disclosure, a sensing attachment is associated with the stent graft and the combination is compressed into the delivery catheter. When the compressed combination of sensing attachment and stent graft is delivered to the site of the AAA, the combination may be expelled from the delivery catheter, whereupon each of the stent graft and the associated sensing attachment expands to their respective shape and size due to the elastic natures of the stent graft and sensing attachment.

In one embodiment, the present disclosure provides a stent graft associated with a sensing attachment. Optionally, the association may place the sensing attachment wholly or partially within the lumen of the graft. The sensor on the attachment may not face towards i.e., contact, the graft of the stent graft, in order that when the graft is implanted in patient, the sensor will contact fluid that passes through the graft. Optionally, the association may place the sensing attachment wholly or partially against the exterior surface of the stent graft, i.e., not wholly within the lumen of the stent graft. In the case, the sensor on the attachment may not face towards i.e., contact, the graft of the stent graft, in order that when the graft is implanted in patient, the sensor will contact fluid that passes around the graft in the region of the aneurysm sac. Optionally, when the sensing attachment is placed adluminally, the sensing attachment may be two or three sensing attachments, placed at various ends of the stent graft. In one embodiment, three sensing attachments are placement adluminally, one at each orifice of the stent graft. In this way, when the sensor is a pressure sensor or other fluid measurement sensor, the sensor can monitor the fluid entering and exiting the stent graft.

In one embodiment the present disclosure provides a method of associating a sensing attachment with a stent graft, where the method includes placing the sensing attachment within the lumen of the stent graft. Optionally, the sensing attachment is in a compressed state when it is placed within the stent graft, and then is released from the compressed state after it is located at a desired position within the stent graft, and held in place within the stent graft by hoop stress. In one embodiment, the present disclosure provides a method of monitoring fluid within a stent graft, the method including surgically placing a stent graft associated with a sensing attachment of the present disclosure in an aneurysm sac, and then monitoring fluid that flows within the stent graft using the sensor of the sensing attachment, as described herein.

In one embodiment the present disclosure provides a method of associating a sensing attachment with a stent graft, where the method includes placing the sensing attachment against the exterior surface of the stent graft. Optionally, the sensing attachment is in an expanded state when it is placed against the outer surface of the stent graft, and then is released from the expanded state after it is located at a desired position around the stent graft, to then adopt its natural, i.e., non-expanded but also non-compressed state, and held in place around the stent graft by hoop stress. In one embodiment, the present disclosure provides a method of monitoring conditions outside of a stent graft, the method including surgically placing a stent graft associated with a sensing attachment of the present disclosure in an aneurysm sac, and then monitoring the conditions within the aneurysm sac but outside of the stent graft, using the sensor of the sensing attachment, as described herein.

In one aspect, the sensing attachment is associated with the medical device during the same procedure whereby the medical device is implanted into the patient. This option will be described for the case where the sensing attachment is a spring shape as in FIG. 5A, 5C or 6, and the medical device is a AAA stent graft, however the same principles apply to other sensing attachments and implantable medical devices as descried herein.

In one aspect, introduction of the sensing attachment to the endovascular graft does not interrupt the standard method of abdominal aortic aneurysm treatment employed by the physician. For example, after the seating of the primary graft section of the AAA graft, a secondary percutaneous delivery system carrying the sensing attachment is entered into the AAA sac and located in the position to deploy the sensing attachment about the maximum diameter of the AAA primary graft and extend down the graft till the sensor system is deployed fully from the percutaneous delivery system. In one embodiment, the sensors may be placed to cover any radian of space in the AAA sac, from 1 degree to 360 degrees in circumference of the AAA repair by the medical device graft. Optionally, the sensing attachment, e.g., having a spring shape, may be released about the outer diameter of the implanted graft and released before or after the final installation of the secondary iliac limb seal is completed.

When the sensing attachment is placed about the outer diameter of the AAA graft treatment system for the abdominal aortic aneurysm, the compression spring force which holds the sensing attachment in place adjacent to the stent graft, may be generated by the shaping of the body of the sensing attachment, e.g., the primary tubular frame construction itself, or in a combination construction of a nitinol tube that makes up the sensing attachment platform base, or the communication antenna, e.g., a platinum iridium wire that makes up the communication antenna. Features of the sensing attachment, particularly metallic features, may be used to achieve the necessary inward spring force that may maintain a circular of single diameter configuration or multiple diameter configuration where there is a major and a minor diameter. The inward spring force should have minimal impact on the AAA inner diameter or the graft material seal function in the human anatomy.

Optionally, the sensing attachment, e.g., having a spring shape, may be released inside the inner diameter of the aortic abdominal graft treatment system and seat itself so not to dislodge below the iliac bifurcation of the AAA treatment graft. In this way, the sensing attachment may sense not only the blood wave form but also detect effects to the wave form through the sensors being placed in the pathway of the blood.

In one method to achieve the situation shown in FIG. 16, the endograft is inserted normally and a sensing attachment is inserted subsequently and placed over the endograft prior to its full deployment in the vascular system, a.k.a like a cigar ring. The sensing attachment is moved into the appropriate position axially along the endograft within the aneurysmal sac, and the endograft is dilated thereby bringing the inner diameter of the sensing attachment into contact with the outer diameter of the endograft such that the inherent hoop stress of the sensing attachment will secure the sensing attachment against the endograft stopping any migration. As a second measure guarding against sensing attachment axial movement, the sensing attachment cannot migrate distally as the aneurysmal sac is “walled off” via the endograft.

In one aspect, the sensing attachment is associated with the medical device after the procedure whereby the medical device is implanted into the patient. This option will be described for the case where the sensing attachment is a has a spring shape and the medical device is a AAA stent graft, however the same principles apply to other sensing attachments and other implantable medical devices as described herein.

In one aspect, the present disclosure provides a sensing attachment in a geometric shape deliverable through a single or multi-tubular constructed catheter entering the vasculature through a delivery system and tracking to the designated site for releasing the sensing attachment in the similar designated area where an implant has been positioned into the vascular structure.

After associating, the sensing attachment shall coil, i.e. wrap about the graft or the vessel wall and maintain a position by interacting with the AAA graft or within the AAA Sac area by opposing forces against the wall, anchoring to the wall or stabling based on the coil length and in conjunction of the non-expanded abdominal aorta transition to the enlargement of the wall and through the aneurysm in contact with the base of the enlargement aneurysm wall in transition to the iliac artery wall.

As mentioned herein, the sensing attachment may be associated with the medical device either pre-operatively, intra-operatively, or post-operatively. In any event, the sensing attachment needs to be implanted in the patient. When the sensing attachment is associated with the medical device pre-operatively, the combination of sensing attachment and associated medical device may be placed within a single delivery system, so that the sensing attachment and associated medical device are co-delivered to the patient. However, when the sensing attachment is associated with the medical device either intra-operatively or post-operatively, then the sensing attachment and medical device are delivered to the patient using separate delivery systems, i.e., one delivery system for the medical device and a separate delivery system for the sensing attachment.

In one embodiment, a catheter delivery system is used to deliver the sensing attachment to the patient. In one embodiment, the catheter delivery system is designed to accommodate either the sensing attachment alone, or the sensing attachment in association with a medical device. Physicians who perform AAA treatment are very familiar with catheter delivery systems for stent grafts. The present disclosure provides a catheter delivery system which is analogous to the catheter delivery system with which physicians are familiar when performing AAA treatment. With this embodiment, the physician may use his or her skills as already developed for treating AAA, to also deliver a sensing attachment of the present disclosure to the patient being treated. This embodiment will be described for the case where the sensing attachment alone is being delivered, however, the same principles apply when a combination of sensing attachment and medical device is being delivered.

To deliver a medical device via a catheter delivery system, an elastic medical device is compressed into a very small size that may be inserted into a femoral artery. This is commonly done in current practice for delivering and implanting a stent or a stent graft via a catheter delivery system. The medical device is compressed into a very small size and then maintained in that small size by the catheter delivery system while it is being delivered to the site of the aneurysm by a progressive movement of the delivery catheter through the artery. The medical device is typically held within the leading end of the delivery catheter. When the leading end of the delivery catheter has reached the location where the physician desires to deploy the carried medical device, a release mechanism on the delivery catheter is activated by the physician, which causes the medical device to be released from the delivery catheter. Due to the elastic nature of the medical device, it will assume a non-compressed size and shape upon being released from the delivery catheter. This same principle is applied to deliver a sensing attachment or a combination of a sensing attachment and an associated medical device, to a desired site within a patient.

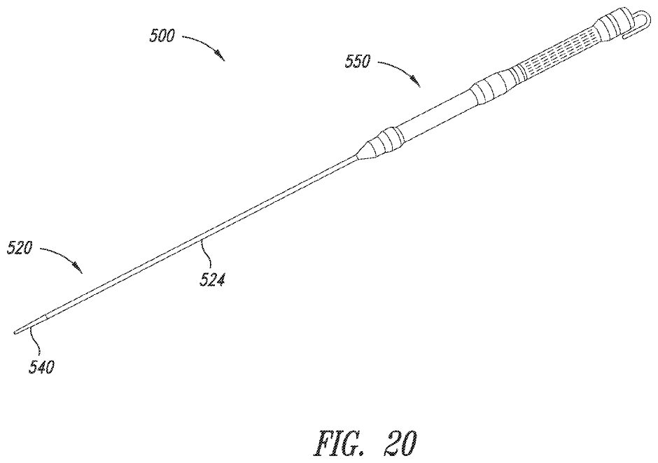

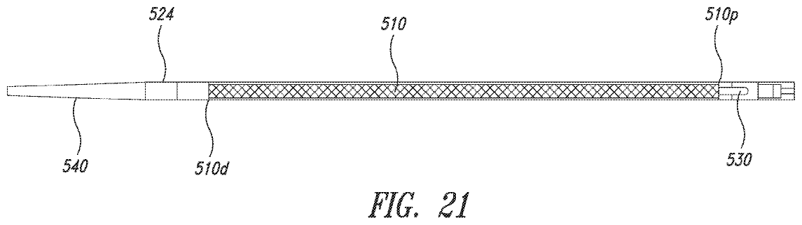

FIGS. 20 and 21 show an example embodiment of a delivery apparatus 500 for a sensing attachment 510 in a compressed state. Although FIGS. 20 and 21 are discussed in relation to the delivery of a sensing attachment 510, the same principles apply when the sensing attachment is in association with a medical device, e.g., a stent graft. Accordingly, in the following discussion, reference to sensing attachment 510 applies equally to a combination of sensing attachment and a stent graft or other medical device.

The delivery apparatus 500 of FIGS. 20 and 21 can include a delivery catheter 520 and handle 550 operably coupled to the delivery catheter 520. The delivery catheter 520 has proximal and distal ends, and also has a lumen extending therethrough, where the lumen has a length and a cross-sectional area. The sensing attachment 510 in a compressed state is located entirely within the lumen of the delivery catheter and extends from 510d at a distal end of the lumen to 510p at a proximal end of the lumen. The delivery apparatus 500 also includes a push rod 530 that is slidably disposed within the lumen of the delivery catheter 520. A portion of the push rod 530 is shown in FIG. 21, where the remainder of the push rod 530 lies behind the sensing attachment 510 and thus cannot be seen in the view of FIG. 21. The push rod 530 is adjacent to but not within the compressed sensing attachment. In other words, the push rod 530 and the sensing attachment 510 are adjacent but separate in that the push rod 530 does not pass into or through the compressed sensing attachment 510.

As shown in FIG. 21, the distal end portion of the delivery catheter 520 can include a distal sheath 524 that covers and constrains at least a portion of, and in one embodiment all of, the compressed sensing attachment 510 in a radially compressed configuration. Thus, the delivery apparatus 500 includes a distal movable sheath 524 that covers a portion of the length of lumen of the delivery catheter, where the portion of the lumen contains a portion of the push rod 530 and a first portion of the sensing attachment 510 in a compressed state.

The slidably disposed push rod 530 is engaged with the distal movable sheath 524 such that sliding of the push rod 530 causes movement of the movable sheath 524, where the movement exposes the compressed sensing attachment 510 and thereby allows the compressed sensing attachment to achieve a less compressed form. In other words, moving the distal sheath 524 in a distal direction can expose the sensing attachment 510, thereby freeing the compressed sensing attachment to achieve a less compressed form. In FIG. 21, the distal movable sheath 524 has moved in a distal direction and occupies the space shown as 524. In embodiments, the push rod is a solid push rod, is a flexible push rod, is a rotatable push rod.

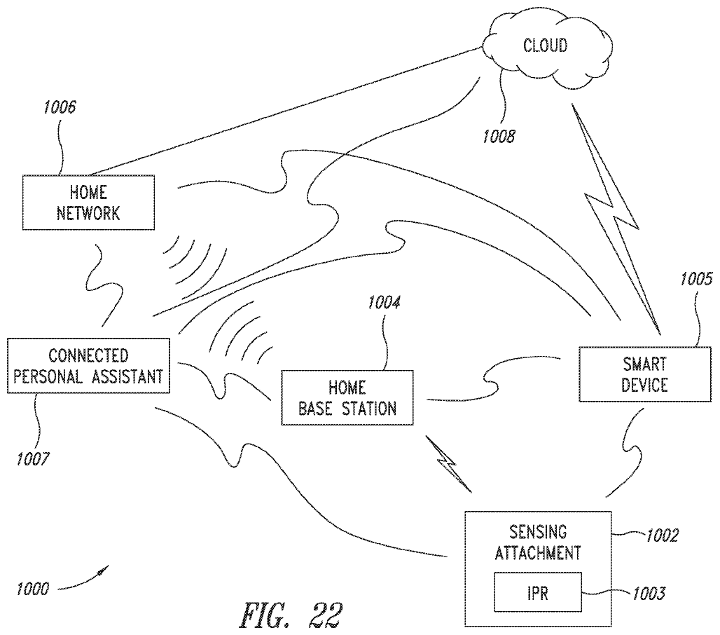

In one embodiment, not shown in FIG. 21 or 22, the delivery apparatus includes a proximal movable sheath, where the proximal movable sheath covers a second portion of the length of lumen of the delivery catheter, where the second portion of the lumen contains a second portion of the push rod and a second portion of the sensing attachment in a compressed state. The handle assembly 550 is engaged with and can cause movement of the proximal movable sheath, such that the movement exposes the second portion of the compressed sensing attachment and thereby allows the compressed sensing attachment to achieve a less compressed form.

For example, a moveable slider screw (which may also be referred to as a linear slider, not shown in FIG. 21 or 22) within the proximal handle 550 may connect the handle 550 to the proximal movable sheath (not shown), to provide for movement of the proximal movable sheath, such that the movement exposes the second portion of the compressed sensing attachment. The proximal movable sheath can be moved by actuating the handle rotation and with a linear screw interaction, to move the proximal outer sheath proximal from its position over the sensor attachment system. As another option, a lock slider and groove configuration may be used to connect the proximal movable sheath to the handle.

The proximal movable sheath shall be able to move longitudinally and independent from the push rod, where the push rod is used to move the distal movable sheath.

In one embodiment, the push rod and the delivery catheter are arranged such that there is not an offset formed at the distal end of said delivery catheter. In one embodiment, the compressed sensing agent is not located within an offset at a distal end of the delivery catheter. In one embodiment, the push rod and the delivery catheter are arranged such that there is not a recess present at the distal end of the delivery catheter. In one embodiment, the compressed sensing agent is not located within a recess at a distal end of the delivery catheter.

In various embodiments, the present disclosure provides:
[1] An apparatus comprising:
a. a delivery catheter having proximal and distal ends and having a lumen extending therethrough, the lumen having a length and a cross-sectional area;
b. a sensing attachment in a compressed state, the compressed sensing attachment located entirely within the lumen of the delivery catheter;
c. a push rod slidably disposed within the lumen of the delivery catheter, the push rod adjacent to and not within the compressed sensing attachment;
d. a distal movable sheath that covers a first portion of the length of lumen of the delivery catheter, where the first portion of the lumen contains a first portion of the push rod and a first portion of the sensing attachment in a compressed state;
e. where the slidably disposed push rod is engaged with the distal movable sheath such that sliding of the push rod causes movement of the movable sheath, where the movement exposes the first portion of the compressed sensing attachment and thereby allows the compressed sensing attachment to achieve a less compressed form.
[2] The apparatus of embodiment 1 where the push rod and the delivery catheter are arranged such that there is not an offset formed at said distal end of said delivery catheter.
[3] The apparatus of embodiment 1 where the compressed sensing attachment is not located within an offset at a distal end of the delivery catheter.
[4] The apparatus of embodiment 1 wherein the push rod is a solid push rod.
[5] The apparatus of embodiment 1 wherein the push rod is a flexible push rod.
[6] The apparatus of embodiment 1 further comprising a handle and a proximal movable sheath, where the handle is engaged with the proximal movable sheath by way of a moveable slider screw, where the proximal movable sheath covers a portion of the length of lumen of the delivery catheter, where the portion of the lumen contains a portion of the push rod and a second portion of the sensing attachment in a compressed state.
[7] The apparatus of embodiment 1 further comprising a marker.
[8] The apparatus of embodiment 1 further comprising a marker detectable by fluoroscopy.
[9] The apparatus of embodiment 1 further comprising a marker present in the distal section of the delivery catheter and a marker present in the proximal section of the delivery catheter.
[10] The apparatus of embodiment 1 further comprising a marker present on the push rod and a marker present on the distal movable sheath.
[11] The apparatus of embodiment 1 further comprising a marker that is visible and positioned to provide direct visual communication on the placement of the distal section of the delivery system and apply a position of the loaded sensor attachment system distal end, for release initiation.
[12] The apparatus of embodiment 1 further comprising a marker that is visible and positioned to provide direct visual communication on the placement of the proximal section of the delivery system and apply a position of the loaded sensor attachment system proximal end, for secondary release initiation.
[13] The apparatus of embodiment 1 further comprising a marker that is visible and positioned to provide direct visual communication on the placement of the distal and proximal edges covering the loaded sensor attachment system, for release initiation.
[14] The apparatus of embodiment 1 further comprising a maker that is visible and positioned to provide direct radial orientation visual communication on the radial placement position of the loaded sensor attachment system distal end, for release initiation.
[15] The apparatus of embodiment 1 further comprising a marker located on the proximal movable sheath to enable a physician to have visible determination during the procedure for radial orientation, linear travel of the proximal shaft.
[16] The apparatus of embodiment 1 wherein the push rod contains a lumen extending through an entire length of the push rod.
[17] The apparatus of embodiment 1 wherein the push rod contains a lumen extending through an entire length of the push rod, and the push rod lumen enables the delivery catheter to travel over a guidewire.
[18] The apparatus of embodiment 1 wherein the push rod contains a lumen extending through an entire length of the push rod, and the push rod lumen enables a physician to flush a AAA sac and thereby ensure no clotting within the AAA sac that could impede the function of the apparatus.
[19] The apparatus of embodiment 1 wherein the distal end of the delivery catheter terminates in a distal tip.
[20] The apparatus of embodiment 1 wherein the distal end of the delivery catheter terminates in a distal tip, where the distal tip has a configuration that provides steerable characteristics during insertion and positioning of the sensing attachment in the patient.
[21] The apparatus of embodiment 1 wherein the distal end of the delivery catheter terminates in a distal tip, where the distal tip comprises a polymeric material with a durometer hardness in the range from 25A through 95A.
[22] The apparatus of embodiment 1 wherein the distal end of the delivery catheter terminates in a distal tip, where the distal tip has proximal and distal ends, and where the proximal end has a diameter that can interface with a distal section of the distal movable sheath, and then extend in a cone configuration and transition to a tubular form, where the tubular form has an outer diameter that is less than the distal movable sheath outer diameter and an inner diameter that enables flushing or a guidewire to be present within the tubular form.
[23] The apparatus of embodiment 1 wherein the distal end of the delivery catheter terminates in a distal tip, where the distal tip has proximal and distal ends, and where the proximal end has a diameter that can interface with a distal section of the distal movable sheath, and then extend in a cone configuration and transition to a tubular form, where the tubular form has an outer diameter that is less than the distal movable sheath outer diameter and an inner diameter that enables flushing or a guidewire to be present within the tubular form, and where cone configuration shall have a length as measured from the larger diameter to the smaller diameter from 5 mm to 60 mm.
[24] The apparatus of embodiment 1 wherein the distal end of the delivery catheter terminates in a distal tip, where the distal tip has a length of 5 mm to 65 mm.
[25] The apparatus of embodiment 1 wherein the distal end of the delivery catheter terminates in a distal tip, where the distal tip has a diametrical configuration selected from concentric and non-concentric, where the diametrical configuration aids in the steerability of the delivery catheter.
[26] The apparatus of embodiment 1 wherein the distal end of the delivery catheter terminates in a distal tip, where the distal tip does not have any markers.
[27] The apparatus of embodiment 1 wherein the distal end of the delivery catheter terminates in a distal tip, where the distal tip comprises a marker.
[28] The apparatus of embodiment 1 wherein the distal end of the delivery catheter terminates in a distal tip, where the distal tip comprises a marker that is detectable by fluoroscopy.

Markers, also known as marker bands, are known for other delivery systems and may be used in the apparatus of the present disclosure. The marker may be a radiopaque marker, which may include a heavy metal having an atomic number of at least about 70, including gold, platinum, tantalum etc. In some cases, the radiopaque marker may include a powdered heavy metal such as bismuth or tantalum. See, e.g., U.S. Pat. Nos. 5,429,617; 5,772,642 and 7,641,647; U.S. Patent Publication Nos. U520060258982 and US20160113796.

Guidewires for guiding a delivery catheter to a desired location in a body of a patient are known for other delivery stems and may be part of, or used in combination with, the apparatus of the present disclosure. See, e.g., U.S. Pat. No. 69/366,065 and U.S. Patent Publication Nos. U520060074477; US20070299502 and US20080172122. In use, the guidewire may be used with a delivery catheter to deploy the sensing attachment, or a combination of a sensing attachment associated with a medical device such as a stent graft, to a desired location in a patient.

In one embodiment, the present disclosure provides a method including packaging and/or preparing, e.g., treating, the assembly, the assembly including the delivery catheter and the sensing attachment system or the assembly and the combination of a sensing attachment associated with a medical device such as a stent graft. This packaging and preparation facilitates the assembly reaching a desired treatment facility and location, e.g., a hospital, ready for use. The sensing attachment may be shipped in a constrained (e.g. a compressed) or unconstrained (natural) configuration, to the desired treatment facility. In one embodiment, the assembly is packaged and shipped in a constrained configuration. The sensor attachment can be external to the delivery catheter or pre-loaded into the delivery catheter. After packaging, but prior to shipping, the assembly may be sterilized by, e.g., gamma radiation or e-beam. Prior to packaging, the assembly may be sterilized by, e.g., a gaseous method such as exposing the assembly to a gas such as ethylene oxide (EO), ozone, mixed oxides of nitrogen, and chlorine dioxide. In one embodiment, the present disclosure provides a sensing assembly in a packaged form, where the sensing assembly has optionally been sterilized. In one embodiment, the present disclosure provides a sensing assembly in combination with a medical device, e.g., a stent graft, in a packaged form, where optionally the sensing assembly and the medical device, e.g., a stent graft, have each been sterilized. Optionally, in one embodiment, the sensing attachment is in a constrained form when it is within the packaging, e.g., the sensing attachment is pre-loaded into the delivery catheter. Optionally, in one embodiment, the sensing attachment is in a non-constrained form when it is within the packaging, e.g., the sensing attachment is external to a delivery catheter also present within the packaging, or the sensing attachment is associated with a graft or stent graft each in a non-constrained form, or the sensing attachment is packaged alone in a non-constrained form, without the presence of a delivery system.

The materials and compression schemes used for insertion of a sensing attachment via catheter according to the present disclosure are similar to those currently used for coronary stents and endovascular grafts. The sensing attachment can be compressed radially to fit into a catheter delivery system. It would be placed into position in its preferred arterial location via catheter delivery similar to that currently used with coronary stent and endovascular stent technology and deployed in a similar fashion. Alternatively, if using shape memory metal for the ring material, the sensing attachment could be assembled in the ring state and cooled prior to insertion into the delivery system to assume a FIG. 8 or other optimized geometric shape to minimize radial dimension and lengthen axial dimension. This shape change would be designed to facilitate ease of insertion via a smaller French catheter. For a sensing attachment to be placed externally to an endovascular graft within an aneurysmal sac, the sensing attachment is placed prior to endograft in the endovascular sac and may be expand segmentally into a non-circular shape to better match the asymmetric shape of the aneurysmal sac if it is desired to have minimal contact with either the vessel wall within the aneurysmal sac or the endograft.

Thus, in one embodiment, the present disclosure provides a sensing attachment delivery system for deploying a sensing attachment within a vessel and about the outside or internal to the endovascular repair graft comprising: a delivery catheter comprising a tubular enclosure at a distal end portion of the catheter; a sensing attachment encapsulated by tubular configuration, constrained within the tubular enclosure, wherein the sensing attachment is configured to transition between an elongated radially compressed state and a shortened radially expanded state. The delivery system may have radiopaque markers and/or tactual feature that assist in identifying the delivery location.

In one aspect, the present disclosure provides methods and systems for monitoring a medical device, particularly an implanted medical device, and/or the environment surrounding the medical device. Such monitoring may provide information pertinent to the status and functioning of the medical device, where this information may be used by a health care provider to inform decisions about the treatment or prognosis of the patient. Such monitoring may also, or alternatively, provide information pertinent to the status of the patient, which again may be used by a health care provider to inform decisions about the treatment or prognosis of the patient. Such information may also, or alternatively, provide information about the environment around which the sensing attachment is placed, for example, in some instances a stent graft may be implanted along with one or more complementary implants such as an arterial embolic unit. Although the sensing attachment is associated with the stent graft, the sensing attachment may detect and/or measure features of the environment that provide information about the operation of a nearby complementary implant.

Operation of a sensing attachment that is associated with a medical device will be illustrated for an embodiment of the present disclosure where the sensing attachment has a spring form and the medical device is an endovascular graft such as a AAA stent graft, however the same principles apply to other sensing attachments and other implantable medical devices as described herein. Thus, in one aspect, a sensing attachment in the shape of a spring complements an endovascular graft such as a AAA stent graft, and converts such a graft from a passive state to a smart active state which can monitor vascular biological physiology in the vicinity of the endovascular graft.

Once the sensing attachment is placed in the desired location, the sensing attachment is active and may be balanced and calibrated in conjunction to the anatomical body outputs measurable by the sensors on the platform. Having multiple sensors on any sensing attachment affords an opportunity to achieve sensor calibration. In one embodiment, the sensing attachment has multiple sensors. Therefore, a pressure reading in one sensor can be compared to those immediately adjacent, averaged, and adjusted to account for any drift. This would be done externally as part of post process signaling. This is useful because as a sensor may come into contact inadvertently with the lumen wall and/or it may have tissue overgrowth that limits its sensitivity. Additionally, for sensing attachment pressure sensors within the arterial blood flow, they can always be calibrated against external BP pressure measurements and algorithmically adjusted to reflect the changes that occur with mean and pulse pressure throughout the arterial system.

The sensing attachment of the present disclosure carries one or more, e.g., an array of, sensors to detect or measure specific descriptive information in the region of the implanted medical device. For example, when the medical device is implanted in the AAA sac, the sensor or sensor array may detect one or more of pressure, vessel vibration, sound, temperature and so on, which can provide suitable indication of acute and latent issues which may be caused by biological, arterial muscular or treatment graft changes and impact the desired outcome of the corrective procedure.

Grafts and stent grafts are commonly utilized in a wide variety of medical procedures to open up and/or maintain the lumen of a body passageway (e.g. artery, gastrointestinal tract, urinary tract). They are most commonly used however for vascular procedures, e.g., in the treatment of aortic aneurysm disease. An aortic aneurysm AA) is a dilatation of the aorta that usually results from underlying disease (typically atherosclerosis) causing weakness in the vessel wall. As the aneurysm progressively grows in size over time, the risk of it bursting or rupturing rapidly increases; a condition which if not promptly treated, leads to massive hemorrhage and death. Stent grafts are inserted into an aneurysm, not only to simply hold open the diseased vessel, but also to bridge across the dilated vascular segment from healthy vessel to healthy vessel.

Presently available stent grafts, however, have a number of limitations such as endoleaks, migration, detachment, wear and durability issues, rupture, stenosis, kinking and malpositioning. For example, current stent grafts are prone to persistent leakage around the area of the stent graft and into the aneurysm sac (a condition known as an “endoleak”). Hence, pressure within the aneurysm sac is not reduced, stays at or near arterial pressure, and is still at risk for rupture. Endoleaks are among the most common and the most clinically dangerous complications of stent graft placement and the early detection and treatment of endoleaks remains a significant medical problem. Sensing attachments of the present disclosure have, within certain embodiments, pressure detecting sensors that are able to detect elevated pressure within the aneurysm sac and warn the patient and/or the attending physician that there may be a potential endoleak. Pressure sensors on a sensing attachment can recognize abluminal (the outer surface of the graft in contact with the blood vessel wall) pressure rising; this is suggestive that pressure within the aneurysm sac is becoming elevated and that the aneurysm is no longer excluded from the circulation. Since most endoleaks are asymptomatic to the patient (rupture is often the first symptom), a gradual or rapid increase in stent graft abluminal pressure (or aneurysm wall pressure) is an important early indicator that medical care should be sought and that investigation into its underlying cause is warranted. A sensing attachment of the present disclosure, properly placed, can monitor this gradual or rapid increase in stent graft abluminal pressure. Currently, there is no such continuous monitoring and early detection system available to recognize endoleaks and embodiments of the present disclosure will greatly facilitate the identification and early treatment of this potentially fatal complication of stent graft treatment.

There are 5 common types of perigraft leakage (endoleak), and corrective measures can vary depending upon the underlying cause. Sensing attachments of the present disclosure have, within certain embodiments, fluid pressure sensors, contact sensors, position sensors, pulse pressure sensors, blood volume sensors, blood flow sensors, chemistry sensors (e.g., for blood and/or other fluids), metabolic sensors (e.g., for blood and/or other fluids), accelerometers, mechanical stress sensors, temperature sensors, and the like, which are capable of providing information useful to the physician for determining which type of endoleak might be present.

The plurality of sensors affixed to a construct located external to the AAA graft (FIGS. 15, 16, 17, 18 and 19) is designed to measure abluminal leakage into the aneurysm sac as a result of at least one of the four types of endovascular leaks (endoleaks). This is critical as with early detection, clinicians can successfully treat the patient. Current standard of care allows or only ultrasound and/or contrast CT imaging of the vascular graft. In not detected prior to an imaging session, the intervening time may result in graft failure and death as few symptoms manifest prior to failure.

A Type I endoleak is a leak that occurs around the top or bottom of the stent graft. Because blood flowing from the top or bottom areas of the stent graft has high flow, Type I leaks are typically treated with a greater sense of urgency once they are identified. Type II endoleaks are the most common. These are leaks that happen when blood flows into the aneurysm sac from branches of the aorta, or other blood vessel treated with a stent. The blood flows into the aneurysm sac cavity through small branches which enter the treated aneurysm. Type III occurs when there is separation of overlapping stent graft components which allows pressurized blood flow to enter the aneurysm cavity. Type IV Occurs when there is blood flow through the pores of the stent graft.

A plurality of pressure sensors may be used to detect endoleaks as an increase in pressure over baseline. In addition, if pressure sensors are arrayed with a geometric pattern around the circumference of the AAA host graft, the location of the leak may be approximated as the pulsatile jet emanating from the leak will have a local effect, i.e. a local high velocity jet will have a local region of lower dynamic pressure. This can be used to assist the clinician in understanding the location and type of endoleak enabling them to develop a cohesive treatment strategy.

Motion sensors can also detect the root cause for Type I endoleaks. For bifurcated grafts, there is a longitudinal force applied to the graft due to the arterial pulse pressure. When the pressure wave reaches the bifurcation, this imparts a cyclic force on the graft that must be counteracted by the hoop stress fixing the graft at the proximal and distal necks. If the proximal neck of AAA grafts fails to maintain its seal on the host aorta due (1) longitudinal force greater than the radial hoop stress imparted by the AAA graft, (2) further dilation of the host aorta as a result of aneurysm disease progression, or (3) a combination of items 1 and 2, a Type I endoleak occurs. Understanding if the proximal (or distal) connections of the AAA graft are moving from their initial insertion reference position can therefore provide a precursor to Type I endoleaks allowing treatment prior to failure.

The first type of endoleak (Type I Endoleak) occurs when there is direct leakage of blood around the stent graft (either proximally or distally) and into the aneurysm sac. This type of endoleak can be persistent from the time of insertion because of poor sealing between the stent graft and vessel wall, or can develop later because the seal is lost. In addition, this problem can develop due to changes in the position or orientation of the stent graft in relation to the aneurysm as the aneurysm grows, shrinks, elongates or shortens with time after treatment. Type I endoleaks also commonly occur if the stent graft “migrates downstream” from its initial point of placement as a result of being shifted distally by the flow of blood and arterial pulsations. Representative sensing attachments associated with a stent graft can have contact and/or position sensors, where the sensing attachments are located at the proximal and distal ends of the stent graft (optionally, as well as within the body of the stent graft) to assist in the identification of a Type I endoleak. Sensing attachments equipped with pressure and/or contact sensors can indicate the suspected presence of an endoleak through the detection of elevated adluminal pressure; furthermore loss of contact with the vessel wall (as detected by the contact sensors) at the proximal and/or distal ends of the graft would suggest the presence of a Type I endoleak, while loss of contact of the body of the stent graft with the vessel wall would suggest the location, size and extent of the endoleak present in the aneurysm sac. Also, sensing attachments having position sensors and/or accelerometers and located at the proximal and/or distal ends of the stent graft (optionally, as well as in the body of the stent graft) can detect movement (migration) of the stent graft from its original point of placement (a common cause of Type I Endoleaks) and also aid in determining the size and location of the endoleak (by detecting deformations of the stent graft wall).

As noted herein, within certain embodiments, the specific sensors fixed to the sensing attachment can be identified by their USI, as well as by their positional location within the sensing attachment. Hence, a more comprehensive image or analysis of the overall function of the stent graft (and of the patient's response to the stent graft) can be ascertained based upon knowledge of the location and activities of a group of sensors collectively. For example, a collection of sensors, when analyzed as a group could be utilized to ascertain the specific type of endoleak, the degree and the location of the endoleak. In addition, the collection of sensors could be utilized to assess a variety of other conditions, including for example, kinking or deformation of the stent graft, and stenosis of the stent graft.

The collection of data from the sensors of a sensing attachment can also be utilized to ensure proper placement of the stent graft (e.g., that no leaks are present at the time of placement), and that the stent graft is appropriately positioned (e.g., and that the side arm is appropriately attached to the main body of the stent graft).

The second type of perigraft leak (Type II Endoleak) can occur because there are side arteries extending out the treated segment of blood vessel (typically the lumbar arteries, testicular arteries and/or the inferior mesenteric artery). Once the aneurysm is excluded by the stent graft, flow can reverse within these blood vessels and continue to fill the aneurysm sac around the stent graft. A sensing attachment of the present disclosure may have contact and/or position sensors, two such sensing attachments may be associated at the proximal and distal ends of the stent graft (optionally, as well as within the body of the stent graft) to assist in the identification of a Type II endoleak. Sensing attachments equipped with pressure and/or contact sensors, and associated with an implanted stent graft, can indicate the suspected presence of an endoleak through the detection of elevated adluminal pressure; furthermore continued contact with the vessel wall (as detected by the contact sensors) at the proximal and/or distal ends of the graft would suggest the endoleak could be a Type II, while loss of contact of the body of the stent graft with the vessel wall would suggest the location, size and extent of the endoleak present in the aneurysm sac. Lastly, sensing attachments located at the proximal and distal ends of the stent graft, and having position sensors and/or accelerometers, would confirm that the stent graft had not migrated from its original point of placement, while those sensors located in the body of the stent graft would aid in determining the size and anatomical location of the endoleak (by detecting deformations of the stent graft wall) which could suggest the blood vessel responsible for the Type II endoleak.

The third type of endoleak (Type III Endoleak) can occur because of disarticulation of the device (in the case of modular or segmented devices). Due to the complicated vascular anatomy, the diversity of aneurysm shapes and the need to custom fit the stent graft to a particular patient, many stent grafts are composed of several segments that are inserted separately and constructed within aorta into their final configuration. Disarticulation of the device at the junction points can develop due to changes in shape of the aneurysm as it grows, shrinks, elongates or shortens with time after treatment. Sensing attachments may be specifically associated with two or more of these segmented devices, where the sensing attachments may have, e.g., contact and/or position sensors. These sensors may be monitored to assist in assessing the integrity of the seal between stent graft segments. During placement of the stent graft, complimentary sensing attachments may have paired/matched contact sensors on the respective sensing attachments that can be used to confirm that a precise and accurate connection has been achieved during construction of the device. Should a Type III endoleak develop, gaps/discontinuities between contact sensors on sensing attachments located on complimentary segments can be detected to ascertain both the location and extent of the endoleak present.

A fourth type of endoleak (Type IV Endoleak) occurs due to the development of holes within the graft material through which blood can leak into the aneurysm sac. Continuous pulsation of the vessel causes the graft material to rub against the metallic stent tynes eventually leading to fabric wear and graft failure. Representative sensing attachments of the present disclosure have fluid pressure sensors, contact sensors, position sensors, pulse pressure sensors, blood volume sensors, blood flow sensors, chemistry sensors (e.g., for blood and/or other fluids), metabolic sensors (e.g., for blood and/or other fluids), accelerometers, mechanical stress sensors, temperature sensors, and the like sensors that can be associated with near the fabric of the body of the stent graft to assist in the identification of a Type IV endoleak. Should a defect develop in the graft material, the associated sensors will aid in determining the size and location of the endoleak by detecting deformations and defects of the stent graft wall. In extreme cases, stent graft wall defects can lead to rupture of the stent graft; a condition that can be detected early as a result of embodiments of this disclosure.

The final type of endoleak (Type V Endoleak) is a leak of unknown origin. Representative sensing attachments equipped with fluid pressure sensors, contact sensors, position sensors, pulse pressure sensors, blood volume sensors, blood flow sensors, chemistry sensors (e.g., for blood and/or other fluids), metabolic sensors (e.g., for blood and/or other fluids), accelerometers, mechanical stress sensors, temperature sensors, and the like can be associated with a stent graft and indicate the suspected presence of an endoleak through the detection of elevated adluminal pressure. Furthermore, loss of contact with the vessel wall detected by contact sensors, changes in position sensors and/or movements detected by accelerometers can detect changes in the stent graft and assist in determining the size and location of the endoleak (by detecting deformations of the stent graft wall).

Sensing attachments associated with stent grafts according to the present disclosure can provide sensing information to serve a variety of important clinical functions. For example, this information is useful to the clinician during initial placement of the stent graft to determine if it is correctly placed anatomically, if there is leakage around the graft, if stent graft segments are correctly assembled, to detect kinking or deformation of the graft, to ascertain if there is uniform blood flow through the device—to name but a few important functions. Malpositioning of the stent graft, either at the time of placement or due to subsequent movement/migration, is a common complication of stent graft therapy. Sensing attachments associated with stent grafts according to the present disclosure may be used to confirm proper initial placement and any ensuing relocation. Detachment of the graft as a whole (from the artery), or detachment of individual graft segments from each other is another problematic complication of stent graft insertion and ongoing therapy. Sensing attachments associated with stent grafts according to the present disclosure may have the ability to detect movement/detachment of the entire stent graft, as well as movement and/or detachment of individual segments, providing the clinician and patient with valuable diagnostic information. Kinking of the stent graft during deployment and/or as the result of subsequent movement after placement is also a significant clinical problem if it develops. Sensing attachments associated with stent grafts according to the present disclosure have position sensors and accelerometers that may be capable of detecting deformation and kinking of the stent graft.

In some cases, the lumen of the stent graft can become narrowed and restrict blood flow through the graft due to external compression (such as an endoleak), stenosis (the growth of thickened vascular tissue called neointimal hyperplasia on the inner surface of the stent graft), or the formation of a blot clot. Sensing attachments associated with stent grafts according to the present disclosure have a variety of sensors capable of detecting and differentiating types of stenosis. Blood flow, fluid pressure and blood volume sensors on a sensing attachment located on the luminal surface of the stent graft are able to detect the presence and location of a stenosis due to the increased blood flow speed and increased blood (and pulse) pressure at the site of a stenosis (relative to normal segments of the graft), as well as stenosis due to external compression (such as the presence of an endoleak as discussed above). Stenosis due to neointimal hyperplasia or clot formation will be detected as “dead spots” and/or altered readings on the luminal surface as blood flow sensors, blood metabolic and/or chemistry sensors (e.g., for blood and/or other fluids) become covered by vascular tissue or clot; while adluminal pressure sensors and accelerometers will not show changes in adluminal pressure or stent graft wall deformation (as would occur with an endoleak). Metabolic sensors and chemistry sensors are capable of determining the difference between stenosis (normal pH and physiologic readings) and clot (lowered pH and altered physiologic readings). The present disclosure provides sensing attachments that can be associated with a stent graft in order to make these determinations, and methods of doing the same.

As mentioned, stent grafts are often placed in arteries (typically the aorta) in anatomic locations where important arterial side branches originate. Of greatest importance are the renal arteries, but the lumbar, testicular, inferior mesenteric and internal iliac arteries can be affected by an aortic aneurysm. To maintain patency of these arteries (and prevent them from being obstructed by the placement of the stent graft), stent grafts with holes (or fenestrations) have been developed that allow blood flow through the graft and into the arteries that branch out from the aorta. FEVAR (fenestrated endovascular aortic aneurysm repair) is a form stent graft design and treatment that maintains the patency of important blood vessels that originate from the aorta. Sensing attachments of the present disclosure have sensors, e.g., blood flow sensors, fluid pressure sensors, pulse pressure sensors, blood volume sensors and/or blood chemistry and metabolic sensors, where the sensing attachments may be associated with the stent graft at the fenestration sites to monitor blood flow through the side branches. Likewise, sensing attachments of the present disclosure may also have position sensors, contact sensors and/or accelerometers, which can be associated at the fenestration sites to monitor patency of the side branches (due to stenosis and/or kinking, migration and obstruction of the arterial branches by the stent graft itself).

In addition, patients requiring stent grafts often have extensive cardiovascular disease resulting in impaired cardiac and circulatory function. For example, patients receiving stent grafts are at an increased risk for myocardial infarction (heart attack), congestive heart failure, renal failure and arrhythmias. The aorta is the largest blood vessel to originate from the heart; therefore, monitoring certain hemodynamic and metabolic parameters within the aorta can provide the clinician with very important information regarding the patient's cardiac, renal and circulatory function. Sensing attachments associated with stent grafts according to the present disclosure contain fluid pressure sensors, contact sensors, position sensors, pulse pressure sensors, blood volume sensors, blood flow sensors, chemistry sensors (e.g., for blood and/or other fluids), metabolic sensors (e.g., for blood and/or other fluids), accelerometers, mechanical stress sensors, temperature sensors, and the like, suitable for such purposes. Representative sensing attachments of the present disclosure may have pressure sensors, pulse pressure sensors, pulse contour sensors, blood volume sensors, blood flow sensors which may be associated with the stent graft, and which provide information which can be used by one of ordinary skill in the art to calculate and monitor important physiologic parameters such as cardiac output (CO), stroke volume (SV), ejection fraction (EV), systolic blood pressure (sBP), diastolic blood pressure (dBP), mean arterial pressure (mAP), systemic vascular resistance (SVR), total peripheral resistance (TPV) and pulse pressure (PP). For example, the FloTrac/Vigileo (Edwards Life Sciences, Irvine, CA) uses pulse contour analysis to calculate stroke volume (SV) and systemic vascular resistance (SVR); the pressure recording analytical method (PRAM) is used by Most Care (Vytech, Padora, Italy) to estimate cardiac output (CO) from analysis of the arterial pressure wave profile. Changes in cardiac output (CO), stroke volume (SV) and ejection fraction (EF) and cardiac index (CI) can be an important in detecting complications such myocardial ischemia and infarction; they can also assist the clinician in implementation and adjusting cardiac medications and dosages. Pulse pressure sensors, pulse contour sensors and heart rate sensors contained as part of a sensing attachment and associated with a stent graft may assist in the detection and monitoring of cardiac arrhythmias and heart rate abnormalities; they too can be used to monitor the patient's response to cardiac medications that effect heart rate and rhythm. Systolic blood pressure (sBP), diastolic blood pressure (dBP), mean arterial pressure (mAP), systemic vascular resistance (SVR) and total peripheral resistance (TPV) readings can be used by the clinician to monitor the dosage and effect of blood pressure lowering medications and pressor (blood pressure increasing) agents.

As described above, patients requiring stent grafts often have concurrent medical problems related to cardiovascular disease such as renal impairment or renal failure. The renal arteries originate from the aorta, often in close approximation to the typical location of stent graft placement; therefore, monitoring certain hemodynamic and metabolic parameters within the aorta can provide the physician and patient with very important “real time” information regarding ongoing renal function. Sensing attachments associated with stent grafts according to the present disclosure can contain circulatory sensors (as described herein) as well as chemistry sensors (e.g., for blood and/or other fluids) and metabolic sensors (e.g., for blood and/or other fluids) suitable for monitoring kidney function. Examples of blood chemistry and metabolic sensors of utility for this embodiment include, but are not limited to, Blood Urea Nitrogen (BUN), Creatinine (Cr) and Electrolytes (Calcium, Potassium, Phosphate, Sodium, etc.) Furthermore, combining metabolic data with hemodynamic data and urinalysis can allow the clinician to calculate the Glomerular Filtration Rate (GFR) which is a very useful measure of kidney function. This information would be of particular utility in the management of dialysis patients to monitor the timing, effectiveness, and frequency of dialysis therapy.

Finally, due to the numerous complications described above, there is long term uncertainty about the entire stent graft technology as a treatment for aortic aneurysm. Although much more invasive and traumatic, standard open surgical aneurysm repair is extremely durable and effective. Uncertainties about endovascular stent grafts include whether they will lower the aneurysm rupture rate, rate of perigraft leak (endoleak), device migration, the ability to effectively exclude aneurysms over a long term, and device rupture or disarticulation. Sensing attachments associated with stent grafts according to the present disclosure, having the ability to detect and monitor many (if not all) of the aforementioned complications, are an important advancement of stent graft therapy as a whole.

In one embodiment, the sensors shall obtain and transfer sensed information to a memory chip. The information is then formed into applicable and determine packets and transferred from the memory chip to a receiver located external of the patient's body for any processing, logging, timestamping or calculating in an algorithm to provide data numerically, pictorially or graphically which enables the trained reviewer to assess the status of the implant and/or surrounding environment and make appropriate decisions based thereon, e.g., making intended correction of the procedure.

In one embodiment, the sensing attachment complements an endovascular graft and converts the graft from passive state to smart active state activity by monitoring vascular biological physiology.

Placing a scaffold with sensors internal to a AAA graft at proximal and distal locations enables a range of hemodynamic assessment. An example sensing attachment placed internal to a AAA graft, i.e., adluminally, is illustrated in FIG. 16, where sensing attachment 122 is entirely within the stent graft at a distal location, and sensing attachment 120 is placed partially adluminally and partially abluminally, i.e., on the outer surface of the stent graft, at a proximal location, where blood flows from the proximal end to the distal end of the stent graft. Although FIG. 15, FIG. 17 and FIG. 18 illustrate the sensing attachment located entirely on the abluminal surface of the stent graft, the sensing attachment could alternatively be located on the adluminal surface of the of the stent graft. Also, although FIG. 15, FIG. 17 and FIG. 18 illustrate the sensing attachment located at about the center of the stent graft, within the aneurysm sac, the sensing attachment could alternatively be located at the proximal end and/or a distal end of the stent graft. Thus, in one embodiment, the stent graft is associated with two sensing attachments, both of which are located adluminally to the stent graft, one at the proximal end of the stent graft and another is located at a distal end of the stent graft.

With pressure and/or flow sensors in these locations, i.e., adluminally at the proximal and distal ends of the stent graft, a full assessment of patient hemodynamic status may be ascertained and provided to both patient and clinician. Data from pressure and/or flow sensors can be used to calculate a range of hemodynamic parameters including heart rate, blood pressure, pulse pressure, cardiac output, stroke volume, total peripheral resistance, and graft patency. In aggregate, these parameters are useful to enable clinicians to manage a range of disease pathologies with pharmacologic intervention including hypertension, congestive heart failure, and atrial fibrillation with a temporal frequency much higher than current standard of care affords through infrequent clinician office visits.

The sensing attachment may be incorporated into an environment which communicates with the sensing attachment. An example environment is an operating room wherein the sensing attachment is being implanted into a patient by a health care professional. Another example environment is the patient's home, in the case where the sensing attachment has already been implanted in the patient. Yet another example environment is a doctor's office, where the patient having the implanted sensing attachment is in the office for, e.g., an evaluation. The following provides a detailed description of an example environment in a patient's home. However, the described features and connectivity are analogously present in other environments within which the patient with the implanted sensing attachment are present, e.g., the operating room and the doctor's office, as also described herein albeit in lesser detail.

FIG. 22 illustrates a context diagram of a sensing attachment environment 1000 including the patient's home. In the environment, a sensing attachment 1002 comprising an implantable reporting processor 1003 has been implanted into a patient (not shown). The implantable reporting processor (IRP) 1003 is arranged and configured to collect data including for example, medical and health data related to a patient which the device is associated, and operational data of the sensing attachment 1002 itself. The sensing attachment 1002 communicates with one or more home base stations 1004 or one or more smart devices 1005 during different stages of monitoring the patient.

The sensing attachment 1002 includes one or more sensors that collect information and data, including medical and health data related to a patient which the sensing attachment is associated, and operational data of the sensing attachment 1002 itself. The sensing attachment 1002 collects data at various different times and at various different rates during a monitoring process of the patient, and may optionally store that data in a memory until it is transmitted outside the body of the patient. In some embodiments, the sensing attachment 1002 may operate in a plurality of different phases over the course of monitoring the patient. For instance, more data may be collected soon after the sensing attachment 1002 is implanted into the patient, but less data is collected at later times.

The amount and type of data collected by the sensing attachment 1002 may be different from patient to patient, and the amount and type of data collected may change for a single patient. For example, a medical practitioner studying data collected by the sensing attachment 1002 of a particular patient may adjust or otherwise control how the sensing attachment 1002 collects future data.

The amount and type of data collected by a sensing attachment 1002 may be different for different types of patient conditions, for different patient demographics, or for other differences. Alternatively, or in addition, the amount and type of data collected may change overtime based on other factors, such as how the patient is healing or feeling, how long the monitoring process is projected to last, how much power remains in the sensing attachment 1002 and should be conserved, the type of movement being monitored, the body part being monitored, and the like. In some cases, the collected data is supplemented with personally descriptive information provided by the patient such as subjective pain data, quality of life metric data, co-morbidities, perceptions or expectations that the patient associates with the sensing attachment 1002, or the like.

Once the sensing attachment 1002 is implanted into the patient and the patient returns home, the sensing attachment may begin communications outside of the patient's body, within the home environment. The communication may be with, e.g., the home base station 1004, the smart device 1005 (e.g., the patient's smart phone), the connected personal assistant 1007, or two or more of the home base station, and the smart device, and the connected personal assistant can communicate with the sensing attachment 1002. The sensing attachment 1002 can collect data at determined rates and times, variable rates and times, or otherwise controllable rates and times. Data collection can start when the sensing attachment 1002 is initialized in the operating room, when directed by a medical practitioner, or at some later point in time. At least some data collected by the sensing attachment 1002 may be transmitted to the home base station 1004 directly, to the smart device 1005 directly, to the connected personal assistant 1007 directly, to the base station via one or both of the smart device and the connected personal assistant, to the smart device via one or both of the base station and the connected personal assistant, or to the connected personal assistant via one or both of the smart device and the base station. Here, “one or both” means via an item alone, and via both items serially or in parallel. For example, data collected by the sensing attachment 1002 may be transmitted to the home base station 1004 via the smart device 1005 alone, via the connected personal assistant 1007 alone, serially via the smart device and the connected personal assistant, serially via the connected personal assistant and the smart device, and directly, and possibly contemporaneously, via both the smart device and the connected personal assistant. Similarly, data collected by the sensing attachment 1002 may be transmitted to the smart device 1005 via the home base station 1004 alone, via the connected personal assistant 1007 alone, serially via the home base station and the connected personal assistant, serially via the connected personal assistant and the home base station, and directly, and possibly contemporaneously, via both the home base station and the connected personal assistant. Further in example, data collected by the sensing attachment 1002 may be transmitted to the connected personal assistant 1007 via the smart device 1005 alone, via the home base station 1004 alone, serially via the smart device and the home base station, serially via the home base station and the smart device, and directly, and possibly contemporaneously, via both the smart device and the home base station.

In various embodiments, one or more of the home base station 1004, the smart device 1005, and the connected personal assistant 1007 pings the sensing attachment 1002 at periodic, predetermined, or other times to determine if the sensing attachment 1002 is within communication range of one or more of the home base station, the smart device, and the connected personal assistant. Based on a response from the sensing attachment 1002, one or more of the home base station 1004, the smart device 1005, and the connected personal assistant 1007 determines that the sensing attachment 1002 is within communication range, and the sensing attachment 1002 can be requested, commanded, or otherwise directed to transmit the data it has collected to one or more of the home base station 1004, the smart device 1005, and the connected personal assistant 1007.

Each of one or more of the home base station 1004, the smart device 1005, and the connected personal assistant 1007 may, in some cases, be arranged with a respective optional user interface. The user interface may be formed as a multimedia interface that unidirectionally or bi-directionally passes one or more types of multimedia information (e.g., video, audio, tactile, etc.). Via the respective user interface of one or more of the home base station 1004, the smart device 1005, and the connected personal assistant 1007, the patient (not shown in FIG. 22) or an associate (not shown in FIG. 22) of the patient may enter other data to supplement the data collected by the sensing attachment 1002. A user, for example, may enter personally descriptive information (e.g., age change, weight change), changes in medical condition, co-morbidities, pain levels, quality of life, an indication of how the sensing attachment 1002 “feels,” or other subjective metric data, personal messages for a medical practitioner, and the like. In these embodiments, the personally descriptive information may be entered with a keyboard, mouse, touch-screen, microphone, wired or wireless computing interface, or some other input means. In cases where the personally descriptive information is collected, the personally descriptive information may include, or otherwise be associated with, one or more identifiers that associate the information with unique identifier of the sensing attachment 1002, the patient, an associated medical practitioner, an associated medical facility, or the like.

In some of these cases, a respective optional user interface of each of one or more of the home base station 1004, the smart device 1005, and the connected personal assistant 1007 may also be arranged to deliver information associated with the sensing attachment 1002 to the user from, for example, a medical practitioner. In these cases, the information delivered to the user may be delivered via a video screen, an audio output device, a tactile transducer, a wired or wireless computing interface, or some other like means.

In embodiments where one or more of the home base station 1004, the smart device 1005, and the connected personal assistant 1007 are arranged with a user interface, which may be formed with an internal user interface arranged for communicative coupling to a patient portal device. The patent portal device may be smartphone, a tablet, a body-worn device, a weight or other health measurement device (e.g., thermometer, bathroom scale, etc.), or some other computing device capable of wired or wireless communication. In these cases, the user is able to enter the personally descriptive information, and the user also may be able to receive information associated with the sensing attachment 1002.

The home base station 1004 utilizes a home network 1006 of the patient to transmit the collected data to cloud 1008. The home network 1006, which may be a local area network, provides access from the home of the patient to a wide area network, such as the internet. In some embodiments, the home base station 1004 may utilize a Wi-Fi connection to connect to the home network 1006 and access the internet. In other embodiments, the home base station 1004 may be connected to a home computer (not shown in FIG. 22) of the patient, such as via a USB connection, which itself is connected to the home network 1006.

The smart device 1005 can communicate with the sensing attachment 1002 directly via, for example, Blue Tooth® compatible signals, and can utilize the home network 1006 of the patient to transmit the collected data to cloud 1008, or can communicate directly with the cloud, for example, via a cellular network. Alternatively, the smart device 1005 is configured to communicate directly with one or both of the home base station 1004 and the connected personal assistant 1007 via, for example, Blue Tooth® compatible signals, and is not configured to communicate directly with the sensing attachment 1002.

Furthermore, the connected personal assistant 1007 can communicate with the sensing attachment 1002 directly via, for example, Blue Tooth® compatible signals, and can utilize the home network 1006 of the patient to transmit the collected data to cloud 1008, or can communicate directly with the cloud, for example, via a modem/internet connection or a cellular network. Alternatively, the connected personal assistant 1007 is configured to communicate directly with one or both of the home base station 1004 and the smart device 1005 via, for example, Blue Tooth® compatible signals, and is not configured to communicate directly with the sensing attachment 1002.

Along with transmitting collected data to the cloud 1008, one or more of the home base station 1004, the smart device 1005, and the connected personal assistant 1007 may also obtain data, commands, or other information from the cloud 1008 directly or via the home network 1006. One or more of the home base station 1004, the smart device 1005, and the connected personal assistant 1007 may provide some or all of the received data, commands, or other information to the sensing attachment 1002. Examples of such information include, but are not limited to, updated configuration information, diagnostic requests to determine if the sensing attachment 1002 is functioning properly, data collection requests, and other information.

The cloud 1008 may include one or more server computers or databases to aggregate data collected from the sensing attachment 1002, and in some cases personally descriptive information collected from a patient (not shown in FIG. 22), with data collected from other sensing attachments (not illustrated), and in some cases personally descriptive information collected from other patients. In this way, the cloud 1008 can create a variety of different metrics regarding collected data from each of a plurality of sensing attachments that are implanted into separate patients. This information can be helpful in determining if the sensing attachments are functioning properly. The collected information may also be helpful for other purposes, such as determining which specific devices may not be functioning properly, determining if a procedure or condition associated with the sensing attachment is helping the patient (e.g., if the stent graft is operating properly), and determining other medical information.

Still referring to FIG. 22, alternate embodiments are contemplated. For example, one or two of the home base station 1004, the smart device 1005, and the connected personal assistant 1007 may be omitted from the sensing attachment environment 1000. Furthermore, each of the home base station 1004, the smart device 1005, and the connected personal assistant 1007 may be configured to communicate with one or both of the sensing attachment 1002 and the cloud 1008 via another one or two of the base station, the smart device, and the connected personal assistant. Moreover, the smart device 1005 can be temporarily contracted as an interface to the sensing attachment 1002, and can be any suitable device other than a smart phone, such as a smart watch, a smart patch, and any IoT device, such as a coffee pot, capable of acting as an interface to the sensing attachment 1002. In addition, one or more of the home base station 1004, smart device 1005, and connected personal assistant 1007 can act as a communication hub for multiple sensing attachments implanted in one or more patients. Furthermore, one or more of the home base station 1004, smart device 1005, and connected personal assistant 1007 can automatically order or reorder prescriptions or medical supplies in response to patient input or sensing attachment input (e.g., pain level, instability level) if a medical professional and insurance company have preauthorized such an order or reorder; alternatively, one or more of the base station, smart device, and connected personal assistant can be configured to request, from a medical professional or an insurance company, authorization to place the order or reorder. Moreover, one or more of the home base station 1004, smart device 1005, and connected personal assistant 1007 can be configured with a personal assistant such as Alexa® or Sin®.

Although the sensing attachment environment has been described in the context of a patient's home, the same principles apply when the environment is an operating room or a doctor's office. For example, in association with a medical procedure, a sensing attachment 1002 may be implanted in the patient's body within an operating room environment. Coetaneous with the medical procedure, the sensing attachment 1002 communicates with an operating room base station (analogous to the home base station). Subsequently, after sufficient recovery from the medical procedure, the patient returns home wherein the sensing attachment 1002 is arranged to communicate with a home base station 1004. Thereafter, at other times, the sensing attachment 1002 is arranged to communicate with a doctor office base station when the patient visits the doctor for a follow-up consultation. In any case, the sensing attachment 1002 communicates with each base station via a short range network protocol, such as the medical implant communication service (MICS), the medical device radio communications service (MedRadio), or some other wireless communication protocol suitable for use with the sensing attachment 1002.

For example, implantation of the sensing attachment 1002 into the patient may occur in an operating room. As used herein, operating room includes any office, room, building, or facility where the sensing attachment 1002 is implanted into the patient. For example, the operating room may be a typical operating room in a hospital, an operating room in a surgical clinic or a doctor's office, or any other operating theater where the sensing attachment 1002 is implanted into the patient.

The operating room base station (analogous to the home base station of FIG. 22) is utilized to configure and initialize the sensing attachment 1002 in association with the sensing attachment 1002 being implanted into the patient. A communicative relationship is formed between the sensing attachment 1002 and the operating room base station, for example, based on a polling signal transmitted by the operating room base station and a response signal transmitted by the sensing attachment 1002.

Upon forming a communicative relationship, which will often occur prior to implantation of the sensing attachment 1002, the operating room base station transmits initial configuration information to the sensing attachment 1002. This initial configuration information may include, but is not limited to, a time stamp, a day stamp, an identification of the type and placement of the sensing attachment 1002, information on other implants associated with the sensing attachment, surgeon information, patient identification, operating room information, and the like.

In some embodiments, the initial configuration information is passed unidirectionally; in other embodiments, initial configuration is passed bidirectionally. The initial configuration information may define at least one parameter associated with the collection of data by the sensing attachment 1002. For example, the configuration information may identify settings for one or more sensors on the sensing attachment 1002 for each of one or more modes of operation. The configuration information may also include other control information, such as an initial mode of operation of the sensing attachment 1002, a particular event that triggers a change in the mode of operation, radio settings, data collection information (e.g., how often the sensing attachment 1002 wakes up to collected data, how long it collects data, how much data to collect), home base station 1004, smart device 1005, and connected personal assistant 1007 identification information, and other control information associated with the implantation or operation of the sensing attachment 1002. Examples of the connected personal assistant 1007, which also can be called a smart speaker, include Amazon Echo®, Amazon Dot®, Google Home®, Philips® patient monitor, Comcast's health-tracking speaker, and Apple HomePod®.

In some embodiments, the configuration information may be pre-stored on the operating room base station or an associated computing device. In other embodiments, a surgeon, surgical technician, or some other medical practitioner may input the control information and other parameters to the operating room base station for transmission to the sensing attachment 1002. In at least one such embodiment, the operating room base station may communicate with an operating room configuration computing device. The operating room configuration computing device includes an application with a graphical user interface that enables the medical practitioner to input configuration information for the sensing attachment 1002. In various embodiments, the application executing on the operating room configuration computing device may have some of the configuration information predefined, which may or may not be adjustable by the medical practitioner.

The operating room configuration computing device communicates the configuration information to the operating room base station via a wired or wireless network connection (e.g., via a USB connection, Bluetooth connection, Bluetooth Low Energy (BTLE) connection, or Wi-Fi connection), which in turn communicates it to the sensing attachment 1002.

The operating room configuration computing device may also display information regarding the sensing attachment 1002 or the operating room base station to the surgeon, surgical technician, or other medical practitioner. For example, the operating room configuration computing device may display error information if the sensing attachment 1002 is unable to store or access the configuration information, if the sensing attachment 1002 is unresponsive, if the sensing attachment 1002 identifies an issue with one of the sensors or radio during an initial self-test, if the operating room base station is unresponsive or malfunctions, or for other reasons.

Although the operating room base station and the operating room configuration computing device are described as separate devices, embodiments are not so limited; rather, the functionality of the operating room configuration computing device and the operating room base station may be included in a single computing device or in separate devices as illustrated. In this way, the medical practitioner may be enabled in one embodiment to input the configuration information directly into the operating room base station.

After the sensing attachment has been implanted in the patient, the patient may periodically visit a doctor's office for follow-up evaluation. In one aspect, the present disclosure provides a doctor's office environment (analogous to the home environment described herein) wherein the implanted sensing attachment communicates with the office environment. During these visits, the data that has been stored in memory may be accessed, and/or specific data may be requested and obtained as part of a monitoring process.

For example, at various times throughout the monitoring process, the patient may be requested to visit a medical practitioner for follow up appointments. This medical practitioner may be the surgeon who implanted the sensing attachment 1002 in the patient or a different medical practitioner that supervises the monitoring process, physical therapy, and recovery of the patient. For a variety of different reasons, the medical practitioner may want to collect real-time data from the sensing attachment 1002 in a controlled environment. In some cases, the request to visit the medical practitioner may be delivered through a respective optional bidirectional user interface of each of one or more of the home base station 1004, the smart device 1005, and the connected personal assistant 1007.

A medical practitioner utilizes the doctor office base station (analogous to the home base station shown in FIG. 22), which communicates with the sensing attachment 1002, to pass additional data between the doctor office base station and the sensing attachment 1002. Alternatively, or in addition, the medical practitioner utilizes the doctor office base station (not shown in FIG. 22) to pass commands to the sensing attachment 1002. In some embodiments, the doctor office base station instructs the sensing attachment 1002 to enter a high-resolution mode to temporarily increase the rate or type of data that is collected for a short time. The high-resolution mode directs the sensing attachment 1002 to collect different (e.g., large) amounts of data during an activity where the medical practitioner is also monitoring the patient.

In some embodiments, the doctor office base station enables the medical practitioner to input event or pain markers, which can be synchronized with the high-resolution data collected by the sensing attachment 1002. For example, the medical practitioner can have the patient walk on a treadmill while the sensing attachment 1002 is in the high-resolution mode. As the patient walks, the patient may complain about pain. The medical practitioner can click a pain marker button on the doctor office base station to indicate the patient's discomfort. The doctor office base station records the marker and the time at which the marker was input. When the timing of this marker is synchronized with the timing of the collected high-resolution data, the medical practitioner can analyze the data to try and determine the cause of the pain.

In other embodiments, the doctor office base station may provide updated configuration information to the sensing attachment 1002. The sensing attachment 1002 can store this updated configuration information, which can be used to adjust the parameters associated with the collection of the data. For example, if the patient is doing well, the medical practitioner can direct a reduction in the frequency at which the sensing attachment 1002 collects data. On the contrary, if the patient is experiencing an unexpected amount of pain, the medical practitioner may direct the sensing attachment 1002 to collect additional data for a determined period of time (e.g., a few days). The medical practitioner may use the additional data to diagnose and treat a particular problem. In some cases, the additional data may include personally descriptive information provided by the patient after the patient has left presence of the medical practitioner and is no longer in range of the doctor office base station. In these cases, the personally descriptive information may be collected and delivered from via one or more of the home base station 1004, the smart device 1005, and the connected personal assistant 1007. Firmware within the sensing attachment and/or the base station will provide safeguards limiting the duration of such enhanced monitoring to ensure the sensing attachment 1002 retains sufficient power to last for the implant's lifecycle.

In various embodiments, the doctor office base station may communicate with a doctor office configuration computing device (analogous to the operating room computing device). The doctor office configuration computing device includes an application with a graphical user interface that enables the medical practitioner to input commands and data. Some or all of the commands, data, and other information may be later transmitted to the sensing attachment 1002 via the doctor office base station. For example, in some embodiments, the medical practitioner can use the graphical user interface to instruct the sensing attachment 1002 to enter its high-resolution mode. In other embodiments, the medical practitioner can use graphical user interface to input or modify the configuration information for the sensing attachment 1002. The doctor office configuration computing device transmits the information (e.g., commands, data, or other information) to the doctor office base station via a wired or wireless network connection (e.g., via a USB connection, Bluetooth connection, or Wi-Fi connection), which in turn transmits some or all of the information to the sensing attachment 1002.

The doctor office configuration computing device may also display, to the medical practitioner, other information regarding the sensing attachment 1002, regarding the patient (e.g., personally descriptive information), or the doctor office base station. For example, the doctor office configuration computing device may display the high-resolution data that is collected by the sensing attachment 1002 and transmitted to the doctor office base station. The doctor office configuration computing device may also display error information if the sensing attachment 1002 is unable to store or access the configuration information, if the sensing attachment 1002 is unresponsive, if the sensing attachment 1002 identifies an issue with one of the sensors or radio, if the doctor office base station is unresponsive or malfunctions, or for other reasons.

In some embodiments, doctor office configuration computing device may have access to the cloud 1008. In at least one embodiment, the medical practitioner can utilize the doctor office configuration computing device to access data stored in the cloud 1008, which was previously collected by the sensing attachment 1002 and transmitted to the cloud 1008 via one or both of the home base station 1004 and smart device 1005. Similarly, the doctor office configuration computing device can transmit the high-resolution data obtain from the sensing attachment 1002 via the doctor office base station to the cloud 1008. In some embodiments, the doctor office base station may have internet access and may be enabled to transmit the high-resolution data directly to the cloud 1008 without the use of the doctor office configuration computing device.

In various embodiments, the medical practitioner may update the configuration information of the sensing attachment 1002 when the patient is not in the medical practitioner's office. In these cases, the medical practitioner can utilize the doctor office configuration computing device (not shown in FIG. 22) to transmit updated configuration information to the sensing attachment 1002 via the cloud 1008. One or more of the home base station 1004, the smart device 1005, and the connected personal assistant 1007 can obtain updated configuration information from the cloud 1008 and pass updated configuration information to the cloud. This can allow the medical practitioner to remotely adjust the operation of the sensing attachment 1002 without needing the patient to come to the medical practitioner's office. This may also permit the medical practitioner to send messages to the patient in response, for example, to personally descriptive information that was provided by the patient and passed through one or more of the home base station 1004, the smart device 1005, and the connected personal assistant 1007 to the doctor office base station (not shown in FIG. 22). For example, if a patient speaks “I feel pain” into the connected personal assistant 1007, then the medical practitioner may issue a prescription for a pain reliever and cause the connected personal assistant to notify the patient by “speaking” “the doctor has called in a prescription for Vicodin® to your preferred pharmacy; the prescription will be ready for pick up at 4 pm.”

Although the doctor office base station (not shown in FIG. 22) and the doctor office configuration computing device (not shown in FIG. 22) are described as separate devices, embodiments are not so limited; rather, the functionality of the doctor office configuration computing device and the doctor office base station may be included in a single computing device or in separate devices (as illustrated). In this way, the medical practitioner may be enabled in one embodiment to input the configuration information or markers directly into the doctor office base station and view the high-resolution data (and synchronized marker information) from a display on the doctor office base station.

In one embodiment, sensor communication, initiation, and function of the communication and power components would be similar to that described in PCT publication WO2017165717. This offers the advantage of being able to collect and monitor a range of useful information relating to the EVAR as well as the patient's general condition to manage the patient's health. The frequency at which the data is collected is based on a power optimization algorithm taking into account the required frequency of data, size limitations associated with battery technology, memory size, and power requirements of all components (e.g. IMU, memory, sensors, radio). Said information includes but is not limited to: battery power level; implant duration; traceability; implant serial number; acute and chronic measurements including intra sac pressure, arterial pressure at multiple locations, hemodynamic parameters, e.g., CO concentration, blood flow rate, heart rate; and activity measurements such as step count and distance. In addition, the present disclosure optionally provides for integration of patient input data such as BMI, co-morbidities, medication, pain, and qualitative life metrics.

It should be noted that not all data may be collected at each interval. Likewise, it should be noted that the acute and chronic measurements noted above, may only need collection for a few seconds in any interval. It is also provided, that should an aneurysmal sac pressure measurements or other measurements indicate a signal, the patient would be directed to clinicians for further assessment via an interface which connects the patient with their clinician.

In one embodiment, the present disclosure provides released signals, which are signals released from the sensor and which contain information sensed by the sensor. In another embodiment, the present disclosure provides for the capture of the released signal, where this capture may occur in the vicinity of the sensor, or at a distant location. In yet another embodiment, the present disclosure provides for processed released signals, where the released signal is processed to provide useful information.

The present disclosure provides a sensor and construct that is separate from a medical device, such as a graft, so that no physical modifications to the medical device (e.g., graft) are necessary in order for the medical device to have sensing capability. The design is in fact generic for obtaining hemodynamic measurements for any arterial vessel with the sensor(s) placed percutaneously or extra-luminally with a laparoscopic or open surgical approach to implantation. For example, such a system as described herein can be placed proximal and/or distal to a coronary stent to determine when occlusion is occurring, thereby alerting the patient and clinicians to intercede prior to an emergency situation. Depending on placement of the sensors, some embodiments can be used to monitor hemodynamics and pressure associated with ancillary co-morbidities such as hypertension with algorithms to adjust from a local vascular pressure measurement to a systemic pressure measurement for real time diagnostic purposes. The latter allows patients/clinicians to titrate medications to manage their hypertension.

In embodiments, the present disclosure provides: a sensor comprising a housing, where the housing surrounds a detector, the housing comprising an extension that allows the sensor to be fixedly attached to a support; a construct comprising a sensor fixedly attached to a support, where the support can securely engage with a medical device; an assembly comprising a sensor, a support for the sensor, and a medical device, wherein the sensor is in direct contact with and is fixedly attached to the support, and wherein the support is in direct contact with and is securely engaged with the medical device, where optionally the sensor is not in direct contact with the medical device.

The following are exemplary numbered embodiments according to the present disclosure:
[1] A sensor comprising a housing, where the housing surrounds a detector, the housing comprising an extension that allows the sensor to be fixedly attached to a support.
[2] A construct comprising a sensor fixedly attached to a support, where the support can securely engage with a medical device.
[3] An assembly comprising a sensor, a support for the sensor, and a medical device, wherein the sensor is in direct contact with and is fixedly attached to the support, and wherein the support is in direct contact with and is securely engaged with the medical device.
[4] The sensor of embodiment 1 which is sterile.
[5] The construct of embodiment 2 which is sterile.
[6] The assembly of embodiment 3 which is sterile.
[7] The sensor of embodiment 1 wherein the detector detects one of pressure, temperature, motion, and acceleration.
[8] The construct of embodiment 2 wherein the sensor detects one of pressure, temperature, motion, and acceleration.
[9] The assembly of embodiment 3 wherein the sensor detects one of pressure, temperature, motion, and acceleration [10] The sensor of embodiment 1 wherein the detector is a non-biological sensor.
[11] The construct of embodiment 2 wherein the sensor is a non-biological sensor.
[12] The assembly of embodiment 3 wherein the sensor is a non-biological sensor.
[13] The sensor of embodiment 1 comprising medical grade material.
[14] The construct of embodiment 2 comprising medical grade material.
[15] The assembly of embodiment 3 comprising medical grade material.
[16] The sensor of embodiment 1 wherein the sensor comprises a housing, the housing comprising a material selected from metal and polyether ether ketone.
[17] The construct of embodiment 2 wherein the sensor comprises a housing, the housing comprising a material selected from metal and polyether ether ketone.
[18] The assembly of embodiment 3, wherein the sensor comprises a housing, the housing comprising a material selected from metal and polyether ether ketone.
[19] The construct of embodiment 2, wherein the support comprises a material selected from metal (e.g., nitinol) and polyether ether ketone.
[20] The assembly of embodiment 3, wherein the support comprises a material selected from metal (e.g., nitinol) and polyether ether ketone.
[21] The assembly of embodiment 3 wherein the medical device is an implantable medical device.
[22] The construct of embodiment 2 comprising a plurality of sensors (e.g., 2-10 sensors)
[23] The construct of embodiment 22 wherein the plurality of sensors are in direct contact with the support.
[24] The assembly of embodiment 3 comprising a plurality of sensors (e.g., 2 to 10 sensors).
[25] The assembly of embodiment 24 wherein the plurality of sensors are in direct contact with the support.
[26] The assembly of embodiment 3 wherein the medical device comprises rails, and the sensor is fixedly attached to a rail.
[27] The sensor of embodiment 1, wherein the sensor comprises any one or more of a battery, a memory, a radio, an antennae and an inertial measurement unit (IMU).
[28] The construct of embodiment 2, wherein the sensor comprises any one or more of a battery, a memory, a radio, an antennae and an inertial measurement unit (IMU).
[29] The assembly of embodiment 3, wherein the sensor comprises any one or more of a battery, a memory, a radio, an antennae and an inertial measurement unit (IMU).
[30] The sensor of embodiment 1, wherein the sensor comprises a housing and the housing comprises an extension, where the extension comprises one or more holes.
[31] The construct of embodiment 2, wherein the sensor comprises a housing and the housing comprises an extension, where the extension comprises one or more holes.
[32] The assembly of embodiment 3, wherein the sensor comprises a housing and the housing comprising an extension, and the extension comprises one or more holes.
[33] The assembly of embodiment 3 comprising a plurality of supports, each of the plurality of supports comprising a sensor [34] The construct of embodiment 2 wherein the support is in the form of a sleeve.
[35] The assembly of embodiment 3 wherein the support is in the form of a sleeve.
[36] A construct comprising a sleeve, the sleeve comprising a luminal side and an abluminal side, the construct further comprising a sensor fixedly attached to the abluminal side of the sleeve.
[37] The construct of embodiment 36 wherein the sleeve comprises a rail and the sensor is fixedly attached to the rail.
[38] The construct of embodiment 36 wherein the sleeve comprises nitinol.
[39] The construct of embodiment 36 wherein the sleeve is expandable in terms of a width of the sleeve.
[40] The construct of embodiment 36 wherein the sleeve is not a stent.
[41] The construct of embodiment 36 wherein the sleeve fits around and securely engages with a stent or a graft.
[42] The construct of embodiment 36 wherein the sleeve has a length of 1 to 3 millimeters.
[43] The construct of embodiment 36 comprising a plurality of sensors fixedly attached to the abluminal side of the sleeve.
[44] A method of forming a construct, where the construct comprises a sensor fixedly attached to a support, and where the support can securely engage with a medical device; the method comprising a) providing a sensor comprising a housing, where the housing surrounds a detector, the housing comprising an extension that allows the sensor to be fixedly attached to a support; b) forming a support that can securely engage with a medical device; c) fixedly attaching the sensor to the support during the process of forming the support.
[45] A method of forming a construct, where the construct comprises a sensor fixedly attached to a support, and where the support can securely engage with a medical device; the method comprising a) providing a sensor comprising a housing, where the housing surrounds a detector, the housing comprising an extension that allows the sensor to be fixedly attached to a support; b) providing a support that can securely engage with a medical device; c) fixedly attaching the sensor to the support prior to securely engaging the support with a medical device.

### Antennas and Communications

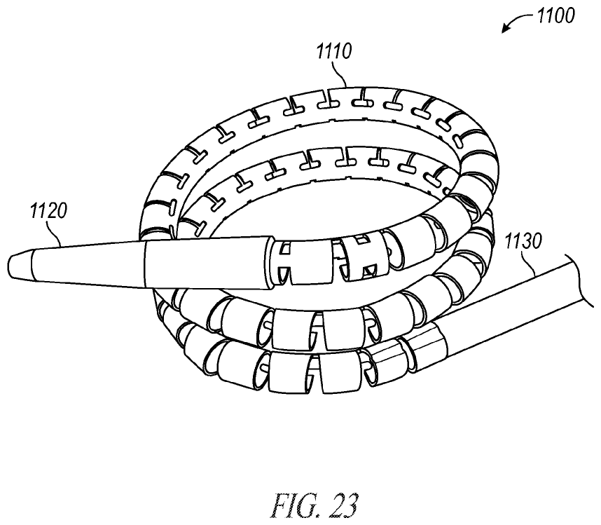

FIG. 23 illustrates a sensing attachment 1100 with a plurality of antennas. The sensing attachment 1100 can be any of the sensing attachments described herein. The sensing attachment 1100 includes a body 1110, which can be any of the bodies described herein (such as, the body 60 illustrated in FIG. 6). The sensing attachment 1100 can include a first antenna 1120 and a second antenna 1130. The first and second antennas can be positioned at opposite sides or ends of the body of 1110. In some cases, one or both of the first antenna 1120 and the second antenna 1130 can be positioned differently. Any of the first antenna 1120 or the second antenna 1130 can include multiple antennas. The first antenna 1120 can be configured to at least one of transmit or receive data (similarly to, for instance, the antenna 130 in FIG. 8). The second antenna 1130 can be configured to receive power in order to, for instance, recharge a power supply of the sensing attachment 1100, such as the power supply 112 (which may be rechargeable). The second antenna 1130 can receive power wirelessly from a transmitter (that can be part of a wireless charger). In some cases, the first antenna 1130 can be configured to receive power and the second antenna 1120 can be configured to at least one of transmit or receive data (sometimes referred to as a telemetry antenna). For example, the second antenna 1120 can be configured to communicate with a base station, another medical device (or sensing attachment), another remote electronic device, as described herein, or the like.

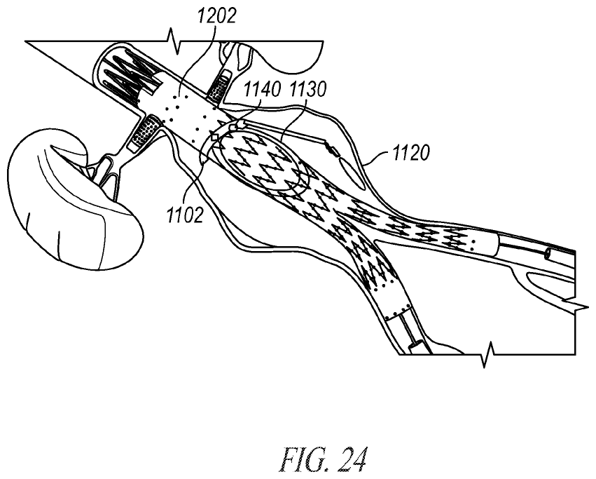

FIG. 24 illustrates placement in a blood vessel of a medical device 1202 and a sensing attachment 1102. The medical device 1202 can be any of the medical devices described herein. For example, the medical device 1202 can be a graft suitable for treating or repairing an abdominal aortic aneurysm (AAA), such as, the endograft 416 of FIGS. 15-19. Such graft may be deployed within the aneurysmal sac of a blood vessel. As described herein, the sensing attachment 1102 can be in contact with the external surface of the medical device 1202. For example, the sensing attachment 1102 can be at least partially supported by the medical device 1202. The sensing attachment 1102 can be implanted in a human body (along with the medical device 1202). The sensing attachment 1202 can be any of the sensing attachments described herein, such as the sensing attachment 1110 of FIG. 23.

The sensing attachment 1102 can include one or more sensors 1140, which can be any of the sensors described herein. For example, the one or more sensors 1140 can sense pressure, flow, or the like. The sensing attachment 1102 can include the first antenna 1120 that can be configured to one or more of receive or transmit data, as described herein. The sensing attachment 1102 can include the second antenna 1130 that can be configured to receive power, as described herein.

It can be challenging to design one or more antennas or communications circuitry for sensing attachments for an AAA graft, particularly when at least one of transmission or reception occurs at higher frequencies. At higher frequencies, there can be more propagation losses than at lower frequencies from transmitting through the body.

Any of the antennas disclosed herein can be an electrically small antenna (or electrically short). Generally, the size of an antenna (such as, length) may be directly proportional to the wavelength (or inversely proportional to the frequency) of a signal that the antenna is configured to one or more of transmit or receive. While the antenna size decreases with increasing frequency of the signal, the antenna size may be too large for many applications in which higher frequency transmission bands are used. For instance, the length of a dipole antenna for transmission and reception of a 400 MHz signal is over 35 centimeters (and the length of a smaller quarter-wavelength dipole antenna is still over 17 centimeters). In view of the constraints described herein, such size may be prohibitively large for one or more of the sensing attachments disclosed herein, such as for one or more sensing attachments for the AAA graft. An electrically small antenna can be an antenna that is much smaller (for example, much shorter) than the wavelength of a signal that the antenna is configured to transmit or receive. For instance, an antenna can be electrically small when its largest dimension is no more than one-tenth of the wavelength. As explained below, there are many challenges associated with designing an electrically small antenna for a sensing attachment.

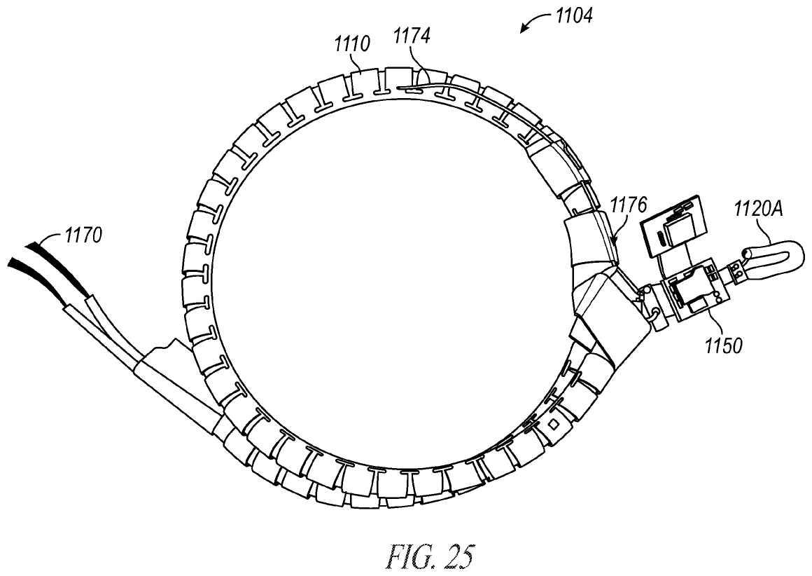

FIG. 25 illustrates a sensing attachment 1104 with a loop antenna 1120A. The sensing attachment 1104 can be any of the sensing attachments described herein, such as the sensing attachment 1100 or 1102. The sensing attachment can include a body, such as the body 1110. The loop antenna 1120A can be electrically connected (via a feed line) to an electronic circuitry 1150 which can be supported by a printed circuit board (PCB). The electronic circuitry 1150 can include one or more components of the IRP 103 of FIG. 8. For example, the electronic circuitry 1150 can include communications circuitry, which can include a transceiver (such as, the RF transceiver 126 of FIG. 8). The electronic circuitry 1150 can include processing circuitry, such as a controller (which can be the controller 132 of FIG. 8). Electric connection 1170 (illustrated as wires) can be used to provide power to the electronic circuitry 1150, for instance, from a power supply (such as, the power supply 112).

As described herein, the electronic circuitry 1150 can operate in a plurality of modes. The electronic circuitry 1150 can operate in a first mode, in which little power is consumed, to save power. The first mode is sometimes referred to as a low-power or sleep mode. The electronic circuitry 1150 can operate in a second mode, in which at least some components of the electronic circuitry 1150 (such as, one or more sensors, controller, etc.) are operational. The second mode is sometimes referred to as an operational mode. In some cases, the electronic circuitry 1150 can transition to from the sleep mode into the operational mode responsive to the loop antenna 1120A receiving one or more signals (or commands) in the second frequency band (of the first frequency band). In the operational mode, the electronic circuitry 1150 can, for instance, sense data with the one or more sensors and transmit the sensed data (or any other data) via the antenna 1120A. The data can be transmitted in a first frequency band (or in a second frequency band). Additionally or alternatively, data can be received via the loop antenna 1120A in the first frequency band. The electronic circuitry 1150 can transition from the second mode into the first mode responsive to a receiving data (such as, a command) via the loop antenna 1120A, responsive to an expiration of a duration of time, or the like.

In some cases, the first frequency band can be medical implant communication service (MICS) band. MICS band can have frequency range (or bandwidth) of about 402 MHz to 405 MHz, with a center frequency of about 403.5 MHz. The second frequency band can be industrial, scientific and medical (ISM) band. ISM band can have frequency range of about 2.4 GHz to 2.5 GHz, with a center frequency of about 2.45 GHz.

Due to the one or more constraints (such as, size or weight) described herein, it may not be feasible to include in a sensing attachment separate antennas for the first and second frequency bands. As a result, any of the antennas configured for one or more of transmission or reception of data, such as the loop antenna 1120A, can be designed to operate in a plurality of frequency bands. For example, the loop antenna 1120A can be a dual-band antenna configured to operate in the first and second frequency bands. To meet the one or more constraints, the loop antenna 1120A can be an electrically small antenna. For example, the diameter of the loop antenna can be about 10 mm (or less or more), which is smaller than one-tenth of about 0.75 meter wavelength of a 403.5 MHz signal or of about 0.12 meter wavelength of a 2.45 GHz signal.

Providing an adequate electrical ground for an electrically small antenna can be important to ensure good performance of the antenna. For instance, without adequate grounding, an electrically small antenna can undesirably reflect electromagnetic waves transmitted at a particular frequency (or in a frequency range) of interest. Any of the antennas configured to one or more of transmit or receive data described herein, such as the antenna 1120A, can be connected to the electrical ground of the electronic circuitry 1150 (such as, the ground plane of a printed circuit board of the electronic circuitry 1150). When the electrical ground of the electronic circuitry 1150 is insufficient to provide a desired antenna performance, additional (or alternative) grounding can be used. As described herein, the body 1110 (or any other of the disclosed bodies) can be made of conductive material. As a result, any of the antennas configured to one or more of transmit or receive data described herein, such as the antenna 1120A, can be electrically connected to the body 1110. As shown in FIG. 25, an electrical connection 1174 (illustrated as a wire) can connect the antenna 1120A to the body 1110. In some cases, one or more fasteners 1176 (which may be conductive) can be used to secure the electrical connection 1174 to the body 1110.

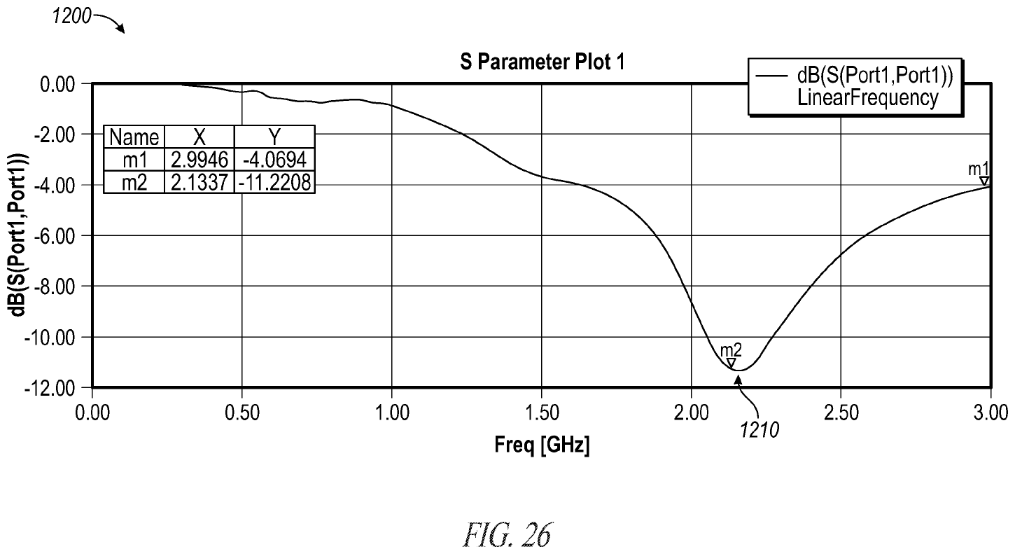

With proper grounding shown in FIG. 25, the loop antenna 1120A can be resonant in (or close to) the second frequency band (for example, the ISM band of 2.4 GHz to 2.5 GHz). FIG. 26 illustrates an s-parameter plot 1200 (specifically, S11 plot) of the loop antenna 1120A. S11 may be a measure of how much power is reflected back at an antenna port due to the mismatch from a transmission line. When connected to a network analyzer, S11 measures the amount of energy returning to the analyzer, which can be indicative of the power not delivered to the antenna. A small S11 value (such as, about −2 decibels (dB), about −3 dB, about −4 dB, or −5 dB or less) can indicate that a significant amount of energy has been delivered to the antenna. With reference to FIG. 26, the plot 1200 illustrates that at about 2.1 GHz (shown by the arrow 1210), the S11 value is almost −12 dB, indicating very good performance of the antenna at this frequency. From the plot 1200, it can be concluded that the loop antenna 1120A resonates at around 2.1 GHz. The plot 1200 illustrates worse performance of the loop antenna 1120A in the first frequency band (for example, the MICS band of 402 MHz to 405 MHz). As described herein, the plot 1200 can take into account one or more dielectric parameters (such as, permittivity or conductivity) of the tissue surrounding the sensing attachment and affecting the performance of the antenna (such as, causing a shift in a resonant frequency(ies) of the antenna). When used with an AAA graft, the tissue can include one or more of muscle, fat, bones, skin, etc.

The loop antenna 1120A can be tuned to improve performance in one or more frequency bands of interest. A matching network (or matching circuitry) can be designed and connected to the loop antenna 1120A to provide one or more of reception or transmission in the first frequency band (such as, the MICS band of 402 MHz to 405 MHz) and in the second frequency band (such as, the ISM band of 2.4 GHz to 2.5 GHz). As described herein, the matching circuitry can be designed to account for the dielectric parameters of the tissue surrounding the sensing attachment.

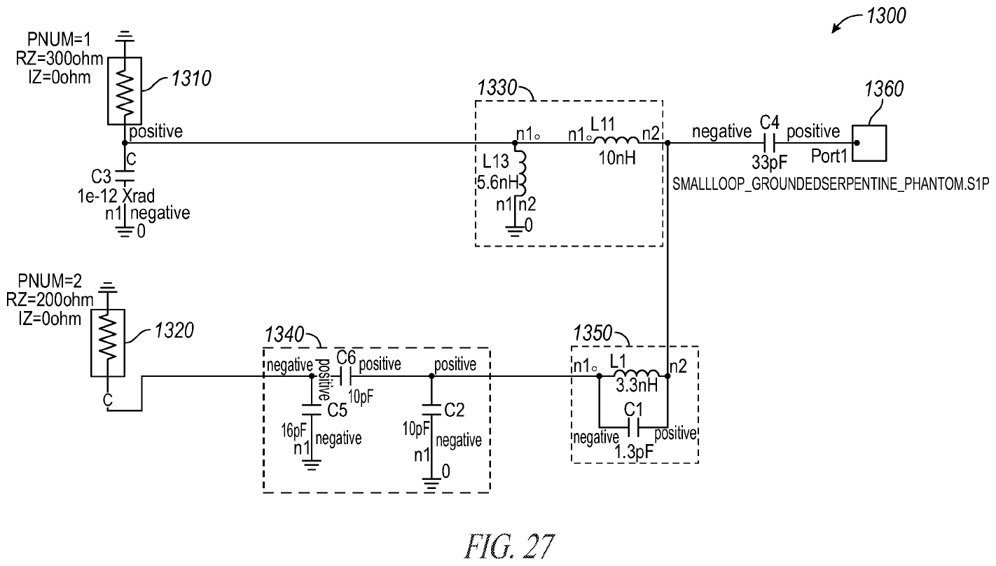

The matching circuitry can be electrically connected to the loop antenna 1120A. The matching circuitry can provide impedance matching between the antenna and transceiver. FIG. 27 illustrates matching circuitry 1300 for the loop antenna 1120A. The top part of the matching circuitry 1300 can process signals received (or transmitted) in the second frequency band. The bottom part of the matching circuitry 1300 can process signals received (or transmitted) in the first frequency band. Ports 1310 and 1320 can indicate the outputs (or inputs) of the antenna 1120A. Ports 1310 and 1320 can be connected to the transceiver, which can operate in first and second frequency bands. In some cases, a filter (such as, the filter 128 illustrated in FIG. 8, which can be a SAW bandpass filter) can be interposed between the loop antenna 1120A and the transceiver. For example, the filter can be connected to the port 1320.

A matching network 1330 can process signals in the second frequency band. The matching network 1330 can be inductive. The matching network 1330 is illustrated as an L-network that includes two inductors (such as, a shunt inductor L13 and a series inductor L11). The matching network 1330 can be designed as illustrated in order to match (or counter) the capacitive reactance of the loop antenna 1120A in the second frequency band (such as, at higher frequencies). A matching network 1340 can process signals in the first frequency band. The matching network 1340 can be capacitive. The matching network 1340 is illustrated as a Pi-network that includes three capacitors (such as, shunt capacitors C5 and C2 and a series capacitor C6). The matching network 1340 can be designed as illustrated in order to match (or counter) the inductive reactance of the loop antenna 1120A in the first frequency band (such as, at lower frequencies).

In some cases, other types of matching networks can be used for matching in one or more of the first or second frequency bands. The matching networks 1330 and 1340 and the selection and values of the components of these matching networks are illustrative. In some implementations, the matching network(s) may not be used.

A band-stop (or notch) filter 1350 can be used to remove higher frequency components from the signals in the first frequency band. The notch filter can remove one or more signal components in the second frequency band from the signals being received (or transmitted) in the first frequency band. The notch filter 1350 is illustrated as a combination of an inductor L1 connected in parallel with a capacitor C1. A notch filter may not be included in the matching network for the second frequency band in order to avoid or reduce undesirable parasitic effects (such as, one or more of parasitic capacitance or inductance) at the higher frequencies of the second frequency band.

Outputs of the top and bottom parts of the matching circuitry 1300 can be connected to the loop antenna 1120A. In some cases, an output port 1360 can connect the output of the loop antenna 1120 to a network analyzer (such as, a vector network analyzer (VNA)). The network analyzer can be used for measuring the s-parameters (such as, S11 parameters) of the loop antenna 1120A.

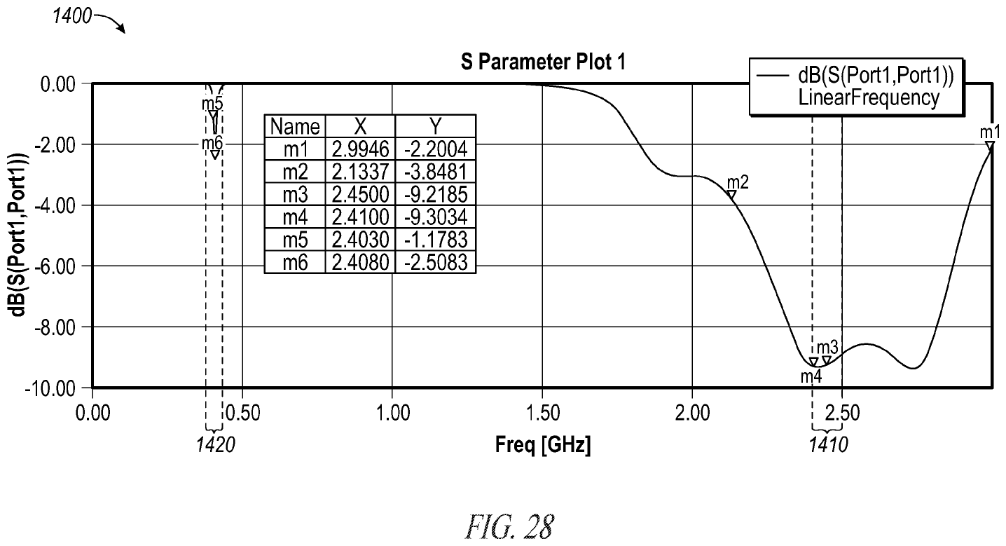

FIG. 28 illustrates an s-parameter plot 1400 (specifically, S11 plot) of the loop antenna 1120A connected to a matching circuitry, such as the matching circuitry 1300. Region 1410 illustrates that the S11 value across the bandwidth of the second frequency band (such as, the ISM band of 2.4 GHz to 2.5 GHz) is around −9 dB, confirming good performance of the antenna in the second frequency band. Region 1410 illustrates good performance across the wide bandwidth (such as, 0.1 GHz) of the second frequency band. Region 1420 illustrates that the S11 value across the bandwidth of the first frequency band (such as, the MICS band of 402 MHz to 405 MHz) is less than −2 dB, confirming good performance of the antenna in first frequency band. In some cases, to further improve the bandwidth of the loop antenna 1120A in the first frequency band, the size of the loop (such as, the diameter) can be increased. As explained herein, the plot 1400 can take into account one or more dielectric parameters of the tissue surrounding the sensing attachment.

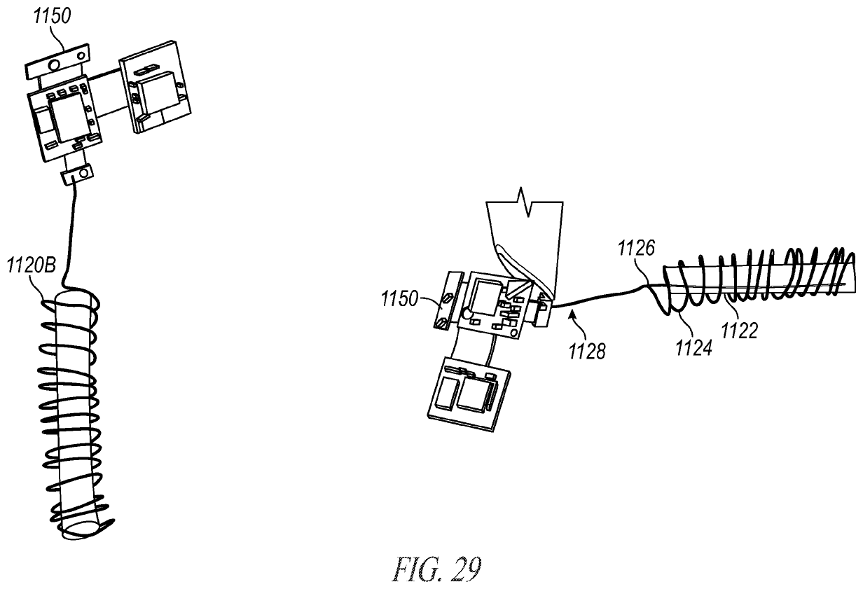

An antenna for at least one of transmitting or receiving data can be a monopole helical antenna 1120B (sometimes referred to as monopole helix antenna) as illustrated in FIG. 29. The monopole helix antenna 1120B can include a monopole 1122 (illustrated as straight wire) and a helix 1124. In some cases, as shown in FIG. 29, the helix 1124 can be wound around the monopole 1122 in order to reduce the size of the antenna. The monopole 1122 and helix 1124 can be electrically connected, for example, at the junction 1126 illustrated in FIG. 29. Other than this electrical connection, the monopole 1122 and the helix 1124 can be electrically insulated from one another. For instance, as shown in FIG. 29, a piece of tape (such as, Kapton tape) separating the monopole 1122 from the helix 1124 wound around the monopole. As shown in FIG. 29, the helix 1124 can be wound around a nonconductive material (such as, polytetrafluoroethylene PTFE or another polymer), which can provide structural support for the antenna. The monopole helix antenna 1120B is illustrated as being connected to the electronic circuitry 1150 (for example, via a feed line 1128, which may be part of the monopole 1122). In some implementations, the monopole 1122 and the helix 1124 can electrically connected, but otherwise positioned separately.

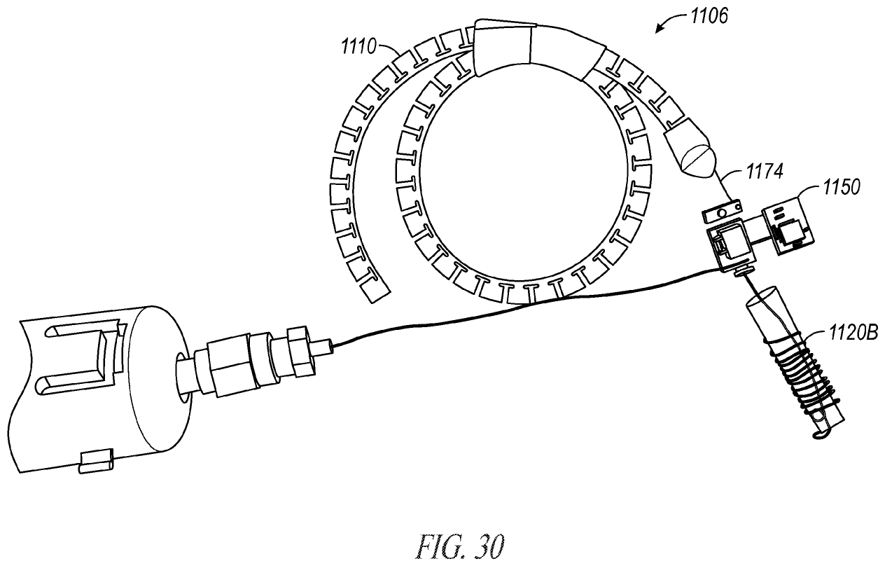

FIG. 30 illustrates a sensing attachment 1106 with the monopole helix antenna 1120B. The sensing attachment 1106 can be similar to the sensing attachment 1104 of FIG. 25 described above with the exception of the difference in the antennas. The monopole helix antenna 1120B can be grounded as described above in connection with the loop antenna 1120A.

The monopole helix antenna 1120B can be an electrically small antenna, as described above. In some cases, for example, the length of the monopole helix antenna can be about 30 mm (or less or more). As described herein, the length of the monopole helix antenna may be no more than 40 mm. In combination, the monopole 1122 and helix 1124 can function as a dual-band antenna that is resonant in the first and second frequency bands (such as, in the MICS and ISM bands). The monopole 1122 and helix 1124 can separately resonate in the first and second frequency bands (or vice versa). For example, it has been determined that the monopole helix antenna 1120B prior to tuning (such as, by designing matching circuitry) can be resonant at about 403 MHz (which is at or close to the center frequency of the MICS band) and at about 2.45 GHz (which is at or close to the center frequency of the ISM band). In some cases, the monopole helix antenna 1120B prior to tuning can have S11 value of about −5 dB at about 403 MHz and of about −3 dB at 2.45 GHz, which indicates very good performance of the antenna in the first and second frequency bands.

One or more of the length or spacing between the turns of the helix can be adjusted to improve performance of the monopole helix antenna 1120B. For example, the length of the helix 1124 can be about the length of a quarter-wavelength dipole antenna configured to one or more of receive or transmit at 403 MHz (for example, about 20 cm). The spacing between the turns of the helix 1124 can be between about 1 mm (or less) and about 2 mm (or more). In some cases, the spacing can be about 1.7 mm.

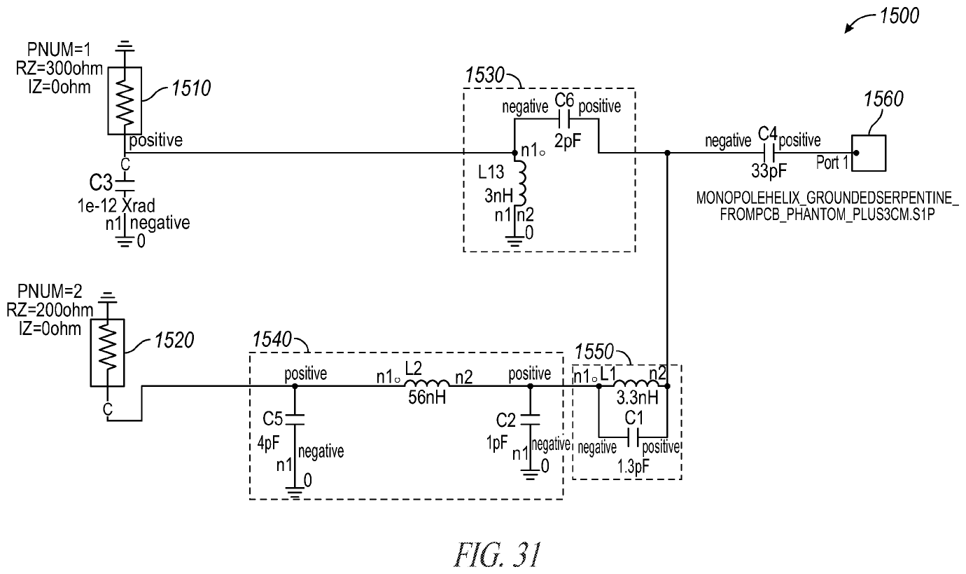

As described above, the monopole helix antenna 1120B can be tuned. A matching network (or matching circuitry) can be designed and electrically connected to the monopole helix antenna 1120B. FIG. 31 illustrates matching circuitry 1500 for the monopole helix antenna 1120B. The matching circuitry 1500 can be designed to match high impedance of a transceiver that may be used with the helix antenna 1120B. The top part of the matching circuitry 1500 can process signals received (or transmitted) in the second frequency band, which can be the ISM band). The bottom part of the matching circuitry 1500 can process signals received (or transmitted) in the first frequency band, which can be the MICS band. The matching circuitry 1500 can be similar to the matching circuitry 1300 for the loop antenna illustrated in FIG. 27. Ports 1510, 1520, and 1560 can be similar to the ports 1310, 1320, and 1360 described above. Notch filter 1550 for processing signals in the first frequency band can be similar to the notch filter 1350 described above.

A matching network 1530 can process signals in the second frequency band. The matching network 1530 can be a step-up impedance high-pass filter (for processing higher frequency signals in the second frequency band). The matching network 1530 is illustrated as an L-network that includes an inductor (such as, a shunt inductor L13) and a capacitor (such as, a series capacitor C6). A matching network 1540 can process signals in the first frequency band. The matching network 1540 can be a step-up impedance low pass filter (for processing lower frequency signals in the first frequency band). The matching network 1540 is illustrated as a Pi-network that includes capacitors (such as, shunt capacitors C5 and C2) and an inductor (such as, a series inductor L2).

In some cases, other types of matching networks can be used for matching in one or more of the first or second frequency bands. The matching networks 1530 and 1540 and the selection and values of the components of these matching networks are illustrative. In some implementations, the matching network(s) may not be used (for instance, when the monopole helix antenna is used with a different transceiver or the antenna is tuned in vivo).

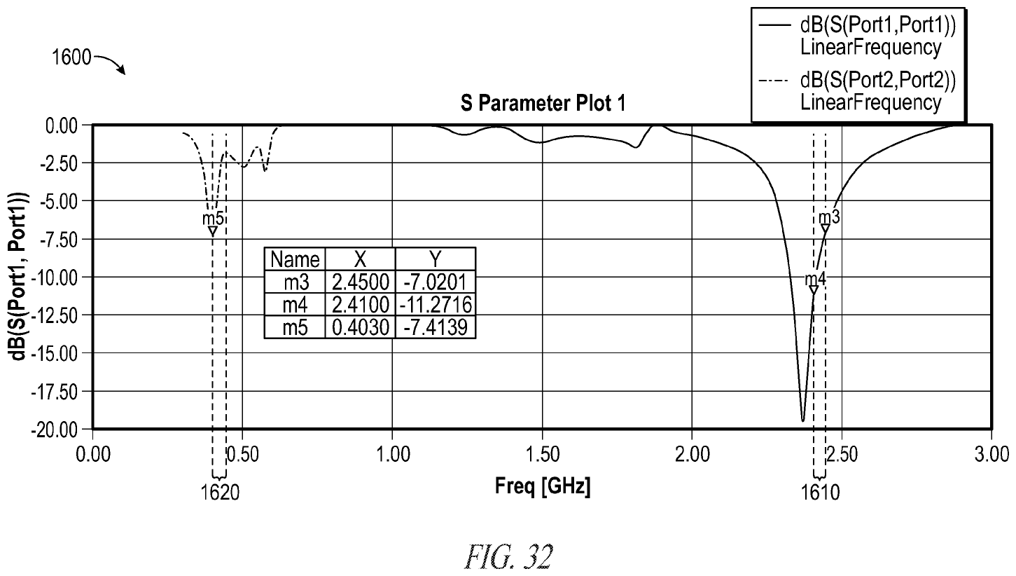

FIG. 32 illustrates an s-parameter plot 1600 (specifically, S11 plot) of the loop antenna 1120B connected to a matching circuitry, such as the matching circuitry 1500. Region 1610 illustrates that the S11 value across the bandwidth of the second frequency band (such as, the ISM band of 2.4 GHz to 2.5 GHz) is less than −5 dB, confirming good performance of the antenna in the second frequency band. Region 1610 illustrates good performance across the bandwidth (such as, 0.1 GHz) of the second frequency band. Region 1620 illustrates that the S11 value across the bandwidth of the first frequency band (such as, the MICS band of 402 MHz to 405 MHz) is less than −5 dB, confirming good performance of the antenna in first frequency band. Region 1620 illustrates good performance across the wide bandwidth (such as, 3 MHz) of the first frequency band. As explained herein, the plot 1600 can take into account one or more dielectric parameters of the tissue surrounding the sensing attachment.

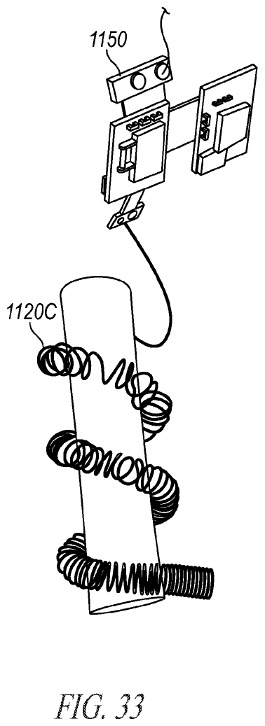

FIG. 33 illustrates a spiral helix antenna 1120C connected to the electronic circuitry 1150. The spiral helix antenna 1120C can include a spiral conductor wound into a helical shape. The spiral conductor can be wound around a nonconductive material, as described above. The spiral helix antenna 1120C can be electrically small. In some cases, the spiral helix antenna 1120C can be used for one or more of receiving or transmitting data. Other types of electrically small antennas, such as slot or patch, can be used for one or more of receiving or transmitting data.

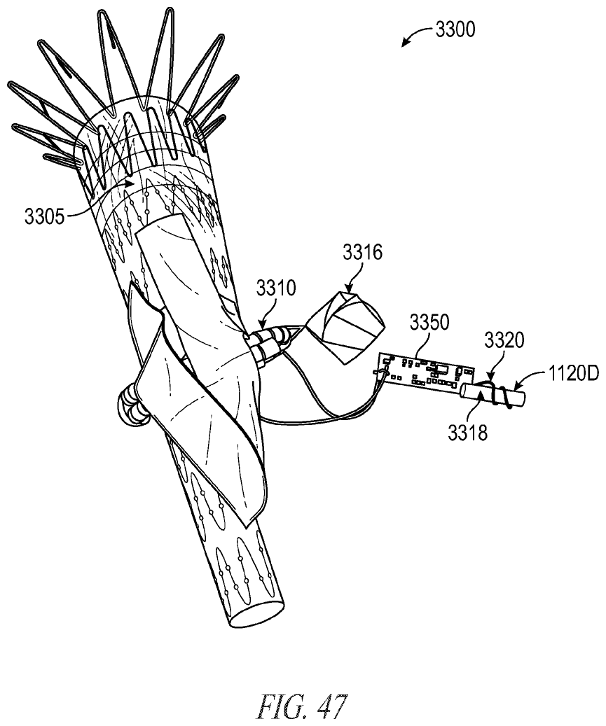

In some cases, data can be received and transmitted wirelessly in a single band. For instance, the Bluetooth frequency band (2400 to 2483.5 MHz) can be utilized. In such case, a helix (or helical) antenna 1120D illustrated in FIG. 47 can be used. The helix antenna 1120D can be an electrically small antenna, as described above. One or more of the length or spacing between the turns of the helix can be adjusted to improve performance of the helix antenna 1120D. In some cases, for example, the length of the conductive portion 3320 of the helix antenna 1120D (or the length of the antenna) when the conductive portion is straight can be about 30 mm (or less or more). In some instances, the length of the helix antenna 1120D when the conductive portion is straight may be no less than 20 mm or no more than 40 mm. With reference to FIG. 47, the number of turns of the conductive portion 3320 can be two (or less or more, such as one, three, four, five, or the like). Having more turns can increase the electrical length of the helix antenna 1120D. Likewise, having less turns can decrease the electrical length of the helix antenna 1120D. The spacing between the turns of the conductive portion 3320 can be between about 1 mm (or less) and about 2 mm (or more). In some cases, the spacing can be about 1.7 mm. The conductive portion 3320 can be wound on a nonconductive material (or substrate) 3318 (such as, PTFE or another polymer), as described above. The material 3318 can provide structural support for antenna 1120D. The material 3318 can be shaped as a tube.

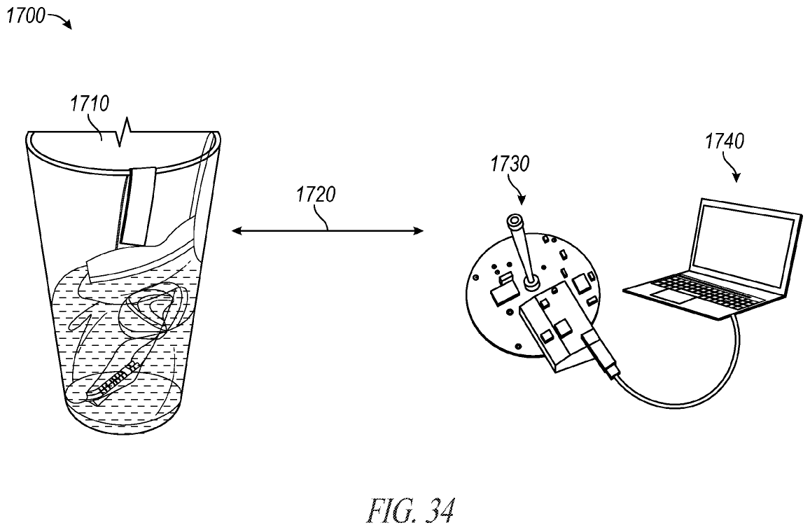

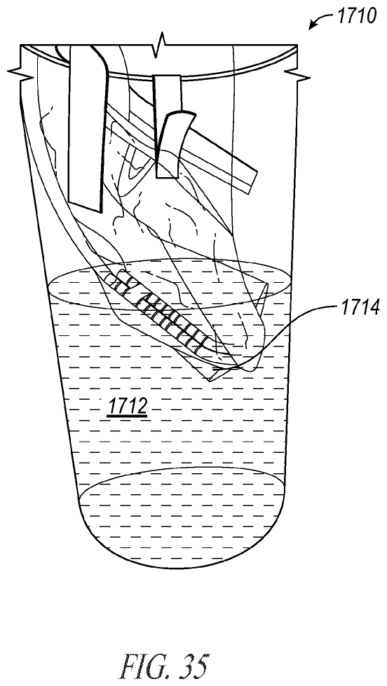

As described above, one or more dielectric parameters (such as, permittivity or conductivity) of the tissue surrounding the sensing attachment can affect the performance of the antenna. As a result, performance of any of the antennas described herein (such as, the loop antenna 1120A, the monopole helix antenna 1120B, the spiral helix antenna 1120C, or the helix antenna 1120D) and verified not only in free space, but also in material designed to simulate the dielectric parameters of the tissue. FIG. 34 illustrates testing arrangement 1700. A container 1710 (such as, a vase) can be filled with a substance or composition designed to simulate the dielectric parameters of the tissue (sometimes referred to as phantom). The container 1710 can be made of glass or another RF transparent material. An antenna connected to electronic circuitry (such as, the electronic circuitry 1150 or 3350) can be placed into a plastic bag or similar enclosure (to electrically insulate the antenna and the electronics) and submerged in the phantom. A base station 1730 can be positioned at a distance 1720 from the antenna. The base station 1730 can be connected to a computer 1740 (such as, a laptop computer). FIG. 35 illustrates a close-up view of the container 1710 filled with the phantom 1712, and of an antenna 1714 submerged in the phantom.

The phantom can be constructed to match one or more dielectric parameters of the human body for the one or more frequency bands of interest. For example, at 2.45 GHz (in the ISM band) the human body (for instance, muscle) can exhibit relative permittivity (or dielectric constant) of about 52.7 and conductivity of about 1.95 Siemens/meter (S/m). As another example, at 450 MHz (which is close to the MICS band), the human body can exhibit relative permittivity of about 56.7 and conductivity of about 0.94 S/m. A combination of one or more of sodium chloride (NaCl), diacetin, glycol, or distilled water (or other types of chemical materials) can be constructed to match these relative permittivities and conductivities. Such phantom or any other phantoms described herein can be liquid (rather than solid) since liquid can better surround and encapsulate the antenna being tested, which would provide a more representative dielectric loading similar to in vivo environment.

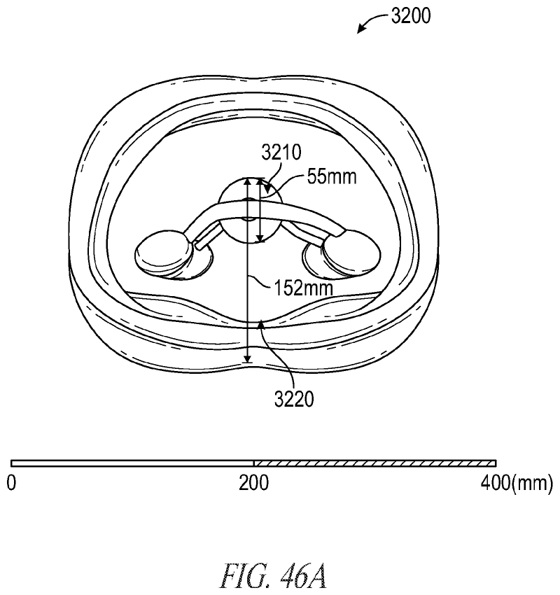

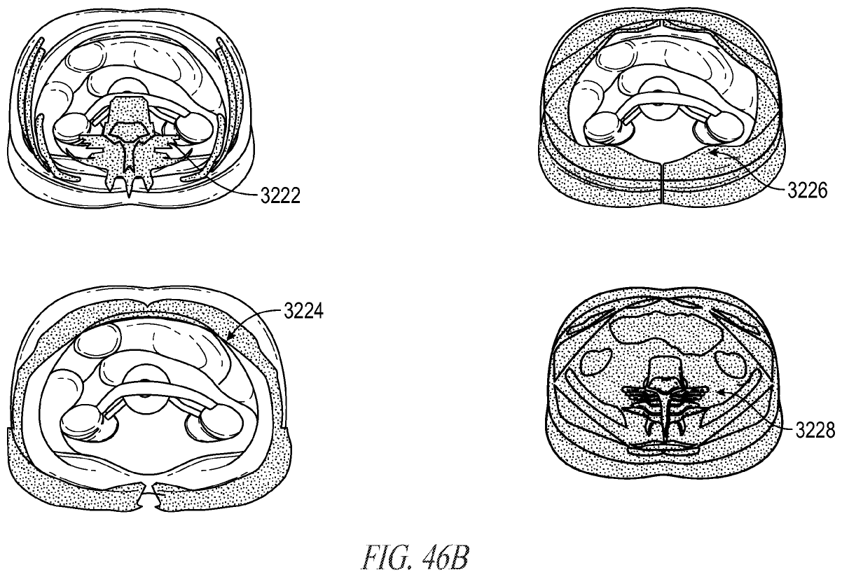

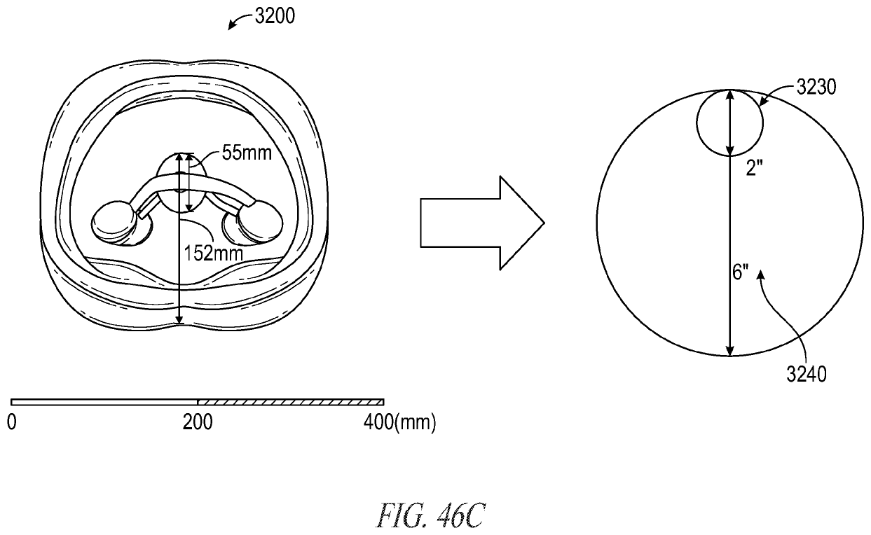

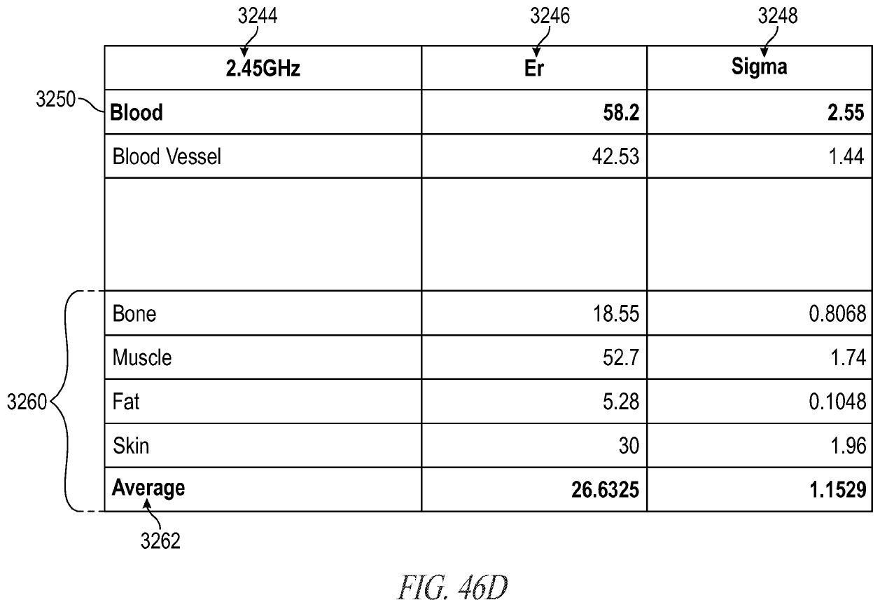

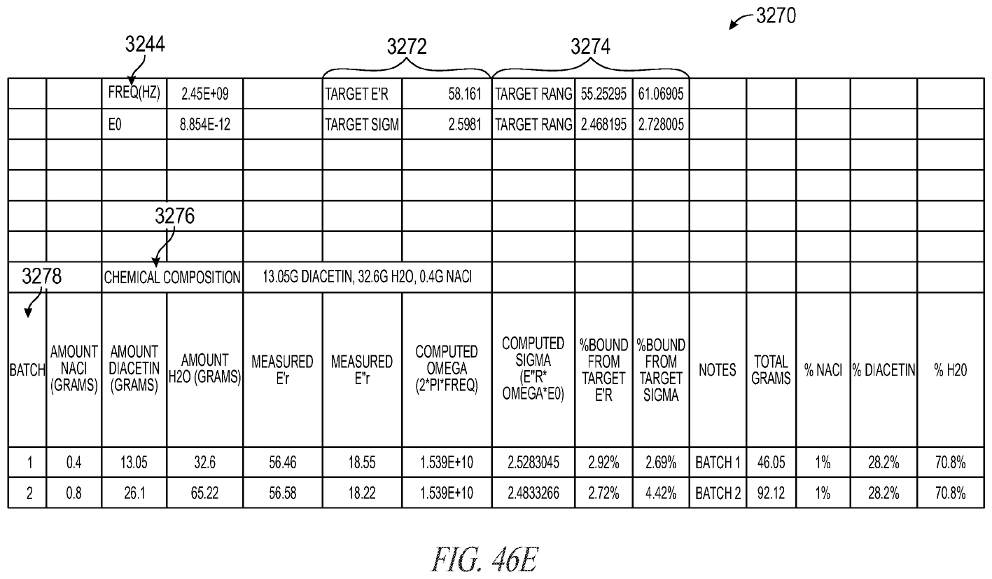

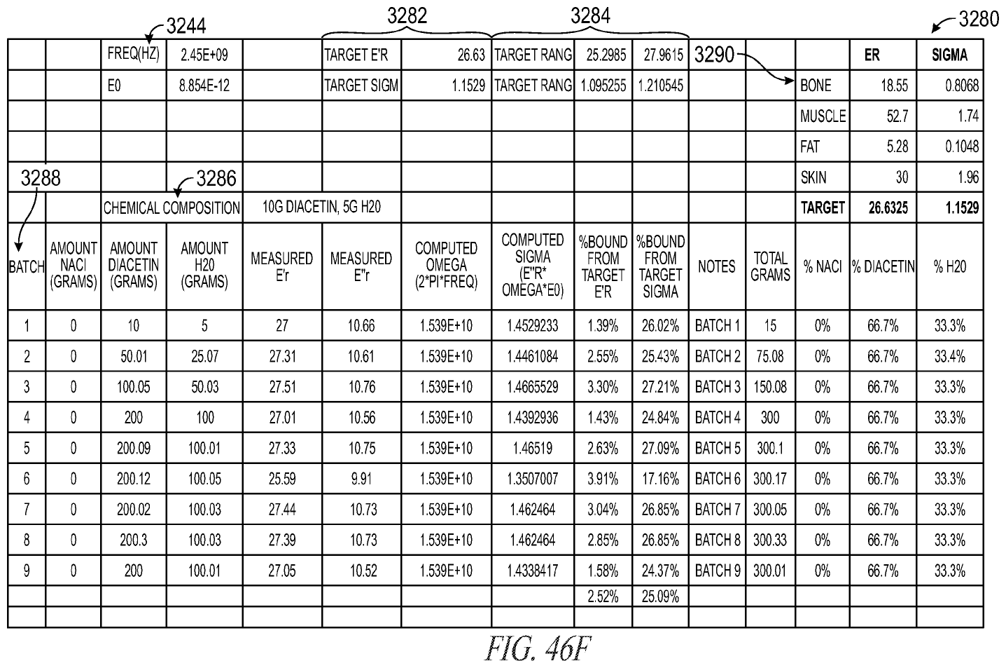

Multiple phantoms can be designed to simulate different part of the human body, such as blood, bone, muscle, fat, or skin. With reference to FIG. 46A, a human body model 3200 for testing antenna performance and tuning the antenna is illustrated. The model 3200 can be representative of positioning a sensing attachment with the antenna in an aneurysmal sac of the abdominal aorta (or in another blood vessel), as described herein. The model 3200 can represent a cross-section of a human torso. The model 3200 can include blood 3210 (for instance, inside a blood vessel) and other tissue surrounding the blood vessel, such as one or more of bone, muscle, fat, and skin. In the illustrated example, the blood vessel can be assumed to be about 2.16 inches (or about 55 mm) in diameter. Distance from the blood vessel to the body surface can be assumed to be about 6 inches (or about 152 mm).

Electromagnetic properties of blood and other tissue can affect performance of the antenna (relative to the performance in free space). For instance, at the frequency of 2.45 GHz (in the Bluetooth band), blood can exhibit relative permittivity of about 58.26 and conductivity of about 2.55 S/m, bone (such as, cancellous bone) can exhibit relative permittivity of about 18.55 and conductivity of about 0.8068 S/m, muscle can exhibit relative permittivity of about 52.7 and conductivity of about 1.74 S/m, fat can exhibit relative permittivity of about 5.28 and conductivity of about 0.1048 S/m, and skin can exhibit relative permittivity of about 30 and conductivity of about 1.96 S/m. Frequency of 2.45 GHz can correspond to the center frequency or target frequency in the Bluetooth band.

Two phantoms can be designed: a phantom for blood and a conglomerate phantom for other surrounding tissues (such as, bone, muscle, fat, and skin). The phantom for blood can simulate electromagnetic properties of blood, and the conglomerate phantom can simulate electromagnetic properties of the other tissues. The relative permittivity and conductivity of bone, muscle, fat, and skin can be averaged for the purposes of designing the conglomerate phantom (resulting in the average relative permittivity of about 26.6325 and average conductivity of about 1.1529 S/m at 2.45 GHz, as shown in FIG. 46D). Two such phantoms can be designed because blood that surrounds the sensing attachment can influence the performance of antenna more than the other tissues (due to higher relative permittivity and conductivity), which can be aggregated into a single conglomerate phantom for simplification.

FIG. 46B illustrates components of the conglomerate phantom. Cancellous bone (such as, a vertebrae) is illustrated as 3222. Relative permittivity of cancellous bone can be of about 18.55 and conductivity can be about 0.8068 S/m at 2.45 GHz. Fat is illustrated as 3224. Relative permittivity of fat can be about 5.28 and conductivity can be about 0.1048 S/m at 2.45 GHz. Muscle is illustrated as 3226. Relative permittivity of muscle can be about 52.7 and conductivity can be about 1.74 S/m at 2.45 GHz. Filling in the remaining voids, skin and other tissue are illustrated as 3228. Relative permittivity of skin and other tissue can be about 30 and conductivity can be about 1.96 S/m at 2.45 GHz. Conglomerate phantom can represent the electromagnetic contributions of the body tissue surrounding the blood vessel.

FIG. 46C illustrates the two phantoms used for testing the performance of the helix antenna 1120D and tuning the helix antenna. The two phantoms can be used for testing the performance of any of the other antennas described herein. Blood phantom 3230 is illustrated as being contained in a blood vessel having diameter of about 2 inches. Conglomerate phantom 3240 is illustrated as spanning a distance of about 6 inches from the boundary of the vessel to the body's exterior surface. In such model, the antenna would need to operate in the range of at least about 8 inches to be able to communicate with a base station, which may be integrated into a chair (such as, supported by a fabric pad on the chair), bed, or the like or be positioned near the patient.

FIG. 46D illustrates a table listing relative permittivity (Er) 3246 and conductivity (Sigma) 3248 values of the blood phantom 3230 and conglomerate phantom 3240 at a frequency 3244 of 2.45 GHz. Row 3250 shows that the blood phantom 3230 can have Er=58.2 and Sigma=2.55 S/m. Rows 3260 show Er and Sigma values for bone, muscle, fat, and skin components of the conglomerate phantom 3240. Taking the average 3262 can result in Er=26.6325 and Sigma=1.1529 S/m.

FIG. 46E illustrates a table 3270 summarizing the development of the blood phantom 3230. Target relative permittivity (58.161) and conductivity (2.5981 S/m) are indicated by the bracket 3272. Target range of relative permittivity and conductivity are indicated by the bracket 3274. To achieve target relative permittivity and conductivity, the blood phantom 3230 in some cases (as illustrated by the arrow 3276) can have a chemical composition of 13.05 g of diacetin, 32.6 g of distilled water, and 0.4 g of sodium chloride (NaCl). More generally, the chemical composition of the blood phantom 3230 can include about 1% of NaCl, 28.2% of diacetin, and 70.8% of water by weight (or by volume). In some cases, glycol can be used additionally or alternatively. Table 3270 illustrates properties of two batches 3278 (batch 1 and batch 2) of such chemical composition that were made. Batch 2 has about twice the quantity of the components (NaCl, diacetin, and distilled water) than batch 1. As is shown in the table 3270, measured relative permittivity (column “Measured E′R”) and conductivity (column “Computed Sigma”) of the batches are within 5% tolerance of the target values (see columns “% Bound from Target E′R” and “% Bound from Target Sigma”).

FIG. 46F illustrates a table 3280 summarizing the development of the conglomerate phantom 3240. Target relative permittivity (28.63) and conductivity (1.1529 S/m) are indicated by the bracket 3282. These correspond to averaged values as illustrated in table 3290 (and described above). Target range of relative permittivity and conductivity are indicated by the bracket 3284. To achieve target relative permittivity and conductivity, the conglomerate phantom 3240 in some cases (as illustrated by the arrow 3286) can have a chemical composition of 10 g of diacetin and 5 g of distilled water. More generally, the chemical composition of the conglomerate phantom 3240 can include about 66.7% of diacetin and 33.3% of distilled water by weight (or by volume). In some cases, one or more of NaCl or glycol can be used additionally or alternatively. Table 3270 illustrates properties of nine batches 3288 (batches 1 to 9) of such chemical composition that were made. These batches vary in weight. As is shown in the table 3280, measured relative permittivity (column “Measured E′R”) of the batches is within 3% tolerance of the target value (see column “% Bound from Target E′R”). Measured conductivity (column “Computed Sigma”) is within 25.1% of the target value (column “% Bound from Target Sigma”), which may be improved with a different chemical composition.

FIG. 47 illustrates a prototype 3300 that includes a sensing attachment 3310 (which can be similar to the sensing attachment 1100) with the helix antenna 1120D. The sensing attachment 3310 is illustrated as being attached to a graft 3305, which can be an AAA graft. The sensing attachment 3310 may be mechanically attached to the graft 3305, while being electrically isolated from the graft 3305 (for instance, by using nonconductive material, such as tape or polymer coating). Electronic circuitry 3350 to which the helix antenna 1120D is electrically connected can be similar to the electronic circuitry 1150 described above. The antenna 1120D can be grounded to the body of the sensing attachment, as described above. Power source 3316 (such as, one or more batteries) can supply power to the electronic circuitry 3350.

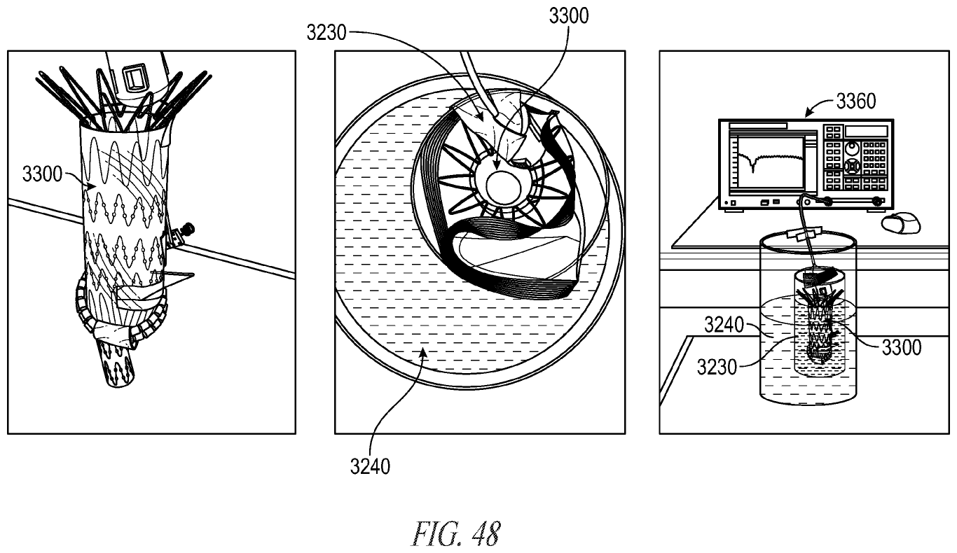

FIG. 48 illustrates a setup for RF testing of the prototype 3300. The prototype 3300 is positioned in a plastic bag or similar enclosure (to electrically insulate the antenna and electronics) and submerged in the blood phantom 3230, which can be contained in a first container. The first container can be submerged in the conglomerate phantom 3240, which can be contained in a second container. One or more of the first or second containers can be made of glass or another RF transparent material. The helix antenna 1120D can be connected to a network analyzer 3360 (such as, VNA) for RF testing the helix antenna 1120D.

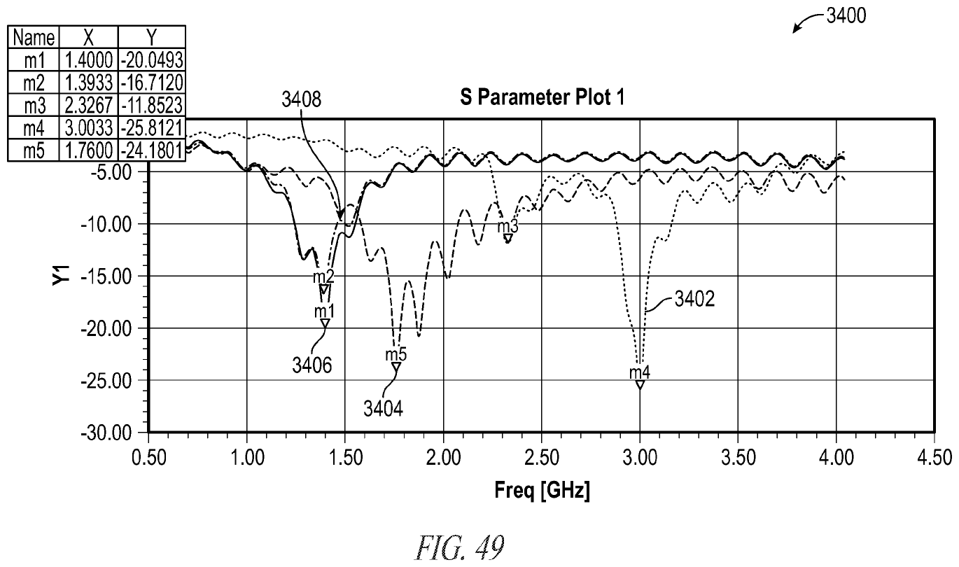

FIG. 49 illustrates an s-parameter plot 3400 (specifically, S11 plot) obtained during RF testing of the prototype 3300. The prototype 3300 was tested in free space (plot 3402), submerged in the blood phantom 3230 and the conglomerate phantom 3240 (plot 3404) as shown in FIG. 48, submerged in the blood phantom 3230 and the conglomerate phantom 3240 but without the material 3318 (plot 3406), and submerged only in the blood phantom 3230 and without the material 3318 (plot 3408). In free space, the helix antenna 1120D resonates at about 3.0 GHz (as depicted by point m4 on the plot 3402), which is close to the Bluetooth frequency band. Addition of one or more of the blood or conglomerate phantoms and the material 3318 can change the resonant frequency of the helix antenna 1120D, as illustrated by plots 3404, 3406, and 3408. Specifically, the resonant frequency can shift down to about 1.39 GHz (as depicted by point m2 on the plot 3408) in the blood phantom 3230 and about 1.4 GHz (as depicted by point m1 on the plot 3406) in the blood phantom 3230 and the conglomerate phantom 3240. This illustrates that the blood phantom 3230 is more dominant for affecting the RF performance of the helix antenna 1120D than the conglomerate phantom 3240. Adding the material 3318 can dampen the shift in the resonant frequency from about 3.0 GHz to about 1.76 GHz (as depicted by point m3 on plot 3404).

The length of the helix antenna 1120D can be shortened (relative to the length in free space) due to downward shifting of the resonant frequency of the helix antenna 1120D described above. Due to the dampening effect of one or more phantoms 3230 or 3240 and/or the material 3318 shifting the resonant frequency down, the electrical length of the helix antenna 1120D can appear lengthened. To tune the helix antenna 1120D to resonate at the frequency of interest (such as 2.45 GHz), the physical length of the helix antenna 1120D may be shortened.

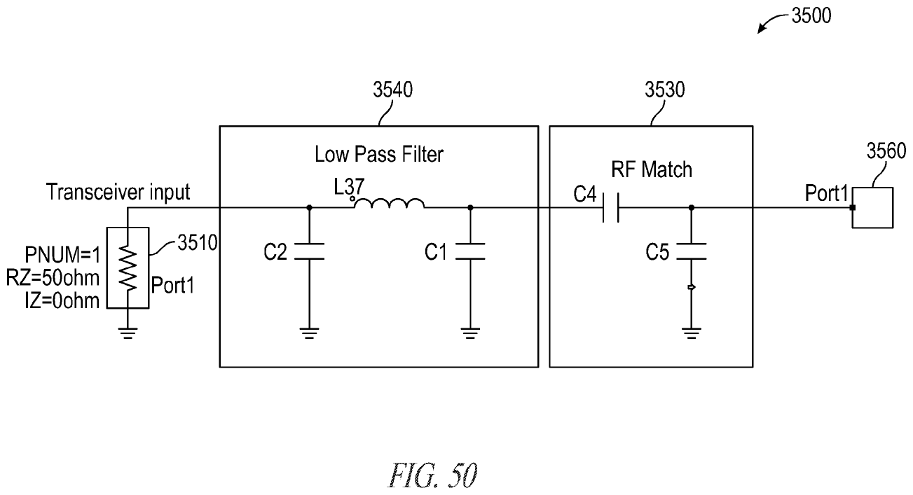

As described above, matching circuitry can be designed to tune the helix antenna 1120D. FIG. 50 illustrates matching circuitry 3500, which can be interposed between the helix antenna 1120D and a transceiver. Similarly to the port 1310 (or port 1320) described above, port 3510 can be connected to the transceiver, which can operate in the Bluetooth frequency band. The transceiver can be part of the electronic circuitry 3350. The matching circuitry 3500 can include a low-pass filter 3540 configured to remove higher frequency components. For instance, the filter 3540 can be a third-order Chebyshev filter with an inductor L37 and two capacitors C1 and C2. A matching network 3530 can be an L-network that includes two capacitors: series capacitor C4 and shunt capacitor C5. The matching network 3530 can be designed as illustrated in order to match (or counter) the inductive reactance of the helix antenna 1120D. Values of the capacitors C4 and C5 can be selected to shift the resonant frequency of the helix antenna 1120D in the environment being tested (for instance, blood and conglomerate phantoms) to the desired frequency (such as, 2.45 GHz for the Bluetooth band). Similarly to the port 1360 described above, port 3560 can connect the helix antenna 1120D to the network analyzer.

Testing of the antenna range (such as, for one or more of reception or transmission) can be performed by varying the distance 1720. The range of the loop antenna 1120A in the first frequency band (such as, in the MICS band) can be between about 1 foot (or less) and about 20 feet (or more) (such as, about 20 feet). The range of the loop antenna 1120A in the second frequency band (such as, in the ISM band) can be between about 1 foot (or less) and about 20 feet (or more) (such as, about 7 feet, about 10 feet, or about 15 feet). The range of the monopole helix antenna 1120B in the first frequency band (such as, in the MICS band) can be between about 1 foot (or less) and about 20 feet (or more) (such as, about 20 feet). The range of the monopole helix antenna 1120B in the second frequency band (such as, in the ISM band) can be between about 1 foot (or less) and about 25 feet (or more) (such as, about 15 feet or about 25 feet).

In some instances, the desired range of the helix antenna 1120D can be at least 1 foot (such that the antenna 1120D can communicate with a base station, which may be integrated into a chair, bed, or the like or be positioned near the patient). FIGS. 51A and 51B illustrate testing the range of the helix antenna 1120D of the prototype 3300. Various tests were performed with the assumption that there is a signal threshold 3610 below which the antenna 1120D would not be able to reliably receive or transmit signals. For example, the threshold 3610 can be −90 decibel milliwatts (dBm). With reference to FIG. 51A, graph 3600A illustrates the captured signal strength 3620A of one or more signals transmitted by the helix antenna 1120D when a Bluetooth receiver (such as, a smartphone) was being moved away from the prototype 3300. As is illustrated, the signal strength 3620A falls below the threshold 3610 when the Bluetooth receiver has been moved too far (such as, more than about 1 foot) from the prototype 3300. FIG. 51B illustrates results of similar tests performed at a different time. Graph 3600B is similar to graph 3600A, and plot 3620B is similar to plot 3620A. As is confirmed by the testing illustrated in FIGS. 51A and 51B, the antenna 1120D can operate at a range of at least 1 foot or more. In some cases, the antenna 1120D can operate at a range of up to 2 feet (or in some cases more than 2 feet).

Any of the antennas described herein (for example, the loop antenna 1120A, the monopole helix antenna 1120B, or spiral helix antenna 1120C, or the helix antenna 1120D) can be made from one or more conductive materials, such as copper, platinum, iridium, platinum and iridium alloys, gold, silver, nitinol, or the like. A monopole (such as, the monopole 1122) and the helix (such as, a helix 1124) of the monopole helix antenna (such as, the antenna 1120B) can be made from similar or different materials.

As described herein, the monopole and the helix can be positioned separately or adjacently (for example, the helix can be wound around the monopole). The monopole and helix can be electrically insulated with the exception of a junction at which an electric connection is made (such as, the junction 1126). In some cases, the junction can be made or positioned in the electronic circuitry (such as, the electronic circuitry 1150). For example, the junction can be made or positioned on a printed circuit board of the electronic circuitry.

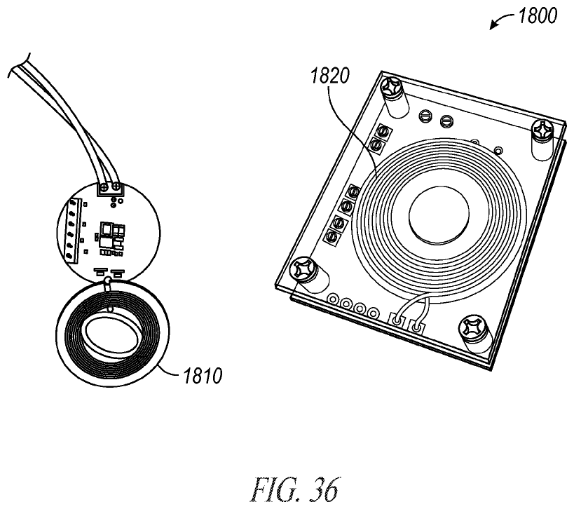

As described herein, any of the sensing attachments can include an antenna configured to receive power to recharge a power supply of the sensing attachment. For example, wireless power charging (WPT) can be used. FIG. 36 illustrates an arrangement 1800 for transmitting and receiving power. The arrangement 1800 can include an antenna 1810 (which can be similar to the antenna 1130) configured to receive power and an antenna 1820 configured to transmit energy (or power). One or more of the antennas 1810 or 1820 can be coil antennas. In operation, the antennas 1810 and 1820 can be inductively coupled to facilitate transmission of power. One or more of the antennas 1810 or 1820 may be positioned around a core of magnetic material (such, as ferrite). The core(s) can facilitate directional reception (or transmission) of the energy (such as, increase the quality factor as described below). The receive antenna 1810 can be part of the sensing attachment. In some cases, the size (such as, diameter) of the receive antenna 1810 may not exceed 45 mm (or less or more). For example, the diameter of the receive antenna 1810 can be 20 mm or 30 mm. The separation distance between the transmit antenna 1820 and the receive antenna 1810 can be between about 1 centimeter (or less) to about 10 centimeters (or more) (such as, 1.5 centimeter). The frequency of the electromagnetic signals for transmission of power can be between about 6 MHz (or less) and about 13 MHz (or more).

One or more of the antennas 1810 or 1820 can be made from one or more conductive material(s), such as nitinol, platinum, gold, silver, copper, nitinol and platinum alloys, platinum and iridium alloys, nitinol, platinum, and iridium alloys, nitinol, nickel, platinum and/or iridium alloys. The one or more materials for making any of the antennas 1810 or 1820 can exhibit one or more properties (such as, conductivity) for efficient energy reception or transmission. The one or more of the antennas 1810 or 1820 can be fabricated using thin film deposition. In some implementations, a core (such as, ferrite core) can be laid onto the thin film and a coil configuration can be formed (such as, using thin film deposition). The diameter of the coil of any of the antennas 1810 or 1820 can vary (such as, increase or decrease) symmetrically or non-symmetrically away (or toward) the core.

One or more of the antennas 1810 or 1820 can be manufactured utilizing a substrate. The substrate can be made from a polymer, such as nylon, polyether block amide (PEBA), base polymer substrate, etc. The substrate can be shaped to mimic the geometry of the one or more antennas (such as, the coil shape). The substrate can serve as a mold (or molds).

In some cases, charging circuitry (with the transmit antenna 1820) can be supported by a chair, bed, or the like. Power can be transferred to the sensing attachment when a patient is sitting down, lying, or the like. The quality factor (Q factor) of one or more of the receive antenna 1810 or the transmit antenna 1820, which may be indicative of energy loss and efficiency, can be between about 80 (or less) and about 200 (or more). In some implementations, the base station (or another remote electronic device) can be similarly supported by a chair, bed, or the like.

### Delivery System

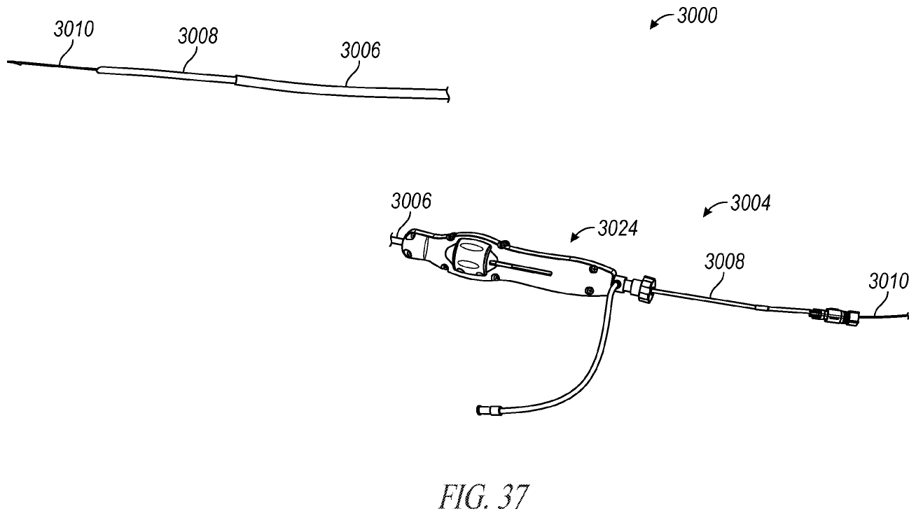

FIG. 37 illustrates a delivery system 3000 that may be used to deliver any of the implantable devices described herein (also referred to herein as sensing constructs, sensing attachments, scaffolds, or auxiliary components). The delivery system 3000 may include an outer sheath 3006, pusher shaft 3008, and/or release shaft 3010 slidably disposed relative to each other. For example, the pusher shaft 3008 may be slidably disposed within a lumen of the outer sheath 3006. The release shaft 3010 may be slidably disposed within a lumen of the pusher shaft 3008. During transport, the implantable device may be disposed radially between the release shaft 3010 and the outer sheath 3006. At least a portion or substantially the entire length of the implantable device may be positioned distal of the pusher shaft 3008. The various components of the delivery system 3000 can act independently or in conjunction to release the implantable device.

A proximal portion 3004 of the delivery system 3000 may include a handle 3024 for controlling one or more functions of the delivery system 3000. The pusher shaft 3008 and the release shaft 3010 may extend proximally of the handle 3024.

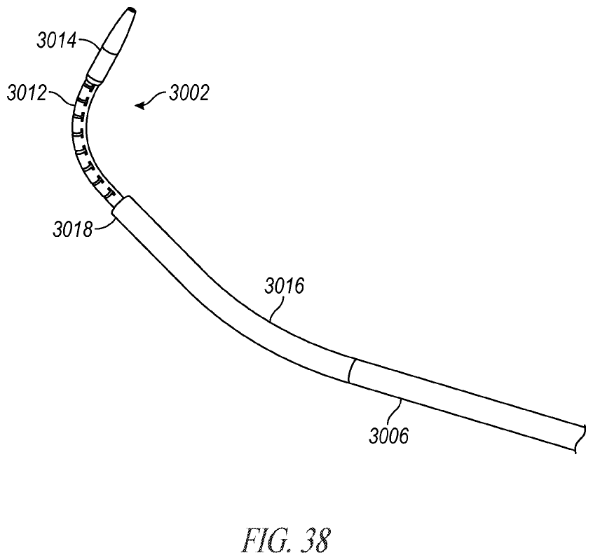

FIG. 38 illustrates a portion of an implantable device 3002 projecting from a distal end 3018 of the outer sheath 3006. The implantable device 3002 may include any of the features of the implantable device shown in FIG. 23 or other sensing attachments, auxiliary components, scaffolds, and sensing constructs described herein. As illustrated, the implantable device 3002 may include a body portion 3012 (also referred to herein as a support structure) and a distal portion 3014. The distal portion 3014 may include any of the sensing, communicating, powering, charging and/or other functions described above. The implantable device 3002 may include a lumen extending through the distal portion 3014 to accommodate a guidewire.

The outer sheath 3006 may constrain the coiled implantable device 3002 into a generally linear configuration. The outer sheath 3006 may have a diameter of less than or equal to about 15 French or less than or equal to about 13 French. As explained above, the body 3012 may be shape set to a coil configuration (see FIG. 23). For delivery, the implantable device 3002 may be loaded into the outer sheath 3006 and constrained in the generally linear configuration. The implantable device 3002 may be coupled to the pusher shaft 3008, for example by a press-fit. When retracted, the pusher shaft 3008 may load the implantable device 3002 into the outer sheath 3008. The pusher shaft 3008 may have a diameter of less than or equal to about 13 French or less than, or equal to about 12 French, or less than or equal to about 10 French. When the implantable device 3002 is fully loaded, the distal portion 3014 of the implantable device 3002 may project from distally from the distal end 3018 of the outer sheath 3006 to form the distal tip of the delivery system 3000. The distal portion 3014 of the implantable device 3002 may form a press-fit or a loose-fit with the distal end of 3018 of the outer sheath 3006. In other implementations, the entire distal portion 3014 may be positioned adjacent the distal end 3018 and entirely within the outer sheath 3006

An implantable device 3002 capable of being loaded into an outer sheath 3006 and deployed to form a coil configuration may exhibit certain mechanical properties. For example, in the coiled configuration, the implantable device 3002 may withstand a sufficient linear compression force to stake the implantable device 3002 within an aneurysmal sac and maintain the position of the implantable device 3002 within the aneurysmal sac in spite of movement of the human anatomy. The implantable device 3002 may also withstand a sufficient linear compression force to enable the implantable device 3002 to maintain an internal diameter sufficient to allow a second delivery system to be advanced through the coil to deliver a treatment device. For example, the implantable device 3002 may be able to withstand a linear compression force of at least about 1.0 N and/or less than or equal to about 30.0 N, for example up to 5.0 N, up to 8.0 N, up to 10.0 N, up to 12.0 N, up to 14.0 N, up to 16.0 N, up to 18.0 N, up to 20.0 N, or up to 25.0 N, prior to failure. The implantable device 3002 may withstand a compression force from about 1.0 N to about 25.0 N, for example, from about 1.0 N to about 5.0 N, from about 5.0 N to about 15.0 N, from about 15.0 N to about 25.0 N, from about 20.0 N to about 30.0 N, or ranges in between. The compression test may be bound by methods required by ISO 25539-2012 and ISO 104065/2/1. The test may be performed at a speed of 2 mm/min to 60 mm/min at a temperature of 22° C. Upon application of a force in the linear direction of the coiled configuration, referred to as the linear compression force, the linear direction being along the axis of the coil or helix, the coil will resist compression, i.e., the pitch of the coil will remain substantially unchanged, upon application of the linear compression force of, in embodiments, 1.0 N to about 30.0 N as explained above. For example, in one embodiment, the present disclosure provides an implantable sensing construct configured to be percutaneously implanted in an aneurysmal sac, the implantable sensing construct comprising: a sensor; and a body comprising a first configuration and a second configuration, wherein in the first configuration, the body comprises a substantially linear shape for transport in a delivery system; and wherein in the second configuration the body comprises a coiled shape when released from the delivery system, wherein the coiled shape has a pitch and the pitch is substantially maintained upon application of a linear compression force of up to 8.0 N.

In the coiled configuration, the implantable device 3002 may withstand a sufficient tension force to allow the implantable device 3002 to be pulled straight within the delivery system 3000. For example, the implantable device 3002 may withstand a tension force of at least about 5.0 N and/or less than or equal to about 105.0 N, for example up to 8.0 N, up to 15.0 N, up to 30.0 N, or up to 105.0 N. The implantable device 3002 may withstand a tension force from about 5.0 N to about 15.0 N, from about 15.0 N to about 30.0 N, from about 30.0 N to about 105.0 N, or ranges in between. The tension test may be bound by methods required ISO 25539-2012 and ISO 104065/2/1. The test may be performed at a speed of 2 mm/min to 60 mm/min and a temperature of 22° C.

The implantable device 3002 may exhibit these mechanical properties for an implant sized to provide minimal to no radial force against the wall of the aneurysmal sac, while still managing position control within the aneurysmal sac. For example, the implantable device 3002 may exhibit these properties for an implantable device implanted in an abdominal aortic aneurysm and having an internal diameter of less than or equal to 50.0 mm, or less than or equal to about 25.0 mm, in the coil configuration. The implantable device 3002 may exhibit these properties for an implantable device having an outer diameter of less than or equal to about 50.0 mm in the coil configuration.

A distal portion 3016 of the outer sheath 3006 may be actively or passively deflectable in at least one direction. For example, the handle 3024 may be used to actively steer the distal portion 3016 of the outer sheath 3006. In some embodiments, the outer sheath 3006 is only steerable in a single direction. In other embodiments, the outer sheath 3006 is steerable in all directions. Steering facilitates proper positioning of the implantable device 3002. Because the implantable device 3002 may have sensing, communicating, powering, charging, and/or other capabilities, the implantable device 3002 may need to be properly oriented to improve functionality. For example, it may be beneficial to position the distal portion 3014 of the implantable device 3002 within a posterior region of an aneurysmal sac to improve antenna communication or inductive charging. There may be a smaller distance between the sac and the patient's backside compared to the sac and the patient's front side.

The outer sheath 3006 may include an internal diameter of less than or equal to 6 mm or less than or equal to 5 mm. The outer sheath 3006 may include any suitable medical grade material, including but not limited to, Pebax® polyethylene, tetrafluoroetheylene, polytetrafluoroethylene, or other polymeric materials.

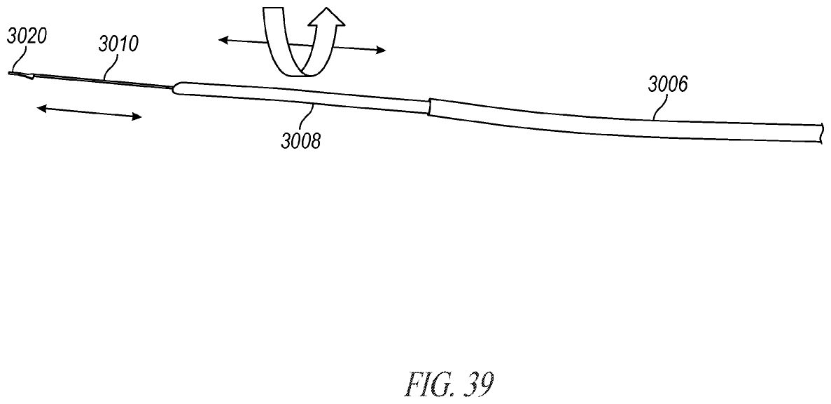

FIG. 39 shows the relative movement of the pusher shaft 3008 and the release shaft 3010 relative to the outer sheath 3006. The pusher shaft 3008 may advance the implantable device 3002 out of the outer sheath 3006 and/or re-sheath the implantable device 3002 into the outer sheath 3006. The pusher shaft 3008 in combination with or independently of the release shaft 3010. The pusher shaft 3008 may be rotatable to provide torque to the implantable device 3002. For example, during transport, the implantable device 3002 may get twisted. Prior to partially or fully advancing the implantable device 3002 out of the outer sheath 3006, the pusher shaft 3008 may be rotated to apply torque to the implantable device 3002 to untwist the implantable device 3002 for proper deployment. In some implementations, the pusher shaft 3008 may be rotated to apply torque to the implantable device 3002 to release the implantable device 3002 from the pusher shaft 3008 when the proximal portion of the implantable device 3002 is outside of the outer sheath 3006.

The pusher shaft 3008 may include an outer diameter of less than or equal to 5 mm or less than or equal to 4 mm. The pusher shaft 3008 may include any suitable medical grade material, including but not limited to, Pebax® polyethylene, tetrafluoroetheylene, polytetrafluoroethylene, or other polymeric materials. The pusher shaft 3008 may include a guidewire lumen sufficient to accommodate a guidewire having an outer diameter of at least 1 mm or at least 1.33 mm.

The release shaft 3010 may be advanced to release the distal portion 3014 of the implantable device 3002 from the outer sheath 3006. As illustrated, the release shaft 3010 may include an enlarged distal portion 3020. When the implantable device 3002 is loaded in the outer sheath 3006, the distal portion 3020 of the release shaft 3010 may be positioned within a lumen of the implantable device 3002 and may not extend beyond the distal portion 3014 of the implantable device 3002 during transport or prior to deployment. When the release shaft 3010 is advanced, the enlarged distal portion 3020 may act on the implantable device 3002 to release the distal portion 3014 of the implantable device 3002 from the outer sheath 3006. For example, the release shaft 3010 may push against an internal surface of the implantable device 3002, for example a ring or other projecting feature on the internal surface of the implantable device 3002. Following release of the distal portion 3014 of the implantable device 3002, the pusher shaft 3008 may further advance the implantable device 3002 out of the outer sheath 3006.

The distal portion 3020 of the release shaft 3010 may be used to transition the distal portion 3014 of the implantable device 3002 between a first configuration during transport and a second configuration when deployed. In the first configuration, the distal portion 3014 of the implantable device 3002 may be compressed or rolled into a cylindrical, conical or other three-dimensional shape to form a distal tip. In the second configuration, the distal portion 3014 of the implantable device 3002 may be expanded or unrolled into a substantially flattened shape compared to the first configuration. In some implementations, the distal portion 3014 of the implantable device 3002 may include an antenna, power or recharging capabilities, or other circuitry to enable the sensing and communication functions of the implantable device. When the release shaft 3010 pushes on the distal portion 3014 of the implantable device, the release shaft 3010 may initiate the transition of the distal portion 3014 of the implantable device 3002 from the first, compressed configuration to the second, expanded configuration.

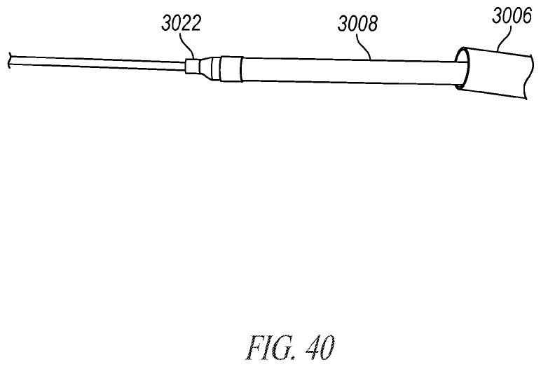

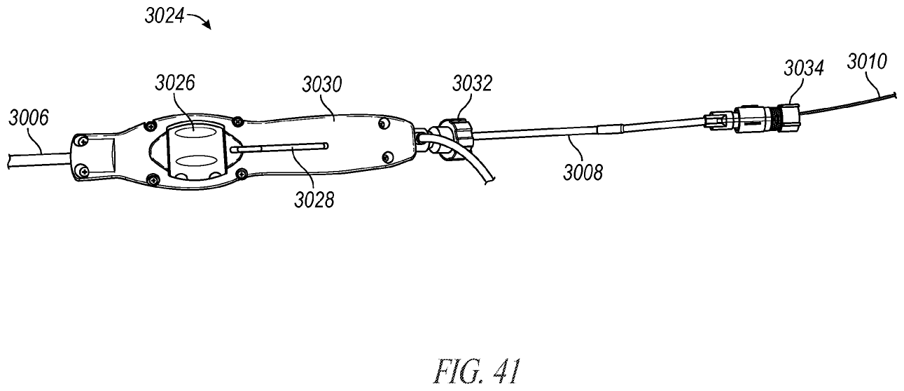

FIG. 40 illustrates an enlarged view of a distal portion 3022 of the pusher shaft 3008. As illustrated, the distal portion 3022 of the pusher shaft 3008 may be shaped to interface with a proximal portion of the implantable device 3002. For example, the distal portion 3022 of the pusher shaft 3008 may be include a reduced diameter to fit within a lumen of the implantable device 3002. The distal portion 3022 of the pusher shaft 3008 may form a press-fit with the implantable device 3002. The distal portion 3022 may be integral with the remainder of the pusher shaft 3008 or an adaptor coupled to the pusher shaft 3008. Other connections between the implantable device 3002 and the pusher shaft 3008 are possible. The pusher shaft 3008 may include a lip to carry a proximal end of the implantable device 3002. The implantable device 3002 and the distal portion 3022 of the pusher shaft 3008 may include one or more interlocking features.

As explained above, rotating the pusher shaft 3008 to apply torque to the implantable device 3002 may release the implantable device 3002 from the pusher shaft 3008. In other implementations, the implantable device 3002 may be released from the pusher shaft 3008 as soon as the proximal portion of the implantable device 3002 extends beyond a distal end of the outer sheath 3006.

FIG. 41 illustrates the handle 3024 that may be used to control one or more functions of the delivery system 3000. The handle 3024 may include a handle body 3030 and one or more user-actuatable controls 3026 disposed in or adjacent to the handle body 3030. Although the illustrated handle 3024 includes one user-actuatable controls 3026, a fewer or greater number of controls may be incorporated into the handle 3024.

The handle 3024 may include a first user-actuatable control 3026 to control deflection of the distal portion 3016 of the outer sheath 3006. Moving the first user-actuatable control 3026 in a first direction may deflect the distal portion 3016 of the outer sheath 3006 in a first direction. Moving the first user-actuatable control 3026 in the opposite direction may deflect the distal portion 3016 of the outer sheath 3006 in the opposite direction. The first user-actuatable control 3026 may drive a pulley system within the handle 3024 to control deflection of the distal portion 3016 of the outer sheath 3006. The handle body 3030 may include a window 3028 to visualize a position of a component of the pulley system and corresponding deflection of the distal portion 3016 of the outer sheath 3006.

The pusher shaft 3008 may be advanced, retracted, and/or rotated by manipulating the portion of the pusher shaft 3008 extending proximally of the handle 3024. The release shaft 3010 may be advanced and/or retracted by manipulating the portion of the release shaft 3010 extending proximally of the handle 3024.

The proximal portion of the delivery system 3000 may include one or more locking feature 3032, 3034, for example a tuohy borst, to prevent relative movement between the outer sheath 3006, pusher shaft 3008, and/or release shaft 3010. For example, the proximal portion may include a first locking feature 3032 to prevent axial and/or rotational movement between the pusher shaft 3008 and outer sheath 3006 during transport. A clinician may choose to prevent movement between the pusher shaft 3008 and the outer sheath 3006 during delivery of other treatment devices. The locking feature 3032 may include a seal to prevent the back flow of fluid. The proximal portion 3004 may include a second locking feature 3034 to prevent axial and/or rotational movement between the release shaft 3010 and the pusher shaft 3008 during transport or during delivery of other treatment device. The second locking mechanism 3034 may include a seal to prevent the back flow of fluid.

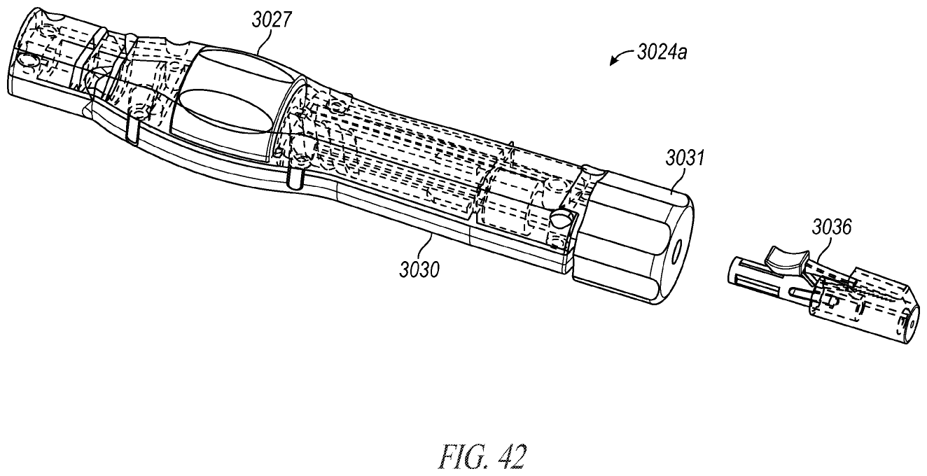

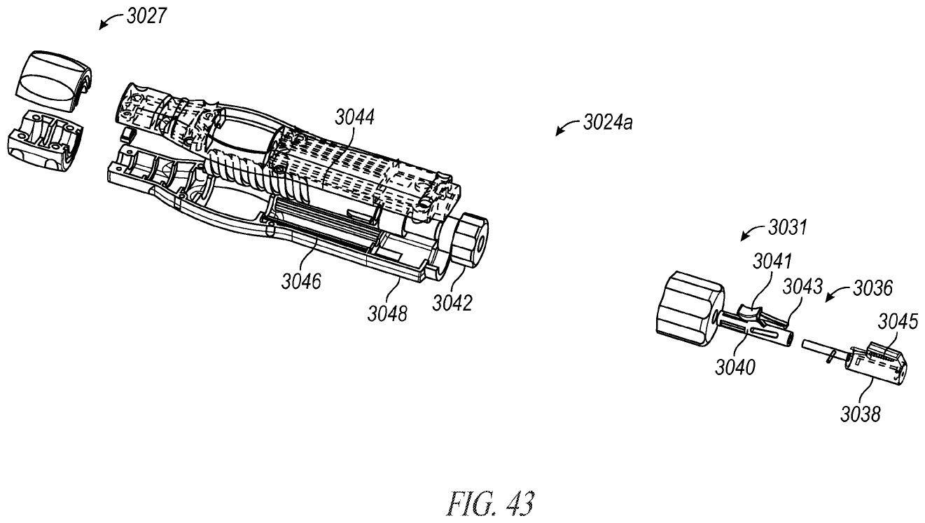

FIGS. 42 and 43 illustrate another handle 3024a that may be used in connection with the delivery system 3000. The handle 3024a may include any of the features of the handle 3024. The handle 3024a may include a handle body 3048 and a plurality of user-actuatable controls 3027, 3031, 3036. Each of the user-actuatable controls 3027, 3031, 3036 may be disposed in the handle body 3048 or adjacent the handle body 3048.

The handle 3024a may include a first-actuatable control 3027 to control deflection of the distal portion 3016 of the outer sheath 3006. The distal portion 3016 may drive one or more worm gears 3044 to deflect the distal portion 3016 of the outer sheath 3006. Moving the first user-actuatable control 3027 in a first direction may deflect the distal portion 3016 of the outer sheath 3006 in a first direction. Moving the first user-actuatable control 3027 in the opposite direction may deflect the distal portion 3016 of the outer sheath 3006 in the opposite direction. The handle body 3048 may include a window 3046 to visualize a position of the worm gear 3044 and corresponding deflection of the distal portion 3016 of the outer sheath 3006.

The handle 3024a may include a second user-actuatable control 3031. Rotating the second user-actuatable control 3031 may rotate the pusher shaft 3008. The second user-actuatable control 3031 may drive a connector 3042 to rotate the pusher shaft 3008. The connector 3042 may be uni-directional to only rotate the pusher shaft 3008 in one direction. The connector 3042 may provide a seal between the pusher shaft 3008 and the release shaft 3010 to prevent proximal leaking to the proximal end of the delivery system 3000.

The handle 3024a may include a third user-actuatable control 3036 to control advancement and/or retraction of the pusher shaft 3008. The third user-actuatable control 3036 may provide discrete and/or continuous axial movement of the pusher shaft 3008. The third user-actuatable control 3036 may include a linear actuator. The linear actuator may include a first portion 3040 capable of sliding relative to a second portion 3038. The first portion 3040 may be capable of sliding within the second portion 3038. The first portion 3040 may include a tooth 3043 or other projecting structure, and the second portion 3038 may include a rack 3045. The first portion 3040 may include a button 3041. Depressing the button 3041 may cause the tooth 3043 to engage or disengage with the rack 3045. When the tooth 3043 is engaged with the rack 3045, axial movement between the pusher shaft 3008 and the outer sheath 3006 may be prevented. A clinician may depress the button 3041 and move the tooth 3043 over one tooth of the rack 3045 for discrete movement of the pusher shaft 3008. A clinician may depress the button 3041 and continuously slide the first portion 3040 relative to the second portion 3038 for continuous movement of the pusher shaft 3008. Although not shown, a similar user-actuatable control may be used to advance and/or retract the release shaft 3010.

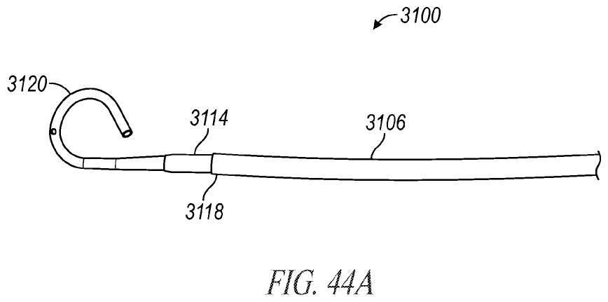

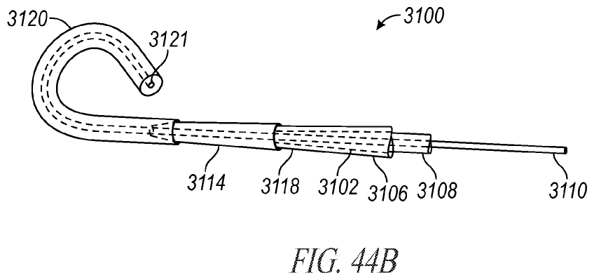

FIGS. 44A and 44B illustrate another delivery system 3100 for delivering an implantable device 3102 including any of the features of the implantable devices described herein (also referred to herein as sensing attachments, sensing constructs, auxiliary components, or scaffolds). The delivery system 3100 may include any of the features of the delivery system 3000. For example, the delivery system 3100 may include any of the control features of the delivery system 3000. Numerals used to identify features of the delivery system 3000 are incremented by a factor of one hundred (100) to identify like features of the delivery system 3100. Any component or step disclosed in any embodiment in this specification can be used in other embodiments.

As shown in FIGS. 44A and 44B, a distal portion 3114 of the implantable device 3102 may project distally from a distal end 3118 of the outer sheath 3106, but may not form a distal tip of the delivery system 3100. Instead, a distal portion 3120 of the release shaft 3110 may form an atraumatic distal tip of the delivery system 3100. The distal portion 3114 of the implantable device 3102 may be positioned between the distal portion 3120 of the release shaft 3110 and the distal end 3118 of the outer sheath 3106. An outer diameter of the distal portion 3120 of the release shaft 3110 may be less than or equal to a diameter of a lumen of the distal portion 3114 of the implantable device 3102. The distal portion 3120 of the release shaft 3110 may abut the distal portion 3114 of the implantable device 3102 or be coupled to the distal portion 3114 of the implantable device 3102, for example with an interference fit, during transport and navigation through the vasculature.

Although FIGS. 44A and 44B illustrate the distal portion 3114 of the implantable device 3102 projecting from the distal end 3118 of the outer sheath 3106, in other embodiments, the entire implantable device 3102 may be carried within the outer sheath 3106 during transport and advancement to the implantation site or the distal portion 3114 of the implantable device 3102 may be carried within the distal portion 3120 of the release shaft 3110. In this configuration, the distal portion 3120 of the release shaft 3110 may abut the distal end 3118 of the outer sheath 3106.

The distal portion 3120 of the release shaft 3110 may include a soft and flexible polymer capable of passive deflection. The distal portion 3120 may be pre-shaped to form an atraumatic curvature. The curvature of the distal portion 3120 may provide tactile feedback to the clinician when the distal portion 3120 contacts a wall of the aneurysmal sac.

The distal portion 3120 of the release shaft 3110 may include a lumen 3121 to accommodate a guidewire. The distal portion 3120 may conform to the shape of the guidewire as the delivery system 3100 is advanced along the guidewire.

Axial movement of the release shaft 3110, as described above, may displace the distal portion 3120 of the release shaft 3110 from the outer sheath 3106 to allow the implantable device 3102 to be deployed from the outer sheath 3106. For example, the release shaft 3110 may be advanced to permit the distal portion 3114 of the implantable device 3102 to be released from the outer sheath 3116 using the pusher shaft 3108. After at least a portion of the implantable device 3102 has been deployed from the outer sheath 3106 (e.g., less than or equal to one turn, or less than or equal to one-half turn), the distal portion 3120 of the release shaft 3110 may be withdrawn through the implantable device 3102, and the pusher shaft 3108 may advance the implantable device 3102 out of the outer sheath 3106. Withdrawing the distal portion 3120 of the release shaft 3110 may cause the distal portion 3114 of the implantable device 3102 to transition from a first, compressed configuration to a second, expanded configuration as described above. For example, the distal portion 3120 of the release shaft 3110 may release an interlock between the distal portion 3114 of the implantable device 3102 and the body of the implantable device 3102.

In other techniques, the release shaft 3110 may be retracted through the implantable device 3102 to permit the distal portion 3114 of the implantable device 3102 to be released from the outer sheath 3116, or the distal portion 3114 of the implantable device 3102 may be advanced over the distal portion 3120 of the release shaft 3110.

Any of the delivery systems described herein may be provided with an adaptor for connection to a robotic surgical system. The clinician may use the robotic surgical system to actively steer the delivery system to the target site. Robotic surgical systems, teleoperated surgical systems, and the like, which may be used or adapted to connect with a delivery system of the present disclosure so as to deliver and implant an implantable device of the present disclosure into a patient, have been commercialized by several companies. One example of such a teleoperated, computer-assisted surgical system (e.g., a robotic system that provides telepresence) with which embodiments of the present disclosure may be used, are the da Vinci Surgical Systems manufactured by Intuitive Surgical, Inc. of Sunnyvale, Calif, USA. See, e.g., U.S. Pat. Nos. 9,358,074; 9,295,524; and 8,852,208; U.S. Patent Publication Nos. 20140128886; 20200253678; 20190192132; 20190254763; 20180318020; 20170312047; 20170172671; 20170172674; 20170000575; 20170172670; 20130204271; and 20120209305; and PCT Publication No. WO2020150165, each of which is incorporated by reference. Another example is Medtronic, Inc. (Minneapolis, MN, USA; and related companies, e.g., Covidien LP, Mansfield MA USA and Medtronic Navigation, Inc., Louisville CO USA) including their Digital Surgery Division and Surgical Robotics Division, which has commercialized various robotic-assisted surgery (RAS) solutions. See, e.g., U.S. Patent Publication Nos. 20200222127; 20190365477; 20190214126; 20190069964; and 20130289439, each of which is incorporated by reference. Yet another example is Auris Health (Redwood City, CA USA; Auris Health, Inc., is part of Johnson & Johnson Medical Devices Companies. Auris Health, Inc. was formerly known as Auris Surgical Robotics, Inc.) which has commercialized their Monarch platform. See, e.g., U.S. Patent Publication Nos. 20200198147; 20200100845; 20200100853; 20200100855; 20200093554; 20200060516; 20200046434; 20200000537; 20190365209; and 20190365486, each of which is incorporated by reference. In addition, Stryker Corp. (Kalamazoo MI USA) discloses robotic surgical systems in, e.g., U.S. Patent Publication Nos. 20160374770 and 20140276949, both of which are incorporated by reference. See also, e.g., U.S. Patent Publication Nos. 20200046978; 20200001053; 20200197111; 20190262084; 20190231447; and 20190090957 and PCT Publication Nos. WO2019195841 and WO2019082224, where each of the identified publications is incorporated by reference. In one embodiment, the handle of the delivery system of the present disclosure is configured to dock with an arm of a robotic surgical system. In one embodiment, the delivery system of the present disclosure integrates with a robotic surgical system to provide robot-assisted delivery and implantation of the implantable device of the present disclosure into a patient. In one embodiment, the present disclosure provides a method for advancing any of the implantable device described herein through the vasculature of a patient, using robotic assistance.

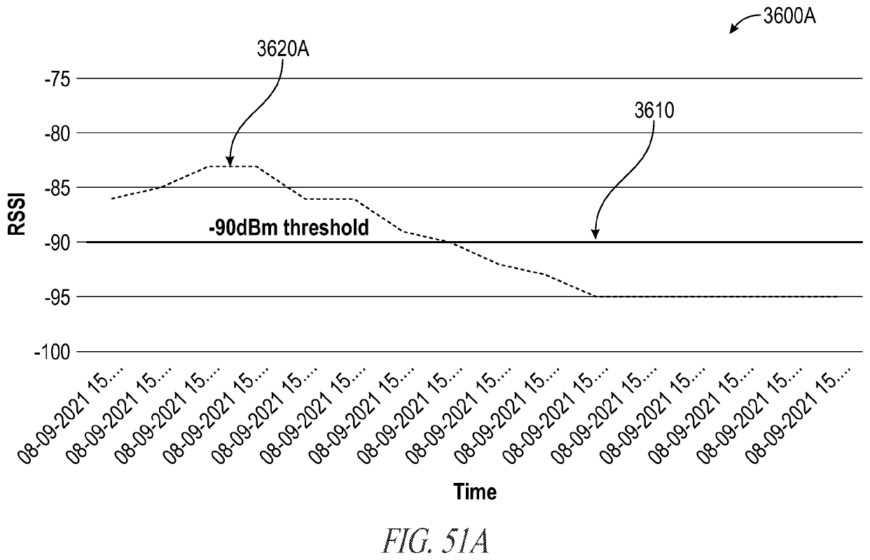

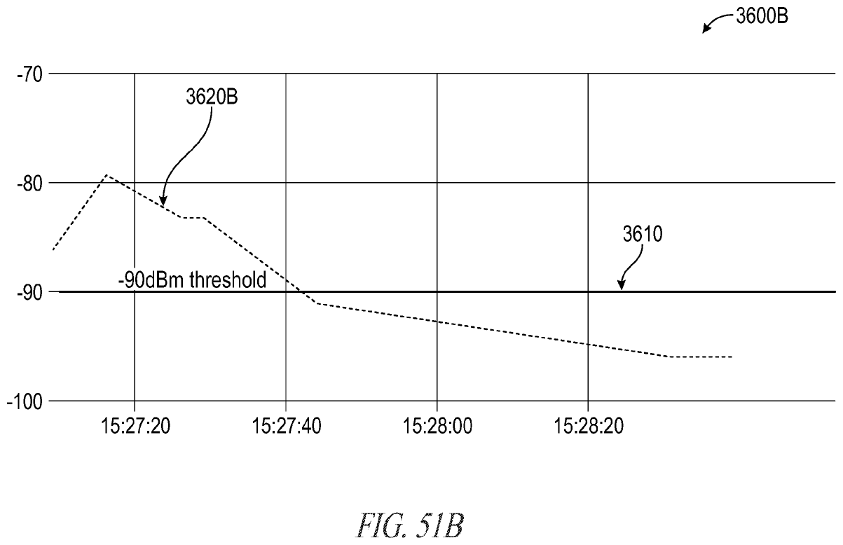

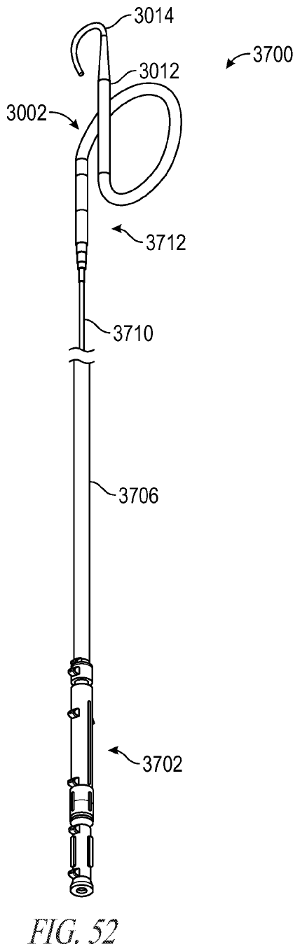

FIG. 52 illustrates another delivery system 3700 that may be used to deliver any of the implantable devices described herein. The delivery system 3700 may include any of the features of the delivery system 3000 described above.

The delivery system 3700 may include a handle 3702 and an outer sheath 3706 extending from the handle 3702. An intermediate tube (not shown in FIG. 52) may extend through the outer sheath 3706 and be releasably coupled to the implantable device 3002, for example by disconnect assembly 3712. An inner tube 3710 may extend through the intermediate tube and the implantable device 3002. During transport, the implantable device 3002 may be disposed radially between the inner tube 3710 and the outer sheath 3706. At least a portion or substantially the entire length of the implantable device 3002 may be positioned distal of the intermediate tube. The various tubes of the delivery system 3700 can act independently or in conjunction to release the implantable device. A distal portion of the outer sheath 3706 may be deflected to access the target location. For example, the outer sheath 3706 may include one or more tension wires within the walls of the outer sheath 3706. In some embodiments, the distal portion of the outer sheath 3706 may only deflect within a single plane, but in other embodiments, the distal portion of the outer sheath 3706 may deflect in multiple planes.

A portion of an implantable device 3002 may project from a distal end of the outer sheath 3706 as the delivery system 3700 is advanced through the vasculature. As illustrated, the implantable device 3002 may include a body portion 3012 and a distal portion 3014. The distal portion 3014 may include any of the sensing, communicating, powering, charging and/or other functions described above. The body portion 3012 may include any of the features described herein, for example body 3812 or 3912.

The outer sheath 3706 may constrain the implantable device 3002 into a generally linear configuration as described above. When the implantable device 3002 is fully loaded, the distal portion 3014 of the implantable device 3002 may project distally from the distal end of the outer sheath 3706 to form the distal tip of the delivery system 3700. The distal portion 3014 of the implantable device 3002 may form a press-fit or a loose-fit with the distal end of the outer sheath 3706. In other implementations, the entire distal portion 3014 may be positioned entirely within the outer sheath 3706.

FIG. 53A illustrates an enlarged view of the handle 3702 shown in FIG. 52. FIG. 53B illustrates a partial exploded view of the handle 3702.

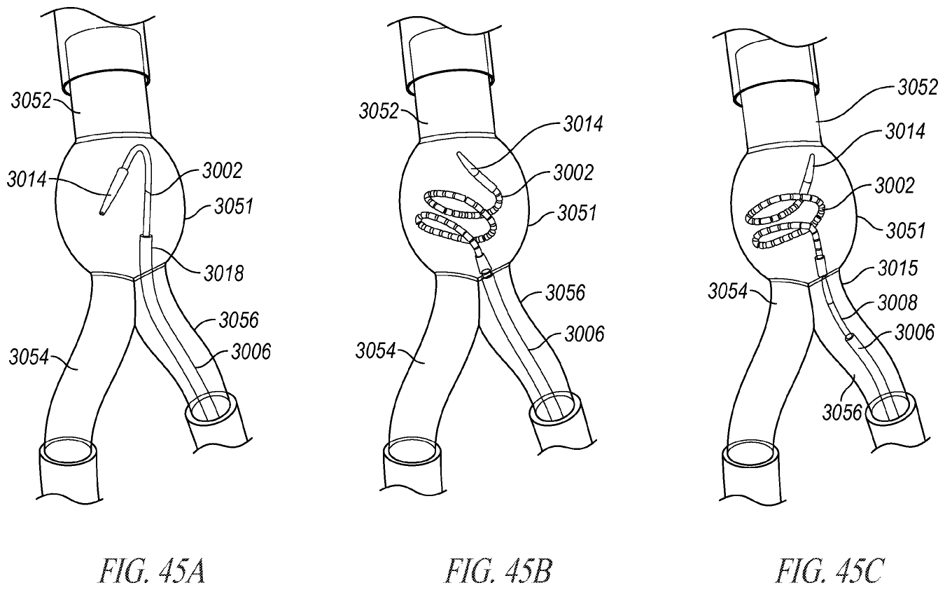

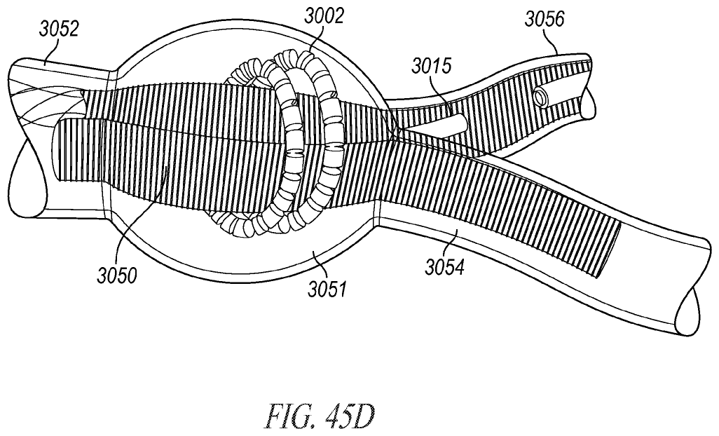

As shown in FIG. 53A, the handle 3702 can include a proximal portion 3720 and a distal portion 3722. The proximal portion 3720 may include an actuator or handle driver 3724, and the distal portion 3722 may include a handle enclosure 3726. The handle driver 3724 may be movable relative to a handle enclosure 3726. For example, a distal portion 3724b of the handle driver 3724 may be rotatably coupled to the proximal portion 3726a of the handle enclosure 3726. As the handle driver 3724 is rotated, the implantable device 3002 may be gradually released from the outer sheath 3706 to form the coil shape (see FIGS. 45A-45C).

As shown in FIG. 53B, the distal end portion 3724 of the handle driver 3724 may form a collar received by a proximal portion 3726a of the handle enclosure 3726. The proximal portion 3726a of the handle enclosure 3726 may include a groove that captures the collar of the handle driver 3724 to maintain an axial position of the handle driver 3724 relative to the handle enclosure 3726. In other configurations, the handle driver 3724 may be axially movable relative to the handle enclosure 3726.

The handle enclosure 3726 may include an elongate slot 3730 along a length of the handle enclosure 3726. An indicator 3738 (shown in FIG. 53B) may travel along the slot 3730 to provide an indication of a location of the implantable device 3002 relative to the outer sheath 3706 or an indication of a length of the implantable device 3002 that has been deployed from the outer sheath 3706. The handle enclosure 3726 may include one or more markers (not shown) along the length of the slot 3730 to provide an indication of the number of turns of the implantable device 3002 that have been released from the outer sheath 3706. The clinician may use the indication to determine when to release the treatment device. For example, some clinicians may desire to release the treatment device as soon as a single turn of the implantable device 3002 has been released from the outer sheath 3706. Other clinicians may desire to release the treatment device after the entire implantable device 3002 has been released from the outer sheath 3706.

Another actuator or collar 3728 may be provided to control deflection of a distal portion of the outer sheath 3706. The collar 3728 may be disposed about a distal portion 3726b of the enclosure 3726. The position of the collar 3728 may be locked in place to maintain deflection of the distal portion of the outer sheath 3706.

Turning to FIG. 53B, the handle 3702 may include a first lead screw 3732 and a second lead screw 3734. The first lead screw 3732 may be positioned to act on or drive the intermediate tube. For example, a proximal end of the intermediate tube may be disposed within the coupler 3733 at a distal end of the first lead screw 3732. The intermediate tube may also extend through an anchor 3740 disposed in the handle enclosure 3726 to maintain alignment of the intermediate tube. The inner tube 3710 may be disposed within a lumen of the intermediate tube and extend proximally of the intermediate tube to the release pin 3736 at the proximal end of the handle 3702.

Upon rotation of the handle driver 3724, the first and/or second lead screws 3732, 3734 may translate in a linear direction to advance or retract the intermediate tube and the implantable device 3002. For example, upon rotation of the handle driver 3724 in a first direction, the first lead screw 3732 may interface with or act on the proximal end of the intermediate tube to drive the intermediate tube forward. Upon rotation of the handle driver 3724 in the opposite direction, the intermediate tube may be retracted.

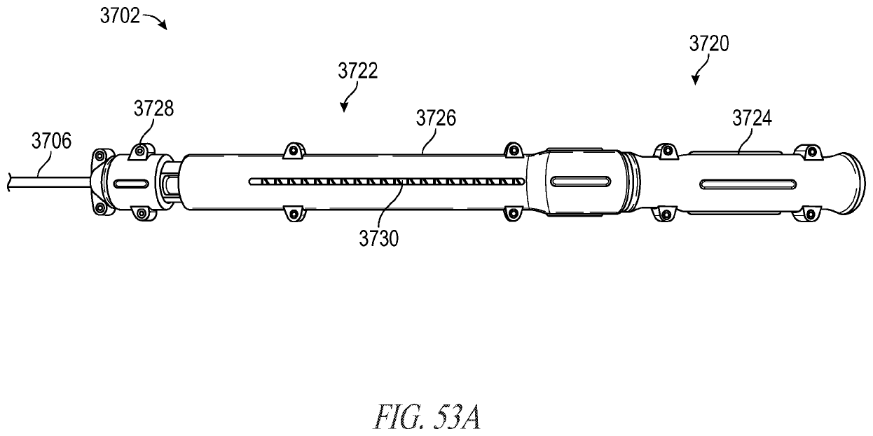

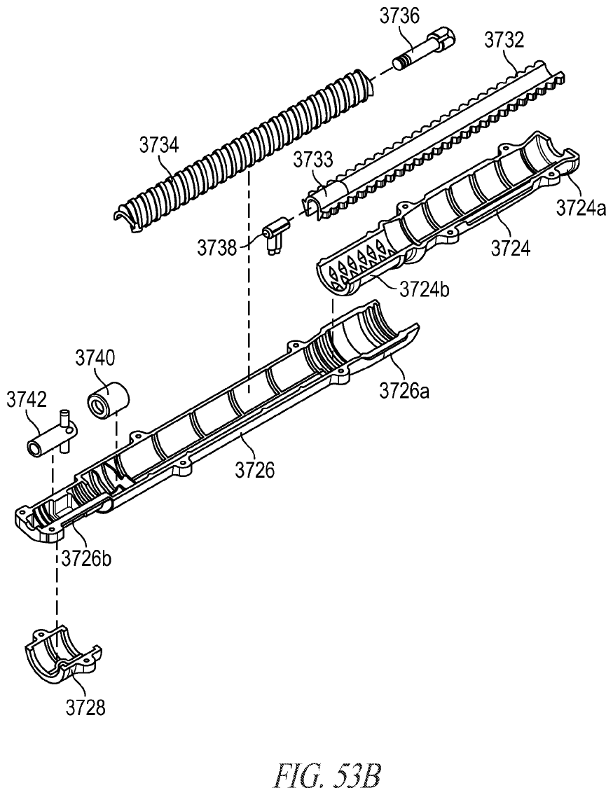

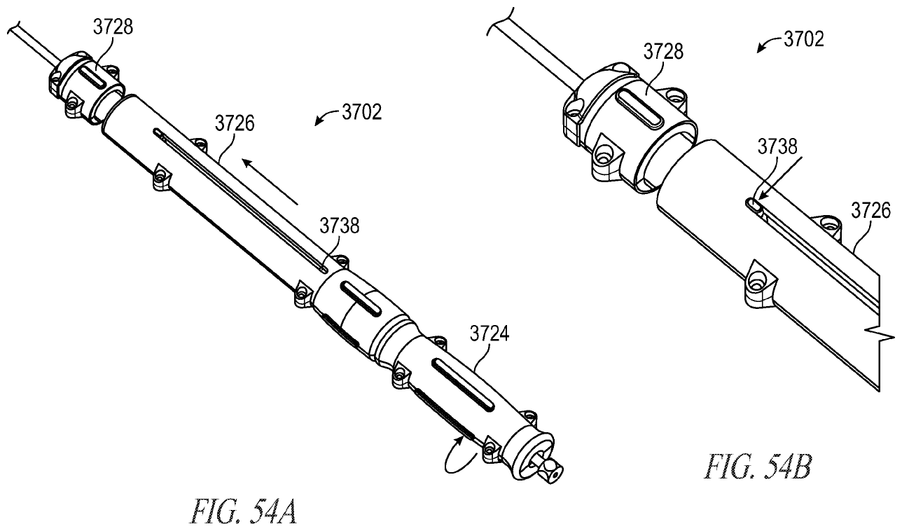

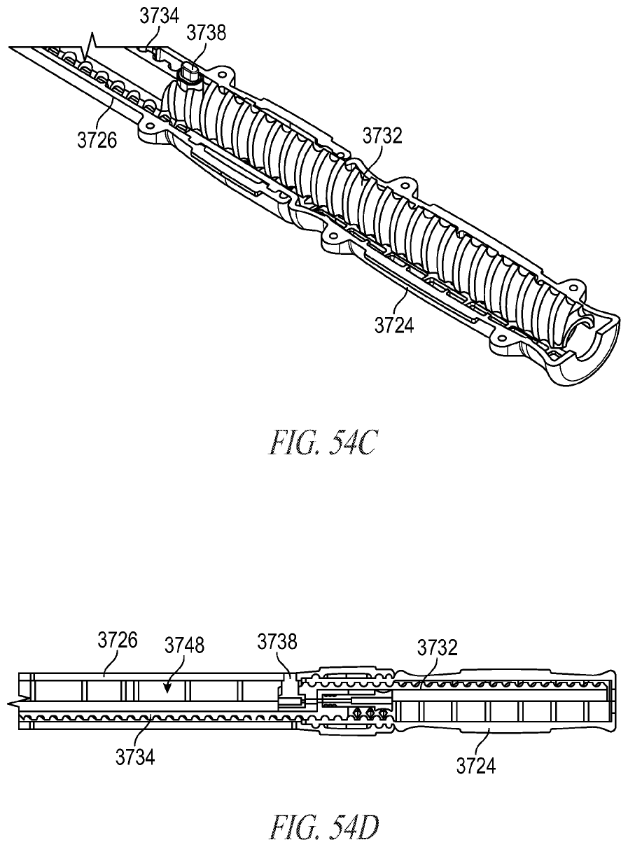

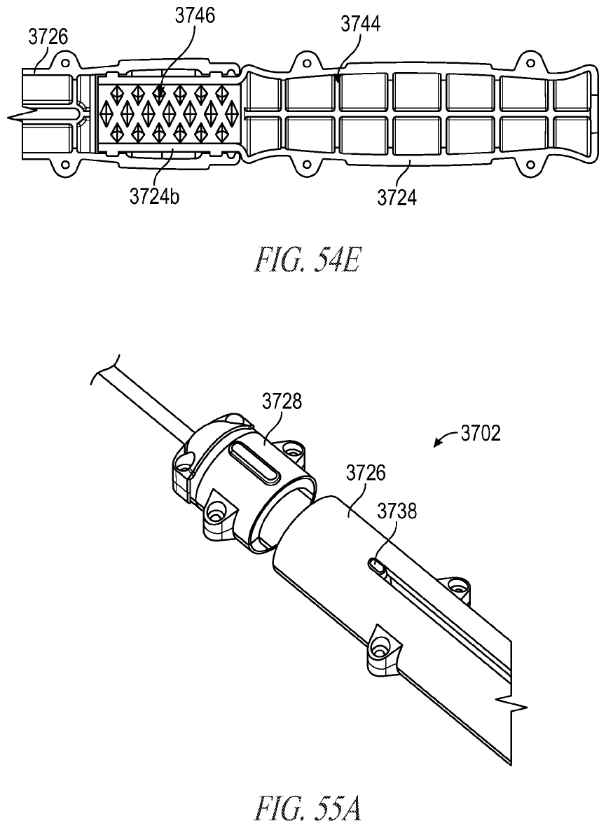

The first lead screw 3732 may be at least partially disposed within the handle driver 3724. The second lead screw 3734 may be at least partially disposed within the handle enclosure 3726. A proximal portion 3724a of the handle driver 3724 may include recesses to provide clearance for the threads on the first lead screw 3732. The handle enclosure 3726 may include recesses to provide clearance for the threads on the second lead screw 3734. The distal portion 3724b may include a threaded pattern 3746 shaped to interface with and drive the first lead screw 3732 and/or the second lead screw 3734 in the forward and backward directions. For example, as more closely shown in FIG. 54E, the distal portion 3724b may include a non-continuous thread pattern 3746. For example, the thread pattern 3746 may include multiple diamond-shaped recesses. The thread pattern 3746 may include multiple rings of diamond shaped recesses with adjacent rings circumferentially offset from each other. Each of the diamond-shaped recesses may be shaped and positioned to drive both the first lead screw 3732 and the second lead screw 3724.

Each of the first lead screw 3732 and the second lead screw 3734 may be only partial body screws truncated along a longitudinal plane, for example hollow, half body screws. The threads on the first lead screw 3732 and the threads on the second lead screw 3734 may be oriented in opposite directions, for example the first lead screw 3732 may be threaded in a clockwise direction and the second lead screw 3734 may be threaded in a counter-clockwise direction, or vice versa. The threads on the first lead screw 3732 and the threads on the second lead screw 3734 may include the same pitch or different pitches. Rotating the driver will translate one of the lead screws forward and the other lead screw backward. Although the first and second lead screws 3732, 3734 are moving in opposite directions, they may travel the same distance at a 1:1 ratio.

As shown in FIG. 53B, the first and second lead screws 3732, 3734 may be circumferentially and/or axially offset from each other. When the implantable device 3002 is loaded in the delivery system, a distal portion of the first lead screw 3732 may abut or overlap with a proximal portion of the second lead screw 3732 (see FIGS. 54C and 54D). The first lead screw 3732 and the second lead screw 3734 may be fixed to each other or float relative to each other. The overlapping portions may be disposed within the distal portion 3724b of the handle driver 3724 having the threaded pattern 3746. Rotation of the handle driver 3724 will drive one of the first lead screw 3732 and the second lead screw 3734 in a forward direction and the other one of the first lead screw 3732 and the second lead screw 3734 in the opposite direction.

The indicator 3738 may be directly or indirectly affixed to a distal end of the first lead screw 3732. The intermediate tube may extend through a lumen of the indicator 3738. As the first lead screw 3732 travels in the forward or backward direction, the indicator 3738 travels along the slot 3730 to provide an indication of the location of the implantable device 3002 relative to the outer sheath 3706. The indicator 3738 may also provide an indication of the rotational position of the implantable device 3002 within the outer sheath 3706.

The handle 3702 may include a seal at the proximal end of the handle 3702 to prevent back flow of fluid. The seal may take the form of a release pin 3736 permanently or releasably coupled to the inner tube 3710. Rotation of the inner tube 3710 by the release pin 3736 may release the distal portion 3014 of the implantable device 3002 from the outer sheath 3706, allowing the distal portion 3014 to expand. In some implementations, rotation of the driver 3724 may cause the second lead screw 3734 to act on the inner tube 3710 to release the distal portion 3014. After the treatment device has been deployed, the blood flow may be sufficiently reduced to remove the release pin 3736, which enables the inner tube 3710 to be withdrawn from the implantable device 3002 and the disconnect assembly 3712.

The handle 3702 may include a steering cam 3742 disposed within the distal portion 3726b of the handle enclosure 3726. Rotation of the collar 3728 rotates the steering cam 3742 to lock the position of the collar 3728 and the shape of the outer sheath 3706.

FIGS. 54A and 54B illustrate actuation of the handle driver 3724. As the handle driver 3724 rotates in a first direction, for example the clockwise direction, the first lead screw 3732 is advanced in an axial direction. As the first lead screw 3732 advances forward, the indicator 3738 travels along the length of the slot 3730. FIG. 54A illustrates the indicator 3738 in a first position indicating that the implantable device 3002 is fully loaded within the outer sheath 3706. FIG. 54B illustrates the indicator 3738 in a second position indicating that the implantable device 3002 has been fully released from the outer sheath 3706. Based on a position of the indicator 3738 within the slot 3730, the clinician will know how far the implantable device 3002 has been deployed from the outer sheath 3706. Rotation of the handle driver 3724 in an opposite direction retracts the intermediate tube and the indicator 3738.

FIGS. 55A and 55B illustrate actuation of the collar 3728 to deflect a distal portion of the outer sheath 3706. The collar 3728 may be capable of moving forward and backwards in an axial direction relative to the handle enclosure 3726. For example, when the collar 3728 is retracted, as shown in FIG. 55B, the wire in the outer sheath 3706 may be tensioned to deflect the distal portion of the outer sheath 3706 from an undeflected configuration to a deflected configuration.

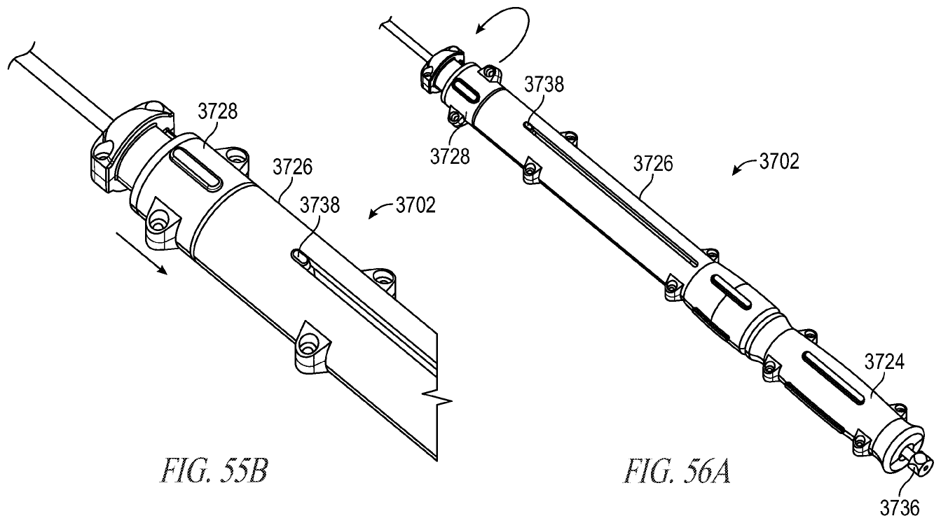

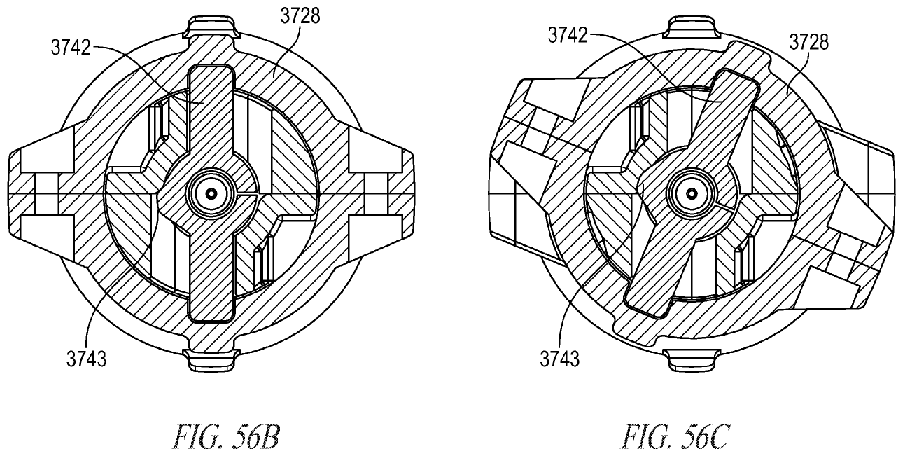

Rotation of the collar 3728, as shown FIG. 56A, may lock the distal portion of the outer sheath 3706 in the undeflected configuration or the deflected configuration. For example, the steering cam 3742 may include a cam feature 3743 that acts on the collar 3728 to prevent rotation of the collar 3728 and maintain the configuration of the outer sheath 3706. Rotating the collar 3728 in the opposite direction releases the collar 3728 and allows the configuration of the outer sheath 3706 to be changed.

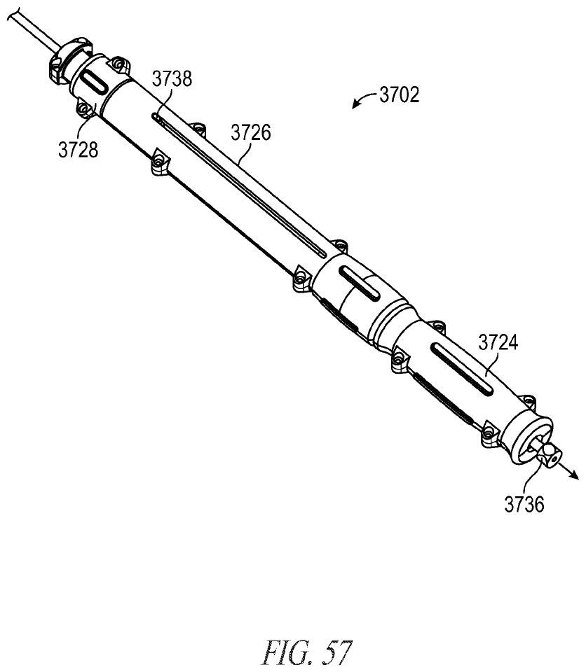

FIG. 57 illustrates a release pin 3736 extending from a proximal end of the handle driver 3724. Rotating the release pin 3736 may release the distal portion 3014 of the implantable device 3002 from the outer sheath 3706. The release pin 3736 may be removed allowing the inner tube 3710 to be withdrawn from the implantable device 3002 and the disconnect assembly 3712.

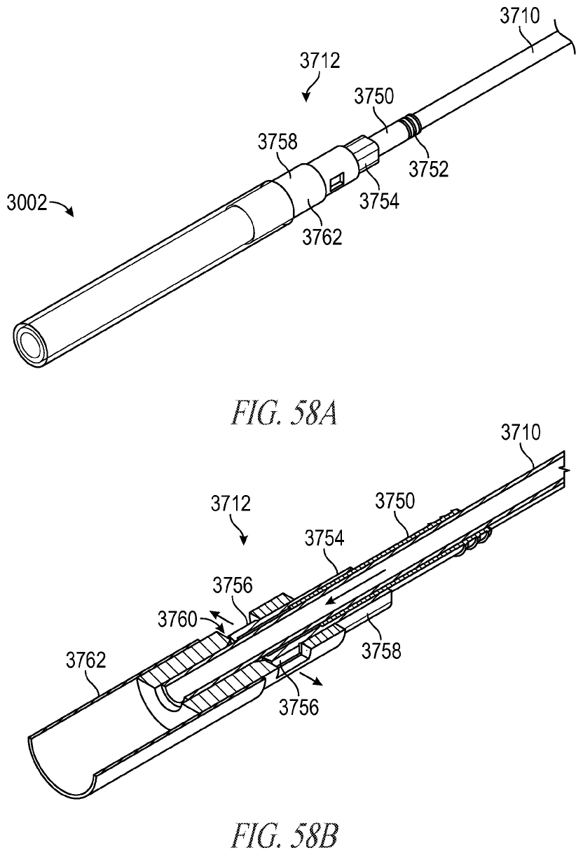

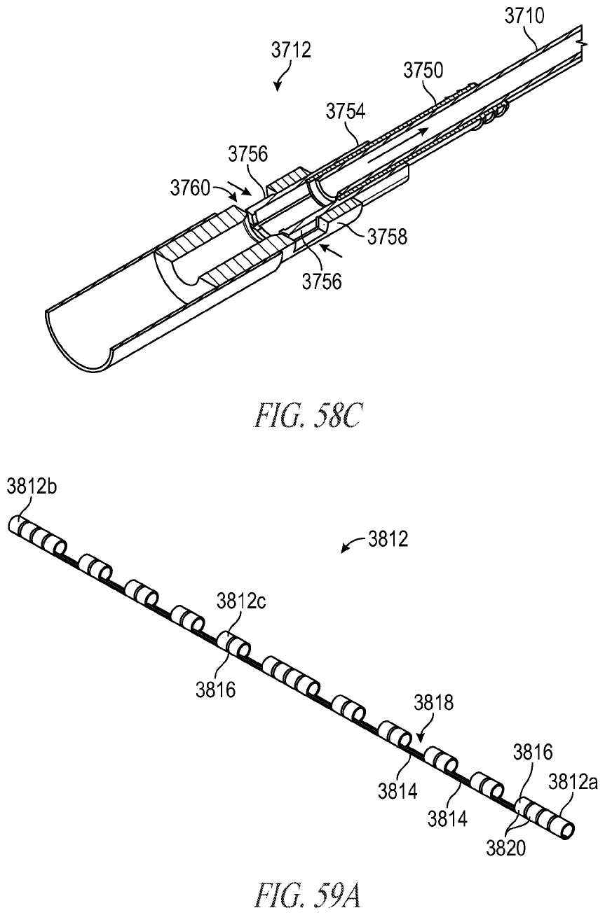

FIGS. 58A, 58B, and 58C illustrate a disconnect assembly 3712 between an implantable device 3002 and the delivery system 3700. The disconnect assembly 3712 may include an outer sleeve 3758 joined to the implantable device 3002 and an inner sleeve 3754 joined to the intermediate tube. The outer sleeve 3758 may be joined to the implantable device 3002 by a coupler 3762. The inner sleeve 3754 may be joined to the intermediate tube by a coupler 3750. The coupler 3750 may include one or more barbs 3752 for engaging the intermediate tube.

The inner sleeve 3754 may include one or more deflectable tabs 3756. The outer sleeve 3758 may include one or more windows 3760 configured o receive the deflectable tabs 3756. When the inner tube 3710 is positioned within the disconnect assembly 3712, the tabs 3756 may be pushed outward to engage with the windows 3760 in the outer sleeve 3758 and maintain the connection between the implantable device 3002 and the intermediate tube. When the inner tube 3710 is withdrawn from the disconnect assembly 3712, the tabs 3756 may be released from the outer sleeve 3758 allowing the intermediate tube to be removed from the assembly.

### Method of Use

FIGS. 45A, 45B, 45C, and 45D illustrate a method of delivering a first implantable device 3002 to an implantation site, for example an aneurysmal sac 3051 of a blood vessel such as the abdominal aorta 3052. FIGS. 45A, 45B, 45C, and 45D illustrate the first implantable device 3002 being delivered using the delivery system 3000, but the first implantable device 3002 may be delivered using any other delivery system described herein, including delivery system 3100. Although the figures illustrate the first implantable device 3002 as the coil-shaped device shown in FIG. 23. The first implantable device 3002 may perform any of the sensing, communicating, powering, and/or charging functions described herein and include any of the features of the sensing attachments, sensing constructs, or other sensing devices described herein. Note that the terms “first” and “second” implantable device or delivery system can be used interchangeably and may refer to any order of delivery. Further, the systems and methods described herein may not require both implantable devices and delivery systems. The systems and methods described herein may only be used to deliver a single implantable device with a sensing constructed integrated with a treatment device such as a graft.

In a first configuration, the first implantable device 3002 may take on a coil shape as shown in FIG. 23. During delivery, the delivery system 3000 may carry the first implantable device 3002 in a second configuration. In the second configuration, the first implantable device 3002 may take on a generally linear shape disposed within the outer sheath 3006. The outer sheath 3006 can maintain the first delivery device 3012 in the first, elongate configuration. The first implantable device 3002 may exhibit certain properties that enable the first implantable device 3002 to be transported within the outer sheath 3006.

The first implantable device 3002 may be loaded into the delivery system 3000 by securing the first implantable device 3002 to the pusher shaft 3008, for example by inserting a distal end 3022 of the pusher shaft 3008 into a lumen of the first implantable device 3002 to form a press fit. The pusher shaft 3008 may then be retracted to load the first implantable device 3002 into the outer sheath 3006. As the first implantable device 3002 is loaded into the outer sheath 3006, the first implantable device 3002 may transition from the coiled configuration to the generally linear configuration.

In use, the delivery system 3000 may be advanced to an implantation site, for example over a guidewire. As shown in FIG. 45A, the delivery system 3000 may be advanced through an iliac artery 3056 to the abdominal aorta 3052. If necessary, a distal portion 3018 of the outer sheath 3006 may be deflected to reach the implantation site and/or orient the first implantable device 3002 within the implantation site. Once the delivery system 3000 is positioned within the implantation site, the distal portion 3014 of the first implantable device 3002 may be released from the outer sheath 3006. For example, the distal portion 3014 of the first implantable device 3002 may be released from the outer sheath 3006 using the release shaft 3010.

As explained above, the distal portion 3014 of the first implantable device 3002 may project from the outer sheath 3006 and form the distal tip of the delivery system 3000 when the delivery system 3000 is advanced to the implantation site. The distal portion 3014 may form a press fit with a distal opening 3018 of the outer sheath 3006. As the release shaft 3010 is advanced through the first implantable device 3002, a distal end 3020 of the release shaft 3010 may act on the distal portion 3014 of the first implantable device 3002 to displace the distal portion 3014 of the first implantable device 3002 from the release shaft 3010. For example, the distal end 3020 of the release shaft 3010 may act on an interior surface of the distal portion 3014 of the first implantable device 3002. The interior surface of the distal portion 3014 may include an internal feature such as a ring or other projection. Even if the entire implantable device 3002 is carried within the outer sheath 3006, the release shaft 3010 may be used to release the distal portion 3014 of the first implantable device 3002 from the outer sheath 3006.

As the first implantable device 3002 is released from the outer sheath 3006, the first implantable device 3002 may begin to transition to a second configuration. In the second configuration, the first implantable device 3002 may take on a coil-shape. As illustrated in FIG. 45A, the first implantable device 3002 begins to coil as the first implantable device 3002 is released from the outer sheath 3006.

Prior to releasing the first implantable device 3002 from the delivery system 3000, the pusher shaft 3008 may be rotated to apply a torque to the first implantable device 3002 to properly orient the first implantable device 3002 within the outer sheath 3006. For example, a clinician may rotate the pusher shaft 3008 to apply torque to the first implantable device 3002 after releasing the distal portion 3014 of the first implantable device 3002, but prior to advancing the first implantable device 3002 using the pusher shaft 3008.

After the distal portion 3014 of the first implantable device 3002 has been released from the outer sheath 3006, at least a partial length of the first implantable device 3002 may be deployed at the implantation site (see FIGS. 45B and 45C). For example, the first implantable device 3002 may be advanced using the pusher shaft 3008. Alternatively, the length of the first implantable device 3002 may be unsheathed by retracting the outer sheath 3006. As illustrated in FIG. 45C, a proximal portion 3015 of the first implantable device 3002 may be released from the outer sheath 3006, while still remaining coupled to another component of the delivery system 3000 such as the pusher shaft 3008. At any time prior to release from the delivery system 3000, the implantable device 3000 may be re-sheathed, for example by retracting the pusher shaft 3008.

The proximal portion 3015 of the first implantable device 3002 may be released from the delivery system 3000, for example by releasing the proximal portion 3015 of the first implantable device 3002 from the pusher shaft 3008. The proximal portion 3015 may be released from the pusher shaft 3008 by applying torque to the first implantable device 3002. In other techniques, the proximal portion 3015 of the first implantable device 3002 may be released from the pusher shaft 3008 as soon as the proximal portion 3015 extends distally of the distal end of the outer sheath 3008.

As described above, the proximal portion 3015 of the implantable device may provide any of the sensing, communication, powering, charging, and/or other functions described herein. For example, the proximal portion 3015 of the first implantable device 3002 may include any of the antenna features described herein.

Optionally, a second implantable device 3050 may be deployed adjacent to the first implantable device 3002. The second implantable device 3050 may be deployed after at least a partial length of the first implantable device 3002 has been deployed at the implantation site, but prior to deploying the entire implantable device 3002 from the delivery system 3000. For example, the second implantable device 3050 may be deployed after releasing less than or equal to three turns of the first implantable device 3002, less than or equal to two turns of the first implantable device 3002, or less than or equal to one turn of the first implantable device 3002. In some techniques, the second implantable device 3050 may be delivered after the proximal portion 3015 of the first implantable device 3002 has been released from outer sheath 3006, but prior to releasing the first implantable device 3002 from the pusher shaft 3008. In some techniques, the second implantable device 3050 may be delivered after the proximal portion 3015 of the first implantable device 3002 has been released from the outer sheath 3008. In some techniques, the second implantable device 3050 may be delivered prior to delivering the implantable device 3002. The second implantable device 3050 may be delivered during the same procedure or during a prior procedure.

The second implantable device 3050 may be a treatment device, for example a stent graft as shown in FIG. 45D. As illustrated, the second implantable device 3050 may be positioned within an interior space defined by the first implantable device 3002. The second implantable device 3050 may remain unconnected and entirely spaced apart from the first implantable device 3002. In other configurations, the second implantable device 3050 may remain unconnected but contact the first implantable device 3002. Alternatively, the second implantable device 3050 may be clipped or otherwise coupled to the first implantable device 3002. Although FIG. 45D illustrates the second implantable device 3050 within an interior space of the first implantable device 3002, the second implantable device 3050 may surround the first implantable device 3002.

When delivering the first implantable device 3002 and the second implantable device 3050 to the aneurysmal sac 3051 of the abdominal aorta 3052, a first delivery system 3000 carrying the first implantable device 3002 may be advanced to the aneurysmal sac 3051. The first delivery system 3000 may have any of the features of the delivery systems described herein. To reach the aneurysmal sac 3051 or orient the distal end of the first delivery system 3000 within the aneurysmal sac 3051, a distal portion of the first delivery system 3000 may be deflected in at least one direction. The outer sheath 3006 may be deflected such that the distal portion 3014 of the first implantable device 3002 is disposed within a posterior region of the aneurysmal sac 3051.

The first implantable device 3002 may be at least partially deployed from the first delivery system 3000 in the aneurysmal sac 3051. The first implantable device 3002 may begin to coil as the first implantable device 3002 is released from the first delivery system 3000. Partial deployment may include releasing the distal portion 3014 of the first implantable device 3002 from the first delivery system 3000. Partial deployment may include releasing at least a partial length of the first implantable device 3002, for example less than or equal to three turns, less than or equal to two turns, or less than or equal to one turn, or otherwise. At any time prior to releasing the first implantable device 3002 from the first delivery system 3000, the first implantable device 3002 may be re-sheathed and deployed again.

The method may include advancing a second delivery system (not shown) carrying the second implantable device 3050. The first delivery system 3000 and the second delivery system may be advanced through different vessels. For example, the first delivery system 3000 may be advanced through a contralateral iliac artery 3056 and the second delivery system may be advanced through an ipsilateral iliac artery 3054. The second delivery system may be advanced through the ipsilateral iliac artery 3054 while the first delivery system 3000 is in the contralateral iliac artery 3056. The second delivery system may deploy the second implantable device 3050 adjacent to the first implantable device 3002, for example within an interior space defined by the first implantable device 3002. The second implantable device 3050 may be implanted in the abdominal aorta 3052 and/or the ipsilateral iliac artery 3054.

After the second implantable device 3050 has been deployed in the abdominal aorta, the first implantable device 3002 may be released from the first delivery system 3000. When released, the proximal portion 3015 of the first implantable device 3002 may be disposed within the aneurysmal sac 3051. After the first delivery system 3000 has been removed, a graft may be deployed in the contralateral iliac artery 3056.

In other techniques, the second delivery system may be advanced to the target site after the first delivery system 3000 has been removed from the subject. In this technique, the second delivery system may be advanced through either iliac artery 3054, 3056. In other techniques, the second delivery system may be advanced to the target site prior to the first delivery system 3000, either during the same procedure or a different procedure.

As mentioned above, the first implantable device 3002 may be delivered after implantation of the second implantable device 3050, either during the same procedure or different procedures. In this technique, the delivery system 3000 may include omnidirectional steering. The distal portion 3016 of the outer sheath 3006 may be actively deflected to coil within the aneurysmal sac 3051. The outer sheath 3006 may then be retracted to unsheath the first implantable device 3002.

If the first implantable device 3002 is delivered using the delivery system 3100, the method may be similar to the method above, except as described below. The release shaft 3110 may be advanced or retracted to permit the distal portion 3014 of the implantable device 3002 to be released from the outer sheath. The implantable device 3002 may be advanced using the pusher shaft 3108. After partial deployment of the implantable device 3002, the release shaft 3110 may be retracted through a lumen of the implantable device 3002. For example, the clinician may retract the release shaft 3110 after confirming proper placement of the distal portion 3014 of the implantable device 3002). Retracting the release shaft 3110 may cause the distal portion 3014 of the implantable device 3002 to transition between a compressed configuration for transport and a substantially flattened configuration.

### Terminology

Disclosed embodiments have been described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the embodiments are described in terms of Markush groups, it is intended, and those skilled in the art will recognize, that the disclosure embraces and is also thereby described in terms of any individual member and any subgroup of members of the Markush group, and Applicant(s) reserve the right to revise the application or claims to refer specifically to any individual member or any subgroup of members of the Markush group.

All references disclosed herein, including patent references and non-patent references, are hereby incorporated by reference in their entirety as if each was incorporated individually. For example, PCT/US2020/026745, Publication No. WO 2020/206373, is hereby incorporated herein in its entirety including its disclosure of implantable sensing constructs and methods of use. PCT Publication No. WO 2017/165717 is incorporated herein for all purposes, including for the disclosure of how to provide power to a sensor as disclosed herein; and how to allow information obtained by a sensor as disclosed herein to be transmitted outside the body of the patient that has received the sensor.

As used herein, the relative terms “posterior” and “anterior” shall be defined according to the anatomy.

The terms “approximately,” “about,” and “substantially” as used herein represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount. As another example, in certain embodiments, the terms “generally linear” and “substantially liner” refer to a value, amount, or characteristic that departs from exactly linear by less than or equal to 5 degrees, 3 degrees, 1 degree, 0.1 degree, or otherwise.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. For example, the term “a sensor” refers to one or more sensors, and the term “a medical device comprising a sensor” is a reference to a medical device that includes at least one sensor, where the medical device comprising a sensor may have, for example, 1 sensor, 2 sensors, 3 sensors, 4 sensors, 5 sensors, 6 sensors, 7 sensors, 8 sensors, 9 sensors, 10 sensors, or more than 10 sensors. A plurality of sensors refers to more than one sensor. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the disclosure or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described embodiments. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the disclosure pertains, and each such referenced document and material is hereby incorporated by reference to the same extent as if it had been incorporated by reference in its entirety individually or set forth herein in its entirety. Applicant(s) reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicant(s) reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicant(s).

### EXAMPLE EMBODIMENTS

The following exemplary embodiments identify some possible permutations of combinations of features disclosed herein, although other permutations of combinations of features are also possible and contemplated by the present disclosure.
1. A delivery system comprising:
   a handle;
   an outer sheath comprising a first lumen;
   a pusher shaft slidably disposed within the first lumen of the outer sheath, the pusher shaft comprising a second lumen;
   a release shaft slidably disposed within in the second lumen of the of the pusher shaft, the release shaft capable of releasing a distal tip from the outer sheath.
2. The delivery system of Embodiment 1, wherein the distal tip forms a part of an implantable device.
3. The delivery system of Embodiment 1, wherein the distal tip comprises an antenna.
4. The delivery system of Embodiment 1 or 2, wherein the outer sheath is deflectable in at least one direction.
5. The delivery system of any one of the preceding Embodiments, wherein the pusher shaft is rotatable to apply torque to an implantable device carried by the delivery system.
6. The delivery system of any one of the preceding Embodiments, wherein a distal end of the pusher shaft is shaped to interface with an implantable device.
7. The delivery system of any one of the preceding Embodiments, wherein the release shaft comprises an enlarged distal end.
8. The delivery system of any one of the preceding Embodiments, wherein the release shaft comprises a guidewire lumen.
9. The delivery system of any one of the preceding Embodiments, further comprising a locking mechanism to prevent movement of the pusher shaft relative to the outer sheath.
10. The delivery system of any one of the preceding Embodiments, further comprising a locking mechanism to prevent movement of the release shaft relative to the pusher shaft.
11. The delivery system of any one of Embodiments 1 and 3 to 10, wherein the distal tip forms a distal portion of the release shaft.
12. A system for delivering an implantable device, the system comprising:
   a handle;
   an outer sheath comprising a lumen carrying the implantable device, a distal portion of the implantable device projecting from a distal end of the outer sheath;
   a pusher shaft slidably disposed within the lumen of the outer sheath, the pusher shaft capable of pushing a proximal portion of the implantable device out of the outer sheath;
   a release shaft slidably disposed within a lumen of the pusher shaft, the release shaft capable of releasing a distal tip of the system from the outer sheath.
13. The system of Embodiment 12, wherein the implantable device forms the distal tip of the system.
14. The system of Embodiment 12, wherein a distal portion of the release shaft forms the distal tip of the system.
15. The system of any one of Embodiments 12 to 14, wherein when the implantable device is loaded in the outer sheath, the distal portion of the implantable device is coupled to the distal end of the outer sheath.
16. The system of Embodiment 15, wherein the distal portion of the implantable device is coupled to the distal end of the outer sheath by a press-fit.
17. The system of any one of Embodiments 12 to 16, wherein the pusher shaft is releasably coupled to the proximal portion of the implantable device.
18. The system of Embodiment 17, wherein the distal end of the pusher shaft is coupled to the proximal portion of the implantable device by a press fit.
19. The system of any one of Embodiments 12 to 18, wherein the pusher shaft is rotatable to apply torque to the implantable device when the implantable device is disposed within the outer sheath.
20. The system of any one of Embodiments 12 to 19, wherein the pusher shaft is rotatable to apply torque to the proximal portion of the implantable device to release the implantable device from the pusher shaft.
21. The system of any one of Embodiments 12 to 20, wherein the pusher shaft is retractable to retract the implantable device into the outer sheath.
22. The system of any one of Embodiments 12 to 21, wherein the releasable shaft is slidably disposed within a lumen of the implantable device.
23. The system of any one of Embodiments 12 to 22, wherein the release shaft is capable of pushing an internal feature of the implantable device to release the distal portion of the implantable device from the outer sheath.
24. The system of any one of Embodiments 12 to 23, wherein the outer sheath is deflectable in at least one direction.
25. The system of any one of Embodiments 12 to 24, wherein the distal portion of the implantable device has a first configuration when the implantable device is disposed within the outer sheath and a second configuration when the implantable device is released from the outer sheath.
26. A delivery system handle comprising:
   a handle body;
   a first user-actuatable control capable of deflecting an outer sheath in at least one direction;
   a second user-actuatable control capable of providing torque control for a pusher shaft;
   a third user-actuatable control capable of advancing the pusher shaft; and
   a fourth user-actuatable control capable of advancing a release shaft.
27. The delivery system of Embodiment 26, wherein the third user-actuatable control is capable of retracting the pusher shaft.
28. The delivery system handle of Embodiment 26 or 27, wherein the first user-actuatable control actuates a worm gear.
29. The delivery system of Embodiment 28, wherein a position of the worm gear is visible in a window of the handle body
30. A method of delivering an implantable device, the method comprising:
   advancing a delivery system over a guidewire, the delivery system comprising an outer sheath carrying an implantable device;
   deflecting a distal portion of the outer sheath to a target site;
   releasing a distal tip from the outer sheath using a release shaft;
   advancing the implantable device using a pusher shaft; and
   releasing a proximal portion of the implantable device from the pusher shaft.
31. The method of Embodiment 30, wherein a distal portion of the implantable device forms the distal tip.
32. The method of Embodiment 30, wherein a distal portion of the release shaft forms the distal tip.
33. The method of any one of Embodiments 30 to 32, wherein releasing the distal tip comprises advancing the release shaft through a lumen of the implantable device.
34. The method of any one of Embodiments 30 to 33, wherein advancing the release shaft causes the release shaft to push on an internal feature of the implantable device.
35. The method of any one of Embodiments 30 to 34, further comprising applying torque to the implantable device when the implantable device is loaded within the outer sheath.
36. The method of Embodiment 35, wherein applying torque to the implantable device occurs prior to advancing the implantable device using the pusher shaft.
37. The method of any one of Embodiments 30 to 36, wherein releasing the proximal portion of the implantable device from the pusher shaft comprising applying torque to the implantable device.
38. The method of any one of Embodiments 30 to 37, further comprising retracting the pusher shaft to re-sheath the implantable device.
39. The method of any one of Embodiments 30 to 38, further comprising deploying a stent graft prior to releasing the proximal portion of the implantable device from the pusher shaft.
40. The method of Embodiment 39, wherein deploying the stent graft comprises deploying the stent graft within an interior space defined by the implantable device.
41. A method of delivering an implantable device, the method comprising:
   advancing a first delivery system carrying the implantable device through a contralateral iliac artery;
   deflecting a distal portion of the first delivery system to a target site within an abdominal aorta;
   partially deploying the implantable device from the first delivery system in the abdominal aorta, the implantable device forming a coil as the implantable device is released from the first delivery system;
   advancing a second delivery system carrying a stent graft through an ipsilateral iliac artery;
   after partially deploying the implantable device, deploying the stent graft within an interior space defined by the coil; and
   after deploying the stent graft, releasing the implantable device from the first delivery system.
42. The method of Embodiment 41, further comprising retracting the implantable device prior to releasing the implantable device from the first delivery system.
43. The method of Embodiment 41 or 42, further comprising deploying a distal portion of the implantable device in a posterior region of an aneurysmal sac of the abdominal aorta.
44. An implantable sensing construct configured to be percutaneously implanted in an aneurysmal sac, the implantable sensing construct comprising:
   a sensor; and
   a body comprising a first configuration and a second configuration, the body configured to withstand a compression load of up to 5.0 N,
   wherein in the first configuration, the body comprises a substantially linear shape for transport in a delivery system; and
   wherein in the second configuration, the body comprises a coiled shape when released from the delivery system.
45. The implantable sensing construct of Embodiment 44, wherein the body configured to withstand a compression load of up to 20.0 N.
46. The implantable sensing construct of Embodiment 44, wherein the body configured to withstand a compression load of up to 25.0 N.
47. An implantable sensing construct configured to be percutaneously implanted in an aneurysmal sac, the implantable sensing construct comprising:
   a sensor; and
   a body comprising a first configuration and a second configuration,
   wherein in the first configuration, the body comprises a substantially linear shape for transport in a delivery system; and
   wherein in the second configuration, the body comprises a coiled shape when released from the delivery system, wherein the coiled shape has a pitch, the pitch being substantially maintained upon application of a linear compression force of up to 8.0 N.
48. The implantable sensing construct of Embodiment 47, wherein the linear compression force is up to 15.0 N.
49. The implantable sensing construct of Embodiment 47, wherein the linear compression force is up to 25.0 N.
50. The implantable sensing construct of Embodiment 47, wherein the linear compression force is up to 105.0 N.
51. An implantable system for use with a stent graft, the system comprising:
   a helix antenna supported by a non-conductive substrate; and
   a communications and processing circuitry electrically connected to the helix antenna via an antenna feed, the communications and processing circuitry supported by a substrate comprising a ground plane to which the helix antenna is electrically connected, the communications and processing circuitry further comprising at least one sensor.
52. The system of Embodiment 51, further comprising a body configured to be attached to or positioned adjacent to the stent graft, the body made at least partially of conductive material, wherein the helix antenna is further electrically connected to the body such that the body provides an additional ground for the helix antenna.
53. The system of any one of Embodiments 51 to 52, wherein the helix antenna is configured to transmit and receive in a Bluetooth frequency band.
54. The system of Embodiment 53, wherein a range of the helix antenna in the Bluetooth frequency band is about 1 foot or more.
55. The system of Embodiment 53, wherein a range of the helix antenna in the Bluetooth frequency band is between about 1 foot and 2 feet.
56. The system of any one of Embodiments 51 to 55, wherein the communications and processing circuitry comprises a matching circuitry electrically connected to the helix antenna.
57. The system of Embodiment 56, wherein the matching circuitry comprises a series capacitor and a shunt capacitor.
58. The system of Embodiment 57, wherein the matching circuitry further comprises a low-pass filter.
59. The system of any one of Embodiments 51 to 58, wherein the non-conductive substrate provides structural support for the helix antenna, and wherein the helix antenna is wound on the non-conductive substrate.
60. The system of any one of Embodiments 51 to 59, wherein the non-conductive substrate comprises a polymer.
61. The system of Embodiment 60, wherein the polymer comprises polytetrafluoroethylene (PTFE).
62. The system of any one of Embodiments 51 to 61, wherein the stent graft comprises an abdominal aortic aneurysm (AAA) stent graft.
63. A method of radio frequency (RF) testing an antenna of an implantable system, the method comprising:
   determining one or more RF properties of the antenna of the implantable system positioned in a first container at least partially filled with a first composition configured to simulate electromagnetic properties of blood, the first container positioned in a second container at least partially filed with a second composition configured to simulate electromagnetic properties of one or more tissues.
64. The method of Embodiment 63, wherein the one or more tissues comprise at least two of bone, muscle, fat, and skin.
65. The method of Embodiment 64, wherein electromagnetic properties of at least two of bone, muscle, fat, and skin are averaged to create the second composition.
66. The method of Embodiment 65, wherein the electromagnetic properties of the one or more tissues comprise relative permittivity and conductivity.
67. The method of any one of Embodiments 63 to 66, wherein the first composition comprises sodium chloride (NaCl), diacetin, and distilled water, and wherein the second composition comprises diacetin and distilled water.
68. The method of any one of Embodiments 63 to 67, wherein the antenna comprises a helix antenna.
69. The method of Embodiment 68, wherein the helix antenna is supported by a non-conductive substrate.
70. The method of any one of Embodiments 63 to 69, wherein the implantable system is configured to be used with a stent graft, and wherein the stent graft comprises an abdominal aortic aneurysm (AAA) stent graft.
71. An implantable system for use with a stent graft, the system comprising:
   a first antenna comprising a straight conductor and a helical conductor, the straight conductor electrically connected to the helical conductor; and
   a communications and processing circuitry electrically connected to the first antenna via an antenna feed, the communications and processing circuitry supported by a substrate comprising a ground plane to which the first antenna is electrically connected, the communications and processing circuitry further comprising at least one sensor.
72. The system of Embodiment 71, further comprising a body configured to be attached to or positioned adjacent to the stent graft, the body made at least partially of conductive material, wherein the first antenna is supported by the body and is further electrically connected to the body such that the body provides an additional ground for the first antenna.
73. The system of any one of Embodiments 71 to 72, wherein the straight conductor comprises a monopole antenna.
74. The system of any one of Embodiments 71 to 73, wherein the first antenna comprises a dual-band antenna that transmits and receives in first and second frequency bands.
75. The system of Embodiment 74, wherein the first antenna resonates at center frequencies of the first and second frequency bands.
76. The system of any one of Embodiments 74 to 75, wherein the first frequency band comprises medical device radiocommunications service (MICS) band and the second frequency band comprises industrial, scientific, and medical (ISM) band.
77. The system of Embodiment 76, wherein a range in the first frequency band is at least about 20 feet, and wherein a range in the second frequency band is at least about 15 feet.
78. The system of Embodiment 76, wherein a range in the first frequency band comprises about 1 foot or more, and wherein a range in the second frequency band comprises about 1 foot or more.
79. The system of any one of Embodiments 76 to 78, wherein the communications and processing circuitry is configured to transition from a first power state to a second power state in which more power is consumed responsive to the first antenna receiving a command in the second frequency band.
80. The system of Embodiment 79, wherein the first power state comprises a sleep state and the second power state comprises an operational state in which the communications and processing circuitry is configured to at least one of transmit or receive data.
81. The system of Embodiment 80, wherein the data comprises data sensed by the at least one sensor, and wherein the communications and processing circuitry is configured to cause the first antenna to transmit the data in the first frequency band.
82. The system of Embodiment 81, wherein the communications and processing circuitry is configured to transmit data sensed by the at least one sensor in the second power state and not in the first power state.
83. The system of any one of Embodiments 71 to 82, wherein the communications and processing circuitry comprises a matching circuitry electrically connected to the first antenna.
84. The system of Embodiment 83, wherein:
   the first antenna is configured to at least one of receive or transmit in first and second frequency bands, the second frequency band associated with higher frequencies than the first frequency band;
   the matching circuitry comprises a first matching circuitry for signals in the first frequency band and a second matching circuitry for signals in the second frequency band; and
   the first matching circuitry comprises a band-stop filter configured to remove one or more signal components in second frequency band.
85. The system of Embodiment 84, wherein the first matching circuitry comprises a step-up impedance low pass filter and the second matching circuitry comprises a setup-up impedance high pass filter.
86. The system of any one of Embodiments 84 to 85, wherein the second matching circuitry does not comprise a band-stop filter configured to remove one or more signal components in the first frequency band.
87. The system of any one of Embodiments 71 to 86, further comprising a rechargeable power source and a second antenna configured to receive power for recharging the rechargeable power source.
88. The system of Embodiment 87, wherein the second antenna comprises a coil configured to be inductively coupled with a coil of an external power transfer device.
89. The system of any one of Embodiments 87 to 88, further comprising a body configured to be attached to or positioned adjacent to the stent graft, wherein the first antenna is supported by the body at a first end of the body and the second antenna is supported by the body at a second end of the body opposite the first end.
90. The system of any one of Embodiments 71 to 89, further comprising a second antenna.
91. The system of Embodiment 90, further comprising a body configured to be attached to or positioned adjacent to the stent graft, wherein the first antenna is supported by the body at a first end of the body and the second antenna is supported by the body at a second end of the body opposite the first end.
92. The system of any one of Embodiments 71 to 91, wherein length of the first antenna is at most about 40 mm and width of the first antenna is at most about 5 mm.
93. The system of any one of Embodiments 71 to 92, wherein spacing between turns of the helical conductor is about 1.7 mm.
94. The system of any one of Embodiments 71 to 93, wherein the helical conductor is wound around the straight conductor.
95. The system of Embodiment 94, wherein the helical conductor is electrically insulated from the straight conductor in a region where the helical conductor is wound around the straight conductor.
96. The system of any one of Embodiments 71 to 95, wherein the straight conductor and the helical conductor are electrically connected to the antenna feed.
97. The system of any one of Embodiments 71 to 96, wherein the stent graft comprises an abdominal aortic aneurysm (AAA) stent graft.
98. An implantable system for use with a stent graft, the system comprising:
   a first antenna comprising a loop; and
   communications and processing circuitry electrically connected to the first antenna via an antenna feed, the communications and processing circuitry supported by a substrate comprising a ground plane to which the first antenna is electrically connected, the communications and processing circuitry further comprising at least one sensor; and
   a matching circuitry of the communications and processing circuitry, the matching circuitry electrically connected to the first antenna and comprising a plurality of capacitors configured to match impedance of the first antenna in a first frequency band.
99. The system of Embodiment 98, wherein the matching circuitry further comprises a plurality of inductors configured to match impedance of the first antenna in a second frequency band associated with higher frequencies than the first frequency band.
100. The system of any one of Embodiments 98 to 99, further comprising a body configured to be attached to or positioned adjacent to the stent graft, the body made at least partially of conductive material, wherein the first antenna is supported by the body and is further electrically connected to the body such that the body provides an additional ground for the first antenna.
101. The system of any one of Embodiments 98 to 100, wherein the first antenna comprises a dual-band antenna that transmits and receives in the first frequency band and in a second frequency band.
102. The system of Embodiment 101, wherein the first antenna resonates in the second frequency band, the second frequency band associated with higher frequencies than the first frequency band.
103. The system of any one of Embodiments 101 to 102, wherein the first frequency band comprises medical device radiocommunications service (MICS) band and the second frequency band comprises industrial, scientific, and medical (ISU) band.
104. The system of Embodiment 103, wherein a range in the first frequency band is at least about 20 feet, and wherein a range in the second frequency band is at least about 7 feet.
105. The system of Embodiment 103, wherein a range in the first frequency band comprises about 1 foot or more, and wherein a range in the second frequency band comprises about 1 foot or more.
106. The system of any one of Embodiments 103 to 105, wherein the communications and processing circuitry is configured to transition from a first power state to a second power state in which more power is consumed responsive to the first antenna receiving a command in the second frequency band.
107. The system of Embodiment 106, wherein the first power state comprises a sleep state and the second power state comprises an operational state in which the communications and processing circuitry is configured to at least one of transmit or receive data.
108. The system of Embodiment 107, wherein the data comprises data sensed by the at least one sensor, and wherein the communications and processing circuitry is configured to cause the first antenna to transmit the data in the first frequency band.
109. The system of Embodiment 106, wherein the communications and processing circuitry is configured to transmit data sensed by the at least one sensor in the second power state and not in the first power state.
110. The system of Embodiment 109, wherein the matching circuitry comprises a band-stop filter configured to remove one or more signal components in the second frequency band.
111. The system of any one of Embodiments 109 to 110, wherein the matching circuitry does not comprise a band-stop filter configured to remove one or more signal components in the first frequency band.
112. The system of any one of Embodiments 98 to 111, further comprising a rechargeable power source and a second antenna configured to receive power for recharging the rechargeable power source.
113. The system of Embodiment 112, wherein the second antenna comprises a coil configured to be inductively coupled with a coil of an external power transfer device.
114. The system of Embodiment 113, further comprising a body configured to be attached to or positioned adjacent to the stent graft, wherein the first antenna is supported by the body at a first end of the body and the second antenna is supported by the body at a second end of the body opposite the first end.
115. The system of any one of Embodiments 98 to 114, further comprising a second antenna.
116. The system of Embodiment 115, further comprising a body configured to be attached to or positioned adjacent to the stent graft, wherein the first antenna is supported by the body at a first end of the body and the second antenna is supported by the body at a second end of the body opposite the first end.
117. The system of any one of Embodiments 98 to 116, wherein diameter of the loop is at most about 40 mm.
118. The system of any one of Embodiments 98 to 117, wherein width of the first antenna is at most about 5 mm.
119. The system of any one of Embodiments 98 to 118, wherein the stent graft comprises an abdominal aortic aneurysm (AAA) stent graft.
120. A method of using or operating the system of any one of Embodiments 71 to 119.
121. A delivery system for delivering an implantable device, the delivery system comprising:
   a handle enclosure;
   a handle driver comprising a collar rotatably coupled to the handle enclosure, an inner surface of the collar comprising a threaded pattern;
   a first lead screw disposed at least partially within the handle driver, the first lead screw threaded in a first direction and configured to interface with the threaded pattern on the collar;
   a second lead screw disposed at least partially within the handle enclosure, the second lead screw axially offset from the first lead screw, the second lead screw threaded in a second direction opposite from the first direction and configured to interface with the threaded pattern on the collar;
   wherein rotation of the handle driver in a first direction advances the implantable device.
122. The delivery system of Embodiment 121, wherein a distal portion of the first lead screw abuts a proximal portion of the second lead screw when the implantable device is loaded in the delivery system.
123. The delivery system of Embodiment 121 or 122, wherein rotation of handle driver drives the first lead screw in a first direction and drives the second lead screw in a second direction opposite the first direction.
124. The delivery system of any one of Embodiments 121 to 123, wherein rotation of the handle driver drives the first lead screw and the second lead screw the same distance.
125. The delivery system of any one of Embodiments 121 to 124, wherein rotation of the handle driver in a second direction, opposite from the first direction, retracts the implantable device.
126. The delivery system of any one of Embodiments 121 to 125, wherein the handle enclosure comprise a groove configured to capture the collar of the handle driver.
127. The delivery system of any one of Embodiments 121 to 126, wherein the first lead screw is a partial body screw, and wherein the second lead screw is a partial body screw.
128. The delivery system of Embodiment 127, wherein the first lead screw is circumferentially offset from the second lead screw.
129. The delivery system of any one of Embodiments 121 to 128, wherein the threaded pattern comprises a pattern of diamond-shaped recesses.
130. The delivery system of any one of Embodiments 121 to 129, further comprising an indicator fixed to the first lead screw, the indicator visible through a slot in the handle enclosure.
131. A method of delivering an implantable device to a patient, the method comprising:
   advancing a delivery system to a target location, the delivery system comprising a handle and an outer sheath carrying the implantable device;
   retracting a first actuator on the handle to deflect a distal portion of the outer sheath to a deflected configuration;
   rotating the first actuator to lock the distal portion of the outer sheath in the deflected configuration;
   rotating a second actuator in a first direction to advance an intermediate tube relative to the outer sheath, the intermediate tube coupled to the implantable device; and
   withdrawing an inner tube to release the implantable device from the intermediate tube.
132. The method of Embodiment 131, wherein retracting the first actuator tensions a wire to deflect the distal portion of the outer sheath.
133. The method of Embodiment 131 or 132, wherein rotating the first actuator rotates a cam to lock the distal portion of the outer sheath in the deflected configuration.
134. The method of any one of Embodiments 131 to 133, further comprising rotating the second actuator in a second direction, opposite from the first direction, to retract the intermediate tube.
135. The method of any one of Embodiments 131 to 134, further comprising rotating the inner tube to release the inner tube from the implantable device.
136. The method of any one of Embodiments 131 to 135, wherein rotating the second actuator causes an indicator to travel along a slot in the handle of the delivery system.
137. The method of any one of Embodiments 131 to 136, further comprising removing a release pin to enable withdrawal of the inner tube.
138. The method of Embodiment 137, wherein removing the release pin occurs after the implantable device has been only partially deployed from the outer sheath.
139. The method of Embodiment 137, wherein removing the release pin occurs after a proximal end of the implantable device has been deployed from the outer sheath.
140. A delivery system for delivering an implantable device, the delivery system comprising:
   a handle comprising:
      a handle enclosure;
      a first actuator movable relative to the handle enclosure;
      a second actuator movable relative to the handle enclosure; and
   an outer sheath extending from the handle;
   an intermediate tube extending through the outer sheath, the intermediate tube configured to engage the implantable device;
   an inner tube extending through the intermediate tube, the inner tube configured to maintain the intermediate tube in engagement with the implantable device when the inner tube extends through the implantable device;
   wherein the first user actuator is configured to deflect a distal portion of the outer sheath from an undeflected configuration to a deflected configuration;
   wherein the second actuator is configured to advance the intermediate tube relative to the outer sheath.
141. The delivery system of Embodiment 140, wherein translation of the first user actuator tensions a wire to deflect the distal portion of the outer sheath from the undeflected configuration to the deflected configuration.
142. The delivery system of Embodiment 141, wherein rotation of the first actuator rotates a cam to lock the distal portion of the outer sheath in the undeflected configuration or the deflected configuration.
143. The delivery system of any one of Embodiments 140 to 142, wherein rotation of the second actuator in a first direction advances the intermediate tube, and wherein rotation of the second actuator in a second direction, opposite the first direction, retracts the intermediate tube.
144. The delivery system of any one of Embodiments 140 to 143, further comprising a release pin at a proximal end of the inner tube.
145. The delivery system of Embodiment 144, wherein rotation of the release pin deploys a distal portion of the implantable device.
146. The delivery system of Embodiment 144, wherein the release pin is removable from the inner tube.
147. The delivery system of any one of Embodiments 140 to 146, further comprising an indicator visible through a slot in the handle enclosure, the indicator indicative of a location of the implantable device relative to the outer sheath.
148. The delivery system of any one of Embodiments 140 to 147, further comprising a disconnect assembly at a distal end of the intermediate tube, the disconnect assembly comprising one or more deflectable tabs configured to engage the implantable device when the inner tube extends through the inner component.
149. An implantable sensing construct configured to be percutaneously implanted in an aneurysmal sac, the implantable sensing construct comprising:
   a sensor; and
   a tubular body comprising a first configuration and a second configuration, the tubular body comprising a plurality of cutouts in a circumferential direction, each of the plurality of cutouts comprising a first end, a second end, and in intermediate portion therebetween;
   wherein in the first configuration, the body comprises a substantially linear shape for transport in a delivery system; and
   wherein in the second configuration, the body comprises a coiled shape when released from the delivery system.
150. The implantable sensing construct of Embodiment 149, wherein a width of each of the first ends and the second ends of the plurality of cutouts is greater than a width of the intermediate portions.
151. The implantable sensing construct of Embodiment 149 or 150, wherein the plurality of cutouts are equally spaced apart along a length of the tubular body.
152. The implantable sensing construct of any one of Embodiments 149 to 151, wherein the tubular body comprises a plurality of tubular segments, the plurality of tubular segments spaced apart from each other and interconnected by a spine, each of the plurality of tubular segments having one or more of the plurality of cutouts.
153. The implantable sensing construct of Embodiment 152, wherein when the tubular body is laid flat as a flattened body with the spine forming opposite lateral edges, the lateral edges form an oblique angle relative to an end of the flattened body.
154. The implantable sensing construct of Embodiment 152 or 153, wherein the plurality of tubular segments comprises: a first tubular segment at a first end of the tubular body, a second tubular segment at a second end of the tubular body, and at least one tubular segment between the first tubular segment and the second tubular segment.
155. The implantable sensing construct of Embodiment 154, wherein the at least one tubular segment is shorter than the first tubular segment and the second tubular segment.

1. A delivery system comprising:
a handle;
an outer sheath comprising a first lumen;
a pusher shaft slidably disposed within the first lumen of the outer sheath, the pusher shaft comprising a second lumen;
a release shaft slidably disposed within in the second lumen of the of the pusher shaft, the release shaft capable of releasing a distal tip from the outer sheath.

2. The delivery system of claim 1, wherein the distal tip forms a part of an implantable device.

3. The delivery system of claim 1, wherein the distal tip comprises an antenna.

4. The delivery system of claim 1, wherein the outer sheath is deflectable in at least one direction.

5. The delivery system of claim 1, wherein the pusher shaft is rotatable to apply torque to an implantable device carried by the delivery system.

6. The delivery system of claim 1, wherein a distal end of the pusher shaft is shaped to interface with an implantable device.

7. The delivery system of claim 1, wherein the release shaft comprises an enlarged distal end.

8. The delivery system of claim 1, wherein the release shaft comprises a guidewire lumen.

9. The delivery system of claim 1, further comprising a locking mechanism to prevent movement of the pusher shaft relative to the outer sheath.

10. The delivery system of claim 1, further comprising a locking mechanism to prevent movement of the release shaft relative to the pusher shaft.

11. The delivery system of claim 1, wherein the distal tip forms a distal portion of the release shaft.

12. A system for delivering an implantable device, the system comprising:
a handle;
an outer sheath comprising a lumen carrying the implantable device, a distal portion of the implantable device projecting from a distal end of the outer sheath;
a pusher shaft slidably disposed within the lumen of the outer sheath, the pusher shaft capable of pushing a proximal portion of the implantable device out of the outer sheath;
a release shaft slidably disposed within a lumen of the pusher shaft, the release shaft capable of releasing a distal tip of the system from the outer sheath.

13. The system of claim 12, wherein the implantable device forms the distal tip of the system.

14. The system of claim 12, wherein a distal portion of the release shaft forms the distal tip of the system.

15. The system of claim 12, wherein when the implantable device is loaded in the outer sheath, the distal portion of the implantable device is coupled to the distal end of the outer sheath.

16. The system of claim 15, wherein the distal portion of the implantable device is coupled to the distal end of the outer sheath by a press-fit.

17. The system of claim 12, wherein the pusher shaft is releasably coupled to the proximal portion of the implantable device.

18. The system of claim 17, wherein the distal end of the pusher shaft is coupled to the proximal portion of the implantable device by a press fit.

19. The system of claim 12, wherein the pusher shaft is rotatable to apply torque to the implantable device when the implantable device is disposed within the outer sheath.

20. The system of claim 12, wherein the pusher shaft is rotatable to apply torque to the proximal portion of the implantable device to release the implantable device from the pusher shaft.

21. The system of claim 12, wherein the pusher shaft is retractable to retract the implantable device into the outer sheath.

22. The system of claim 12, wherein the releasable shaft is slidably disposed within a lumen of the implantable device.

23. The system of claim 12, wherein the release shaft is capable of pushing an internal feature of the implantable device to release the distal portion of the implantable device from the outer sheath.

24. The system of claim 12, wherein the outer sheath is deflectable in at least one direction.

25. The system of claim 12, wherein the distal portion of the implantable device has a first configuration when the implantable device is disposed within the outer sheath and a second configuration when the implantable device is released from the outer sheath.

26. A delivery system handle comprising:
a handle body;
a first user-actuatable control capable of deflecting an outer sheath in at least one direction;
a second user-actuatable control capable of providing torque control for a pusher shaft;
a third user-actuatable control capable of advancing the pusher shaft; and
a fourth user-actuatable control capable of advancing a release shaft.

27. The delivery system of claim 26, wherein the third user-actuatable control is capable of retracting the pusher shaft.

28. The delivery system handle of claim 26, wherein the first user-actuatable control actuates a worm gear.

29. The delivery system of claim 28, wherein a position of the worm gear is visible in a window of the handle body

30. A method of delivering an implantable device, the method comprising:
advancing a delivery system over a guidewire, the delivery system comprising an outer sheath carrying an implantable device;
deflecting a distal portion of the outer sheath to a target site;
releasing a distal tip from the outer sheath using a release shaft;
advancing the implantable device using a pusher shaft; and
releasing a proximal portion of the implantable device from the pusher shaft.

31. The method of claim 30, wherein a distal portion of the implantable device forms the distal tip.

32. The method of claim 30, wherein a distal portion of the release shaft forms the distal tip.

33. The method of claim 30, wherein releasing the distal tip comprises advancing the release shaft through a lumen of the implantable device.

34. The method of claim 30, wherein advancing the release shaft causes the release shaft to push on an internal feature of the implantable device.

35. The method of claim 30, further comprising applying torque to the implantable device when the implantable device is loaded within the outer sheath.

36. The method of claim 35, wherein applying torque to the implantable device occurs prior to advancing the implantable device using the pusher shaft.

37. The method of claim 30, wherein releasing the proximal portion of the implantable device from the pusher shaft comprising applying torque to the implantable device.

38. The method of claim 30, further comprising retracting the pusher shaft to re-sheath the implantable device.

39. The method of claim 30, further comprising deploying a stent graft prior to releasing the proximal portion of the implantable device from the pusher shaft.

40. The method of claim 39, wherein deploying the stent graft comprises deploying the stent graft within an interior space defined by the implantable device.

41. A method of delivering an implantable device, the method comprising:
advancing a first delivery system carrying the implantable device through a contralateral iliac artery;
deflecting a distal portion of the first delivery system to a target site within an abdominal aorta;
partially deploying the implantable device from the first delivery system in the abdominal aorta, the implantable device forming a coil as the implantable device is released from the first delivery system;
advancing a second delivery system carrying a stent graft through an ipsilateral iliac artery;
after partially deploying the implantable device, deploying the stent graft within an interior space defined by the coil; and
after deploying the stent graft, releasing the implantable device from the first delivery system.

42. The method of claim 41, further comprising retracting the implantable device prior to releasing the implantable device from the first delivery system.

43. The method of claim 41, further comprising deploying a distal portion of the implantable device in a posterior region of an aneurysmal sac of the abdominal aorta.

44. An implantable sensing construct configured to be percutaneously implanted in an aneurysmal sac, the implantable sensing construct comprising:
a sensor; and
a body comprising a first configuration and a second configuration, the body configured to withstand a compression load of up to 5.0 N,
wherein in the first configuration, the body comprises a substantially linear shape for transport in a delivery system; and
wherein in the second configuration, the body comprises a coiled shape when released from the delivery system.

45. The implantable sensing construct of claim 44, wherein the body configured to withstand a compression load of up to 20.0 N.

46. The implantable sensing construct of claim 44, wherein the body configured to withstand a compression load of up to 25.0 N.

47. An implantable sensing construct configured to be percutaneously implanted in an aneurysmal sac, the implantable sensing construct comprising:
a sensor; and
a body comprising a first configuration and a second configuration,
wherein in the first configuration, the body comprises a substantially linear shape for transport in a delivery system; and
wherein in the second configuration, the body comprises a coiled shape when released from the delivery system, wherein the coiled shape has a pitch and the pitch is substantially maintained upon a linear compression force of up to 8.0 N.

48. The implantable sensing construct of claim 47, wherein the linear compression force is up to 15.0 N.

49. The implantable sensing construct of claim 47, wherein the linear compression force is up to 25.0 N.

50. The implantable sensing construct of claim 47, wherein the linear compression force is up to 105.0 N.

51. An implantable system for use with a stent graft, the system comprising:
a helix antenna supported by a non-conductive substrate; and
a communications and processing circuitry electrically connected to the helix antenna via an antenna feed, the communications and processing circuitry supported by a substrate comprising a ground plane to which the helix antenna is electrically connected, the communications and processing circuitry further comprising at least one sensor.

52. The system of claim 51, further comprising a body configured to be attached to or positioned adjacent to the stent graft, the body made at least partially of conductive material, wherein the helix antenna is further electrically connected to the body such that the body provides an additional ground for the helix antenna.

53. The system of claim 51, wherein the helix antenna is configured to transmit and receive in a Bluetooth frequency band.

54. The system of claim 53, wherein a range of the helix antenna in the Bluetooth frequency band is about 1 foot or more.

55. The system of claim 53, wherein a range of the helix antenna in the Bluetooth frequency band is between about 1 foot and 2 feet.

56. The system of claim 51, wherein the communications and processing circuitry comprises a matching circuitry electrically connected to the helix antenna.

57. The system of claim 56, wherein the matching circuitry comprises a series capacitor and a shunt capacitor.

58. The system of claim 57, wherein the matching circuitry further comprises a low-pass filter.

59. The system of claim 51, wherein the non-conductive substrate provides structural support for the helix antenna, and wherein the helix antenna is wound on the non-conductive substrate.

60. The system of claim 51, wherein the non-conductive substrate comprises a polymer.

61. The system of claim 60, wherein the polymer comprises polytetrafluoroethylene (PTFE).

62. The system of claim 51, wherein the stent graft comprises an abdominal aortic aneurysm (AAA) stent graft.

63. A method of radio frequency (RF) testing an antenna of an implantable system, the method comprising:
determining one or more RF properties of the antenna of the implantable system positioned in a first container at least partially filled with a first composition configured to simulate electromagnetic properties of blood, the first container positioned in a second container at least partially filed with a second composition configured to simulate electromagnetic properties of one or more tissues.

64. The method of claim 63, wherein the one or more tissues comprise at least two of bone, muscle, fat, and skin.

65. The method of claim 64, wherein electromagnetic properties of at least two of bone, muscle, fat, and skin are averaged to create the second composition.

66. The method of claim 65, wherein the electromagnetic properties of the one or more tissues comprise relative permittivity and conductivity.

67. The method of claim 63, wherein the first composition comprises sodium chloride (NaCl), diacetin, and distilled water, and wherein the second composition comprises diacetin and distilled water.

68. The method of claim 63, wherein the antenna comprises a helix antenna.

69. The method of claim 68, wherein the helix antenna is supported by a non-conductive substrate.

70. The method of claim 63, wherein the implantable system is configured to be used with a stent graft, and wherein the stent graft comprises an abdominal aortic aneurysm (AAA) stent graft.

71. An implantable system for use with a stent graft, the system comprising:
a first antenna comprising a straight conductor and a helical conductor, the straight conductor electrically connected to the helical conductor; and
a communications and processing circuitry electrically connected to the first antenna via an antenna feed, the communications and processing circuitry supported by a substrate comprising a ground plane to which the first antenna is electrically connected, the communications and processing circuitry further comprising at least one sensor.

72. The system of claim 71, further comprising a body configured to be attached to or positioned adjacent to the stent graft, the body made at least partially of conductive material, wherein the first antenna is supported by the body and is further electrically connected to the body such that the body provides an additional ground for the first antenna.

73. The system of claim 71, wherein the straight conductor comprises a monopole antenna.

74. The system of claim 71, wherein the first antenna comprises a dual-band antenna that transmits and receives in first and second frequency bands.

75. The system of claim 74, wherein the first antenna resonates at center frequencies of the first and second frequency bands.

76. The system of claim 74, wherein the first frequency band comprises medical device radiocommunications service (MICS) band and the second frequency band comprises industrial, scientific, and medical (ISM) band.

77. The system of claim 76, wherein a range in the first frequency band is at least about 20 feet, and wherein a range in the second frequency band is at least about 15 feet.

78. The system of claim 76, wherein a range in the first frequency band comprises about 1 foot or more, and wherein a range in the second frequency band comprises about 1 foot or more.

79. The system of claim 76, wherein the communications and processing circuitry is configured to transition from a first power state to a second power state in which more power is consumed responsive to the first antenna receiving a command in the second frequency band.

80. The system of claim 79, wherein the first power state comprises a sleep state and the second power state comprises an operational state in which the communications and processing circuitry is configured to at least one of transmit or receive data.

81. The system of claim 80, wherein the data comprises data sensed by the at least one sensor, and wherein the communications and processing circuitry is configured to cause the first antenna to transmit the data in the first frequency band.

82. The system of claim 81, wherein the communications and processing circuitry is configured to transmit data sensed by the at least one sensor in the second power state and not in the first power state.

83. The system of claim 71, wherein the communications and processing circuitry comprises a matching circuitry electrically connected to the first antenna.

84. The system of claim 83, wherein:
the first antenna is configured to at least one of receive or transmit in first and second frequency bands, the second frequency band associated with higher frequencies than the first frequency band;
the matching circuitry comprises a first matching circuitry for signals in the first frequency band and a second matching circuitry for signals in the second frequency band; and
the first matching circuitry comprises a band-stop filter configured to remove one or more signal components in second frequency band.

85. The system of claim 84, wherein the first matching circuitry comprises a step-up impedance low pass filter and the second matching circuitry comprises a setup-up impedance high pass filter.

86. The system of claim 84, wherein the second matching circuitry does not comprise a band-stop filter configured to remove one or more signal components in the first frequency band.

87. The system of claim 71, further comprising a rechargeable power source and a second antenna configured to receive power for recharging the rechargeable power source.

88. The system of claim 87, wherein the second antenna comprises a coil configured to be inductively coupled with a coil of an external power transfer device.

89. The system of claim 87, further comprising a body configured to be attached to or positioned adjacent to the stent graft, wherein the first antenna is supported by the body at a first end of the body and the second antenna is supported by the body at a second end of the body opposite the first end.

90. The system of claim 71, further comprising a second antenna.

91. The system of claim 90, further comprising a body configured to be attached to or positioned adjacent to the stent graft, wherein the first antenna is supported by the body at a first end of the body and the second antenna is supported by the body at a second end of the body opposite the first end.

92. The system of claim 71, wherein length of the first antenna is at most about 40 mm and width of the first antenna is at most about 5 mm.

93. The system of claim 71, wherein spacing between turns of the helical conductor is about 1.7 mm.

94. The system of claim 71, wherein the helical conductor is wound around the straight conductor.

95. The system of claim 94, wherein the helical conductor is electrically insulated from the straight conductor in a region where the helical conductor is wound around the straight conductor.

96. The system of claim 71, wherein the straight conductor and the helical conductor are electrically connected to the antenna feed.

97. The system of claim 71, wherein the stent graft comprises an abdominal aortic aneurysm (AAA) stent graft.

98. An implantable system for use with a stent graft, the system comprising:
a first antenna comprising a loop; and
communications and processing circuitry electrically connected to the first antenna via an antenna feed, the communications and processing circuitry supported by a substrate comprising a ground plane to which the first antenna is electrically connected, the communications and processing circuitry further comprising at least one sensor; and
a matching circuitry of the communications and processing circuitry, the matching circuitry electrically connected to the first antenna and comprising a plurality of capacitors configured to match impedance of the first antenna in a first frequency band.

99. The system of claim 98, wherein the matching circuitry further comprises a plurality of inductors configured to match impedance of the first antenna in a second frequency band associated with higher frequencies than the first frequency band.

100. The system of claim 98, further comprising a body configured to be attached to or positioned adjacent to the stent graft, the body made at least partially of conductive material, wherein the first antenna is supported by the body and is further electrically connected to the body such that the body provides an additional ground for the first antenna.

101. The system of claim 98, wherein the first antenna comprises a dual-band antenna that transmits and receives in the first frequency band and in a second frequency band.

102. The system of claim 101, wherein the first antenna resonates in the second frequency band, the second frequency band associated with higher frequencies than the first frequency band.

103. The system of claim 101, wherein the first frequency band comprises medical device radiocommunications service (MICS) band and the second frequency band comprises industrial, scientific, and medical (ISU) band.

104. The system of claim 103, wherein a range in the first frequency band is at least about 20 feet, and wherein a range in the second frequency band is at least about 7 feet.

105. The system of claim 103, wherein a range in the first frequency band comprises about 1 foot or more, and wherein a range in the second frequency band comprises about 1 foot or more.

106. The system of claim 103, wherein the communications and processing circuitry is configured to transition from a first power state to a second power state in which more power is consumed responsive to the first antenna receiving a command in the second frequency band.

107. The system of claim 106, wherein the first power state comprises a sleep state and the second power state comprises an operational state in which the communications and processing circuitry is configured to at least one of transmit or receive data.

108. The system of claim 107, wherein the data comprises data sensed by the at least one sensor, and wherein the communications and processing circuitry is configured to cause the first antenna to transmit the data in the first frequency band.

109. The system of claim 106, wherein the communications and processing circuitry is configured to transmit data sensed by the at least one sensor in the second power state and not in the first power state.

110. The system of claim 109, wherein the matching circuitry comprises a band-stop filter configured to remove one or more signal components in the second frequency band.

111. The system of claim 109, wherein the matching circuitry does not comprise a band-stop filter configured to remove one or more signal components in the first frequency band.

112. The system of claim 98, further comprising a rechargeable power source and a second antenna configured to receive power for recharging the rechargeable power source.

113. The system of claim 112, wherein the second antenna comprises a coil configured to be inductively coupled with a coil of an external power transfer device.

114. The system of claim 112, further comprising a body configured to be attached to or positioned adjacent to the stent graft, wherein the first antenna is supported by the body at a first end of the body and the second antenna is supported by the body at a second end of the body opposite the first end.

115. The system of claim 98, further comprising a second antenna.

116. The system of claim 115, further comprising a body configured to be attached to or positioned adjacent to the stent graft, wherein the first antenna is supported by the body at a first end of the body and the second antenna is supported by the body at a second end of the body opposite the first end.

117. The system of claim 98, wherein diameter of the loop is at most about 40 mm.

118. The system of claim 98, wherein width of the first antenna is at most about 5 mm.

119. The system of claim 98, wherein the stent graft comprises an abdominal aortic aneurysm (AAA) stent graft.

120. A method of using or operating the system of any of the claims 50 to 119.

121. A delivery system for delivering an implantable device, the delivery system comprising:
a handle enclosure;
a handle driver comprising a collar rotatably coupled to the handle enclosure, an inner surface of the collar comprising a threaded pattern;
a first lead screw disposed at least partially within the handle driver, the first lead screw threaded in a first direction and configured to interface with the threaded pattern on the collar;
a second lead screw disposed at least partially within the handle enclosure, the second lead screw axially offset from the first lead screw, the second lead screw threaded in a second direction opposite from the first direction and configured to interface with the threaded pattern on the collar;
wherein rotation of the handle driver in a first direction advances the implantable device.

122. The delivery system of claim 121, wherein a distal portion of the first lead screw abuts a proximal portion of the second lead screw when the implantable device is loaded in the delivery system.

123. The delivery system of claim 121, wherein rotation of handle driver drives the first lead screw in a first direction and drives the second lead screw in a second direction opposite the first direction.

124. The delivery system of claim 123, wherein rotation of the handle driver drives the first lead screw and the second lead screw the same distance.

125. The delivery system of claim 121, wherein rotation of the handle driver in a second direction, opposite from the first direction, retracts the implantable device.

126. The delivery system of claim 121, wherein the handle enclosure comprise a groove configured to capture the collar of the handle driver.

127. The delivery system of claim 121, wherein the first lead screw is a partial body screw, and wherein the second lead screw is a partial body screw.

128. The delivery system of claim 127, wherein the first lead screw is circumferentially offset from the second lead screw.

129. The delivery system of claim 121, wherein the threaded pattern comprises a pattern of diamond-shaped recesses.

130. The delivery system of claim 121, further comprising an indicator fixed to the first lead screw, the indicator visible through a slot in the handle enclosure.

131. A method of delivering an implantable device to a patient, the method comprising:
advancing a delivery system to a target location, the delivery system comprising a handle and an outer sheath carrying the implantable device;
retracting a first actuator on the handle to deflect a distal portion of the outer sheath to a deflected configuration;
rotating the first actuator to lock the distal portion of the outer sheath in the deflected configuration;
rotating a second actuator in a first direction to advance an intermediate tube relative to the outer sheath, the intermediate tube coupled to the implantable device; and
withdrawing an inner tube to release the implantable device from the intermediate tube.

132. The method of claim 131, wherein retracting the first actuator tensions a wire to deflect the distal portion of the outer sheath.

133. The method of claim 131, wherein rotating the first actuator rotates a cam to lock the distal portion of the outer sheath in the deflected configuration.

134. The method of claim 131, further comprising rotating the second actuator in a second direction, opposite from the first direction, to retract the intermediate tube.

135. The method of claim 131, further comprising rotating the inner tube to release the inner tube from the implantable device.

136. The method of claim 131, wherein rotating the second actuator causes an indicator to travel along a slot in the handle of the delivery system.

137. The method of claim 131, further comprising removing a release pin to enable withdrawal of the inner tube.

138. The method of claim 137, wherein removing the release pin occurs after the implantable device has been only partially deployed from the outer sheath.

139. The method of claim 137, wherein removing the release pin occurs after a proximal end of the implantable device has been deployed from the outer sheath.

140. A delivery system for delivering an implantable device, the delivery system comprising:
a handle comprising:
a handle enclosure;
a first actuator movable relative to the handle enclosure;
a second actuator movable relative to the handle enclosure; and
an outer sheath extending from the handle;
an intermediate tube extending through the outer sheath, the intermediate tube configured to engage the implantable device;
an inner tube extending through the intermediate tube, the inner tube configured to maintain the intermediate tube in engagement with the implantable device when the inner tube extends through the implantable device;
wherein the first user actuator is configured to deflect a distal portion of the outer sheath from an undeflected configuration to a deflected configuration;
wherein the second actuator is configured to advance the intermediate tube relative to the outer sheath.

141. The delivery system of claim 140, wherein translation of the first user actuator tensions a wire to deflect the distal portion of the outer sheath from the undeflected configuration to the deflected configuration.

142. The delivery system of claim 141, wherein rotation of the first actuator rotates a cam to lock the distal portion of the outer sheath in the undeflected configuration or the deflected configuration.

143. The delivery system of claim 140, wherein rotation of the second actuator in a first direction advances the intermediate tube, and wherein rotation of the second actuator in a second direction, opposite the first direction, retracts the intermediate tube.

144. The delivery system of claim 140, further comprising a release pin at a proximal end of the inner tube.

145. The delivery system of claim 144, wherein rotation of the release pin deploys a distal portion of the implantable device.

146. The delivery system of claim 144, wherein the release pin is removable from the inner tube.

147. The delivery system of claim 140, further comprising an indicator visible through a slot in the handle enclosure, the indicator indicative of a location of the implantable device relative to the outer sheath.

148. The delivery system of claim 140, further comprising a disconnect assembly at a distal end of the intermediate tube, the disconnect assembly comprising one or more deflectable tabs configured to engage the implantable device when the inner tube extends through the inner component.

149. An implantable sensing construct configured to be percutaneously implanted in an aneurysmal sac, the implantable sensing construct comprising:
a sensor; and
a tubular body comprising a first configuration and a second configuration, the tubular body comprising a plurality of cutouts in a circumferential direction, each of the plurality of cutouts comprising a first end, a second end, and in intermediate portion therebetween;
wherein in the first configuration, the body comprises a substantially linear shape for transport in a delivery system; and
wherein in the second configuration, the body comprises a coiled shape when released from the delivery system.

150. The implantable sensing construct of claim 149, wherein a width of each of the first ends and the second ends of the plurality of cutouts is greater than a width of the intermediate portions.

151. The implantable sensing construct of claim 149, wherein the plurality of cutouts are equally spaced apart along a length of the tubular body.

152. The implantable sensing construct of claim 149, wherein the tubular body comprises a plurality of tubular segments, the plurality of tubular segments spaced apart from each other and interconnected by a spine, each of the plurality of tubular segments having one or more of the plurality of cutouts.

153. The implantable sensing construct of claim 152, wherein when the tubular body is laid flat as a flattened body with the spine forming opposite lateral edges, the lateral edges form an oblique angle relative to an end of the flattened body.

154. The implantable sensing construct of claim 152, wherein the plurality of tubular segments comprises: a first tubular segment at a first end of the tubular body, a second tubular segment at a second end of the tubular body, and at least one tubular segment between the first tubular segment and the second tubular segment.

155. The implantable sensing construct of claim 134, wherein the at least one tubular segment is shorter than the first tubular segment and the second tubular segment.
